(19) United States
(12) Patent Application Publication
(10) Pub. No.: US 2009/0252681 A1
(43) Pub. Date: Oct. 8, 2009

(54) Nanobodies and Polypeptides Against EGFR and IGF-IR

(75) Inventors: Toon Laeremans, Dworp-Beersel (BE); Hans De Haard, Oudelande (NL); Hendricus Renerus Jacobus Mattheus Hoogenboom, Maastricht (NL)

Correspondence Address: WOLF GREENFIELD & SACKS, P.C., 600 ATLANTIC AVENUE, BOSTON, MA, 02210-2206, US

(73) Assignee: Ablynx N.V., Ghent-Zwijnaarde (BE)

(21) Appl. No.: 12/083,406

(22) PCT Filed: Oct. 11, 2006
(86) PCT No.: PCT/EP2006/009840

§ 371 Date: Apr. 11, 2008

Related U.S. Application Data
(60) Provisional application No. 60/725,919, filed on Oct. 11, 2005.

(51) Int. Cl. A61K 39/395; C07K 16/28; C07K 2/00; C12P 21/00; C07H 21/04; C12N 1/21; C12N 5/10; C12N 1/15; A61K 49/00; A61P 35/00; A61P 29/00; A61P 17/00; G01N 33/53

(52) U.S. Cl. 424/9.1; 530/387.1; 530/300; 435/71.1; 536/23.53; 435/252.33; 435/325; 435/358; 435/254.2; 435/69.1; 424/130.1; 435/7.2

(57) ABSTRACT

The invention relates to polypeptides and Nanobodies against Epidermal Growth Factor Receptor (EGFR) and/or Insulin Growth Factor-I Receptor (IGF-IR). The invention also relates to nucleic acids encoding such Nanobodies and polypeptides; to methods for preparing such Nanobodies and polypeptides; to host cells expressing or capable of expressing such Nanobodies or polypeptides; to compositions, and in particular to pharmaceutical compositions, that comprise such Nanobodies, polypeptides, nucleic acids and/or host cells; and to uses of such Nanobodies, polypeptides, nucleic acids, host cells and/or compositions, in particular for prophylactic, therapeutic or diagnostic purposes.

The present invention relates to polypeptides and Nanobodies™ against Epidermal Growth Factor Receptor and/or the Insulin Growth Factor-I Receptor (“EGFR” and “IGF-IR”, respectively). [Note: Nanobody™, Nanobodies™ and Nanoclone™ are trademarks of Ablynx N.V.]

The invention also relates to nucleic acids encoding such Nanobodies and polypeptides; to methods for preparing such Nanobodies and polypeptides; to host cells expressing or capable of expressing such Nanobodies or polypeptides; to compositions, and in particular to pharmaceutical compositions, that comprise such Nanobodies, polypeptides, nucleic acids and/or host cells; and to uses of such Nanobodies, polypeptides, nucleic acids, host cells and/or compositions, in particular for prophylactic, therapeutic or diagnostic purposes, such as the prophylactic, therapeutic or diagnostic purposes mentioned herein.

Other aspects, embodiments, advantages and applications of the invention will become clear from the further description herein.

The international applications WO 05/044858 and WO 04/041867 by applicant describe Nanobodies against EGFR, polypeptides comprising the same, compositions comprising such Nanobodies and polypeptides, and methods for preparing and using of such Nanobodies, polypeptides and compositions.

Generally, it is an object of the invention to provide Nanobodies against EGFR and polypeptides comprising the same that are an alternative to, and that preferably have improved properties compared to, the Nanobodies and polypeptides described in WO 05/044858 and WO 04/041867.

In particular, it is an object of the invention to provide a range of improved Nanobodies and polypeptides against EGFR. For example, in one preferred, but non-limiting embodiment, the invention provides Nanobodies against EGFR that bind to ectodomain EGFR, compete with the EGF, TGFα but not with the Erbitux binding sites on EGFR. In another preferred, but non-limiting embodiment, the invention provides Nanobodies that bind to ectodomain EGFR and compete with the EGF, Erbitux and TGFα-binding sites on EGFR. In yet another preferred, but non-limiting embodiment, the invention provides Nanobodies that bind to ectodomain EGFR, compete with the TGFα but not the EGF or Erbitux-binding sites on EGFR. As further described herein, all these different types of Nanobodies and polypeptides may find different (therapeutic or diagnostic) utility, depending on the desired properties for use.

Generally, the Nanobodies and polypeptides described herein, and in particular the improved Nanobodies against EGFR described herein, can be used for all applications and uses described in WO 05/044858 and WO 04/041867 for the Nanobodies and polypeptides disclosed therein. Thus, for example, the Nanobodies described herein can be used instead of the Nanobodies against EGFR described in WO 05/044858 and WO 04/041867 in preparing the polypeptides and Nanobody constructs described in WO 05/044858 and WO 04/041867. Also, the Nanobodies and polypeptides described herein can be formulated and used as described in WO 05/044858 and WO 04/041867.

In particular, the Nanobodies, polypeptides and compositions described herein can be used in the prevention, diagnosis and/or treatment of diseases and disorders associated with EGFR and/or IGF-IR, and in particular with diseases and disorders associated with or characterised by an over-expression of EGFR and/or IGF-IR (i.e. in certain tissues or cells). Examples of such diseases and disorders will be clear to the skilled person, and include various forms of cancers and tumors, such as those mentioned herein, as well as certain inflammatory diseases of the skin.

The anti-IGF-IR Nanobodies described herein can be used per se in the treatment of cancer and of other diseases and disorders associated with (the overexpression of) IGF-IR, but can also be used to enhance any anti-EGFR therapy (e.g. therapy with anti-EGFR compounds, anti-EGFR antibodies (including antibody fragments) or with anti-EGFR Nanobodies or polypeptides), to reduce the amount of anti-EGFR compounds or antibodies used in such therapy (and thus for example to reduce the side-effects associated therewith) and/or to prevent or reduce resistance against anti-EGFR therapy. Examples of such anti-EGFR therapies will be clear to the skilled person, for example from the pertinent prior art cited herein. Reference is for example also made to Friess et al., Clin. Cancer Res, 11(14), 2005, 5300 and to Thaker et al., Clin. Cancer Res., 11(13), 2005, 4923. Several mouse monoclonal antibodies have already been succesfully introduced into pre-clinical and clinical application such as IMC-C225 (Erbitux or Cetuximab) from Imclone systems, EMD7200 (Matuzumab) from Merck, ABX-EGF (Panitumumab) from Abgenix and 2F8 from Genmab.

Also, generally, the in vitro and/or in vivo activity and/or efficacy of the Nanobodies and polypeptides described herein may be determined using one or more of the in vitro assays, in vivo assays, cell-based assays or animal models described in WO 05/044858 and WO 04/041867. Reference is also made to Roovers et al., “Efficient inhibition of EGFR signalling and of tumor growth by antagonisitic anti-EGFR Nanobodies” (manuscript submitted for publication). Other assays, models and techniques for assessing the efficacy of the Nanobodies, polypeptides and compositions described herein, in particular in treating tumors and/or in inhibiting the growth or proliferation of tumor cells, will be clear to the skilled person, for example from the prior art related to EGFR and IGF-IR referred to herein.

It is also an object of the present invention to provide Nanobodies against IGF-IR and polypeptides comprising the same.

For the role of the IGF-I receptor in cancer and strategies for targeting IGF-IR in the therapy of cancer, reference is inter alia made to Hofman and Garcia-Echeverria, Drug Discovery Today, Vol. 10, 15, August 2005, 1041; Papa et al., Cancer Res., 1993, August 15; 53(16):3736-40; Arteaga et al., J. Clin. Invest., Vol. 84., November 1989, 1418-1423; Maloney et al., Cancer Research 63, 5073-5083, 2003; Baehr and Groner, Growth Factors, March 2005, 23(1) 1-14; Ulfarsson et al., Clin. Cancer Res., 2005, 11(13), 2005; Burtrtm et al., Cancer Research, 63, 8912-8921 (2003); Miyamoto et al., Clin. Cancer Res., 2005, 11(9), 2005; Hailey et al., Molecular Cancer Therapeutics, Vol. 1, 1349-1353, December 2002.

It has also been reported that the IGF-I receptor plays a role in the development of resistance to Herceptin (Altundag et al., Mol. Cancer. Ther., 2005, (4)₇, 1136; Monnier et al., Bull. Cancer., 2004, September; 91(9):685-94; Chakravarti et al., Cancer Research 62, 200-207, 2002). The synergistic effect of EGF and IGF-I is for example described in Faisal et al., Journal of Surgical Research, 69, 354-358 (1997), in Adams et al., Growth Factors, June 2004, Vol. 22(2), 89-95, and by Knowlden, Endocrinology, 2005, July 21 (e-publication ahead of print). The IGF potentiation of EGFR signalling is described by Chong et al., Biochem. Biophys. Res. Commun., 2004, 322(2):535-41. The link between EGFR and IGF-IR expression in breast cancer is for example described by van den Berg et al., Br. J. Cancer, 1996, 73(4):477-81. Combination therapies for cancer using a monoclonal antibody against IGF-IR is described by Cohen et al., Clinical Cancer Research Vol. 11, 2063-2073 (2005). Lu et al., JBC, Vol. 279., No. 4, 2856-2865 (2004) describe the influence on the growth of cancer cells of simultaneous blockade of EGFR and IGF-IR using a bispecific monoclonal antibody. Manes et al., Endocrinology, Vol. 138, No. 3, 905 describe epitope mapping of IGF-IR.

In the invention, generally, these objects are achieved by the use of the Nanobodies and polypeptides provided herein.

Thus, it is one object of the present invention to provide improved Nanobodies against EGFR and/or polypeptides and Nanobodies against IGF-IR, in particular against EGFR or IGF-IR, respectively, from a warm-blooded animal, more in particular against EGFR or IGF-IR, respectively, from a mammal, and especially against human EGFR or IGF-IR, respectively, and to provide proteins and polypeptides comprising or essentially consisting of at least one such Nanobody.

In particular, it is an object of the present invention to provide such Nanobodies and such proteins and/or polypeptides that are suitable for prophylactic, therapeutic and/or diagnostic use in a warm-blooded animal, and in particular in a mammal, and more in particular in a human being.

More in particular, it is an object of the present invention to provide such Nanobodies and such proteins and/or polypeptides that can be used for the prevention, treatment, alleviation and/or diagnosis of one or more diseases, disorders or conditions associated with EGFR or IGF-IR, respectively, and/or mediated by EGFR or IGF-IR, respectively, (such as the diseases, disorders and conditions mentioned herein) in a warm-blooded animal, in particular in a mammal, and more in particular in a human being.

It is also an object of the invention to provide such Nanobodies and such proteins and/or polypeptides that can be used in the preparation of a pharmaceutical or veterinary composition for the prevention and/or treatment of one or more diseases, disorders or conditions associated with and/or mediated by EGFR or IGF-IR, respectively, (such as the diseases, disorders and conditions mentioned herein) in a warm-blooded animal, in particular in a mammal, and more in particular in a human being.

One specific but non-limiting object of the invention is to provide Nanobodies, proteins and/or polypeptides against EGFR or IGF-IR, respectively, that have improved therapeutic and/or pharmacological properties and/or other advantageous properties (such as, for example, improved ease of preparation and/or reduced costs of goods), compared to conventional antibodies against EGFR or IGF-IR, respectively, or fragments thereof, such as Fab′ fragments, F(ab′)₂ fragments, ScFv constructs, “diabodies” and/or other classes of (single) domain antibodies, such as the “dAb's described by Ward et al (infra), and also compared to the anti EGFR Nanobodies and polypeptides described in WO 05/044858 and WO 04/041867. These improved and advantageous properties will become clear from the further description herein.

These objects are achieved by the Nanobodies, proteins and polypeptides described herein. These Nanobodies are also referred to herein as “Nanobodies of the invention”; and these proteins and polypeptides are also collectively referred to herein “polypeptides of the invention”.

Thus, in a first aspect, the invention relates to improved Nanobodies against EGFR, and in particular to a Nanobody against EGFR from a warm-blooded animal, and more in particular to improved Nanobodies against EGFR from a mammal, and especially to improved Nanobodies against human EGFR, wherein said improved Nanobodies are as defined below.

In another aspect, the invention relates to a binding polypeptide of less than 15 kDa directed against IGF-IR. In one embodiment, such binding polyepeptdie is able to inhibit IGF-I interaction with IGF-IR. In a preferred embodiment, such polypeptides is a single domain antibody, a domain antibody, a “dAb”, a VH, a VHH or a Nanobody.

Accordingly, the invention preferably relates to a Nanobody against IGF-IR, and in particular to a Nanobody against IGF-IR from a warm-blooded animal, and more in particular to a Nanobody against IGF-IR from a mammal, and especially to a Nanobody against human IGF-IR.

In another aspect, the invention relates to a protein or polypeptide that comprises or essentially consists of at least one such Nanobody against EGFR or IGF-IR, respectively.

According to another embodiment, the invention provides Nanobodies and polypeptides against EGFR and Nanobodies against EGFR for use in a combination therapy, in particular for the treatment of cancer. In such a combination therapy, the Nanobodies and polypeptides against IGF-IR described herein can be used together with the anti-EGFR Nanobodies and polypeptides described in WO 05/044858 and WO 04/041867, and/or together with the anti-EGFR Nanobodies and polypeptides described herein.

According to one specific, but non-limiting embodiment, a polypeptide as described herein comprises at least one Nanobody against EGFR and at least one Nanobody against IGF-IR. In such a bispecific Nanobody construct, the Nanobodies and polypeptides against IGF-IR described herein can be combined with one or more of the anti-EGFR Nanobodies and polypeptides described in WO 05/044858 and WO 04/041867, and/or with one or more of the anti-EGFR Nanobodies and polypeptides described herein.

It will be clear to the skilled person that for pharmaceutical use, the Nanobodies and polypeptides of the invention are preferably directed against human EGFR or IGF-IR, respectively, whereas for veterinary purposes, the Nanobodies and polypeptides of the invention are preferably directed against EGFR or IGF-IR, respectively, from the species to be treated.

Also, according to the invention, Nanobodies and polypeptides that are directed against EGFR or IGF-IR, respectively, from a first species of warm-blooded animal may or may not show cross-reactivity with EGFR or IGF-IR, respectively, from one or more other species of warm-blooded animals. For example, Nanobodies and polypeptides directed against human EGFR or IGF-IR, respectively, may or may not show cross reactivity with EGFR or IGF-IR, respectively, from one or more other species of primates and/or with EGFR or IGF-IR, respectively, from one or more species of animals that are often used in animal models for diseases (for example mouse, rat, rabbit, pig or dog), and in particular in animal models for diseases and disorders associated with EGFR or IGF-IR, respectively, (such as the species and animal models mentioned herein). In this respect, it will be clear to the skilled person that such cross-reactivity, when present, may have advantages from a drug development point of view, since it allows the Nanobodies and polypeptides against human EGFR or IGF-IR, respectively, to be tested in such disease models.

More generally, it is also encompassed within the scope of the invention that Nanobodies and polypeptides directed against EGFR or IGF-IR, respectively, from one species of animal (such as Nanobodies and polypeptides against human EGFR or IGF-IR, respectively) are used in the treatment of another species of animal, as long as the use of the Nanobodies and/or polypeptides provide the desired effects in the species to be treated.

The present invention is in its broadest sense also not particularly limited to or defined by a specific antigenic determinant, epitope, part, domain, subunit or confirmation (where applicable) of EGFR or IGF-IR, respectively, against which the Nanobodies and polypeptides of the invention are directed. However, in particular, the Nanobodies and polypeptides of the invention may be directed against epitopes that are exposed on the cell surface.

It is also within the scope of the invention that, where applicable, a Nanobody of the invention can bind to two or more antigenic determinants, epitopes, parts, domains, subunits or confirmations of EGFR or IGF-IR, respectively. In such a case, the antigenic determinants, epitopes, parts, domains or subunits of EGFR or IGF-IR, respectively, to which the Nanobodies and/or polypeptides of the invention bind may be the essentially same (for example, if EGFR or IGF-IR, respectively, contains repeated structural motifs or is present as a multimer) or may be different (and in the latter case, the Nanobodies and polypeptides of the invention may bind to such different antigenic determinants, epitopes, parts, domains, subunits of EGFR or IGF-IR, respectively, with an affinity and/or specificity which may be the same or different). Also, for example, when EGFR or IGF-IR, respectively, exists in an activated conformation and in an inactive conformation, the Nanobodies and polypeptides of the invention may bind to either one of these confirmation, or may bind to both these confirmations (i.e. with an affinity and/or specificity which may be the same or different). Also, for example, the Nanobodies and polypeptides of the invention may bind to a conformation of EGFR or IGF-IR, respectively, in which it is bound to a pertinent ligand, may bind to a conformation of EGFR or IGF-IR, respectively, in which it not bound to a pertinent ligand, or may bind to both such conformations (again with an affinity and/or specificity which may be the same or different).

It is also expected that the Nanobodies and polypeptides of the invention will generally bind to all naturally occurring or synthetic analogs, variants, mutants, alleles, parts and fragments of EGFR or IGF-IR, respectively, or at least to those analogs, variants, mutants, alleles, parts and fragments of EGFR or IGF-IR, respectively, that contain one or more antigenic determinants or epitopes that are essentially the same as the antigenic determinant(s) or epitope(s) to which the Nanobodies and polypeptides of the invention bind in EGFR or IGF-IR, respectively, (e.g. in wild-type EGFR or IGF-IR, respectively). Again, in such a case, the Nanobodies and polypeptides of the invention may bind to such analogs, variants, mutants, alleles, parts and fragments with an affinity and/or specificity that are the same as, or different from (i.e. higher than or lower than), the affinity and specificity with which the Nanobodies of the invention bind to (wild-type) EGFR or IGF-IR, respectively. It is also included within the scope of the invention that the Nanobodies and polypeptides of the invention bind to some analogs, variants, mutants, alleles, parts and fragments of EGFR or IGF-IR, respectively, but not to others.

When EGFR or IGF-IR, respectively, exists in a monomeric form and in one or more multimeric forms, it is within the scope of the invention that the Nanobodies and polypeptides of the invention only bind to EGFR or IGF-IR, respectively, in monomeric form, or that the Nanobodies and polypeptides of the invention in addition also bind to one or more of such multimeric forms. Also, when EGFR or IGF-IR, respectively, can associate with other proteins or polypeptides to form protein complexes, it is within the scope of the invention that the Nanobodies and polypeptides of the invention bind to EGFR or IGF-IR, respectively, in its non-associated state, bind to EGFR or IGF-IR, respectively, in its associated state, or bind to both. In all these cases, the Nanobodies and polypeptides of the invention may bind to such multimers or associated protein complexes with an affinity and/or specificity that may be the same as or different from (i.e. higher than or lower than) the affinity and/or specificity with which the Nanobodies and polypeptides of the invention bind to EGFR or IGF-IR, respectively, in its monomeric and non-associated state.

Generally, the Nanobodies and polypeptides of the invention will at least bind to those forms (including monomeric, multimeric and associated forms) that are the most relevant from a biological and/or therapeutic point of view, as will be clear to the skilled person.

It is also within the scope of the invention to use parts, fragments, analogs, mutants, variants, alleles and/or derivatives of the Nanobodies and polypeptides of the invention, and/or to use proteins or polypeptides comprising or essentially consisting of the same, as long as these are suitable for the uses envisaged herein. Such parts, fragments, analogs, mutants, variants, alleles, derivatives, proteins and/or polypeptides will be described in the further description herein.

As discussed in more detail herein, the Nanobodies of the invention generally comprise a single amino acid chain, that can be considered to comprise “framework sequences” or “FR” (which are generally as described herein) and “complementarity determining regions” or CDR's. Some preferred CDR's present in the Nanobodies of the invention are as described herein.

More generally, for the Nanobodies against IGF-IR and with reference to the further definitions given herein, the CDR sequences present in the Nanobodies of the invention are obtainable/can be obtained by a method comprising the steps of:
a) providing at least one V_{HH} domain directed against IGF-IR, by a method generally comprising the steps of (i) immunizing a mammal belonging to the Camelidae with IGF-IR or a part or fragment thereof, so as to raise an immune response and/or antibodies (and in particular heavy chain antibodies) against IGF-IR; (ii) obtaining a biological sample from the mammal thus immunized, wherein said sample comprises heavy chain antibody sequences and/or V_{HH} sequences that are directed against IGF-IR; and (iii) obtaining (e.g isolating) heavy chain antibody sequences and/or V_{HH} sequences that are directed against IGF-IR from said biological sample; and/or by a method generally comprising the steps of (i) screening a library comprising heavy chain antibody sequences and/or V_{HH} sequences for heavy chain antibody sequences and/or V_{HH} sequences that are directed against IGF-IR or against at least one part or fragment thereof; and (ii) obtaining (e.g. isolating) heavy chain antibody sequences and/or V_{HH} sequences that are directed against IGF-IR from said library;
b) optionally subjecting the heavy chain antibody sequences and/or V_{HH} sequences against IGF-IR thus obtained to affinity maturation, to mutagenesis (e.g. random mutagenesis or site-directed mutagenesis) and/or any other technique(s) for increasing the affinity and/or specificity of the heavy chain antibody sequences and/or V_{HH} sequences for IGF-IR;
c) determining the sequences of the CDR's of the heavy chain antibody sequences and/or V_{HH} sequences against IGF-IR thus obtained; and optionally
d) providing a Nanobody in which at least one, preferably at least two, and more preferably all three of the CDR's (i.e. CDR1, CDR2 and CDR3, and in particular at least CDR3) has a sequence that has been determined in step c).

Usually, in step d), all CDR sequences present in a Nanobody of the invention will be derived from the same heavy chain antibody or V_{HH} sequence. However, the invention in its broadest sense is not limited thereto. It is for example also possible (although often less preferred) to suitably combine, in a Nanobody of the invention, CDR's from two or three different heavy chain antibodies or V_{HH} sequences against IGF-IR and/or to suitably combine, in a Nanobody of the invention, one or more CDR's derived from heavy chain antibodies or V_{HH} sequences (an in particular at least CDR3) with one or more CDR's derived from a different source (for example synthetic CDR's or CDR's derived from a human antibody or VH domain).

According to a non-limiting but preferred embodiment of the invention, the CDR sequences in the Nanobodies of the invention are such that the Nanobody of the invention binds to EGFR or IGF-IR, respectively, with an dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹ and/or with an affinity less than 500 nM, preferably less than 200 nM, more preferably less than 10 nM, such as less than 500 pM. The affinity of the Nanobody of the invention against EGFR or IGF-IR, respectively, can be determined in a manner known per se, for example using the assay described herein.

In a preferred but non-limiting aspect, the invention relates to a Nanobody (as defined herein) against EGFR, which consist of 4 framework regions (FR1 to FR4 respectively) and 3 complementarity determining regions (CDR1 to CDR3 respectively), in which:
(a) CDR1 is an amino acid sequence chosen from the group consisting of:

| | |
|---|---|
| TYTMA | [SEQ ID NO:42] |
| SYGMG | [SEQ ID NO:43] |
| GFAMG | [SEQ ID NO:44] |
| SNNMG | [SEQ ID NO:45] |
| GDVMG | [SEQ ID NO:46] |
| SYVVG | [SEQ ID NO:47] |
| DYNMA | [SEQ ID NO:48] |
| TYTMA | [SEQ ID NO:49] |
| NNAMA | [SEQ ID NO:50] |
| SYVMG | [SEQ ID NO:51] |
| SYAMG | [SEQ ID NO:52] |
| A | [SEQ ID NO:53] |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s); and/or from the group consisting of amino acid sequences that have 2 or only 1 “amino acid difference(s)” (as defined herein) with one of the above amino acid sequences, in which:
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
   and/or in which:
(b) CDR2 is an amino acid sequence chosen from the group consisting of:

| | |
|---|---|
| GISRSDGGTYDADSVKG | [SEQ ID NO: 54] |
| GISWRGDSTGYADSVKG | [SEQ ID NO: 55] |
| AISWSGGSLLYVDSVKG | [SEQ ID NO: 56] |
| AIGWGGLETHYSDSVKG | [SEQ ID NO: 57] |
| GFSRSTSTTHYADSVKG | [SEQ ID NO: 58] |
| GIAWGDGITYYADSVKG | [SEQ ID NO: 59] |
| HISWLGGRTYYRDSVKG | [SEQ ID NO: 60] |
| GFSGSGGATYYAHSVEG | [SEQ ID NO: 61] |
| AISWRGGSTYYADSVKG | [SEQ ID NO: 62] |
| AINWSSGSTYYADSVKG | [SEQ ID NO: 63] |
| TIAWDSGSTYYADSVKG | [SEQ ID NO: 64] |
| GLSWSADSTYYADSVKG | [SEQ ID NO: 65] |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
   and/or in which:
(c) CDR3 is an amino acid sequence chosen from the group consisting of:

| | |
|---|---|
| ASVKLVYVNPNRYSY | [SEQ ID NO: 66] |
| AAGSAWYGTLYEYDY | [SEQ ID NO: 67] |
| AAGSTWYGTLYEYDY | [SEQ ID NO: 68] |
| MVGPPPRSLDYGLGNHYEYDY | [SEQ ID NO: 69] |
| SSTRTVIYTLPRMYNY | [SEQ ID NO: 70] |
| NSRSSWVIFTIKGQYDR | [SEQ ID NO: 71] |
| RPGMIITTIQATYGF | [SEQ ID NO: 72] |
| GSPYGTELPYTRIEQYAY | [SEQ ID NO: 73] |
| ANEYWVYVNPNRYTY | [SEQ ID NO: 74] |
| RRKSGEVVFTIPARYDY | [SEQ ID NO: 75] |
| VYRVGAISEYSGTDYYTDEYDY | [SEQ ID NO: 76] |
| GYQINSGNYNFKDYEYDY | [SEQ ID NO: 77] |
| SYNVYYNNYYYPISRDEYDY | [SEQ ID NO: 78] |
| HRRPFASVFTTTRMYDY | [SEQ ID NO: 79] |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s).

In another preferred but non-limiting aspect, the invention relates to a Nanobody (as defined herein) against IGF-IR, which consist of 4 framework regions (FR1 to FR4 respectively) and 3 complementarity determining regions (CDR1 to CDR3 respectively), in which:
(a) CDR1 is an amino acid sequence chosen from the group consisting of:

| | |
|---|---|
| FNAMG | [SEQ ID NO: 94] |
| INYMA | [SEQ ID NO: 95] |
| NYAMG | [SEQ ID NO: 96] |
| RTAMA | [SEQ ID NO: 97] |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or from the group consisting of amino acid sequences that have 2 or only 1 “amino acid difference(s)” (as defined herein) with one of the above amino acid sequences, in which:
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
   and/or in which:
(b) CDR2 is an amino acid sequence chosen from the group consisting of:

| | |
|---|---|
| VIISGGSTHYVDSVKG | [SEQ ID NO: 98] |
| EITRSGRTNYVDSVKG | [SEQ ID NO: 99] |
| AINWNSRSTYYADSVKG | [SEQ ID NO: 100] |
| TITWNSGTTRYADSVKG | [SEQ ID NO: 101] |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
   and/or in which:
(c) CDR3 is an amino acid sequence chosen from the group consisting of:

| | |
|---|---|
| KKFGDY | [SEQ ID NO: 102] |
| IDGSWREY | [SEQ ID NO: 103] |
| SHDSDYGGTNANLYDY | [SEQ ID NO: 104] |
| TAAAVITPTRGYYNY | [SEQ ID NO: 105] |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution is preferably a conservative amino acid substitution (as defined herein); and/or
ii) said amino acid sequence preferably only contains amino acid substitutions, and no amino acid deletions or insertions, compared to the above amino acid sequence(s).

Thus, some particularly preferred, but non-limiting CDR sequences and combinations of CDR sequences that are present in the Nanobodies of the invention are as listed in Table A-1 for EGFR or IGF-IR, respectively, below.

TABLE A-1

Nanobodies against EGFR and IGF-IR
Nanobodies against EGFR

| | | |
|---|---|---|
| CDR1 | CDR2 | CDR3 |

| | | | | | | |
|---|---|---|---|---|---|---|
| Clone | | SEQ ID | | SEQ ID | | SEQ |
| designation | Sequence | NO | Sequence | NO | Sequence | ID NO |

| | | | | | | |
|---|---|---|---|---|---|---|
| EGR 27-10-E8 | TYTMA | 42 | GISRSDGGTYDADSVKG | 54 | ASVKLVYVNPNRYSY | 66 |
| EGR PMP7D12 | SYGMG | 43 | GISWRGDSTGYADSVKG | 55 | AAGSAWYGTLYEYDY | 67 |
| EGR PMP7C12 | SYGMG | 43 | GISWRGDSTGYADSVKG | 56 | AAGSTWYGTLYEYDY | 68 |
| EGR PMP9C1 | GFAMG | 44 | AISWSGGSLLYVDSVKG | 56 | MVGPPPRSLDYGLGNHYEYDY | 69 |
| EGR PMP7A5 | SNNMG | 45 | AIGWGGLETHYSDSVKG | 57 | SSTRTVIYTLPRMYNY | 70 |
| EGR PMP9A7 | GDVMG | 46 | GFSRSTSTTHYADSVKG | 58 | NSRSSWVIFTIKGQYDR | 71 |
| EGR PMP8B5 | SYVVG | 47 | GIAWGDGITYYADSVKG | 59 | RPGMIITTIQATYGF | 72 |
| EGR PMP11C9 | DYNMA | 48 | HISWLGGRTYYRDSVKG | 60 | GSPYGTELPYTRIEQYAY | 73 |
| EGR PMP11H6 | TYTMA | 49 | GISRSDGGTYDADSVKG | 54 | ANEYWVYVNPNRYTY | 74 |
| EGR PMP7E12 | NNAMA | 50 | GFSGSGGATYYAHSVEG | 61 | RRKSGEVVFTIIPARYDY | 75 |
| EGR PMP8C7 | SYVMG | 51 | AISWRGGSTYYADSVKG | 62 | VYRVGAISEYSGTDYYTDEYDY | 76 |
| EGR PMP9G8 | SYAMG | 52 | AINWSSGSTYYADSVKG | 63 | GYQINSGNYNFKDYEYDY | 77 |
| EGR PMP38G7 | SYVMG | 51 | TIAWDSGSTYYADSVKG | 64 | SYNVYYNNYYYPISRDEYDY | 78 |
| EGR 27-1-H7 | A | 53 | GLSWSADSTYYADSVKG | 65 | HRRPFASVFTTTRMYDY | 79 |
| IGR PMP4B11 | FNAMG | 94 | VIISGGSTHYVDSVKG | 98 | KKFGDY | 102 |
| IGR PMP3G71 | NVMA | 95 | EITRSGRTNYVDSVKG | 99 | IDGSWREY | 103 |
| IGR PMP2C7 | NYAMG | 96 | AINWNSRSTYYADSVKG | 100 | SHDSDYGGTNANLYDY | 104 |
| IGR PMP1C7 | RTAMA | 97 | TITWNSGTTRYADSVKG | 101 | TAAAVITPTRGYYNY | 105 |

Thus, in the Nanobodies of the invention, at least one of the CDR1, CDR2 and CDR3 sequences present is chosen from the group consisting of the CDR1, CDR2 and CDR3 sequences, respectively, listed in Table A-1 for EGFR or IGF-IR, respectively, or from the group of CDR1, CDR2 and CDR3 sequences, respectively, that have at least 80%, preferably at least 90%, more preferably at least 95%, even more preferably at least 99% “sequence identity” (as defined herein) with at least one of the CDR1, CDR2 and CDR3 sequences, respectively, listed in Table A-1 for EGFR or IGF-IR, respectively, and/or from the group consisting of the CDR1, CDR2 and CDR3 sequences, respectively, that have 3, 2 or only 1 “amino acid difference(s)” (as defined herein) with at least one of the CDR1, CDR2 and CDR3 sequences, respectively, listed in Table A-1 for EGFR or IGF-IR, respectively.

In particular, in the Nanobodies of the invention, at least the CDR3 sequence present is chosen from the group consisting of the CDR3 sequences listed in Table A-1 for EGFR or IGF-IR, respectively, or from the group of CDR3 sequences that have at least 80%, preferably at least 90%, more preferably at least 95%, even more preferably at least 99% sequence identity with at least one of the CDR3 sequences listed in Table A-1 for EGFR or IGF-IR, respectively, and/or from the group consisting of the CDR3 sequences that have 3, 2 or only 1 amino acid difference(s) with at least one of the CDR3 sequences listed in Table A-1 for EGFR or IGF-IR, respectively.

Preferably, in the Nanobodies of the invention, at least two of the CDR1, CDR2 and CDR3 sequences present are chosen from the group consisting of the CDR1, CDR2 and CDR3 sequences, respectively, listed in Table A-1 for EGFR or IGF-IR, respectively or from the group consisting of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table A-1 for EGFR or IGF-IR, respectively, and/or from the group consisting of the CDR1, CDR2 and CDR3 sequences, respectively, that have 3, 2 or only 1 “amino acid difference(s)” with at least one of the CDR1, CDR2 and CDR3 sequences, respectively, listed in Table A-1 for EGFR or IGF-IR, respectively.

In particular, in the Nanobodies of the invention, at least the CDR3 sequence present is chosen from the group consisting of the CDR3 sequences listed in Table A-1 for EGFR or IGF-IR, respectively, or from the group of CDR3 sequences that have at least 80%, preferably at least 90%, more preferably at least 95%, even more preferably at least 99% sequence identity with at least one of the CDR3 sequences listed in Table A-1 for EGFR or IGF-IR, respectively; and at least one of the CDR1 and CDR2 sequences present is chosen from the group consisting of the CDR1 and CDR2 sequences, respectively, listed in Table A-1 for EGFR or IGF-IR, respectively, or from the group of CDR1 and CDR2 sequences, respectively, that have at least 80%, preferably at least 90%, more preferably at least 95%, even more preferably at least 99% sequence identity with at least one of the CDR1 and CDR2 sequences, respectively, listed in Table A-1 for EGFR or IGF-IR, respectively, and/or from the group consisting of the CDR1 and CDR2 sequences, respectively, that have 3, 2 or only 1 amino acid difference(s) with at least one of the CDR1 and CDR2 sequences, respectively, listed in Table A-1 for EGFR or IGF-IR, respectively.

Most preferably, in the Nanobodies of the invention, all three CDR1, CDR2 and CDR3 sequences present are chosen from the group consisting of the CDR1, CDR2 and CDR3 sequences, respectively, listed in Table A-1 for EGFR or IGF-IR, respectively, or from the group of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table A-1 for EGFR or IGF-IR, respectively, and/or from the group consisting of the CDR1, CDR2 and CDR3 sequences, respectively, that have 3, 2 or only 1 amino acid difference(s) with at least one of the CDR1, CDR2 and CDR3 sequences, respectively, listed in Table A-1 for EGFR or IGF-IR, respectively.

Even more preferably, in the Nanobodies of the invention, at least one of the CDR1, CDR2 and CDR3 sequences present is chosen from the group consisting of the CDR1, CDR2 and CDR3 sequences, respectively, listed in Table A-1 for EGFR or IGF-IR, respectively. Preferably, in this embodiment, at least one or preferably both of the other two CDR sequences present are chosen from CDR sequences that that have at least 80%, preferably at least 90%, more preferably at least 95%, even more preferably at least 99% sequence identity with at least one of the corresponding CDR sequences, respectively, listed in Table A-1 for EGFR or IGF-IR, respectively, and/or from the group consisting of the CDR sequences that have 3, 2 or only 1 amino acid difference(s) with at least one of the corresponding sequences, respectively, listed in Table A-1 for EGFR or IGF-IR, respectively.

In particular, in the Nanobodies of the invention, at least the CDR3 sequence present is chosen from the group consisting of the CDR3 listed in Table A-1 for EGFR or IGF-IR, respectively. Preferably, in this embodiment, at least one and preferably both of the CDR1 and CDR2 sequences present are chosen from the groups of CDR1 and CDR2 sequences, respectively, that that have at least 80%, preferably at least 90%, more preferably at least 95%, even more preferably at least 99% sequence identity with the CDR1 and CDR2 sequences, respectively, listed in listed in Table A-1 for EGFR or IGF-IR, respectively, and/or from the group consisting of the CDR1 and CDR2 sequences, respectively, that have 3, 2 or only 1 amino acid difference(s) with at least one of the CDR1 and CDR2 sequences, respectively, listed in Table A-1 for EGFR or IGF-IR, respectively.

Even more preferably, in the Nanobodies of the invention, at least two of the CDR1, CDR2 and CDR3 sequences present are chosen from the group consisting of the CDR1, CDR2 and CDR3 sequences, respectively, listed in Table A-1 for EGFR or IGF-IR, respectively. Preferably, in this embodiment, the remaining CDR sequence present are chosen from the group of CDR sequences that that have at least 80%, preferably at least 90%, more preferably at least 95%, even more preferably at least 99% sequence identity with at least one of the corresponding CDR sequences listed in Table A-1 for EGFR or IGF-IR, respectively, and/or from the group consisting of CDR sequences that have 3, 2 or only 1 amino acid difference(s) with at least one of the corresponding sequences listed in Table A-1 for EGFR or IGF-IR, respectively.

In particular, in the Nanobodies of the invention, at least the CDR3 sequence is chosen from the group consisting of the CDR3 sequences listed in Table A-1 for EGFR or IGF-IR, respectively, and either the CDR1 sequence or the CDR2 sequence is chosen from the group consisting of the CDR1 and CDR2 sequences, respectively, listed in Table A-1 for EGFR or IGF-IR, respectively. Preferably, in this embodiment, the remaining CDR sequence present are chosen from the group of CDR sequences that that have at least 80%, preferably at least 90%, more preferably at least 95%, even more preferably at least 99% sequence identity with at least one of the corresponding CDR sequences listed in Table A-1 for EGFR or IGF-IR, respectively; and/or from the group consisting of CDR sequences that have 3, 2 or only 1 amino acid difference(s) with the corresponding CDR sequences listed in Table A-1 for EGFR or IGF-IR, respectively.

Even more preferably, in the Nanobodies of the invention, all three CDR1, CDR2 and CDR3 sequences present are chosen from the group consisting of the CDR1, CDR2 and CDR3 sequences, respectively, listed in Table A-1 for EGFR or IGF-IR, respectively.

Also, generally, the combinations of CDR's listed in Table A-1 for EGFR or IGF-IR, respectively, (i.e. those mentioned on the same line in Table A-1) are preferred. Thus, it is generally preferred that, when a CDR in a Nanobody of the invention is a CDR sequence mentioned in Table A-1 or is chosen from the group of CDR sequences that have at least 80%, preferably at least 90%, more preferably at least 95%, even more preferably at least 99% sequence identity with a CDR sequence listed in Table A-1 for EGFR or IGF-IR, respectively, and/or from the group consisting of CDR sequences that have 3, 2 or only 1 amino acid difference(s) with a CDR sequence listed in Table A-1 for EGFR or IGF-IR, respectively, that at least one and preferably both of the other CDR's are chosen from the CDR sequences that belong to the same combination in Table A-1 (i.e. mentioned on the same line in Table A-1) or are chosen from the group of CDR sequences that have at least 80%, preferably at least 90%, more preferably at least 95%, even more preferably at least 99% sequence identity with the CDR sequence(s) belonging to the same combination and/or from the group consisting of CDR sequences that have 3, 2 or only 1 amino acid difference(s) with the CDR sequence(s) belonging to the same combination. The other preferences indicated in the above paragraphs also apply to the combinations of CDR's mentioned in Table A-1.

Thus, by means of non-limiting examples, a Nanobody of the invention can for example comprise a CDR1 sequence that has more than 80% sequence identity with one of the CDR1 sequences mentioned in Table A-1, a CDR2 sequence that has 3, 2 or 1 amino acid difference with one of the CDR2 sequences mentioned in Table A-1 (but belonging to a different combination), and a CDR3 sequence.

Some preferred Nanobodies of the invention may for example comprise: (1) a CDR1 sequence that has more than 80% sequence identity with one of the CDR1 sequences mentioned in Table A-1; a CDR2 sequence that has 3, 2 or 1 amino acid difference with one of the CDR2 sequences mentioned in Table A-1 (but belonging to a different combination); and a CDR3 sequence that has more than 80% sequence identity with one of the CDR3 sequences mentioned in Table A-1 (but belonging to a different combination); or (2) a CDR1 sequence that has more than 80% sequence identity with one of the CDR1 sequences mentioned in Table A-1; a CDR2 sequence, and one of the CDR3 sequences listed in Table A-1 for EGFR or IGF-IR, respectively, or (3) a CDR1 sequence; a CDR2 sequence that has more than 80% sequence identity with one of the CDR2 sequence listed in Table A-1 for EGFR or IGF-IR, respectively, and a CDR3 sequence that has 3, 2 or 1 amino acid differences with the CDR3 sequence mentioned in Table A-1 that belongs to the same combination as the CDR2 sequence.

Some particularly preferred Nanobodies of the invention may for example comprise: (1) a CDR1 sequence that has more than 80% sequence identity with one of the CDR1 sequences mentioned in Table A-1; a CDR2 sequence that has 3, 2 or 1 amino acid difference with the CDR2 sequence mentioned in Table A-1 that belongs to the same combination; and a CDR3 sequence that has more than 80% sequence identity with the CDR3 sequence mentioned in Table A-1 that belongs to the same combination; (2) a CDR1 sequence; a CDR 2 listed in Table A-1 for EGFR or IGF-IR, respectively, and a CDR3 sequence listed in Table A-1 for EGFR or IGF-IR, respectively, (in which the CDR2 sequence and CDR3 sequence may belong to different combinations).

Some even more preferred Nanobodies of the invention may for example comprise: (1) a CDR1 sequence that has more than 80% sequence identity with one of the CDR1 sequences mentioned in Table A-1; the CDR2 sequence listed in Table A-1 for EGFR or IGF-IR, respectively, that belongs to the same combination; and a CDR3 sequence mentioned in Table A-1 that belongs to a different combination; or (2) a CDR1 sequence mentioned in Table A-1; a CDR2 sequence that has 3, 2 or 1 amino acid differences with the CDR2 sequence mentioned in Table A-1 that belongs to the same combination; and a CDR3 sequence that has more than 80% sequence identity with the CDR3 sequence listed in Table A-1 for EGFR or IGF-IR, respectively, that belongs to same or different combination.

Particularly preferred Nanobodies of the invention may for example comprise a CDR1 sequence mentioned in Table A-1, a CDR2 sequence that has more than 80% sequence identity with the CDR2 sequence mentioned in Table A-1 that belongs to the same combination; and the CDR3 sequence mentioned in Table A-1 that belongs to the same combination.

In the most preferred Nanobodies of the invention, the CDR1, CDR2 and CDR3 sequences present are chosen from one of the combinations of CDR1, CDR2 and CDR3 sequences, respectively, listed in Table A-1 for EGFR or IGF-IR, respectively.

Preferably, when a CDR sequence is chosen from the group of CDR sequences that have at least 80%, preferably at least 90%, more preferably at least 95%, even more preferably at least 99% sequence identity (as defined herein) with one of the CDR sequences listed in Table A-1 for EGFR or IGF-IR, respectively, and/or when a CDR sequence is chosen from the group consisting of CDR sequences that have 3, 2 or only 1 amino acid difference(s) with one of the CDR sequences listed in Table A-1 for EGFR or IGF-IR, respectively,
i) any amino acid substitution is preferably a conservative amino acid substitution (as defined herein); and/or
ii) said amino acid sequence preferably only contains amino acid substitutions, and no amino acid deletions or insertions, compared to the CDR sequence listed in Table A-1 for EGFR or IGF-IR, respectively.

According to a non-limiting but preferred embodiment of the invention, the CDR sequences in the Nanobodies of the invention are as defined above and are also such that the Nanobody of the invention binds to EGFR or IGF-IR, respectively, with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹ and/or with an affinity less than 500 nM, preferably less than 200 nM, more preferably less than 10 nM, such as less than 500 pM. The affinity of the Nanobody of the invention against EGFR or IGF-IR, respectively, can be determined in a manner known per se, for example using the assay described herein.

According to another preferred, but non-limiting embodiment of the invention (a) CDR1 has a length of between 1 and 12 amino acid residues, and usually between 2 and 9 amino acid residues, such as 5, 6 or 7 amino acid residues; and/or (b) CDR2 has a length of between 13 and 24 amino acid residues, and usually between 15 and 21 amino acid residues, such as 16 or 17 amino acid residues; and/or (c) CDR3 has a length of between 2 and 35 amino acid residues, usually between 3 and 30 amino acid residues, such as between 6 and 23 amino acid residues.

Nanobodies with the above CDR sequences preferably have framework sequences that are as further defined herein.

In another aspect, the invention relates to a Nanobody with an amino acid sequence that is chosen from the group consisting of SEQ ID NO's: 80-93 or from the group consisting of amino acid sequences that have more than 80%, preferably more than 90%, more preferably more than 95%, such as 99% or more sequence identity (as defined herein) with one or more of the amino acid sequences of SEQ ID NO's: 80-93.

In another aspect, the invention relates to a Nanobody with an amino acid sequence that is chosen from the group consisting of SEQ ID NO's: 106-109 or from the group consisting of amino acid sequences that have more than 80%, preferably more than 90%, more preferably more than 95%, such as 99% or more sequence identity (as defined herein) with one or more of the amino acid sequences of SEQ ID NO's106-109.

According to a specific, but non-limiting embodiment, the latter amino acid sequences have been “humanized”, as further described herein.

The polypeptides of the invention comprise or essentially consist of at least one Nanobody of the invention. Some preferred, but non-limiting examples of polypeptides of the invention are given in SEQ ID NO's: 110-143.

Generally, proteins or polypeptides that comprise or essentially consist of a single Nanobody (such as a single Nanobody of the invention) will be referred to herein as “monovalent” proteins or polypeptides or as “monovalent constructs”. Proteins and polypeptides that comprise or essentially consist of two or more Nanobodies (such as at least two Nanobodies of the invention or at least one Nanobody of the Invention and at least one other Nanobody) will be referred to herein as “multivalent” proteins or polypeptides or as “multivalent constructs”, and these may provide certain advantages compared to the corresponding monovalent Nanobodies of the invention. Some non-limiting examples of such multivalent constructs will become clear from the further description herein.

According to one specific, but non-limiting embodiment, a polypeptide of the invention comprises or essentially consists of at least two Nanobodies of the invention, such as two or three Nanobodies of the invention. As further described herein, such multivalent constructs can provide certain advantages compared to a protein or polypeptide comprising or essentially consisting of a single Nanobody of the invention, such as a much improved affinity and/or specificity for EGFR or IGF-IR, respectively.

According to another specific, but non-limiting embodiment, a polypeptide of the invention comprises or essentially consists of at least one Nanobody of the invention and at least one other Nanobody (i.e. directed against another epitope, antigen, target, protein or polypeptide). Such proteins or polypeptides are also referred to herein as “multispecific” proteins or polypeptides or as “multispecific constructs”, and these may provide certain advantages compared to the corresponding monovalent Nanobodies of the invention. Again, some non-limiting examples of such multispecific constructs will become clear from the further description herein.

According to yet another specific, but non-limiting embodiment, a polypeptide of the invention comprises or essentially consists of at least one Nanobody of the invention, optionally one or more further Nanobodies, and at least one other amino acid sequence (such as a protein or polypeptide) that confers at least one desired property to the Nanobody of the invention and/or to the resulting fusion protein. Again, such fusion proteins may provide certain advantages compared to the corresponding monovalent Nanobodies of the invention. Some non-limiting examples of such amino acid sequences and of such fusion constructs will become clear from the further description herein.

It is also possible to combine two or more of the above embodiments, for example to provide a trivalent bispecific construct comprising two Nanobodies of the invention and one other Nanobody, and optionally one or more other amino acid sequences. Further non-limiting examples of such constructs, as well as some constructs that are particularly preferred within the context of the present invention, will become clear from the further description herein.

In the above constructs, the one or more Nanobodies and/or other amino acid sequences may be directly linked or linked via one or more linker sequences. Some suitable but non-limiting examples of such linkers will become clear from the further description herein.

In one preferred embodiment of the invention, a polypeptide of the invention comprises one or more (such as two or preferably one) Nanobodies of the invention linked (optionally via one or more suitable linker sequences) to one or more (such as two and preferably one) amino acid sequences that allow the resulting polypeptide of the invention to cross the blood brain barrier. In particular, said one or more amino acid sequences that allow the resulting polypeptides of the invention to cross the blood brain barrier may be one or more (such as two and preferably one) Nanobodies, such as the Nanobodies described in WO 02/057445, of which FC44 (SEQ ID NO: 35) and FC5 (SEQ ID NO:36) are some preferred non-limiting examples.

In another preferred embodiment of the invention, a polypeptide of the invention comprises one or more (such as two or preferably one) Nanobodies of the invention linked (optionally via one or more suitable linker sequences) to one or more (such as two and preferably one) amino acid sequences that confer an increased half-life in vivo to the resulting polypeptide of the invention. In particular, said amino acid sequences that confer an increased half-life in vivo to the resulting polypeptide of the invention may be one or more (such as two and preferably one) Nanobodies, and in particular Nanobodies directed against a human serum protein such as human serum albumin, of which PMP6A6 (“ALB-1”, SEQ ID NO: 32), ALB-8 (a humanized version of ALB-1, SEQ ID NO:33) and PMP6A8 (“ALB-2”, SEQ ID NO:34) are some preferred non-limiting examples. Other examples of suitable Nanobodies against mouse or human serum albumin are described in the applications by applicant referred to below.

In yet another preferred embodiment of the invention, a polypeptide of the invention comprises one or more (such as two or preferably one) Nanobodies of the invention, one or more (such as two and preferably one) amino acid sequences that allow the resulting polypeptide of the invention to cross the blood brain barrier, and one or more (such as two and preferably one) amino acid sequences that confer an increased half-life in vivo to the resulting polypeptide of the invention (optionally linked via one or more suitable linker sequences). Again, said one or more amino acid sequences that allow the resulting polypeptides of the invention to cross the blood brain barrier may be one or more (such as two and preferably one) Nanobodies (as mentioned herein), and said amino acid sequences that confer an increased half-life in vivo to the resulting polypeptide of the invention may be one or more (such as two and preferably one) Nanobodies (also as mentioned herein).

According to a non-limiting but preferred embodiment of the invention, the polypeptides of the invention are preferably such that they bind to EGFR or IGF-IR with an dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹ and/or with an affinity less than 500 nM, preferably less than 200 nM, more preferably less than 10 mM, such as less than 500 pM. The affinity of the polypeptide of the invention against EGFR or IGF-IR can be determined in a manner known per se, for example using the assay described herein.

Some preferred, but non-limiting examples of polypeptides of the invention are the polypeptides of SEQ ID NO's: 110-143, in which:
SEQ ID NO's: 134-135 are some examples of multivalent (and in particular bivalent) polypeptides of the invention against IGF-IR;
SEQ ID NO's: 110-133 and 141-143 are some examples of multivalent (and in particular bivalent) polypeptides of the invention against EGFR;
of these, SEQ ID NO's: 141-143 are some examples of multivalent (and in particular bivalent) biparatopic polypeptides of the invention against EGFR;
SEQ ID NO's: 136-140 are some examples of bispecific polypeptides of the invention, comprising one Nanobody against EGFR and one Nanobody against IGF-IR.

Other polypeptides of the invention may for example be chosen from the group consisting of amino acid sequences that have more than 80%, preferably more than 90%, more preferably more than 95%, such as 99% or more “sequence identity” (as defined herein) with one or more of the amino acid sequences of SEQ ID NO's: 110-143, in which the Nanobodies comprised within said amino acid sequences are preferably as defined herein.

In another aspect, the invention relates to a nucleic acid that encodes a Nanobody of the invention and/or a polypeptide of the invention. Such a nucleic acid will also be referred to herein as a “nucleic acid of the invention” and may for example be in the form of a genetic construct, as defined herein.

In another aspect, the invention relates to host or host cell that expresses or that is capable of expressing a Nanobody of the invention and/or a polypeptide of the invention; and/or that contains a nucleic acid of the invention. Some preferred but non-limiting examples of such hosts or host cells will become clear from the further description herein.

The invention further relates to a product or composition containing or comprising at least one Nanobody of the invention, at least one polypeptide of the invention and/or at least one nucleic acid of the invention, and optionally one or more further components of such compositions known per se, i.e. depending on the intended use of the composition. Such a product or composition may for example be a pharmaceutical composition (as described herein), a veterinary composition or a product or composition for diagnostic use (as also described herein). Some preferred but non-limiting examples of such products or compositions will become clear from the further description herein.

The invention further relates to methods for preparing or generating the Nanobodies, polypeptides, nucleic acids, host cells, products and compositions described herein. Some preferred but non-limiting examples of such methods will become clear from the further description herein.

The invention further relates to applications and uses of the Nanobodies, polypeptides, nucleic acids, host cells, products and compositions described herein, as well as to methods for the prevention and/or treatment for diseases and disorders associated with EGFR or IGF-IR, respectively. Some preferred but non-limiting applications and uses will become clear from the further description herein.

Other aspects, embodiments, advantages and applications of the invention will also become clear from the further description hereinbelow.

### DETAILED DESCRIPTION OF THE INVENTION

The above and other aspects, embodiments and advantages of the invention will become clear from the further description hereinbelow, in which:
a) Unless indicated or defined otherwise, all terms used have their usual meaning in the art, which will be clear to the skilled person. Reference is for example made to the standard handbooks, such as Sambrook et al, “Molecular Cloning: A Laboratory Manual” (2nd.Ed.), Vols. 1-3, Cold Spring Harbor Laboratory Press (1989); F. Ausubel et al, eds., “Current protocols in molecular biology”, Green Publishing and Wiley Interscience, New York (1987); Lewin, “Genes II”, John Wiley & Sons, New York, N.Y., (1985); Old et al., “Principles of Gene Manipulation: An Introduction to Genetic Engineering”, 2nd edition, University of California Press, Berkeley, Calif. (1981); Roitt et al., “Immunology” (6th. Ed.), Mosby/Elsevier, Edinburgh (2001); Roitt et al., Roitt's Essential Immunology, 10^{th} Ed. Blackwell Publishing, UK (2001); and Janeway et al., “Immunobiology” (6th Ed.), Garland Science Publishing/Churchill Livingstone, N.Y. (2005), as well as to the general background art cited herein;
b) Unless indicated otherwise, the term “immunoglobulin sequence”—whether it used herein to refer to a heavy chain antibody or to a conventional 4-chain antibody—is used as a general term to include both the full-size antibody, the individual chains thereof, as well as all parts, domains or fragments thereof (including but not limited to antigen-binding domains or fragments such as V_{HH} domains or V_{H}/V_{L} domains, respectively). In addition, the term “sequence” as used herein (for example in terms like “immunoglobulin sequence”, “antibody sequence”, “variable domain sequence”, “V_{HH} sequence” or “protein sequence”), should generally be understood to include both the relevant amino acid sequence as well as nucleic acid sequences or nucleotide sequences encoding the same, unless the context requires a more limited interpretation;
c) Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein;
d) Amino acid residues will be indicated according to the standard three-letter or one-letter amino acid code, as mentioned in Table A-2;

TABLE A-2
one-letter and three-letter amino acid code

| | | | |
|---|---|---|---|
| Nonpolar, | Alanine | Ala | A |
| uncharged | Valine | Val | V |
| (at pH 6.0-7.0)⁽³⁾ | Leucine | Leu | L |
| | Isoleucine | Ile | I |
| | Phenylalanine | Phe | F |
| | Methionine⁽¹⁾ | Met | M |
| | Tryptophan | Trp | W |
| | Proline | Pro | P |
| Polar, | Glycine⁽²⁾ | Gly | G |
| uncharged | Serine | Ser | S |
| (at pH 6.0-7.0) | Threonine | Thr | T |
| | Cysteine | Cys | C |
| | Asparagine | Asn | N |
| | Glutamine | Gln | Q |
| | Tyrosine | Tyr | Y |
| Polar, | Lysine | Lys | K |
| charged | Arginine | Arg | R |
| (at pH 6.0-7.0) | Histidine⁽⁴⁾ | His | H |
| | Aspartate | Asp | D |
| | Glutamate | Glu | E |

Notes:
⁽¹⁾Sometimes also considered to be a polar uncharged amino acid.
⁽²⁾Sometimes also considered to be a nonpolar uncharged amino acid.
⁽³⁾As will be clear to the skilled person, the fact that an amino acid residue is referred to in this Table as being either charged or uncharged at pH 6.0 to 7.0 does not reflect in any way on the charge said amino acid residue may have at a pH lower than 6.0 and/or at a pH higher than 7.0; the amino acid residues mentioned in the Table can be either charged and/or uncharged at such a higher or lower pH, as will be clear to the skilled person.
⁽⁴⁾As is known in the art, the charge of a His residue is greatly dependant upon even small shifts in pH, but a His residu can generally be considered essentially uncharged at a pH of about 6.5.

e) For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position).

Alternatively, the degree of sequence identity between two or more nucleotide sequences may be calculated using a known computer algorithm for sequence alignment such as NCBI Blast v2.0, using standard settings.

Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A.

Usually, for the purpose of determining the percentage of “sequence identity” between two nucleotide sequences in accordance with the calculation method outlined hereinabove, the nucleotide sequence with the greatest number of nucleotides will be taken as the “first” nucleotide sequence, and the other nucleotide sequence will be taken as the “second” nucleotide sequence;
f) For the purposes of comparing two or more amino acid sequences, the percentage of “sequence identity” between a first amino acid sequence and a second amino acid sequence may be calculated by dividing [the number of amino acid residues in the first amino acid sequence that are identical to the amino acid residues at the corresponding positions in the second amino acid sequence] by [the total number of amino acids in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e. as an “amino acid difference” as defined herein.

Alternatively, the degree of sequence identity between two amino acid sequences may be calculated using a known computer algorithm, such as those mentioned above for determining the degree of sequence identity for nucleotide sequences, again using standard settings.

Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-2 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein.

Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Nad. Acad. Sci. USA 81: 140-144, 1984; Kyte & Doolittle; J. Molec. Biol. 157: 105-132, 198 1, and Goldman et al., Ann. Rev. Biophys. Chem. 15: 321-353, 1986, all incorporated herein in their entirety by reference. Information on the primary, secondary and tertiary structure of Nanobodies is given in the description herein and in the general background art cited above. Also, for this purpose, the crystal structure of a V_{HH} domain from a llama is for example given by Desmyter et al., Nature Structural Biology, Vol. 3, 9, 803 (1996); Spinelli et al., Natural Structural Biology (1996); 3, 752-757; and Decanniere et al., Structure, Vol. 7, 4, 361 (1999). Further information about some of the amino acid residues that in conventional V_{H} domains form the V_{H}/V_{L} interface and about potential camelizing substitutions on these positions is described in WO 94/04678, WO 96/34103, WO 03/035694, Muyldermans et al., Protein Eng. 1994 September; 7(9): 1129-3, Davies and Riechmann (1994 and 1996).
g) Amino acid sequences and nucleic acid sequences are said to be “exactly the same” if they have 100% sequence identity (as defined herein) over their entire length;
h) When comparing two amino acid sequences, the term “amino acid difference” refers to an insertion, deletion or substitution of a single amino acid residue on a position of the first sequence, compared to the second sequence; it being understood that two amino acid sequences can contain one, two or more such amino acid differences;
i) A nucleic acid sequence or amino acid sequence is considered to be “(in) essentially isolated (form)”—for example, compared to its native biological source and/or the reaction medium or cultivation medium from which it has been obtained—when it has been separated from at least one other component with which it is usually associated in said source or medium, such as another nucleic acid, another protein/polypeptide, another biological component or macromolecule or at least one contaminant, impurity or minor component. In particular, a nucleic acid sequence or amino acid sequence is considered “essentially isolated” when it has been purified at least 2-fold, in particular at least 10-fold, more in particular at least 100-fold, and up to 1000-fold or more. A nucleic acid sequence or amino acid sequence that is “in essentially isolated form” is preferably essentially homogeneous, as determined using a suitable technique, such as a suitable chromatographical technique, such as polyacrylamide-gel electrophoresis;
j) The term “domain” as used herein generally refers to a globular region of an antibody chain, and in particular to a globular region of a heavy chain antibody, or to a polypeptide that essentially consists of such a globular region. Usually, such a domain will comprise peptide loops (for example 3 or 4 peptide loops) stabilized, for example, as a sheet or by disulfide bonds.
k) The term ‘antigenic determinant’ refers to the epitope on the antigen recognized by the antigen-binding molecule (such as a Nanobody or a polypeptide of the invention) and more in particular by the antigen-binding site of said molecule. The terms “antigenic determinant” and “epitope’ may also be used interchangeably herein.
l) An amino acid sequence (such as a Nanobody, an antibody, a polypeptide of the invention, or generally an antigen binding protein or polypeptide or a fragment thereof) that can bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.
m) The term “specificity” refers to the number of different types of antigens or antigenic determinants to which a particular antigen-binding molecule or antigen-binding protein (such as a Nanobody or a polypeptide of the invention) molecule can bind. The specificity of an antigen-binding protein can be determined based on affinity and/or avidity. The affinity, represented by the equilibrium constant for the dissociation of an antigen with an antigen-binding protein (K_{D}), is a measure for the binding strength between an antigenic determinant and an antigen-binding site on the antigen-binding protein: the lesser the value of the K_{D}, the stronger the binding strength between an antigenic determinant and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as a Nanobody or polypeptide of the invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the Nanobodies and/or polypeptides of the invention) will bind with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹. Any K_{D} value greater than 10⁻⁴ liters/mol is generally considered to indicate non-specific binding. Preferably, a Nanobody or polypeptide of the invention will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art.
n) As further described herein, the amino acid sequence and structure of a Nanobody can be considered—without however being limited thereto—to be comprised of four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively;
o) As also further described herein, the total number of amino acid residues in a Nanobody can be in the region of 110-120, is preferably 112-115, and is most preferably 113. It should however be noted that parts, fragments, analogs or derivatives (as further described herein) of a Nanobody are not particularly limited as to their length and/or size, as long as such parts, fragments, analogs or derivatives meet the further requirements outlined herein and are also preferably suitable for the purposes described herein;
p) The amino acid residues of a Nanobody are numbered according to the general numbering for V_{H} domains given by Kabat et al. (“Sequence of proteins of immunological interest”, US Public Health Services, NIH Bethesda, Md., Publication No. 91), as applied to V_{HH} domains from Camelids in the article of Riechmann and Muyldermans, referred to herein (see for example FIG. 2 of said reference). According to this numbering, FR1 of a Nanobody comprises the amino acid residues at positions 1-30, CDR1 of a Nanobody comprises the amino acid residues at positions 31-35, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-113. [In this respect, it should be noted that—as is well known in the art for V_{H} domains and for V_{HH} domains—the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR's, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.].

Alternative methods for numbering the amino acid residues of V_{H} domains, which methods can also be applied in an analogous manner to V_{HH} domains from Camelids and to Nanobodies, are the method described by Chothia et al. (Nature 342, 877-883 (1989)), the so-called “AbM definition” and the so-called “contact definition”. However, in the present description, claims and figures, the numbering according to Kabat as applied to V_{HH} domains by Riechmann and Muyldermans will be followed, unless indicated otherwise; and
q) The Figures, Sequence Listing and the Experimental Part/Examples are only given to further illustrate the invention and should not be interpreted or construed as limiting the scope of the invention and/or of the appended claims in any way, unless explicitly indicated otherwise herein.
   For a general description of heavy chain antibodies and the variable domains thereof, reference is inter alia made to the following references, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and applicant; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551 by applicant and the further published patent applications by applicant; Hamers-Casterman et al., Nature 1993 June 3; 363 (6428): 446-8; Davies and Riechmann, FEBS Lett. 1994 Feb. 21; 339(3): 285-90; Muyldermans et al., Protein Eng. 1994 September; 7(9): 1129-3; Davies and Riechmann, Biotechnology (NY) 1995 May; 13(5): 475-9; Gharoudi et al., 9th Forum of Applied Biotechnology, Med. Fac. Landbouw Univ. Gent. 1995; 60/4a part I: 2097-2100; Davies and Riechmann, Protein Eng. 1996 June; 9(6): 531-7; Desmyter et al., Nat Struct Biol. 1996 September; 3(9): 803-11; Sheriff et al., Nat Struct Biol. 1996 September; 3(9): 733-6; Spinelli et al., Nat Struct Biol. 1996 September; 3(9): 752-7; Arbabi Ghahroudi et al., FEBS Lett. 1997 Sep. 15; 414(3): 521-6; Vu et al., Mol. Immunol. 1997 November-December; 34(16-17): 1121-31; Atarhouch et al., Journal of Camel Practice and Research 1997; 4: 177-182; Nguyen et al., J. Mol. Biol. 1998 Jan. 23; 275(3): 413-8; Lauwereys et al., EMBO J. 1998 Jul. 1; 17(13): 3512-20; Frenken et al., Res Immunol. 1998 July-August; 149(6):589-99; Transue et al., Proteins 1998 Sep. 1; 32(4): 515-22; Muyldermans and Lauwereys, J. Mol. Recognit. 1999 March-April; 12 (2): 131-40; van der Linden et al., Biochim. Biophys. Acta 1999 Apr. 12; 1431(1): 37-46; Decanniere et al., Structure Fold. Des. 1999 Apr. 15; 7(4): 361-70; Ngyuen et al., Mol. Immunol. 1999 June; 36(8): 515-24; Woolven et al., Immunogenetics 1999 October; 50 (1-2): 98-101; Riechmann and Muyldermans, J. Immunol. Methods 1999 Dec. 10; 231 (1-2): 25-38; Spinelli et al., Biochemistry 2000 Feb. 15; 39(6): 1217-22; Frenken et al., J. Biotechnol. 2000 Feb. 28; 78(1): 11-21; Nguyen et al., EMBO J. 2000 Mar. 1; 19(5): 921-30; van der Linden et al., J. Immunol. Methods 2000 Jun. 23; 240 (1-2): 185-95; Decanniere et al., J. Mol. Biol. 2000 Jun. 30; 300 (1): 83-91; van der Linden et al., J. Biotechnol. 2000 Jul. 14; 80(3): 261-70; Harinsen et al., Mol. Immunol. 2000 August; 37(10): 579-90; Pérez et al., Biochemistry 2001 Jan. 9; 40(1): 74-83; Conrath et al., J. Biol. Chem. 2001 Mar. 9; 276 (10): 7346-50; Muyldermans et al., Trends Biochem Sci. 2001 April; 26(4):230-5; Muyldermans S., J. Biotechnol. 2001 June; 74 (4): 277-302; Desmyter et al., J. Biol. Chem. 2001 July 13; 276 (28): 26285-90; Spinelli et al., J. Mol. Biol. 2001 Aug. 3; 311 (1): 123-9; Conrath et al., Antimicrob Agents Chemother. 2001 October; 45 (10): 2807-12; Decanniere et al., J. Mol. Biol. 2001 Oct. 26; 313(3): 473-8; Nguyen et al., Adv Immunol. 2001; 79: 261-96; Muruganandam et al., FASEB J. 2002 February; 16 (2): 240-2; Ewert et al., Biochemistry 2002 Mar. 19; 41 (11): 3628-36; Dumoulin et al., Protein Sci. 2002 March; 11 (3): 500-15; Cortez-Retamozo et al., Int. J. Cancer. 2002 Mar. 20; 98 (3): 456-62; Su et al., Mol. Biol. Evol. 2002 March; 19 (3): 205-15; van der Vaart J M., Methods Mol. Biol. 2002; 178: 359-66; Vranken et al., Biochemistry 2002 Jul. 9; 41 (27): 8570-9; Nguyen et al., Immunogenetics 2002 April; 54 (1): 39-47; Renisio et al., Proteins 2002 Jun. 1; 47 (4): 546-55; Desmyter et al., J. Biol. Chem. 2002 Jun. 28; 277 (26): 23645-50; Ledeboer et al., J. Dairy Sci. 2002 June; 85 (6): 1376-82; De Genst et al., J. Biol. Chem. 2002 Aug. 16; 277 (33): 29897-907; Ferrat et al., Biochem. J. 2002 Sep. 1; 366 (Pt 2): 415-22; Thomassen et al., Enzyme and Microbial Technol. 2002; 30: 273-8; Harmsen et al., Appl. Microbiol. Biotechnol. 2002 December; 60 (4): 449-54; Jobling et al., Nat. Biotechnol. 2003 January; 21 (1): 77-80; Conrath et al., Dev. Comp. Immunol. 2003 February; 27 (2): 87-103; Pleschberger et al., Bioconjug. Chem. 2003 March-April; 14 (2): 440-8; Lah et al., J. Biol. Chem. 2003 Apr. 18; 278 (16): 14101-11; Nguyen et al., Immunology. 2003 May; 109 (1): 93-101; Joosten et al., Microb. Cell Fact. 2003 Jan. 30; 2 (1): 1; Li et al., Proteins 2003 Jul. 1; 52 (1): 47-50; Loris et al., Biol. Chem. 2003 Jul. 25; 278 (30): 28252-7; van Koningsbruggen et al., J. Immunol. Methods. 2003 August; 279 (1-2): 149-61; Dumoulin et al., Nature. 2003 Aug. 14; 424 (6950): 783-8; Bond et al., J. Mol. Biol. 2003 Sep. 19; 332 (3): 643-55; Yau et al., J. Immunol. Methods. 2003 Oct. 1; 281 (1-2): 161-75; Dekker et al., J. Virol. 2003 November; 77 (22): 12132-9; Meddeb-Mouelhi et al., Toxicon. 2003 December; 42 (7): 785-91; Verheesen et al., Biochim. Biophys. Acta 2003 Dec. 5; 1624 (1-3): 21-8; Zhang et al., J Mol. Biol. 2004 Jan. 2; 335 (1): 49-56; Stijlemans et al., J Biol. Chem. 2004 Jan. 9; 279 (2): 1256-61; Cortez-Retamozo et al., Cancer Res. 2004 Apr. 15; 64 (8): 2853-7; Spinelli et al., FEBS Lett. 2004 Apr. 23; 564 (1-2): 35-40; Pleschberger et al., Bioconjug. Chem. 2004 May-June; 15 (3): 664-71; Nicaise et al., Protein Sci. 2004 July; 13 (7): 1882-91; Omidfar et al., Tumour Biol. 2004 July-August; 25 (4): 179-87; Omidfar et al., Tumour Biol. 2004 September-December; 25(5-6): 296-305; Szynol et al., Antimicrob Agents Chemother. 2004 September; 48(9):3390-5; Saerens et al., J. Biol. Chem. 2004 Dec. 10; 279 (50): 51965-72; De Genst et al., J. Biol. Chem. 2004 Dec. 17; 279 (51): 53593-601; Dolk et al., Appl. Environ. Microbiol. 2005 January; 71(1): 442-50; Joosten et al., Appl Microbiol Biotechnol. 2005 January; 66(4): 384-92; Dumoulin et al., J. Mol. Biol. 2005 Feb. 25; 346 (3): 773-88; Yau et al., J Immunol Methods. 2005 February; 297 (1-2): 213-24; De Genst et al., J. Biol. Chem. 2005 Apr. 8; 280 (14): 14114-21; Huang et al., Eur. J. Hum. Genet. 2005 Apr. 13; Dolk et al., Proteins. 2005 May 15; 59 (3): 555-64; Bond et al., J. Mol. Biol. 2005 May 6; 348(3):699-709; Zarebski et al., J. Mol. Biol. 2005 Apr. 21; [E-publication ahead of print].

In accordance with the terminology used in the above references, the variable domains present in naturally occurring heavy chain antibodies will also be referred to as “V_{HH} domains”, in order to distinguish them from the heavy chain variable domains that are present in conventional 4-chain antibodies (which will be referred to hereinbelow as “V_{H} domains”) and from the light chain variable domains that are present in conventional 4-chain antibodies (which will be referred to hereinbelow as “V_{L} domains”).

As mentioned in the prior art referred to above, V_{HH} domains have a number of unique structural characteristics and functional properties which make isolated V_{HH} domains (as well as Nanobodies based thereon, which share these structural characteristics and functional properties with the naturally occurring V_{HH} domains) and proteins containing the same highly advantageous for use as functional antigen-binding domains or proteins. In particular, and without being limited thereto, V_{HH} domains (which have been “designed” by nature to functionally bind to an antigen without the presence of, and without any interaction with, a light chain variable domain) and Nanobodies can function as a single, relatively small, functional antigen-binding structural unit, domain or protein. This distinguishes the V_{HH} domains from the V_{H} and V_{L} domains of conventional 4-chain antibodies, which by themselves are generally not suited for practical application as single antigen-binding proteins or domains, but need to be combined in some form or another to provide a functional antigen-binding unit (as in for example conventional antibody fragments such as Fab fragments; in ScFv's fragments, which consist of a V_{H} domain covalently linked to a V_{L} domain).

Because of these unique properties, the use of V_{HH} domains and Nanobodies as single antigen-binding proteins or as antigen-binding domains (i.e. as part of a larger protein or polypeptide) offers a number of significant advantages over the use of conventional V_{H} and V_{L} domains, scFv's or conventional antibody fragments (such as Fab- or F(ab′)₂-fragments):
only a single domain is required to bind an antigen with high affinity and with high selectivity, so that there is no need to have two separate domains present, nor to assure that these two domains are present in the right spacial conformation and configuration (i.e. through the use of especially designed linkers, as with scFv's);
V_{HH} domains and Nanobodies can be expressed from a single gene and require no post-translational folding or modifications;
V_{HH} domains and Nanobodies can easily be engineered into multivalent and multispecific formats (as further discussed herein);
V_{HH} domains and Nanobodies are highly soluble and do not have a tendency to aggregate (as with the mouse-derived antigen-binding domains described by Ward et al., Nature, Vol. 341, 1989, p. 544);
V_{HH} domains and Nanobodies are highly stable to heat, pH, proteases and other denaturing agents or conditions (see for example Ewert et al, supra);
V_{HH} domains and Nanobodies are easy and relatively cheap to prepare, even on a scale required for production. For example, V_{HH} domains, Nanobodies and proteins/polypeptides containing the same can be produced using microbial fermentation (e.g. as further described below) and do not require the use of mammalian expression systems, as with for example conventional antibody fragments;
V_{HH} domains and Nanobodies are relatively small (approximately 15 kDa, or 10 times smaller than a conventional IgG) compared to conventional 4-chain antibodies and antigen-binding fragments thereof, and therefore show high(er) penetration into tissues (including but not limited to solid tumors and other dense tissues) than such conventional 4-chain antibodies and antigen-binding fragments thereof;
V_{HH} domains and Nanobodies can show so-called cavity-binding properties (inter alia due to their extended CDR3 loop, compared to conventional V_{H} domains) and can therefore also access targets and epitopes not accessable to conventional 4-chain antibodies and antigen-binding fragments thereof. For example, it has been shown that V_{HH} domains and Nanobodies can inhibit enzymes (see for example WO 97/49805; Transue et al., (1998), supra; Lauwereys et al., (1998), supra.

As mentioned above, the invention generally relates to Nanobodies directed against EGFR or IGF-IR, respectively, as well as to polypeptides comprising or essentially consisting of one or more of such Nanobodies, that can be used for the prophylactic, therapeutic and/or diagnostic purposes described herein.

As also further described herein, the invention further relates to nucleic acids encoding such Nanobodies and polypeptides, to methods for preparing such Nanobodies and polypeptides, to host cells expressing or capable of expressing such Nanobodies or polypeptides, to compositions comprising such Nanobodies, polypeptides, nucleic acids or host cells, and to uses of such Nanobodies, polypeptides, nucleic acids, host cells or compositions.

Generally, it should be noted that the term Nanobody as used herein in its broadest sense is not limited to a specific biological source or to a specific method of preparation. For example, as will be discussed in more detail below, the Nanobodies of the invention can generally be obtained: (1) by isolating the V_{HH} domain of a naturally occurring heavy chain antibody; (2) by expression of a nucleotide sequence encoding a naturally occurring V_{HH} domain; (3) by “humanization” (as described herein) of a naturally occurring V_{HH} domain or by expression of a nucleic acid encoding a such humanized V_{HH} domain; (4) by “camelization” (as described herein) of a naturally occurring V_{H} domain from any animal species, and in particular a from species of mammal, such as from a human being, or by expression of a nucleic acid encoding such a camelized V_{H} domain; (5) by “camelisation” of a “domain antibody” or “Dab” as described by Ward et al (supra), or by expression of a nucleic acid encoding such a camelized V_{H} domain; (6) by using synthetic or semi-synthetic techniques for preparing proteins, polypeptides or other amino acid sequences known per se; (7) by preparing a nucleic acid encoding a Nanobody using techniques for nucleic acid synthesis known per se, followed by expression of the nucleic acid thus obtained; and/or (8) by any combination of one or more of the foregoing. Suitable methods and techniques for performing the foregoing will be clear to the skilled person based on the disclosure herein and for example include the methods and techniques described in more detail herein.

One preferred class of Nanobodies corresponds to the V_{HH} domains of naturally occurring heavy chain antibodies directed against EGFR or IGF-IR, respectively. As further described herein, such V_{HH} sequences can generally be generated or obtained by suitably immunizing a species of Camelid with EGFR or IGF-IR, respectively, (i.e. so as to raise an immune response and/or heavy chain antibodies directed against EGFR or IGF-IR, respectively), by obtaining a suitable biological sample from said Camelid (such as a blood sample, serum sample or sample of B-cells), and by generating V_{HH} sequences directed against EGFR or IGF-IR, respectively, starting from said sample, using any suitable technique known per se. Such techniques will be clear to the skilled person and/or are further described herein.

Alternatively, such naturally occurring V_{HH} domains against EGFR or IGF-IR, respectively, can be obtained from naïve libraries of Camelid V_{HH} sequences, for example by screening such a library using EGFR or IGF-IR, respectively, or at least one part, fragment, antigenic determinant or epitope thereof using one or more screening techniques known per se. Such libraries and techniques are for example described in WO 99/37681, WO 01/90190, WO 03/025020 and WO 03/035694. Alternatively, improved synthetic or semi-synthetic libraries derived from naïve V_{HH} libraries may be used, such as V_{HH} libraries obtained from naïve V_{HH} libraries by techniques such as random mutagenesis and/or CDR shuffling, as for example described in WO 00/43507.

Yet another technique for obtaining V_{HH} sequences directed against EGFR or IGF-IR, respectively, involves suitably immunizing a transgenic mammal that is capable of expressing heavy chain antibodies (i.e. so as to raise an immune response and/or heavy chain antibodies directed against EGFR or IGF-IR, respectively), obtaining a suitable biological sample from said transgenic mammal (such as a blood sample, serum sample or sample of B-cells), and then generating V_{HH} sequences directed against EGFR or IGF-IR, respectively, starting from said sample, using any suitable technique known per se. For example, for this purpose, the heavy chain antibody-expressing mice and the further methods and techniques described in WO 02/085945 and in WO 04/049794 can be used.

A particularly preferred class of Nanobodies of the invention comprises Nanobodies with an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the further description herein and the prior art on humanization referred to herein. Again, it should be noted that such humanized Nanobodies of the invention can be obtained in any suitable manner known per se (i.e. as indicated under points (1)-(8) above) and thus are not strictly limited to polypeptides that have been obtained using a polypeptide that comprises a naturally occurring V_{HH} domain as a starting material.

Another particularly preferred class of Nanobodies of the invention comprises Nanobodies with an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the further description herein. Such “camelizing” substitutions are preferably inserted at amino acid positions that form and/or are present at the V_{H}-V_{L} interface, and/or at the so-called Camelidae hallmark residues, as defined herein (see for example WO 94/04678 and Davies and Riechmann (1994 and 1996), supra). Preferably, the V_{H} sequence that is used as a starting material or starting point for generating or designing the camelized Nanobody is preferably a V_{H} sequence from a mammal, more preferably the V_{H} sequence of a human being, such as a V_{H}3 sequence. However, it should be noted that such camelized Nanobodies of the invention can be obtained in any suitable manner known per se (i.e. as indicated under points (1)-(8) above) and thus are not strictly limited to polypeptides that have been obtained using a polypeptide that comprises a naturally occurring V_{H} domain as a starting material.

For example, again as further described herein, both “humanization” and “camelization” can be performed by providing a nucleotide sequence that encodes a naturally occurring V_{HH} domain or V_{H} domain, respectively, and then changing, in a manner known per se, one or more codons in said nucleotide sequence in such a way that the new nucleotide sequence encodes a “humanized” or “camelized” Nanobody of the invention, respectively. This nucleic acid can then be expressed in a manner known per se, so as to provide the desired Nanobody of the invention. Alternatively, based on the amino acid sequence of a naturally occurring V_{HH} domain or V_{H} domain, respectively, the amino acid sequence of the desired humanized or camelized Nanobody of the invention, respectively, can be designed and then synthesized de novo using techniques for peptide synthesis known per se. Also, based on the amino acid sequence or nucleotide sequence of a naturally occurring V_{HH} domain or V_{H} domain, respectively, a nucleotide sequence encoding the desired humanized or camelized Nanobody of the invention, respectively, can be designed and then synthesized de novo using techniques for nucleic acid synthesis known per se, after which the nucleic acid thus obtained can be expressed in a manner known per se, so as to provide the desired Nanobody of the invention.

Other suitable methods and techniques for obtaining the Nanobodies of the invention and/or nucleic acids encoding the same, starting from naturally occurring V_{H} sequences or preferably V_{HH} sequences, will be clear from the skilled person, and may for example comprise combining one or more parts of one or more naturally occurring V_{H} sequences (such as one or more FR sequences and/or CDR sequences), one or more parts of one or more naturally occurring V_{HH} sequences (such as one or more FR sequences or CDR sequences), and/or one or more synthetic or semi-synthetic sequences, in a suitable manner, so as to provide a Nanobody of the invention or a nucleotide sequence or nucleic acid encoding the same.

According to one preferred, but non-limiting aspect of the aspect of the invention, a Nanobody in its broadest sense can be generally defined as a polypeptide comprising:
(a) an amino acid sequence that is comprised of four framework regions/sequences interrupted by three complementarity determining regions/sequences, in which the amino acid residue at position 108 according to the Kabat numbering is Q;
   and/or:
(b) an amino acid sequence that is comprised of four framework regions/sequences interrupted by three complementarity determining regions/sequences, in which the amino acid residue at position 45 according to the Kabat numbering is a charged amino acid (as defined herein) or a cysteine residue, and position 44 is preferably an E;
   and/or:
(c) an amino acid sequence that is comprised of four framework regions/sequences interrupted by three complementarity determining regions/sequences, in which the amino acid residue at position 103 according to the Kabat numbering is chosen from the group consisting of P, R and S, and is in particular chosen from the group consisting of R and S.

Thus, in a first preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which
(a) the amino acid residue at position 108 according to the Kabat numbering is Q;
   and/or in which:
(b) the amino acid residue at position 45 according to the Kabat numbering is a charged amino acid or a cysteine and the amino acid residue at position 44 according to the Kabat numbering is preferably E;
   and/or in which:
(c) the amino acid residue at position 103 according to the Kabat numbering is chosen from the group consisting of P, R and S, and is in particular chosen from the group consisting of R and S;
   and in which:
(d) CDR1, CDR2 and CDR3 are as defined herein, and are preferably as defined according to one of the preferred embodiments herein, and are more preferably as defined according to one of the more preferred embodiments herein.

In particular, a Nanobody in its broadest sense can be generally defined as a polypeptide comprising:
(a) an amino acid sequence that is comprised of four framework regions/sequences interrupted by three complementarity determining regions/sequences, in which the amino acid residue at position 108 according to the Kabat numbering is Q;
   and/or:
(b) an amino acid sequence that is comprised of four framework regions/sequences interrupted by three complementarity determining regions/sequences, in which the amino acid residue at position 44 according to the Kabat numbering is E and in which the amino acid residue at position 45 according to the Kabat numbering is an R;
   and/or:
(c) an amino acid sequence that is comprised of four framework regions/sequences interrupted by three complementarity determining regions/sequences, in which the amino acid residue at position 103 according to the Kabat numbering is chosen from the group consisting of P, R and S, and is in particular chosen from the group consisting of R and S.

Thus, according to a preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which
(a) the amino acid residue at position 108 according to the Kabat numbering is Q; and/or in which:
(b) the amino acid residue at position 44 according to the Kabat numbering is E and in which the amino acid residue at position 45 according to the Kabat numbering is an R;
   and/or in which:
(c) the amino acid residue at position 103 according to the Kabat numbering is chosen from the group consisting of P, R and S, and is in particular chosen from the group consisting of R and S;
   and in which:
(d) CDR1, CDR2 and CDR3 are as defined herein, and are preferably as defined according to one of the preferred embodiments herein, and are more preferably as defined according to one of the more preferred embodiments herein.

In particular, a Nanobody against EGFR or IGF-IR, respectively, according to the invention may have the structure:

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which
(a) the amino acid residue at position 108 according to the Kabat numbering is Q;
   and/or in which:
(b) the amino acid residue at position 44 according to the Kabat numbering is E and in which the amino acid residue at position 45 according to the Kabat numbering is an R;
   and/or in which:
(c) the amino acid residue at position 103 according to the Kabat numbering is chosen from the group consisting of P, R and S, and is in particular chosen from the group consisting of R and S;
   and in which:
(d) CDR1, CDR2 and CDR3 are as defined herein, and are preferably as defined according to one of the preferred embodiments herein, and are more preferably as defined according to one of the more preferred embodiments herein.

In particular, according to one preferred, but non-limiting aspect of the aspect of the invention, a Nanobody can generally be defined as a polypeptide comprising an amino acid sequence that is comprised of four framework regions/sequences interrupted by three complementarity determining regions/sequences, in which;
(a-1) the amino acid residue at position 44 according to the Kabat numbering is chosen from the group consisting of A, G, E, D, G, Q, R, S, L; and is preferably chosen from the group consisting of G, E or Q; and
(a-2) the amino acid residue at position 45 according to the Kabat numbering is chosen from the group consisting of L, R or C; and is preferably chosen from the group consisting of L or R; and
(a-3) the amino acid residue at position 103 according to the Kabat numbering is chosen from the group consisting of W, R or S; and is preferably W or R, and is most preferably W;
(a-4) the amino acid residue at position 108 according to the Kabat numbering is Q;
   or in which:
(b-1) the amino acid residue at position 44 according to the Kabat numbering is chosen from the group consisting of E and Q; and
(b-2) the amino acid residue at position 45 according to the Kabat numbering is R; and
(b-3) the amino acid residue at position 103 according to the Kabat numbering is chosen from the group consisting of W, R and S; and is preferably W;
(b-4) the amino acid residue at position 108 according to the Kabat numbering is chosen from the group consisting of Q and L; and is preferably Q;
   or in which:
(c-1) the amino acid residue at position 44 according to the Kabat numbering is chosen from the group consisting of A, G, E, D, Q, R, S and L; and is preferably chosen from the group consisting of G, E and Q; and
(c-2) the amino acid residue at position 45 according to the Kabat numbering is chosen from the group consisting of L, R and C; and is preferably chosen from the group consisting of L and R; and
(c-3) the amino acid residue at position 103 according to the Kabat numbering is chosen from the group consisting of P, R and S; and is in particular chosen from the group consisting of R and S; and
(c-4) the amino acid residue at position 108 according to the Kabat numbering is chosen from the group consisting of Q and L; is preferably Q;
   and in which
(d) CDR1, CDR2 and CDR3 are as defined herein, and are preferably as defined according to one of the preferred embodiments herein, and are more preferably as defined according to one of the more preferred embodiments herein.

Thus, in another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the amino acid residue at position 44 according to the Kabat numbering is chosen from the group consisting of A, G, E, D, G, Q, R, S, L; and is preferably chosen from the group consisting of G, E or Q;
   and in which:
(b) the amino acid residue at position 45 according to the Kabat numbering is chosen from the group consisting of L, R or C; and is preferably chosen from the group consisting of L or R;
   and in which:
(c) the amino acid residue at position 103 according to the Kabat numbering is chosen from the group consisting of W, R or S; and is preferably W or R, and is most preferably W;
   and in which
(d) the amino acid residue at position 108 according to the Kabat numbering is Q; and in which:
(e) CDR1, CDR2 and CDR3 are as defined herein, and are preferably as defined according to one of the preferred embodiments herein, and are more preferably as defined according to one of the more preferred embodiments herein.

In another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the amino acid residue at position 44 according to the Kabat numbering is chosen from the group consisting of E and Q;
   and in which:
(b) the amino acid residue at position 45 according to the Kabat numbering is R; and in which:
(c) the amino acid residue at position 103 according to the Kabat numbering is chosen from the group consisting of W, R and S; and is preferably W;
   and in which:
(d) the amino acid residue at position 108 according to the Kabat numbering is chosen from the group consisting of Q and L; and is preferably Q;
   and in which:
(e) CDR1, CDR2 and CDR3 are as defined herein, and are preferably as defined according to one of the preferred embodiments herein, and are more preferably as defined according to one of the more preferred embodiments herein.

In another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the amino acid residue at position 44 according to the Kabat numbering is chosen from the group consisting of A, G, E, D, Q, R, S and L; and is preferably chosen from the group consisting of G, E and Q;
   and in which:
(b) the amino acid residue at position 45 according to the Kabat numbering is chosen from the group consisting of L, R and C; and is preferably chosen from the group consisting of Land R;
   and in which:
(c) the amino acid residue at position 103 according to the Kabat numbering is chosen from the group consisting of P, R and S; and is in particular chosen from the group consisting of R and S;
   and in which:
(d) the amino acid residue at position 108 according to the Kabat numbering is chosen from the group consisting of Q and L; is preferably Q;
   and in which:
(e) CDR1, CDR2 and CDR3 are as defined herein, and are preferably as defined according to one of the preferred embodiments herein, and are more preferably as defined according to one of the more preferred embodiments herein.

Two particularly preferred, but non-limiting groups of the Nanobodies of the invention are those according to a) above; according to (a-1) to (a-4) above; according to b) above; according to (b-1) to (b-4) above; according to (c) above; and/or according to (c-1) to (c-4) above, in which;
a) the amino acid residues at positions 44-47 according to the Kabat numbering form the sequence GLEW (or a GLEW-like sequence as defined herein) and the amino acid residue at position 108 is Q or L;
   or in which:
b) the amino acid residues at positions 43-46 according to the Kabat numbering form the sequence KERE or KQRE (or a KERE-like sequence) and the amino acid residue at position 108 is Q or L, and is preferably Q.

Thus, in another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the amino acid residues at positions 44-47 according to the Kabat numbering form the sequence GLEW (or a GLEW-like sequence as defined herein) and the amino acid residue at position 108 is Q or L;
   and in which:
(b) CDR1, CDR2 and CDR3 are as defined herein, and are preferably as defined according to one of the preferred embodiments herein, and are more preferably as defined according to one of the more preferred embodiments herein.

In another preferred, but non-limiting aspect, a Nanobody of the invention may have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the amino acid residues at positions 43-46 according to the Kabat numbering form the sequence KERE or KQRE (or a KERE-like sequence) and the amino acid residue at position 108 is Q or L, and is preferably Q;
   and in which:
(b) CDR1, CDR2 and CDR3 are as defined herein, and are preferably as defined according to one of the preferred embodiments herein, and are more preferably as defined according to one of the more preferred embodiments herein.

In the Nanobodies of the invention in which the amino acid residues at positions 43-46 according to the Kabat numbering form the sequence KERE or KQRE, the amino acid residue at position 37 is most preferably F. In the Nanobodies of the invention in which the amino acid residues at positions 44-47 according to the Kabat numbering form the sequence GLEW, the amino acid residue at position 37 is chosen from the group consisting of Y, H, I, L, V or F, and is most preferably V.

Thus, without being limited hereto in any way, on the basis of the amino acid residues present on the positions mentioned above, the Nanobodies of the invention can generally be classified on the basis of the following three groups:
a) The “GLEW-group”: Nanobodies with the amino acid sequence GLEW at positions 44-47 according to the Kabat numbering and Q or L at position 108 according to the Kabat numbering. As further described herein, Nanobodies within this group usually have a V at position 37, and can have a W, P, R or S at position 103, and preferably have a W at position 103. The GLEW group also comprises some GLEW-like sequences such as those mentioned in Table A-3 below;
b) The “KERE-group”: Nanobodies with the amino acid sequence KERE or KQRE at positions 43-46 according to the Kabat numbering and Q or L at position 108 according to the Kabat numbering. As further described herein, Nanobodies within this group usually have a F at position 37, an L or F at position 47; and can have a W, P, R or S at position 103, and preferably have a W at position 103;
c) The “103 P, R, S-group”: Nanobodies with a P, R or S at position 103. These Nanobodies can have either the amino acid sequence GLEW at positions 44-47 of the Kabat numbering or the amino acid sequence KERE or KQRE at positions 43-46 according to the Kabat numbering, the latter most preferably in combination with an F at position 37 and an L or an F at position 47 (as defined for the KERE-group); and can have Q or L at position 108 according to the Kabat numbering, and preferably have Q.

Thus, in another preferred, but non-limiting aspect, a Nanobody of the invention may be a Nanobody belonging to the GLEW-group (as defined herein), and in which CDR1, CDR2 and CDR3 are as defined herein, and are preferably as defined according to one of the preferred embodiments herein, and are more preferably as defined according to one of the more preferred embodiments herein.

In another preferred, but non-limiting aspect, a Nanobody of the invention may be a Nanobody belonging to the KERE-group (as defined herein), and CDR1, CDR2 and CDR3 are as defined herein, and are preferably as defined according to one of the preferred embodiments herein, and are more preferably as defined according to one of the more preferred embodiments herein.

Thus, in another preferred, but non-limiting aspect, a Nanobody of the invention may be a Nanobody belonging to the 103 P, R, S-group (as defined herein), and in which CDR1, CDR2 and CDR3 are as defined herein, and are preferably as defined according to one of the preferred embodiments herein, and are more preferably as defined according to one of the more preferred embodiments herein.

Also, more generally and in addition to the 108Q, 43E/44R and 103P,R,S residues mentioned above, the Nanobodies of the invention can contain, at one or more positions that in a conventional V_{H} domain would form (part of) the V_{H/}V_{L} interface, one or more amino acid residues that are more highly charged than the amino acid residues that naturally occur at the same position(s) in the corresponding naturally occurring V_{H} sequence, and in particular one or more charged amino acid residues (as mentioned in Table A-2). Such substitutions include, but are not limited to, the GLEW-like sequences mentioned in Table A-3 below; as well as the substitutions that are described in the International Application WO 00/29004 for so-called “microbodies”, e.g. so as to obtain a Nanobody with Q at position 108 in combination with KLEW at positions 44-47. Other possible substitutions at these positions will be clear to the skilled person based upon the disclosure herein.

In one embodiment of the Nanobodies of the invention, the amino acid residue at position 83 is chosen from the group consisting of L, M, S, V and W; and is preferably L.

Also, in one embodiment of the Nanobodies of the invention, the amino acid residue at position 83 is chosen from the group consisting of R, K, N, E, G, I, T and Q; and is most preferably either K or E (for Nanobodies corresponding to naturally occurring V_{HH} domains) or R (for “humanized” Nanobodies, as described herein). The amino acid residue at position 84 is chosen from the group consisting of P, A, R, S, D T, and V in one embodiment, and is most preferably P (for Nanobodies corresponding to naturally occurring V_{HH} domains) or R (for “humanized” Nanobodies, as described herein).

Furthermore, in one embodiment of the Nanobodies of the invention, the amino acid residue at position 104 is chosen from the group consisting of G and D; and is most preferably G.

Collectively, the amino acid residues at positions 11, 37, 44, 45, 47, 83, 84, 103, 104 and 108, which in the Nanobodies are as mentioned above, will also be referred to herein as the “Hallmark Residues”. The Hallmark Residues and the amino acid residues at the corresponding positions of the most closely related human V_{H} domain, V_{H}3, are summarized in Table A-3.

Some especially preferred but non-limiting combinations of these Hallmark Residues as occur in naturally occurring V_{HH} domains are mentioned in Table A-4. For comparison, the corresponding amino acid residues of the human V_{H}3 called DP-47 have been indicated in italics.

TABLE A-3
Hallmark Residues in Nanobodies

| Position | Human V_{H}3 | Hallmark Residues |
|---|---|---|
| 11 | L, V; | L, M, S, V, W; preferably L |
| | predominantly L | |
| 37 | V, I, F; usually V | F⁽¹⁾, Y, H, I, L or V, preferably F⁽¹⁾ or Y |
| 44⁽⁷⁾ | G | G⁽²⁾, E⁽³⁾, A, D, Q, R, S, L; |
| | | preferably G⁽²⁾, E⁽³⁾ or Q; |
| | | most preferably G⁽²⁾ or E⁽³⁾. |
| 45⁽⁷⁾ | L | L⁽²⁾, R⁽³⁾, C, I, L, P, Q, V; preferably L⁽²⁾ |
| | | or R⁽³⁾ |
| 47⁽⁷⁾ | W, Y | W⁽²⁾, L⁽¹⁾ or F⁽¹⁾, A, G, I, M, R, S, V or |
| | | Y; preferably W⁽²⁾, L⁽¹⁾, F⁽¹⁾ or R |
| 83 | R or K; usually R | R, K⁽⁵⁾, N, E⁽⁵⁾, G, I, M, Q or T; |
| | | preferably K or R; most preferably K |
| 84 | A, T, D; | P⁽⁵⁾, A, L, R, S, T, D, V; preferably P |
| | predominantly A | |
| 103 | W | W⁽⁴⁾, P⁽⁶⁾, R⁽⁶⁾, S; preferably W |
| 104 | G | G or D; preferably G |
| 108 | L, M or T; | Q, L or R; preferably Q or L |
| | predominantly L | |

Notes:
⁽¹⁾In particular, but not exclusively, in combination with KERE or KQRE at positions 43-46.
⁽²⁾Usually as GLEW at positions 44-47.
⁽³⁾Usually as KERE or KQRE at positions 43-46, e.g. as KEREL, KEREF, KQREL, KQREF or KEREG at positions 43-47. Alternatively, also sequences such as TERE (for example TEREL), KECE (for example KECEL or KECER), RERE (for example REREG), QERE (for example QEREG), KGRE (for example KGREG), KDRE (for example KDREV) are possible. Some other possible, but less preferred sequences include for example DECKL and NVCEL.
⁽⁴⁾With both GLEW at positions 44-47 and KERE or KQRE at positions 43-46.
⁽⁵⁾Often as KP or EP at positions 83-84 of naturally occurring V_{HH} domains.
⁽⁶⁾In particular, but not exclusively, in combination with GLEW at positions 44-47.
⁽⁷⁾The GLEW group also contains GLEW-like sequences at positions 44-47, such as for example GVEW, EPEW, GLER, DQEW, DLEW, GIEW, ELEW, GPEW, EWLP, GPER, GLER and ELEW.

TABLE A-4
Some preferred but non-limiting combinations of
Hallmark Residues in naturally occurring Nanobodies.
For humanization of these combinations,
reference is made to the specification.

| | 11 | 37 | 44 | 45 | 47 | 83 | 84 | 103 | 104 | 108 |
|---|---|---|---|---|---|---|---|---|---|---|
| DP-47 (human) | M | V | G | L | W | R | A | W | G | L |
| “KERE” group | L | F | E | R | L | K | P | W | G | Q |
| | L | F | E | R | F | E | P | W | G | Q |
| | L | F | E | R | F | K | P | W | G | Q |
| | L | Y | Q | R | L | K | P | W | G | Q |
| | L | F | L | R | V | K | P | Q | G | Q |
| | L | F | Q | R | L | K | P | W | G | Q |
| | L | F | E | R | F | K | P | W | G | Q |
| “GLEW” group | L | V | G | L | W | K | S | W | G | Q/L |
| | M | V | G | L | W | K | P | R | G | Q/L |

In the Nanobodies, each amino acid residue at any other position than the Hallmark Residues can be any amino acid residue that naturally occurs at the corresponding position (according to the Kabat numbering) of a naturally occurring V_{HH} domain.

Such amino acid residues will be clear to the skilled person. Tables A-5-A-8 mention some non-limiting residues that can be present at each position (according to the Kabat numbering) of the FR1, FR2, FR3 and FR4 of naturally occurring V_{HH} domains. For each position, the amino acid residue that most frequently occurs at each position of a naturally occurring V_{HH} domain (and which is the most preferred amino acid residue for said position in a Nanobody) is indicated in bold; and other preferred amino acid residues for each position have been underlined (note: the number of amino acid residues that are found at positions 26-30 of naturally occurring V_{HH} domains supports the hypothesis underlying the numbering Chothia (supra) that the residues at these positions already form part of CDR1).

In Tables A-5-A-8, some of the non-limiting residues that can be present at each position of a human V_{H}3 domain have also been mentioned. Again, for each position, the amino acid residue that most frequently occurs at each position of a naturally occurring human V_{H}3 domain is indicated in bold; and other preferred amino acid residues have been underlined.

For reference only, Table A-5 also contains data on the V_{HH} entropy (“V_{HH} Ent.”) and V_{HH} variability (“V_{HH} Var.”) at each amino acid position for a representative sample of 1118 V_{HH} sequences (data kindly provided by David Lutje Hulsing and Prof. Theo Verrips of Utrecht University). The values for the V_{HH} entropy and the V_{HH} variability provide a measure for the variability and degree of conservation of amino acid residues between the 1118 V_{HH} sequences analyzed: low values (i.e. <1, such as <0.5) indicate that an amino acid residue is highly conserved between the V_{HH} sequences (i.e. little variability). For example, the G at position 8 and the G at position 9 have values for the V_{HH} entropy of 0.1 and 0 respectively, indicating that these residues are highly conserved and have little variability (and in case of position 9 is G in all 1118 sequences analysed), whereas for residues that form part of the CDR's generally values of 1.5 or more are found (data not shown). Note that (1) the amino acid residues listed in the second column of Table A-5 are based on a bigger sample than the 1118 V_{HH} sequences that were analysed for determining the V_{HH} entropy and V_{HH} variability referred to in the last two columns; and (2) the data represented below supports the hypothesis that the amino acid residues at positions 27-30 and maybe even also at positions 93 and 94 already form part of the CDR's (although the invention is not limited to any specific hypothesis or explanation, and as mentioned above, herein the numbering according to Kabat is used). For a general explanation of sequence entropy, sequence variability and the methodology for determining the same, see Oliveira et al., PROTEINS: Structure, Function and Genetics, 52: 544-552 (2003).

TABLE A-5
Non-limiting examples of amino acid residues in FR1 (for the footnotes,
see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 1 | E, Q | Q, A, E | — | — |
| 2 | V | V | 0.2 | 1 |
| 3 | Q | Q, K | 0.3 | 2 |
| 4 | L | L | 0.1 | 1 |
| 5 | V, L | Q, E, L, V | 0.8 | 3 |
| 6 | E | E, D, Q, A | 0.8 | 4 |
| 7 | S, T | S, F | 0.3 | 2 |
| 8 | G, R | G | 0.1 | 1 |
| 9 | G | G | 0 | 1 |
| 10 | G, V | G, D, R | 0.3 | 2 |
| 11 | Hallmark residue: L, M, S, V, W; preferably L | | 0.8 | 2 |
| 12 | V, I | V, A | 0.2 | 2 |
| 13 | Q, K, R | Q, E, K, P, R | 0.4 | 4 |
| 14 | P | A, Q, A, G, P, S, T, V | 1 | 5 |
| 15 | G | G | 0 | 1 |
| 16 | G, R | G, A, E, D | 0.4 | 3 |
| 17 | S | S, F | 0.5 | 2 |
| 18 | L | L, V | 0.1 | 1 |
| 19 | R, K | R, K, L, N, S, T | 0.6 | 4 |
| 20 | L | L, F, I, V | 0.5 | 4 |
| 21 | S | S, A, F, T | 0.2 | 3 |
| 22 | C | C | 0 | 1 |
| 23 | A, T | A, D, E, P, S, T, V | 1.3 | 5 |
| 24 | A | A, I, L, S, T, V | 1 | 6 |
| 25 | S | S, A, F, P, T | 0.5 | 5 |
| 26 | G | G, A, D, E, R, S, T, V | 0.7 | 7 |
| 27 | F | S, F, R, L, P, G, N, | 2.3 | 13 |
| 28 | T | N, T, E, D, S, I, R, A, G, R, F, Y | 1.7 | 11 |
| 29 | F, V | F, L, D, S, I, G, V, A | 1.9 | 11 |
| 30 | S, D, G | N, S, E, G, A, D, M, T | 1.8 | 11 |

TABLE A-6
Non-limiting examples of amino acid residues in FR2
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 36 | W | W | 0.1 | 1 |
| 37 | Hallmark residue: F⁽¹⁾, H, I, L, Y or V, | | 1.1 | 6 |
| | preferably F⁽¹⁾ or Y | | | |
| 38 | R | R | 0.2 | 1 |
| 39 | Q | Q, H, P, R | 0.3 | 2 |
| 40 | A | A, F, G, L, P, T, V | 0.9 | 7 |
| 41 | P, S, T | P, A, L, S | 0.4 | 3 |
| 42 | G | G, E | 0.2 | 2 |
| 43 | K | K, D, E, N, Q, R, T, V | 0.7 | 6 |
| 44 | Hallmark residue: G⁽²⁾, E⁽³⁾, A, D, Q, R, S, L; | | 1.3 | 5 |
| | preferably G⁽²⁾, E⁽³⁾ or Q; | | | |
| | most preferably G⁽²⁾ or E⁽³⁾. | | | |
| 45 | Hallmark residue: L⁽²⁾, R⁽³⁾, C, I, L, P, Q, V; | | 0.6 | 4 |
| | preferably L⁽²⁾ or R⁽³⁾ | | | |
| 46 | E, V | E, D, K, Q, V | 0.4 | 2 |
| 47 | Hallmark residue: W⁽²⁾, L⁽¹⁾ or F⁽¹⁾, A, | | 1.9 | 9 |
| | G, I, M, R, S, V or Y; preferably W⁽²⁾, | | | |
| | L⁽¹⁾, F⁽¹⁾ or R | | | |
| 48 | V | V, I, L | 0.4 | 3 |
| 49 | S, A, G | A, S, G, T, V | 0.8 | 3 |

TABLE A-7
Non-limiting examples of amino acid residues in FR3
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 66 | R | R | 0.1 | 1 |
| 67 | F | F, L, V | 0.1 | 1 |
| 68 | T | T, A, N, S | 0.5 | 4 |
| 69 | I | I, L, M, V | 0.4 | 4 |
| 70 | S | S, A, F, T | 0.3 | 4 |
| 71 | R | R, G, H, I, L, K, Q, S, T, W | 1.2 | 8 |
| 72 | D, E | D, E, G, N, V | 0.5 | 4 |
| 73 | N, D, G | N, A, D, F, I, K, L, R, S, T, V, Y | 1.2 | 9 |
| 74 | A, S | A, D, G, N, P, S, T, V | 1 | 7 |
| 75 | K | K, A, E, K, L, N, Q, R | 0.9 | 6 |
| 76 | N, S | N, D, K, R, S, T, Y | 0.9 | 6 |
| 77 | S, T, I | T, A, E, I, M, P, S | 0.8 | 5 |
| 78 | L, A | V, L, A, F, G, I, M | 1.2 | 5 |
| 79 | Y, H | Y, A, D, F, H, N, S, T | 1 | 7 |
| 80 | L | L, F, V | 0.1 | 1 |
| 81 | Q | Q, E, I, L, R, T | 0.6 | 5 |
| 82 | M | M, I, L, V | 0.2 | 2 |
| 82a | N, G | N, D, G, H, S, T | 0.8 | 4 |
| 82b | S | S, N, D, G, R, T | 1 | 6 |
| 82c | L | L, P, V | 0.1 | 2 |
| 83 | Hallmark residue: R, K⁽⁵⁾, N, E⁽⁵⁾, G, I, M, O or T; | | 0.9 | 7 |
| | preferably K or R; most preferably K | | | |
| 84 | Hallmark residue: P⁽⁵⁾, A, D, L, R, S, T, V; | | 0.7 | 6 |
| | preferably P | | | |
| 85 | E, G | E, D, G, Q | 0.5 | 3 |
| 86 | D | D | 0 | 1 |
| 87 | T, M | T, A, S | 0.2 | 3 |
| 88 | A | A, G, S | 0.3 | 2 |
| 89 | V, L | V, A, D, I, L, M, N, R, T | 1.4 | 6 |
| 90 | Y | Y, F | 0 | 1 |
| 91 | Y, H | Y, D, F, H, L, S, T, V | 0.6 | 4 |
| 92 | C | C | 0 | 1 |
| 93 | A, K, T | A, N, G, H, K, N, R, S, T, V, Y | 1.4 | 10 |
| 94 | K, R, T | A, V, C, F, G, I, K, L, R, S or T | 1.6 | 9 |

TABLE A-8
Non-limiting examples of amino acid residues in FR4
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 103 | Hallmark residue: W⁽⁴⁾, P⁽⁶⁾, R⁽⁶⁾, S; preferably W | | 0.4 | 2 |
| 104 | Hallmark residue: G or D; preferably G | | 0.1 | 1 |
| 105 | Q, R | Q, E, K, P, R | 0.6 | 4 |
| 106 | G | G | 0.1 | 1 |
| 107 | T | T, A, I | 0.3 | 2 |
| 108 | Hallmark residue: Q, L⁽⁷⁾ or R; preferably Q or L⁽⁷⁾ | | 0.4 | 3 |
| 109 | V | V | 0.1 | 1 |
| 110 | T | T, I, A | 0.2 | 1 |
| 111 | V | V, A, I | 0.3 | 2 |
| 112 | S | S, F | 0.3 | 1 |
| 113 | S | S, A, L, P, T | 0.4 | 3 |

Thus, in another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) the Hallmark residues are as defined herein;
   and in which:
(b) CDR1, CDR2 and CDR3 are as defined herein, and are preferably as defined according to one of the preferred embodiments herein, and are more preferably as defined according to one of the more preferred embodiments herein.

In another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) FR1 is chosen from the group consisting of the amino acid sequence:

| | |
|---|---|
| [1] QVQLQESGGGXVQAGGSLRLSCAASG [26] | [SEQ ID NO: 1] |

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s);
   and in which:
(b) FR2 is chosen from the group consisting of the amino acid sequence:

| | |
|---|---|
| [36] WXRQAPGKXXEXVA [49] | [SEQ ID NO: 2] |

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions and no amino acid deletions or insertions, compared to the above amino acid sequence(s);
   and in which:
(c) FR3 is chosen from the group consisting of the amino acid sequence:

[SEQ ID NO: 3]

[66] RFTISRDNAKNTVYLQMNSLXXEDTAVYYCAA [94]

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s);
   and in which:
(d) FR4 is chosen from the group consisting of the amino acid sequence:

| | |
|---|---|
| [103] XXQGTXVTVSS [113] | [SEQ ID NO: 4] |

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s);
   and in which:
(e) CDR1, CDR2 and CDR3 are as defined herein, and are preferably as defined according to one of the preferred embodiments herein, and are more preferably as defined according to one of the more preferred embodiments herein;
   in which the Hallmark Residues are indicated by “X” and are as defined hereinabove and in which the numbers between brackets refer to the amino acid positions according to the Kabat numbering.

In another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) FR1 is chosen from the group consisting of the amino acid sequence:

| | |
|---|---|
| [1] QVQLQESGGGLVQAGGSLRLSCAASG [26] | [SEQ ID NO: 5] |

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s); and
iii) the Hallmark residue at position 11 is as indicated in the sequence above;
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s); and
iii) the Hallmark residue at position 11 is as indicated in the sequence above;
   and in which:
(b) FR2 is chosen from the group consisting of the amino acid sequences:

| | |
|---|---|
| [36] WFRQAPGKERELVA [49] | [SEQ ID NO: 6] |
| [36] WFRQAPGKEREFVA [49] | [SEQ ID NO: 7] |
| [36] WFRQAPGKEREGA [49] | [SEQ ID NO: 8] |
| [36] WFRQAPGKQRELVA [49] | [SEQ ID NO: 9] |
| [36] WFRQAPGKQREFVA [49] | [SEQ ID NO: 10] |
| [36] WYRQAPGKGLEWA [49] | [SEQ ID NO: 11] |

or from the group consisting of amino acid sequences that have at least 80%, preferably at least 90%, more preferably at least 95%, even more preferably at least 99% sequence identity (as defined herein) with one of the above amino acid sequences; in which
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s); and
iii) the Hallmark residues at positions 37, 44, 45 and 47 are as indicated in each of the sequences above;
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s); and
iii) the Hallmark residues at positions 37, 44, 45 and 47 are as indicated in each of the sequences above;
   and in which:
(c) FR3 is chosen from the group consisting of the amino acid sequence:

[SEQ ID NO: 12]

[66] RFTISRDNAKNTVYLQMNSLKPEDTAVYYCAA [94]

or from the group consisting of amino acid sequences that have at least 80%, preferably at least 90%, more preferably at least 95%, even more preferably at least 99% sequence identity (as defined herein) with the above amino acid sequence; in which
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s); and
iii) the Hallmark residues at positions 83 and 84 are as indicated in each of the sequences above;
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s); and
iii) the Hallmark residues at positions 83 and 84 are as indicated in each of the sequences above;
   and in which:
(d) FR4 is chosen from the group consisting of the amino acid sequences:

| | |
|---|---|
| [103] WGQGTQVTVSS [113] | [SEQ ID NO: 13] |
| [103] WGQGTLVTVSS [113] | [SEQ ID NO: 14] |

or from the group consisting of amino acid sequences that have at least 80%, preferably at least 90%, more preferably at least 95%, even more preferably at least 99% sequence identity (as defined herein) with one of the above amino acid sequence; in which
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s); and
iii) the Hallmark residues at positions 103, 104 and 108 are as indicated in each of the sequences above;
and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s); and
iii) the Hallmark residues at positions 103, 104 and 108 are as indicated in each of the sequences above;
   and in which:
(e) CDR1, CDR2 and CDR3 are as defined herein, and are preferably as defined according to one of the preferred embodiments herein, and are more preferably as defined according to one of the more preferred embodiments herein.

In another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) FR1 is chosen from the group consisting of the amino acid sequence:

| | |
|---|---|
| [1] QVQLQESGGGLVQAGGSLRLSCAASG [26] | [SEQ ID NO: 5] |

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-5; and/or
ii) said amino acid sequence preferably only contains amino acid substitutions, and no amino acid deletions or insertions, compared to the above amino acid sequence(s); and
iii) the Hallmark residue at position 11 is as indicated in the sequence above;
   and in which:
(b) FR2 is chosen from the group consisting of the amino acid sequences:

| | |
|---|---|
| [36] WFRQAPGKERELVA [49] | [SEQ ID NO: 6] |
| [36] WFRQAPGKEREFVA [49] | [SEQ ID NO: 7] |
| [36] WFRQAPGKEREGA [49] | [SEQ ID NO: 8] |
| [36] WFRQAPGKQRELVA [49] | [SEQ ID NO: 9] |
| [36] WFRQAPGKQREFVA [49] | [SEQ ID NO: 10] |

and/or from the group consisting of amino acid sequences that have 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s); and
iii) the Hallmark residues at positions 37, 44, 45 and 47 are as indicated in each of the sequences above;
   and in which:
(c) FR3 is chosen from the group consisting of the amino acid sequence:

[SEQ ID NO: 12]

[66] RFTISRDNAKNTVYLQMNSLKPEDTAVYYCAA [94]

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s); and
iii) the Hallmark residues at positions 83 and 84 are as indicated in each of the sequences above;
   and in which:
(d) FR4 is chosen from the group consisting of the amino acid sequences:

| | |
|---|---|
| [103] WGQGTQVTVSS [113] | [SEQ ID NO: 13] |
| [103] WGQGTLVTVSS [113] | [SEQ ID NO: 14] |

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s); and
iii) the Hallmark residues at positions 103, 104 and 108 are as indicated in each of the sequences above;
   and in which:
(e) CDR1, CDR2 and CDR3 are as defined herein, and are preferably as defined according to one of the preferred embodiments herein, and are more preferably as defined according to one of the more preferred embodiments herein.

In another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) FR1 is chosen from the group consisting of the amino acid sequence:

| | |
|---|---|
| [1] QVQLQESGGGLVQAGGSLRLSCAASG [26] | [SEQ ID NO: 5] |

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-S; and/or
ii) said amino acid sequence preferably only contains amino acid substitutions, and no amino acid deletions or insertions, compared to the above amino acid sequence(s); and
iii) the Hallmark residue at position 11 is as indicated in the sequence above;
   and in which:
(b) FR2 is chosen from the group consisting of the amino acid sequence:

| | |
|---|---|
| [36] WYRQAPGKGLEWA [49] | [SEQ ID NO: 11] |

and/or from the group consisting of amino acid sequences that have 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-6; and/or
ii) said amino acid sequence preferably only contains amino acid substitutions, and no amino acid deletions or insertions, compared to the above amino acid sequence(s); and
iii) the Hallmark residues at positions 37, 44, 45 and 47 are as indicated in each of the sequences above;
   and in which:
(c) FR3 is chosen from the group consisting of the amino acid sequence:

[SEQ ID NO: 12]

[66] RFTISRDNAKNTVYLQMNSLKPEDTAVYYCAA [94]

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-7; and/or
ii) said amino acid sequence preferably only contains amino acid substitutions, and no amino acid deletions or insertions, compared to the above amino acid sequence(s); and
iii) the Hallmark residues at positions 83 and 84 are as indicated in each of the sequences above;
   and in which:
(d) FR4 is chosen from the group consisting of the amino acid sequence:

| | |
|---|---|
| [103] WGQGTQVTVSS [113] | [SEQ ID NO: 13] |

and/or from the group consisting of amino acid sequences that have 3, 2 or only 1 “amino acid difference(s)” (as defined herein) with one of the above amino acid sequences, in which:
i) any amino acid substitution at any position other than a Hallmark position is preferably either a conservative amino acid substitution (as defined herein) and/or an amino acid substitution as defined in Table A-8; and/or
ii) said amino acid sequence preferably only contains amino acid substitutions, and no amino acid deletions or insertions, compared to the above amino acid sequence(s); and
iii) the Hallmark residues at positions 103, 104 and 108 are as indicated in each of the sequences above;
   and in which:
(e) CDR1, CDR2 and CDR3 are as defined herein, and are preferably as defined according to one of the preferred embodiments herein, and are more preferably as defined according to one of the more preferred embodiments herein.

Some other framework sequences that can be present in the Nanobodies of the invention can be found in the European patent EP 656 946 mentioned above (see for example also the granted equivalent U.S. Pat. No. 5,759,808),

In another preferred, but not limiting aspect, a Nanobody of the invention can have the structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which:
(a) FR1 is chosen from the group consisting of the FR1 sequences present in the Nanobodies of SEQ ID NO's: 80-93 or 106-109, respectively, or from the group consisting of amino acid sequences that have at least 80%, preferably at least 90%, more preferably at least 95%, even more preferably at least 99% sequence identity (as defined herein) with one of said FR1 sequences; in which
   i) any amino acid substitution at any position other than a Hallmark position is preferably either a conservative amino acid substitution (as defined herein) and/or an amino acid substitution as defined in Table A-5; and/or
   ii) said amino acid sequence preferably only contains amino acid substitutions, and no amino acid deletions or insertions, compared to said FR1 sequence; and
   iii) the Hallmark residue at position 11 is as indicated in said FR1 sequence;
   and/or from the group consisting of amino acid sequences that have 3, 2 or only 1 “amino acid difference(s)” (as defined herein) with one of said FR1 sequences, in which:
   i) any amino acid substitution at any position other than a Hallmark position is preferably either a conservative amino acid substitution (as defined herein) and/or an amino acid substitution as defined in Table A-5; and/or
   ii) said amino acid sequence preferably only contains amino acid substitutions, and no amino acid deletions or insertions, compared to said FR1 sequence; and
   iii) the Hallmark residue at position 11 is as indicated in said FR1 sequence;
      and in which:
(b) FR2 is chosen from the group consisting of the FR2 sequences present in the Nanobodies of SEQ ID NO's: 80-93 or 106-109, respectively, or from the group consisting of amino acid sequences that have at least 80%, preferably at least 90%, more preferably at least 95%, even more preferably at least 99% sequence identity (as defined herein) with one of said FR2 sequences; in which
   i) any amino acid substitution at any position other than a Hallmark position is preferably either a conservative amino acid substitution (as defined herein) and/or an amino acid substitution as defined in Table A-6; and/or
   ii) said amino acid sequence preferably only contains amino acid substitutions, and no amino acid deletions or insertions, compared to said FR2 sequence; and
   iii) the Hallmark residues at positions 37, 44, 45 and 47 are as indicated in said FR2 sequence;
   and/or from the group consisting of amino acid sequences that have 3, 2 or only 1 “amino acid difference(s)” (as defined herein) with one of said FR2 sequences, in which:
   i) any amino acid substitution at any position other than a Hallmark position is preferably either a conservative amino acid substitution (as defined herein) and/or an amino acid substitution as defined in Table A-6; and/or
   ii) said amino acid sequence preferably only contains amino acid substitutions, and no amino acid deletions or insertions, compared to said FR2 sequence; and
   iii) the Hallmark residues at positions 37, 44, 45 and 47 are as indicated in said FR2 sequence;
      and in which:
(c) FR3 is chosen from the group consisting of the FR3 sequences present in the Nanobodies of SEQ ID NO's: 80-93 or 106-109, respectively, or from the group consisting of amino acid sequences that have at least 80%, preferably at least 90%, more preferably at least 95%, even more preferably at least 99% sequence identity (as defined herein) with one of said FR3 sequences; in which
   i) any amino acid substitution at any position other than a Hallmark position is preferably either a conservative amino acid substitution (as defined herein) and/or an amino acid substitution as defined in Table A-7; and/or
   ii) said amino acid sequence preferably only contains amino acid substitutions, and no amino acid deletions or insertions, compared to said FR3 sequence; and
   iii) the Hallmark residues at positions 83 and 84 are as indicated in said FR3 sequence;
      and/or from the group consisting of amino acid sequences that have 3, 2 or only 1 “amino acid difference(s)” (as defined herein) with one of said FR3 sequences, in which:
   i) any amino acid substitution at any position other than a Hallmark position is preferably either a conservative amino acid substitution (as defined herein) and/or an amino acid substitution as defined in Table A-7; and/or
   ii) said amino acid sequence preferably only contains amino acid substitutions, and no amino acid deletions or insertions, compared to said FR3 sequence; and
   iii) the Hallmark residues at positions 83 and 84 are as indicated in said FR3 sequence;
      and in which:
(d) FR4 is chosen from the group consisting of the FR4 sequences present in the Nanobodies of SEQ ID NO's: 80-93 or 106-109, respectively, or from the group consisting of amino acid sequences that have at least 80%, preferably at least 90%, more preferably at least 95%, even more preferably at least 99% sequence identity (as defined herein) with one of said FR4 sequences; in which
   i) any amino acid substitution at any position other than a Hallmark position is preferably either a conservative amino acid substitution (as defined herein) and/or an amino acid substitution as defined in Table A-8; and/or
   ii) said amino acid sequence preferably only contains amino acid substitutions, and no amino acid deletions or insertions, compared to said FR4 sequence; and
   iii) the Hallmark residues at positions 103, 104 and 108 are as indicated in said FR3 sequence;
   and/or from the group consisting of amino acid sequences that have 3, 2 or only 1 “amino acid difference(s)” (as defined herein) with one of said FR4 sequences, in which:
   i) any amino acid substitution at any position other than a Hallmark position is preferably either a conservative amino acid substitution (as defined herein) and/or an amino acid substitution as defined in Table A-8; and/or
   ii) said amino acid sequence preferably only contains amino acid substitutions, and no amino acid deletions or insertions, compared to said FR4 sequence; and
   iii) the Hallmark residues at positions 103, 104 and 108 are as indicated in said FR4 sequence;
      and in which:
(e) CDR1, CDR2 and CDR3 are as defined herein, and are preferably as defined according to one of the preferred embodiments herein, and are more preferably as defined according to one of the more preferred embodiments herein.

Some particularly preferred Nanobodies of the invention can be chosen from the group consisting of the amino acid sequences of SEQ ID NO's: 80-93 and 106-109, respectively, or from the group consisting of amino acid sequences that have at least 80%, preferably at least 90%, more preferably at least 95%, even more preferably at least 99% sequence identity (as defined herein) with one of said amino acid sequences; in which
i) the Hallmark residues can be as indicated in Table A-3 above;
ii) any amino acid substitution at any position other than a Hallmark position is preferably either a conservative amino acid substitution (as defined herein) and/or an amino acid substitution as defined in Tables A-5-A-8; and/or
iii) said amino acid sequence preferably only contains amino acid substitutions, and no amino acid deletions or insertions, compared to the above amino acid sequence(s).

Some even more particularly preferred Nanobodies of the invention can be chosen from the group consisting of the amino acid sequences of SEQ ID NO's 80-93 or 106-109, respectively, or from the group consisting of amino acid sequences that have at least 80%, preferably at least 90%, more preferably at least 95%, even more preferably at least 99% sequence identity (as defined herein) with one of said amino acid sequences; in which
(1) the Hallmark residues are as indicated in the pertinent sequence from SEQ ID NO's 80-93 or 106-109;
(2) any amino acid substitution at any position other than a Hallmark position is preferably either a conservative amino acid substitution (as defined herein) and/or an amino acid substitution as defined in Tables A-5-A-8; and/or
(3) said amino acid sequence preferably only contains amino acid substitutions, and no amino acid deletions or insertions, compared to the pertinent sequence chosen from SEQ ID NO's 80-93 or 106-109.

Some of the most preferred Nanobodies of the invention against EGFR and IGF-IR, respectively, can be chosen from the group consisting of the amino acid sequences of SEQ ID NO's 80-93 or 106-109, respectively.

Preferably, the CDR sequences and FR sequences in the Nanobodies of the invention are such that the Nanobody of the invention binds to EGFR or IGF-IR, respectively, with an dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹ and/or with an affinity less than 500 nM, preferably less than 200 nM, more preferably less than 10 mM, such as less than 500 pM. The affinity of the Nanobody of the invention against EGFR or IGF-IR, respectively, can be determined in a manner known per se, for example using the assay described herein.

According to one non-limiting aspect of the invention, a Nanobody may be as defined herein, but with the proviso that it has at least “one amino acid difference” (as defined herein) in at least one of the framework regions compared to the corresponding framework region of a naturally occurring human VH domain, and in particular compared to the corresponding framework region of DP-47. More specifically, according to one non-limiting aspect of the invention, a Nanobody may be as defined herein, but with the proviso that it has at least “one amino acid difference” (as defined herein) at least one of the Hallmark residues (including those at positions 108, 103 and/or 45) compared to the corresponding framework region of a naturally occurring human VH domain, and in particular compared to the corresponding framework region of DP-47. Usually, a Nanobody will have at least one such amino acid difference with a naturally occurring VH domain in at least one of FR2 and/or FR4, and in particular at least one of the Hallmark residues in FR2 and/or FR4 (again, including those at positions 108, 103 and/or 45).

Also, a humanized Nanobody of the invention may be as defined herein, but with the proviso that it has at least “one amino acid difference” (as defined herein) in at least one of the framework regions compared to the corresponding framework region of a naturally occurring V_{HH} domain. More specifically, according to one non-limiting aspect of the invention, a Nanobody may be as defined herein, but with the proviso that it has at least “one amino acid difference” (as defined herein) at least one of the Hallmark residues (including those at positions 108, 103 and/or 45) compared to the corresponding framework region of a naturally occurring V_{HH} domain. Usually, a Nanobody will have at least one such amino acid difference with a naturally occurring V_{HH} domain in at least one of FR2 and/or FR4, and in particular at least one of the Hallmark residues in FR2 and/or FR4 (again, including those at positions 108, 103 and/or 45).

As will be clear from the disclosure herein, it is also within the scope of the invention to use natural or synthetic analogs, mutants, variants, alleles, homologs and orthologs (herein collectively referred to as “analogs”) of the Nanobodies of the invention as defined herein, and in particular analogs of the Nanobodies of SEQ ID NO's 80-93 or SEQ ID NO's 106-109.

Thus, according to one embodiment of the invention, the term “Nanobody of the invention” in its broadest sense also covers such analogs.

Generally, in such analogs, one or more amino acid residues may have been replaced, deleted and/or added, compared to the Nanobodies of the invention as defined herein. Such substitutions, insertions or deletions may be made in one or more of the framework regions and/or in one or more of the CDR's. When such substitutions, insertions or deletions are made in one or more of the framework regions, they may be made at one or more of the Hallmark residues and/or at one or more of the other positions in the framework residues, although substitutions, insertions or deletions at the Hallmark residues are generally less preferred (unless these are suitable humanizing substitutions as described herein).

By means of non-limiting examples, a substitution may for example be a conservative substitution (as described herein) and/or an amino acid residue may be replaced by another amino acid residue that naturally occurs at the same position in another V_{HH} domain (see Tables A-5-A-8 for some non-limiting examples of such substitutions), although the invention is generally not limited thereto. Thus, any one or more substitutions, deletions or insertions, or any combination thereof, that either improve the properties of the Nanobody of the invention or that at least do not detract too much from the desired properties or from the balance or combination of desired properties of the Nanobody of the invention (i.e. to the extent that the Nanobody is no longer suited for its intended use) are included within the scope of the invention. A skilled person will generally be able to determine and select suitable substitutions, deletions or insertions, or suitable combinations of thereof, based on the disclosure herein and optionally after a limited degree of routine experimentation, which may for example involve introducing a limited number of possible substitutions and determining their influence on the properties of the Nanobodies thus obtained.

For example, and depending on the host organism used to express the Nanobody or polypeptide of the invention, such deletions and/or substitutions may be designed in such a way that one or more sites for post-translational modification (such as one or more glycosylation sites) are removed, as will be within the ability of the person skilled in the art.

Alternatively, substitutions or insertions may be designed so as to introduce one or more sites for attachment of functional groups (as described herein), for example to allow site-specific pegylation (again as described herein).

As can be seen from the data on the V_{HH} entropy and V_{HH} variability given in Tables A-5-A-8 above, some amino acid residues in the framework regions are more conserved than others. Generally, although the invention in its broadest sense is not limited thereto, any substitutions, deletions or insertions are preferably made at positions that are less conserved. Also, generally, amino acid substitutions are preferred over amino acid deletions or insertions.

The analogs are preferably such that they can bind to EGFR or IGF-IR, respectively, with an dissociation constant (K_{D}) of 10⁻⁵ to 10⁻¹² moles/liter or less, and preferably 10⁻⁷ to 10-12 moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹ and/or with an affinity less than 500 mM, preferably less than 200 mM, more preferably less than 10 nM, such as less than 500 pM. The affinity of the analog against EGFR or IGF-IR, respectively, can be determined in a manner known per se, for example using the assay described herein.

The analogs are preferably also such that they retain the favourable properties the Nanobodies, as described herein.

Also, according to one preferred embodiment, the analogs have a degree of sequence identity of at least 70%, preferably at least 80%, more preferably at least 90%, such as at least 95% or 99% or more; and/or preferably have at most 20, preferably at most 10, even more preferably at most 5, such as 4, 3, 2 or only 1 amino acid difference (as defined herein), with one of the Nanobodies of SEQ ID NOs 80-93 or SEQ ID NO's 106-109.

Also, the framework sequences and CDR's of the analogs are preferably such that they are in accordance with the preferred embodiments defined herein. More generally, as described herein, the analogs will have (a) a Q at position 108; and/or (b) a charged amino acid or a cysteine residue at position 45 and preferably an E at position 44, and more preferably E at position 44 and R at position 45; and/or (c) P, R or S at position 103.

One preferred class of analogs of the Nanobodies of the invention comprise Nanobodies that have been humanized (i.e. compared to the sequence of a naturally occurring Nanobody of the invention). As mentioned in the background art cited herein, such humanization generally involves replacing one or more amino acid residues in the sequence of a naturally occurring V_{HH} with the amino acid residues that occur at the same position in a human V_{H} domain, such as a human V_{H}3 domain. Examples of possible humanizing substitutions or combinations of humanizing substitutions will be clear to the skilled person, for example from the Tables herein, from the possible humanizing substitutions mentioned in the background art cited herein, and/or from a comparision between the sequence of a Nanobody and the sequence of a naturally occurring human V_{H} domain.

The humanizing substitutions should be chosen such that the resulting humanized Nanobodies still retain the favourable properties of Nanobodies as defined herein, and more preferably such that they are as described for analogs in the preceding paragraphs. A skilled person will generally be able to determine and select suitable humanizing substitutions or suitable combinations of humanizing substitutions, based on the disclosure herein and optionally after a limited degree of routine experimentation, which may for example involve introducing a limited number of possible humanizing substitutions and determining their influence on the properties of the Nanobodies thus obtained.

Generally, as a result of humanization, the Nanobodies of the invention may become more “human-like”, while still retaining the favorable properties of the Nanobodies of the invention as described herein. As a result, such humanized Nanobodies may have several advantages, such as a reduced immunogenicity, compared to the corresponding naturally occurring V_{HH} domains. Again, based on the disclosure herein and optionally after a limited degree of routine experimentation, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring V_{HH} domains on the other hand.

The humanized and other analogs, and nucleic acid sequences encoding the same, can be provided in any manner known per se. For example, the analogs can be obtained by providing a nucleic acid that encodes a naturally occurring V_{HH} domain, changing the codons for the one or more amino acid residues that are to be substituted into the codons for the corresponding desired amino acid residues (e.g. by site-directed mutagenesis or by PCR using suitable mismatch primers), expressing the nucleic acid/nucleotide sequence thus obtained in a suitable host or expression system; and optionally isolating and/or purifying the analog thus obtained to provide said analog in essentially isolated form (e.g. as further described herein).

This can generally be performed using methods and techniques known per se, which will be clear to the skilled person, for example from the handbooks and references cited herein, the background art cited herein and/or from the further description herein. Alternatively, a nucleic acid encoding the desired analog can be synthesized in a manner known per se (for example using an automated apparatus for synthesizing nucleic acid sequences with a predefined amino acid sequence) and can then be expressed as described herein. Yet another technique may involve combining one or more naturally occurring and/or synthetic nucleic acid sequences each encoding a part of the desired analog, and then expressing the combined nucleic acid sequence as described herein. Also, the analogs can be provided using chemical synthesis of the pertinent amino acid sequence using techniques for peptide synthesis known per se, such as those mentioned herein.

In this respect, it will be also be clear to the skilled person that the Nanobodies of the invention (including their analogs) can be designed and/or prepared starting from human V_{H} sequences (i.e. amino acid sequences or the corresponding nucleotide sequences), such as for example from human V_{H}3 sequences such as DP-47, DP-51 or DP-29, i.e. by introducing one or more camelizing substitutions (i.e. changing one or more amino acid residues in the amino acid sequence of said human V_{H} domain into the amino acid residues that occur at the corresponding position in a V_{HH} domain), so as to provide the sequence of a Nanobody of the invention and/or so as to confer the favourable properties of a Nanobody to the sequence thus obtained. Again, this can generally be performed using the various methods and techniques referred to in the previous paragraph, using an amino acid sequence and/or nucleotide sequence for a human V_{H} domain as a starting point.

Some preferred, but non-limiting camelizing substitutions can be derived from Tables A-5-A-8. It will also be clear that camelizing substitutions at one or more of the Hallmark residues will generally have a greater influence on the desired properties than substitutions at one or more of the other amino acid positions, although both and any suitable combination thereof are included within the scope of the invention. For example, it is possible to introduce one or more camelizing substitutions that already confer at least some the desired properties, and then to introduce further camelizing substitutions that either further improve said properties and/or confer additional favourable properties. Again, the skilled person will generally be able to determine and select suitable camelizing substitutions or suitable combinations of camelizing substitutions, based on the disclosure herein and optionally after a limited degree of routine experimentation, which may for example involve introducing a limited number of possible camelizing substitutions and determining whether the favourable properties of Nanobodies are obtained or improved (i.e. compared to the original V_{H} domain).

Generally, however, such camelizing substitutions are preferably such that the resulting amino acid sequence at least contains (a) a Q at position 108; and/or (b) a charged amino acid or a cysteine residue at position 45 and preferably also an E at position 44, and more preferably E at position 44 and R at position 45; and/or (c) P, R or S at position 103; and optionally one or more further camelizing substitutions. More preferably, the camelizing substitutions are such that they result in a Nanobody of the invention and/or in an analog thereof (as defined herein), such as in a humanized analog and/or preferably in an analog that is as defined in the preceding paragraphs.

As will also be clear from the disclosure herein, it is also within the scope of the invention to use parts or fragments, or combinations of two or more parts or fragments, of the Nanobodies of the invention as defined herein, and in particular parts or fragments of the Nanobodies of SEQ ID NO's 80-93 or SEQ ID NO's 106-109. Thus, according to one embodiment of the invention, the term “Nanobody of the invention” in its broadest sense also covers such parts or fragments.

Generally, such parts or fragments of the Nanobodies of the invention (including analogs thereof) have amino acid sequences in which, compared to the amino acid sequence of the corresponding full length Nanobody of the invention (or analog thereof), one or more of the amino acid residues at the N-terminal end, one or more amino acid residues at the C-terminal end, one or more contiguous internal amino acid residues, or any combination thereof, have been deleted and/or removed.

The parts or fragments are preferably such that they can bind to EGFR or IGF-IR, respectively, with an dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹ and/or with an affinity less than 500 nM, preferably less than 200 nM, more preferably less than 10 nM, such as less than 500 pM. The affinity of the parts or fragments against EGFR or IGF-IR, respectively, can be determined in a manner known per se, for example using the assay described herein.

Any part or fragment is preferably such that it comprises at least 10 contiguous amino acid residues, preferably at least 20 contiguous amino acid residues, more preferably at least 30 contiguous amino acid residues, such as at least 40 contiguous amino acid residues, of the amino acid sequence of the corresponding full length Nanobody of the invention.

Also, any part or fragment is such preferably that it comprises at least one of CDR1, CDR2 and/or CDR3 or at least part thereof (and in particular at least CDR3 or at least part thereof). More preferably, any part or fragment is such that it comprises at least one of the CDR's (and preferably at least CDR3 or part thereof) and at least one other CDR (i.e. CDR1 or CDR2) or at least part thereof, preferably connected by suitable framework sequence(s) or at least part thereof. More preferably, any part or fragment is such that it comprises at least one of the CDR's (and preferably at least CDR3 or part thereof) and at least part of the two remaining CDR's, again preferably connected by suitable framework sequence(s) or at least part thereof.

According to another particularly preferred, but non-limiting embodiment, such a part or fragment comprises at least CDR3, such as FR3, CDR3 and FR4 of the corresponding full length Nanobody of the invention, i.e. as for example described in the International application WO 03/050531 (Lasters et al.).

As already mentioned above, it is also possible to combine two or more of such parts or fragments (i.e. from the same or different Nanobodies of the invention), i.e. to provide an analog (as defined herein) and/or to provide further parts or fragments (as defined herein) of a Nanobody of the invention. It is for example also possible to combine one or more parts or fragments of a Nanobody of the invention with one or more parts or fragments of a human V_{H} domain.

According to one preferred embodiment, the parts or fragments have a degree of sequence identity of at least 50%, preferably at least 60%, more preferably at least 70%, even more preferably at least 80%, such as at least 90%, 95% or 99% or more with one of the Nanobodies of SEQ ID NOs 80-93 or SEQ ID NO's 106-109

The parts and fragments, and nucleic acid sequences encoding the same, can be provided and optionally combined in any manner known per se. For example, such parts or fragments can be obtained by inserting a stop codon in a nucleic acid that encodes a full-sized Nanobody of the invention, and then expressing the nucleic acid thus obtained in a manner known per se (e.g. as described herein). Alternatively, nucleic acids encoding such parts or fragments can be obtained by suitably restricting a nucleic acid that encodes a full-sized Nanobody of the invention or by synthesizing such a nucleic acid in a manner known per se. Parts or fragments may also be provided using techniques for peptide synthesis known per se.

The invention in its broadest sense also comprises derivatives of the Nanobodies of the invention. Such derivatives can generally be obtained by modification, and in particular by chemical and/or biological (e.g enzymatical) modification, of the Nanobodies of the invention and/or of one or more of the amino acid residues that form the Nanobodies of the invention.

Examples of such modifications, as well as examples of amino acid residues within the Nanobody sequence that can be modified in such a manner (i.e. either on the protein backbone but preferably on a side chain), methods and techniques that can be used to introduce such modifications and the potential uses and advantages of such modifications will be clear to the skilled person.

For example, such a modification may involve the introduction (e.g. by covalent linking or in an other suitable manner) of one or more functional groups, residues or moieties into or onto the Nanobody of the invention, and in particular of one or more functional groups, residues or moieties that confer one or more desired properties or functionalities to the Nanobody of the invention. Example of such functional groups will be clear to the skilled person.

For example, such modification may comprise the introduction (e.g. by covalent binding or in any other suitable manner) of one or more functional groups that increase the half-life, the solubility and/or the absorption of the Nanobody of the invention, that reduce the immunogenicity and/or the toxicity of the Nanobody of the invention, that eliminate or attenuate any undesirable side effects of the Nanobody of the invention, and/or that confer other advantageous properties to and/or reduce the undesired properties of the Nanobodies and/or polypeptides of the invention; or any combination of two or more of the foregoing. Examples of such functional groups and of techniques for introducing them will be clear to the skilled person, and can generally comprise all functional groups and techniques mentioned in the general background art cited hereinabove as well as the functional groups and techniques known per se for the modification of pharmaceutical proteins, and in particular for the modificafion of antibodies or antibody fragments (including ScFv's and single domain antibodies), for which reference is for example made to Remington's Pharmaceutical Sciences, 16th ed., Mack Publishing Co., Easton, Pa. (1980). Such functional groups may for example be linked directly (for example covalently) to a Nanobody of the invention, or optionally via a suitable linker or spacer, as will again be clear to the skilled person.

One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Generally, any suitable form of pegylation can be used, such as the pegylation used in the art for antibodies and antibody fragments (including but not limited to (single) domain antibodies and ScFv's); reference is made to for example Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO 04/060965. Various reagents for pegylation of proteins are also commercially available, for example from Nektar Therapeutics, USA.

Preferably, site-directed pegylation is used, in particular via a cysteine-residue (see for example Yang et al., Protein Engineering, 16, 10, 761-770 (2003). For example, for this purpose, PEG may be attached to a cysteine residue that naturally occurs in a Nanobody of the invention, a Nanobody of the invention may be modified so as to suitably introduce one or more cysteine residues for attachment of PEG, or an amino acid sequence comprising one or more cysteine residues for attachment of PEG may be fused to the N- and/or C-terminus of a Nanobody of the invention, all using techniques of protein engineering known per se to the skilled person.

Preferably, for the Nanobodies and proteins of the invention, a PEG is used with a molecular weight of more than 5000, such as more than 10,000 and less than 200,000, such as less than 100,000; for example in the range of 20,000-80,000.

Another, usually less preferred modification comprises N-linked or O-linked glycosylation, usually as part of co-translational and/or post-translational modification, depending on the host cell used for expressing the Nanobody or polypeptide of the invention.

Yet another modification may comprise the introduction of one or more detectable labels or other signal-generating groups or moieties, depending on the intended use of the labelled Nanobody. Suitable labels and techniques for attaching, using and detecting them will be clear to the skilled person, and for example include, but are not limited to, fluorescent labels (such as fluorescein, isothiocyanate, rhodamine, phycoerythrin, phycocyanin, allophycocyanin, o-phthaldehyde, and fluorescamine and fluorescent metals such as ¹⁵²Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs ), radio-isotopes (such as ³H, ¹²⁵I, ³²P, ³⁵S, ¹⁴C, ⁵¹Cr, ³⁶Cl, ⁵⁷Co, ⁵⁸Co, ⁵⁹Fe, and ⁷⁵Se), metals, metals chelates or metallic cations (for example metallic cations such as ^{99m}Tc, ¹²³I, ¹¹¹In, ¹³¹I, ⁹⁷Ru, ⁶⁷Cu, ⁶⁷Ga, and ⁶⁸Ga or other metals or metallic cations that are particularly suited for use in in vivo, in vitro or in situ diagnosis and imaging, such as (¹⁵⁷Gd, ⁵⁵M, ¹⁶²Dy, ⁵²Cr, and ⁵⁶Fe), as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, β-galactosidase, ribonuclease, urease, catalase, glucose-VI-phosphate dehydrogenase, glucoamylase and acetylcholine esterase). Other suitable labels will be clear to the skilled person, and for example include moieties that can be detected using NMR or ESR spectroscopy.

Such labelled Nanobodies and polypeptides of the invention may for example be used for in vitro, in vivo or in situ assays (including immunoassays known per se such as ELISA, RIA, EIA and other “sandwich assays”, etc.) as well as in vivo diagnostic and imaging purposes, depending on the choice of the specific label.

As will be clear to the skilled person, another modification may involve the introduction of a chelating group, for example to chelate one of the metals or metallic cations referred to above. Suitable chelating groups for example include, without limitation, diethyl-enetriaminepentaacetic acid (DTPA) or ethylenediaminetetraacetic acid (EDTA).

Yet another modification may comprise the introduction of a functional group that is one part of a specific binding pair, such as the biotin-(strept)avidin binding pair. Such a functional group may be used to link the Nanobody of the invention to another protein, polypeptide or chemical compound that is bound to the other half of the binding pair, i.e. through formation of the binding pair. For example, a Nanobody of the invention may be conjugated to biotin, and linked to another protein, polypeptide, compound or carrier conjugated to avidin or streptavidin. For example, such a conjugated Nanobody may be used as a reporter, for example in a diagnostic system where a detectable signal-producing agent is conjugated to avidin or streptavidin. Such binding pairs may for example also be used to bind the Nanobody of the invention to a carrier, including carriers suitable for pharmaceutical purposes. One non-limiting example are the liposomal formulations described by Cao and Suresh, Journal of Drug Targetting, 8, 4, 257 (2000). Such binding pairs may also be used to link a therapeutically active agent to the Nanobody of the invention.

For some applications, in particular for those applications in which it is intended to kill a cell that expresses the target against which the Nanobodies of the invention are directed (e.g. in the treatment of cancer), or to reduce or slow the growth and/or proliferation such a cell, the Nanobodies of the invention may also be linked to a toxin or to a toxic residue or moiety.

Examples of toxic moieties, compounds or residues which can be linked to a Nanobody of the invention to provide—for example—a cytotoxic compound will be clear to the skilled person and can for example be found in the prior art cited above and/or in the further description herein. One example is the so-called ADEPT™ technology WO 03/055527.

Other potential chemical and enzymatical modifications will be clear to the skilled person. Such modifications may also be introduced for research purposes (e.g. to study function-activity relationships). Reference is for example made to Lundblad and Bradshaw, Biotechnol. Appl. Biochem., 26, 143-151 (1997).

Preferably, the derivatives are such that they bind to EGFR or IGF-IR, respectively, with an dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹ and/or with an affinity less than 500 pM, preferably less than 200 nM, more preferably less than 10 nM, such as less than 500 pM. The affinity of a derivative of a Nanobody of the invention against EGFR or IGF-IR, respectively, can be determined in a manner known per se, for example using the assay described herein.

As mentioned above, the invention also relates to proteins or polypeptides that essentially consist of or comprise at least one Nanobody of the invention. By “essentially consist of” is meant that the amino acid sequence of the polypeptide of the invention either is exactly the same as the amino acid sequence of a Nanobody of the invention or corresponds to the amino acid sequence of a Nanobody of the invention which has a limited number of amino acid residues, such as 1-20 amino acid residues, for example 1-10 amino acid residues and preferably 1-6 amino acid residues, such as 1, 2, 3, 4, 5 or 6 amino acid residues, added at the amino terminal end, at the carboxy terminal end, or at both the amino terminal end and the carboxy terminal end of the amino acid sequence of the Nanobody.

Said amino acid residues may or may not change, alter or otherwise influence the (biological) properties of the Nanobody and may or may not add further functionality to the Nanobody. For example, such amino acid residues:
a) can comprise an N-terminal Met residue, for example as result of expression in a heterologous host cell or host organism.
b) may form a signal sequence or leader sequence that directs secretion of the Nanobody from a host cell upon synthesis. Suitable secretory leader peptides will be clear to the skilled person, and may be as further described herein. Usually, such a leader sequence will be linked to the N-terminus of the Nanobody, although the invention in its broadest sense is not limited thereto;
c) may form a sequence or signal that allows the Nanobody to be directed towards and/or to penetrate or enter into specific organs, tissues, cells, or parts or compartments of cells, and/or that allows the Nanobody to penetrate or cross a biological barrier such as a cell membrane, a cell layer such as a layer of epithelial cells, a tumor including solid tumors, or the blood-brain-barrier. Examples of such amino acid sequences will be clear to the skilled person. Some non-limiting examples are the small peptide vectors (“Pep-trans vectors”) described in WO 03/026700 and in Temsamani et al., Expert Opin. Biol. Ther., 1, 773 (2001); Temsamani and Vidal, Drug Discov. Today, 9, 1012 (004) and Rousselle, J. Pharmacol. Exp. Ther., 296, 124-131 (2001), and the membrane translocator sequence described by Zhao et al., Apoptosis, 8, 631-637 (2003). C-terminal and N-terminal amino acid sequences for intracellular targeting of antibody fragments are for example described by Cardinale et al., Methods, 34, 171 (2004). Other suitable techniques for intracellular targeting involve the expression and/or use of so-called “intrabodies” comprising a Nanobody of the invention, as mentioned below;
d) may form a “tag”, for example an amino acid sequence or residue that allows or facilitates the purification of the Nanobody, for example using affinity techniques directed against said sequence or residue. Thereafter, said sequence or residue may be removed (e.g. by chemical or enzymatical cleavage) to provide the Nanobody-sequence (for this purpose, the tag may optionally be linked to the Nanobody sequence via a cleavable linker sequence or contain a cleavable motif). Some preferred, but non-limiting examples of such residues are multiple histidine residues, glutatione residues and a myc-tag such as AAAEQKLISEEDLNGAA [SEQ ID NO:31];
e) may be one or more amino acid residues that have been functionalized and/or that can serve as a site for attachment of functional groups. Suitable amino acid residues and functional groups will be clear to the skilled person and include, but are not limited to, the amino acid residues and functional groups mentioned herein for the derivatives of the Nanobodies of the invention.

According to another embodiment, a polypeptide of the invention comprises a Nanobody of the invention, which is fused at its amino terminal end, at its carboxy terminal end, or both at its amino terminal end and at its carboxy terminal end to at least one further amino acid sequence, i.e. so as to provide a fusion protein comprising said Nanobody of the invention and the one or more further amino acid sequences. Such a fusion will also be referred to herein as a “Nanobody fusion”.

The one or more further amino acid sequence may be any suitable and/or desired amino acid sequences. The further amino acid sequences may or may not change, alter or otherwise influence the (biological) properties of the Nanobody, and may or may not add further functionality to the Nanobody or the polypeptide of the invention. Preferably, the further amino acid sequence is such that it confers one or more desired properties or functionalities to the Nanobody or the polypeptide of the invention.

Example of such amino acid sequences will be clear to the skilled person, and may generally comprise all amino acid sequences that are used in peptide fusions based on conventional antibodies and fragments thereof (including but not limited to ScFv's and single domain antibodies). Reference is for example made to the review by Holliger and Hudson, Nature Biotechnology, 23, 9, 1126-1136 (2005),

For example, such an amino acid sequence may be an amino acid sequence that increases the half-life, the solubility, or the absorption, reduces the immunogenicity or the toxicity, eliminates or attenuates undesirable side effects, and/or confers other advantageous properties to and/or reduces the undesired properties of the polypeptides of the invention, compared to the Nanobody of the invention per se. Some non-limiting examples of such amino acid sequences are serum proteins, such as human serum albumin (see for example WO 00/27435) or haptenic molecules (for example haptens that are recognized by circulating antibodies, see for example WO 98/22141).

The further amino acid sequence may also provide a second binding site, which binding site may be directed against any desired protein, polypeptide, antigen, antigenic determinant or epitope (including but not limited to the same protein, polypeptide, antigen, antigenic determinant or epitope against which the Nanobody of the invention is directed, or a different protein, polypeptide, antigen, antigenic determinant or epitope). For example, the further amino acid sequence may provide a second binding site that is directed against a serum protein (such as, for example, human serum albumin or another serum protein such as IgG), so as to provide increased half-life in serum. Reference is for example made to EP 0 368 684, WO 91/01743, WO 01/45746 and WO 04/003019 (in which various serum proteins are mentioned), WO 06/040153, as well as to Harmsen et al., Vaccine, 23 (41); 4926-42.

According to another embodiment, the one or more further amino acid sequences may comprise one or more parts, fragments or domains of conventional 4-chain antibodies (and in particular human antibodies) and/or of heavy chain antibodies. For example, although usually less preferred, a Nanobody of the invention may be linked to a conventional (preferably human) V_{H} or V_{L} domain domain or to a natural or synthetic analog of a V_{H} or V_{L} domain, again optionally via a linker sequence (including but not limited to other (single) domain antibodies, such as the dAb's described by Ward et al.).

The at least one Nanobody may also be linked to one or more (preferably human) CH₁, CH₂ and/or CH₃ domains, optionally via a linker sequence. For instance, a Nanobody linked to a suitable CH₁ domain could for example be used—together with suitable light chains—to generate antibody fragments/structures analogous to conventional Fab fragments or F(ab′)2 fragments, but in which one or (in case of an F(ab′)2 fragment) one or both of the conventional V_{H} domains have been replaced by a Nanobody of the invention. Also, two Nanobodies could be linked to a CH3 domain (optionally via a linker) to provide a construct with increased half-life in vivo.

According to one specific embodiment of a polypeptide of the invention, one or more Nanobodies of the invention may linked to one or more antibody parts, fragments or domains that confer one or more effector functions to the polypeptide of the invention and/or may confer the ability to bind to one or more Fc receptors. For example, for this purpose, and without being limited thereto, the one or more further amino acid sequences may comprise one or more CH₂ and/or CH₃ domains of an antibody, such as from a heavy chain antibody (as described herein) and more preferably from a conventional human 4-chain antibody; and/or may form (part of) and Fc region, for example from IgG, from IgE or from another human Ig. For example, WO 94/04678 describes heavy chain antibodies comprising a Camelid V_{HH} domain or a humanized derivative thereof (i.e. a Nanobody), in which the Camelidae CH₂ and/or CH₃ domain have been replaced by human CH₂ and CH₃ domains, so as to provide an immunoglobulin that consists of 2 heavy chains each comprising a Nanobody and human CH2 and CH3 domains (but no CH1 domain), which immunoglobulin has the effector function provided by the CH2 and CH3 domains and which immunoglobulin can function without the presence of any light chains. Other amino acid sequences that can be suitably linked to the Nanobodies of the invention so as to provide an effector function will be clear to the skilled person, and may be chosen on the basis of the desired effector function(s). Reference is for example made to WO 04/058820, WO 99/42077 and WO 05/017148, as well as the review by Holliger and Hudson, supra. Coupling of a Nanobody of the invention to an Fc portion may also lead to an increased half-life, compared to the corresponding Nanobody of the invention. For some applications, the use of an Fc portion and/or of constant domains (i.e. CH₂ and/or CH₃ domains) that confer increased half-life without any biologically significant effector function may also be suitable or even preferred. Other suitable constructs comprising one or more Nanobodies and one or more constant domains with increased half-life in vivo will be clear to the skilled person, and may for example comprise two Nanobodies linked to a CH3 domain, optionally via a linker sequence. Generally, any fusion protein or derivatives with increased half-life will preferably have a molecular weight of more than 50 kD, the cut-off value for renal absorption.

The further amino acid sequences may also form a signal sequence or leader sequence that directs secretion of the Nanobody or the polypeptide of the invention from a host cell upon synthesis (for example to provide a pre-, pro- or prepro- form of the polypeptide of the invention, depending on the host cell used to express the polypeptide of the invention).

The further amino acid sequence may also form a sequence or signal that allows the Nanobody or polypeptide of the invention to be directed towards and/or to penetrate or enter into specific organs, tissues, cells, or parts or compartments of cells, and/or that allows the Nanobody or polypeptide of the invention to penetrate or cross a biological barrier such as a cell membrane, a cell layer such as a layer of epithelial cells, a tumor including solid tumors, or the blood-brain-barrier. Suitable examples of such amino acid sequences will be clear to the skilled person, and for example include, but are not limited to, the “Peptrans” vectors mentioned above, the sequences described by Cardinale et al. and the amino acid sequences and antibody fragments known per se that can be used to express or produce the Nanobodies and polypeptides of the invention as so-called “intrabodies”, for example as described in WO 94/02610, WO 95/22618, U.S. Pat. No. 6,004,940, WO 03/014960, WO 99/07414; WO-05/01690; EP 1 512 696; and in Cattaneo, A. & Biocca, S. (1997) Intracellular Antibodies: Development and Applications. Landes and Springer-Verlag; and in Kontermann, Methods 34, (2004), 163-170, and the further references described therein.

For some applications, in particular for those applications in which it is intended to kill a cell that expresses the target against which the Nanobodies of the invention are directed (e.g. in the treatment of cancer), or to reduce or slow the growth and/or proliferation such a cell, the Nanobodies of the invention may also be linked to a (cyto)toxic protein or polypeptide. Examples of such toxic proteins and polypeptides which can be linked to a Nanobody of the invention to provide—for example—a cytotoxic polypeptide of the invention will be clear to the skilled person and can for example be found in the prior art cited above and/or in the further description herein. One example is the so-called ADEPT™ technology WO 03/055527.

According to one preferred, but non-limiting embodiment, said one or more further amino acid sequences comprise at least one further Nanobody, so as to provide a polypeptide of the invention that comprises at least two, such as three, four, five or more Nanobodies, in which said Nanobodies may optionally be linked via one or more linker sequences (as defined herein). Polypeptides of the invention that comprise two or more Nanobodies, of which at least one is a Nanobody of the invention, will also be referred to herein as “multivalent” polypeptides of the invention, and the Nanobodies present in such polypeptides will also be referred to herein as being in a “multivalent format”. For example a “bivalent” polypeptide of the invention comprises two Nanobodies, optionally linked via a linker sequence, whereas a “trivalent” polypeptide of the invention comprises three Nanobodies, optionally linked via two linker sequences; etc.; in which at least one of the Nanobodies present in the polypeptide, and up to all of the Nanobodies present in the polypeptide, is/are a Nanobody of the invention.

In a multivalent polypeptide of the invention, the two or more Nanobodies may be the same or different, and may be directed against the same antigen or antigenic determinant (for example against the same part(s) or epitope(s) or against different parts or epitopes) or may alternatively be directed against different antigens or antigenic determinants; or any suitable combination thereof. For example, a bivalent polypeptide of the invention may comprise (a) two identical Nanobodies; (b) a first Nanobody directed against a first antigenic determinant of a protein or antigen and a second Nanobody directed against the same antigenic determinant of said protein or antigen which is different from the first Nanobody; (c) a first Nanobody directed against a first antigenic determinant of a protein or antigen and a second Nanobody directed against another antigenic determinant of said protein or antigen; or (d) a first Nanobody directed against a first protein or antigen and a second Nanobody directed against a second protein or antigen (i.e. different from said first antigen). Similarly, a trivalent polypeptide of the invention may, for example and without being limited thereto. comprise (a) three identical Nanobodies; (b) two identical Nanobody against a first antigenic determinant of an antigen and a third Nanobody directed against a different antigenic determinant of the same antigen; (c) two identical Nanobody against a first antigenic determinant of an antigen and a third Nanobody directed against a second antigen different from said first antigen; (d) a first Nanobody directed against a first antigenic determinant of a first antigen, a second Nanobody directed against a second antigenic determinant of said first antigen and a third Nanobody directed against a second antigen different from said first antigen; or (e) a first Nanobody directed against a first antigen, a second Nanobody directed against a second antigen different from said first antigen, and a third Nanobody directed against a third antigen different from said first and second antigen.

In a preferred embodiment, the invention provides a bivalent polypeptide comprising or essentially consisting of two identical Nanobody against EFGR. Non-limiting examples of such bivalent polypeptides are provided in SEQ ID NO's: 122-123. In another preferred embodiment, the invention provides a bivalent polypeptide comprising or essentially consisting of two identical Nanobodies against IGF-IR. Non-limiting examples of such bivalent polypeptides are provided in SEQ ID NO's: 134-135.

Polypeptides of the invention that contain at least two Nanobodies, in which at least one Nanobody is directed against a first antigen (i.e. against EGFR or IGF-IR, respectively) and at least one Nanobody is directed against a second antigen (i.e. different from EGFR or IGF-IR, respectively), will also be referred to as “multispecific” polypeptides of the invention, and the Nanobodies present in such polypeptides will also be referred to herein as being in a “multivalent format”. Thus, for example, a “bispecific” polypeptide of the invention is a polypeptide that comprises at least one Nanobody directed against a first antigen (i.e. EGFR or IGF-IR, respectively) and at least one further Nanobody directed against a second antigen (i.e. different from EGFR or IGF-IR, respectively), whereas a “trispecific” polypeptide of the invention is a polypeptide that comprises at least one Nanobody directed against a first antigen (i.e. EGFR or IGF-IR, respectively), at least one further Nanobody directed against a second antigen (i.e. different from EGFR or IGF-IR, respectively) and at least one further Nanobody directed against a third antigen (i.e. different from both EGFR or IGF-IR, respectively, and the second antigen); etc.

Accordingly, in its simplest form, a bispecific polypeptide of the invention is a bivalent polypeptide of the invention (as defined herein), comprising a first Nanobody directed against EGFR or IGF-IR, respectively, and a second Nanobody directed against a second antigen, in which said first and second Nanobody may optionally be linked via a linker sequence (as defined herein); whereas a trispecific polypeptide of the invention in its simplest form is a trivalent polypeptide of the invention (as defined herein), comprising a first Nanobody directed against EGFR or IGF-IR, respectively, a second Nanobody directed against a second antigen and a third Nanobody directed against a third antigen, in which said first, second and third Nanobody may optionally be linked via one or more, and in particular one and more in particular two, linker sequences.

However, as will be clear from the description hereinabove, the invention is not limited thereto, in the sense that a multispecific polypeptide of the invention may comprise at least one Nanobody against EGFR or IGF-IR, respectively, and any number of Nanobodies directed against one or more antigens different from EGFR or IGF-IR, respectively.

According to one specific, but non-limiting embodiment, a polypeptide as described herein comprises at least one Nanobody against EGFR and at least one Nanobody against IGF-IR, optionally linked using one or more suitable linkers. In such a bispecific Nanobody construct, the Nanobodies and polypeptides against IGF-IR described herein can be combined with one or more of the anti-EGFR Nanobodies and polypeptides described in WO 05/044858 and WO 04/041867, and/or with one or more of the anti-EGFR Nanobodies and polypeptides described herein. Non-limiting examples of such bispecific Nanobody constructs include SEQ ID NOs: 136-140.

Bispecific polypeptides that comprise two binding moieties, wherein each binding moiety is specific for a tumor associated antigen (i.e. an antigen expressed on a tumor cell, also called ‘tumor marker’), are highly advantageous in tumor targeting. Such bispecific polypeptides are capable of simultaneously targeting two tumor associated antigens, resulting in enhanced tumor specificity. It is known that most tumor markers are not truly tumor specific but also occur (mostly at lower levels) on normal tissues or cells. Monospecific binding moieties, Nanobodies or polypeptides against only one tumor marker will therefore also recognize those normal tissues or cells resulting in a non-specific cell arrest or killing. Polypeptides that are specific for two or more markers on one or more tumor cells will be much more tumor specific and provide a better specific binding. They can thus block simultaneously multiple receptor activation and downstream signal transduction pathways, and provide a better inhibition of tumor proliferation and arrest or—killing of the tumor cells.

Accordingly, the present invention also relates to a bispecific or multispecific polypeptide, comprising or essentially consisting of at least two binding moieties, wherein each of said at least two binding moieties is directed against a tumor associated antigen or epitope. In an embodiment, each binding moiety is directed against a different epitope on the same tumor associated antigen (also called biparatopic binding moieties). In another embodiment, each binding moiety is directed against a different tumor associated antigen.

Each binding moiety can be directed against a different tumor associated antigen on either a single or adjacent tumor cell. In a preferred embodiment, said at least two binding moieties have a moderate or low affinity to their individual tumor associated antigen or epitope and, accordingly, have only a reduced retention on normal tissues or cells expressing one of the tumor associated antigens. Those at least two binding moieties, however preferentially target (have a high avidity for) tumor cells that express both antigens recognized by the bispecific or multispecific polypeptide.

A binding moiety according to the present invention can be any peptide or nucleotide containing moiety having a known binding affinity for at least one antigen. The moiety can be a protein, a polypeptide, a protein fragment (such as an antibody fragment) or one or more subunit(s) of any protein. A typical example of a binding moiety would be an enzyme, a receptor or a transport protein. It can also be a carrier protein such as albumin or an antibody. The binding moiety can also be, or include, a sequence of DNA or RNA.

In a preferred embodiment, one or more of the binding moieties on the bispecific or multispecific polypeptide of the invention is a polypeptide below 15 kDa. In an even more preferred embodiment, one or more of the binding moieties on the bispecific or multispecific polypeptide of the invention is a VH, a VHH, a domain antibody, a single domain antibody, a “dAb” or a Nanobody. In a most preferred embodiment, the binding moieties on the bispecific or multispecific polypeptide of the invention are Nanobodies.

Different EGFR family members (EGFR, HER2, HER3, HER4), for example, are overexpressed on tumors in breast cancer, colon cancer, ovarian cancer, lung cancer and head and neck cancer. By simultaneous targeting two of these tumor associated antigens, or different epitopes on one of these tumor associated antigens, a much more selective and/or enhanced tumor targeting will be obtained.

Therefore, in a preferred embodiment, the invention also provides a bispecific polypeptide comprising or essentially consisting of a Nanobody directed against EGFR and a Nanobody directed against another member of the EGFR family. The polypeptide of the invention may comprise or essentially consist of a Nanobody directed against EGFR and a Nanobody directed against HER2. In another embodiment, the polypeptide of the invention may comprise or essentially consist of a Nanobody directed against EGFR and a Nanobody directed against HER3. In another embodiment, the polypeptide of the invention may comprise or essentially consist of a Nanobody directed against EGFR and a Nanobody directed against HER4. In a further embodiment, the polypeptide may also comprise or essentially consist of a Nanobody directed against HER2 and a Nanobody directed against another member of the EGFR family. The polypeptide of the invention may comprise or essentially consist of a Nanobody directed against HER2 and a Nanobody directed against HER3. In another embodiment, the polypeptide of the invention may comprise or essentially consist of a Nanobody directed against HER2 and a Nanobody directed against HER4. In another further embodiment, the polypeptide may also comprise or essentially consist of a Nanobody directed against HER3 and a Nanobody directed against another member of the EGFR family. The polypeptide of the invention may comprise or essentially consist of a Nanobody directed against HER3 and a Nanobody directed against HER4.

In another preferred embodiment, the invention provides a bispecific polypeptide comprising or essentially consisting of a Nanobody directed against a specific epitope of EGFR and a Nanobody directed against another specific epitope of EGFR. Non-limiting examples of such bispecific/biparatopic polypeptides are SEQ ID NOs: 141-143.

Another non-limiting example is the CD138 and CD38 tumor markers expressed on multiple myeloma cells. By simultaneous targeting CD138 and CD38 a much more selective targeting of those multiple myeloma cells is obtained.

Tumor markers that can be simultaneously targeted via the bispecific or multispecific polypeptides of the invention include—but are not limited to—EGFR, IGF-IR, HER2, HER3, HER4, CEA, VEGF, CD38, CD138.

In another preferred embodiment, the invention relates to a trispecific or multispecific polypeptide, comprising or essentially consisting of at least three binding moieties, wherein two of said at least three binding moieties are directed against a tumor associated antigen or epitope and the other binding moiety is directed against another target or antigen. Preferably this target or antigen is a molecule which can increase the half-life of the polypeptide in vivo (as further described) or a molecule with an effector function such as CD3, the Fc receptor or a complement protein. In an embodiment, the two binding moieties directed against a tumor associated antigen or epitope are directed against a different epitope on the same tumor associated antigen (also called biparatopic binding moieties). In another embodiment, the two binding moiety directed against a tumor associated antigen or epitope are directed against a different tumor associated antigen. Each of these binding moiety can be directed against a different tumor associated antigen on either a single or adjacent tumor cell.

In a preferred embodiment, one or more of the binding moieties on the trispecific or multispecific polypeptide of the invention is a polypeptide below 15 kDa. In an even more preferred embodiment, one or more of the binding moieties on the trispecific or multispecific polypeptide of the invention is a VH, a VHH, a domain antibody, a single domain antibody, a “dAb” or a Nanobody. In a most preferred embodiment, the binding moieties on the trispecific or multispecific polypeptide of the invention are Nanobodies.

In an embodiment, the invention provides trispecific polypeptides comprising or essentially consisting of a Nanobody against EGFR, a Nanobody against IGF-IR and a Nanobody against human serum albumin, such as non-limiting examples SEQ ID NO's: 138-140. In another embodiment, the invention provides trispecific polypeptides comprising or essentially consisting of a Nanobody against a specific epitope on EGFR, a Nanobody against another specific epitope on EGFR and a Nanobody against human serum albumin, such as non-limiting examples SEQ ID NO's: 142-143.

Furthermore, although it is encompassed within the scope of the invention that the specific order or arrangement of the various Nanobodies in the polypeptides of the invention may have some influence on the properties of the final polypeptide of the invention (including but not limited to the affinity, specificity or avidity for EGFR or IGF-IR, respectively, or against the one or more other antigens), said order or arrangement is usually not critical and may be suitably chosen by the skilled person, optionally after on some limited routine experiments based on the disclosure herein. Thus, when reference is made to a specific multivalent or multispecific polypeptide of the invention, it should be noted that this encompasses any order or arrangements of the relevant Nanobodies, unless explicitly indicated otherwise.

Also encompassed within the scope of the present invention are bispecific or multispecific polypeptides comprising or essentially consisting of at least two Nanobodies of which one of said at least two Nanobodies has a decreased or increased affinity for its antigen, upon binding by the other Nanobodies to its antigen. Such binding is called ‘conditional bispecific or multispecific binding’. Such bispecific or multispecific polypeptide is also called ‘a conditionally binding bispecific or multispecific polypeptide of the invention’.

Binding of the antigen by the first of said at least two Nanobodies may modulate, such as enhance, reduce or inhibit, binding of the antigen by the second of said at least two Nanobodies. In an embodiment, binding by the first of said at least two Nanobodies stimulates binding by the second of said at least two Nanobodies. In another embodiment, binding by the first of said at least two Nanobodies at least partially inhibits binding by the second of said at least two Nanobodies. In another embodiment, binding by the first of said at least two Nanobodies inhibits binding by the second of said at least two Nanobodies. In such an embodiment, the polypeptide of the invention may, for example, be maintained in the body of a subject organism in vivo through binding to a protein which increases the half-life of the polypeptide until such a time as it becomes bound to its second target antigen and dissociates from the half-life increasing protein.

Modulation of binding in the above context is achieved as a consequence of the structural proximity of the antigen binding sites of the Nanobodies relative to one another. Such structural proximity can be achieved by the nature of the structural components linking the two or more antigen binding sites, e.g. by the provision of a linker with a relatively rigid structure that holds the antigen binding sites in close proximity. Advantageously, the two or more antigen binding sites are in physically close proximity to one another such that one site modulates the binding of the antigen at another site by a process which involves steric hindrance and/or confirmational changes within the polypeptide.

Thus, in one aspect, the invention also relates to a method for producing a conditional binding bispecific or multispecific polypeptide of the invention comprising the steps of:
a) selecting a first Nanobody by its ability to bind to a first epitope,
b) selecting a second Nanobody by its ability to bind to a second epitope,
c) combining the Nanobodies, optionally via a linker sequence; and
d) selecting the conditional binding bispecific or multispecific polypeptide of the invention by its ability to bind to said first epitope and said second epitope.

In one embodiment, the conditional binding bispecific or multispecific polypeptide of the invention can be selected for its ability to bind to said first epitope and said second epitope, wherein binding to one of said epitopes enhances binding to the other epitope. Advantageously, binding is enhanced by 25% or more, advantageously 40%, 50%, 60%, 70%, 80%, 90% or more, and preferably by 100% or more.

In another embodiment, the conditional binding bispecific or multispecific polypeptide of the invention can be selected for its ability to bind to said first epitope and said second epitope, wherein binding to one of said epitopes reduces binding to the other epitope. In a preferred aspect of this embodiment, the conditional binding bispecific or multispecific polypeptide of the invention can be selected for its ability to bind to said first epitope and said 30 second epitope, but not to both said first and second epitopes simultaneously. In this case the first and second Nanobody compete for epitope binding. Advantageously, binding is reduced by 25% or more, advantageously 40%, 50%, 60%, 70%, 80%, 90% or more, and preferably up to 100% or nearly so, such that binding is completely inhibited. Binding of epitopes can be measured by conventional antigen binding assays, such as ELISA, by fluorescence based techniques, including FRET, or by techniques such as surface plasmon resonance which measure the mass of molecules.

In a further embodiment of the above method, a further step is provided comprising selecting a third or further Nanobody by its ability to bind to a third or further epitope. In this way the multispecific polypeptide produced comprises more than two Nanobodies. In this aspect of the invention, at least two of said Nanobodies provide a conditional bispecific binding (i.e. binding of the antigen by the first of said at least two Nanobodies modulates, such as enhances, reduces or inhibits, binding of the antigen by the second of said at least two Nanobodies). The other one or more Nanobody may also provide a conditional binding (also called conditional multispecific binding) or may be free to associate independently with its epitope(s).

In a preferred embodiment, the bispecific conditional binding polypeptide may comprise a first Nanobody binding a target molecule and a second Nanobody binding a molecule or group which extends the half-life of the polypeptide (examples of such molecules or groups are further described hereafter). In one embodiment, the first Nanobody may be capable of binding the target molecule only when the half-life enhancing molecule or group is bound to the second Nanobody. In another embodiment, the first Nanobody may be capable of binding the target molecule only on displacement of the half-life enhancing molecule or group from the second Nanobody. Thus, for example, the bispecific conditional binding polypeptide is maintained in circulation in the bloodstream of a subject by a bulky molecule such as HSA. When a target molecule is encountered, competition between the binding domains of the bispecific conditional binding polypeptide results in displacement of the HSA and binding of the target.

Finally, it is also within the scope of the invention that the polypeptides of the invention contain two or more Nanobodies and one or more further amino acid sequences (as mentioned herein).

For multivalent and multispecific polypeptides containing one or more V_{HH} domains and their preparation, reference is also made to Conrath et al., J. Biol. Chem., Vol. 276, 10. 7346-7350, 2001, as well as to for example WO 96/34103 and WO 99/23221. Some other examples of some specific multispecific and/or multivalent polypeptidee of the invention can be found in the applications by applicant referred to herein.

One preferred, but non-limiting example of a multispecific polypeptide of the invention comprises at least one Nanobody of the invention and at least one Nanobody that provides for an increased half-life. Some preferred, but non-limiting examples of such Nanobodies include Nanobodies directed against serum proteins, such as human serum albumin, thyroxine-binding protein, (human) transferrin, fibrinogen, an immunoglobulin such as IgG, IgE or IgM, or one of the other serum proteins listed in WO 04/003019.

For example, for experiments in mice, Nanobodies against mouse serum albumin (MSA) can be used, whereas for pharmaceutical use, Nanobodies against human serum albumin can be used.

Another embodiment of the present invention is a polypeptide construct as described above wherein said at least one (human) serum protein is any of (human) serum albumin, (human) serum immunoglobulins, (human) thyroxine-binding protein, (human) transferrin, (human) fibrinogen, etc.

According to a specific, but non-limiting aspect of the invention, the polypeptides of the invention contain, besides the one or more Nanobodies of the invention, at least one Nanobody against human serum albumin. Although these Nanobodies against human serum albumin may be as generally described in the applications by applicant cited above (see for example WO4/062551), according to a particularly preferred, but non-limiting embodiment, said Nanobody against human serum albumin consists of 4 framework regions (FR1 to FR4 respectively) and 3 complementarity determining regions (CDR1 to CDR3 respectively), in which:

i) CDR1 is an amino acid sequence chosen from the group consisting of:

| | |
|---|---|
| SFGMS | [SEQ ID NO: 15] |
| LNLMG | [SEQ ID NO: 16] |
| INLLG | [SEQ ID NO: 17] |
| NYWMY; | [SEQ ID NO: 18] |

and/or from the group consisting of amino acid sequences that have 2 or only 1 “amino acid difference(s)” (as defined herein) with one of the above amino acid sequences, in which:
(1) any amino acid substitution is preferably a conservative amino acid substitution (as defined herein); and/or
(2) said amino acid sequence preferably only contains amino acid substitutions, and no amino acid deletions or insertions, compared to the above amino acid sequences; and in which:
ii) CDR2 is an amino acid sequence chosen from the group consisting of:

| | |
|---|---|
| SISGSGSDTLYADSVKG | [SEQ ID NO: 19] |
| TITVGDSTNYADSVKG | [SEQ ID NO: 20] |
| TITVGDSTSYADSVKG | [SEQ ID NO: 21] |
| SINGRGDDTRYADSVKG | [SEQ ID NO: 22] |
| AISADSSTKNYADSVKG | [SEQ ID NO: 23] |
| AISADSSDKRYADSVKG | [SEQ ID NO: 24] |
| RISTGGGYSYYADSVKG | [SEQ ID NO: 25] |

or from the group consisting of amino acid sequences that have at least 80%, preferably at least 90%, more preferably at least 95%, even more preferably at least 99% sequence identity (as defined herein) with one of the above amino acid sequences; in which
(1) any amino acid substitution is preferably a conservative amino acid substitution (as defined herein); and/or
(2) said amino acid sequence preferably only contains amino acid substitutions, and no amino acid deletions or insertions, compared to the above amino acid sequences; and/or from the group consisting of amino acid sequences that have 3, 2 or only 1 “amino acid difference(s)” (as defined herein) with one of the above amino acid sequences, in which:
(1) any amino acid substitution is preferably a conservative amino acid substitution (as defined herein); and/or
(2) said amino acid sequence preferably only contains amino acid substitutions, and no amino acid deletions or insertions, compared to the above amino acid sequences;
and in which:
iii) CDR3 is an amino acid sequence chosen from the group consisting of:

| | |
|---|---|
| DREAQVDTLDFDY | [SEQ ID NO: 26] |

or from the group consisting of amino acid sequences that have at least 80%, preferably at least 90%, more preferably at least 95%, even more preferably at least 99% sequence identity (as defined herein) with one of the above amino acid sequences; in which
(1) any amino acid substitution is preferably a conservative amino acid substitution (as defined herein); and/or
(2) said amino acid sequence preferably only contains amino acid substitutions, and no amino acid deletions or insertions, compared to the above amino acid sequences; and/or from the group consisting of amino acid sequences that have 3, 2 or only 1 “amino acid difference(s)” (as defined herein) with one of the above amino acid sequences, in which:
(1) any amino acid substitution is preferably a conservative amino acid substitution (as defined herein); and/or
(2) said amino acid sequence preferably only contains amino acid substitutions, and no amino acid deletions or insertions, compared to the above amino acid sequences;

or from the group consisting of:

| | |
|---|---|
| GGSLSR | [SEQ ID NO: 27] |
| RRTWHSEL | [SEQ ID NO: 28] |
| GRSVSRS | [SEQ ID NO: 29] |
| GRGSP | [SEQ ID NO: 30] |

and/or from the group consisting of amino acid sequences that have 3, 2 or only 1 “amino acid difference(s)” (as defined herein) with one of the above amino acid sequences, in which:
(1) any amino acid substitution is preferably a conservative amino acid substitution (as defined herein); and/or
(2) said amino acid sequence preferably only contains amino acid substitutions, and no amino acid deletions or insertions, compared to the above amino acid sequences.

In another aspect, the invention relates to a Nanobody against human serum albumin, which consist of 4 framework regions (FR1 to FR4 respectively) and 3 complementarity determining regions (CDR1 to CDR3 respectively), which is chosen from the group consisting of Nanobodies with the one of the following combinations of CDR1, CDR2 and CDR3, respectively:

| | |
|---|---|
| CDR1: SFGMS; | CDR2: SISGSGSDTLYADSVKG; |
| CDR3: GGSLSR; | |
| CDR1: LNLMG; | CDR2: TITVGDSTNYADSVKG; |
| CDR3: RRTWHSEL; | |
| CDR1: INLLG; | CDR2: TITVGDSTSYADSVKG; |
| CDR3: RRTWHSEL; | |
| CDR1: SFGMS; | CDR2: SINGRGDDTRYADSVKG; |
| CDR3: GRSVSRS; | |
| CDR1: SFGMS; | CDR2: AISADSSDKRYADSVKG; |
| CDR3: GRGSP; | |
| CDR1: SFGMS; | CDR2: AISADSSDKRYADSVKG; |
| CDR3: GRGSP; | |
| CDR1: NYWMY; | CDR2: RISTGGGYSYYADSVKG; |
| CDR3: DREAQVDTLDFDY. | |

In the Nanobodies of the invention that comprise the combinations of CDR's mentioned above, each CDR can be replaced by a CDR chosen from the group consisting of amino acid sequences that have at least 80%, preferably at least 90%, more preferably at least 95%, even more preferably at least 99% sequence identity (as defined herein) with the mentioned CDR's; in which

(1) any amino acid substitution is preferably a conservative amino acid substitution (as defined herein); and/or
(2) said amino acid sequence preferably only contains amino acid substitutions, and no amino acid deletions or insertions, compared to the above amino acid sequences; and/or chosen from the group consisting of amino acid sequences that have 3, 2 or only 1 (as indicated in the preceding paragraph) “amino acid difference(s)” (as defined herein) with the mentioned CDR(s) in one of the above amino acid sequences, in which:
(1) any amino acid substitution is preferably a conservative amino acid substitution (as defined herein); and/or
(2) said amino acid sequence preferably only contains amino acid substitutions, and no amino acid deletions or insertions, compared to the above amino acid sequences.

However, of the Nanobodies of the invention that comprise the combinations of CDR's mentioned above, Nanobodies comprising one or more of the CDR's listed above are particularly preferred; Nanobodies comprising two or more of the CDR's listed above are more particularly preferred; and Nanobodies comprising three of the CDR's listed above are most particularly preferred.

In these Nanobodies against human serum albumin, the Framework regions FR1 to FR4 are preferably as defined hereinabove for the Nanobodies of the invention.

Some preferred, but non-limiting examples of Nanobodies directed against human serum albumin that can be used in the polypeptides of the invention are listed in Table A-9 below. ALB-8 is a humanized version of ALB-1.

TABLE A-9

Preferred, but non-limiting examples
of albumin-binding Nanobodies

<Name, SEQ ID #; PRT (protein); ->
Sequence
<PMP 6A6 (ALB11), SEQ ID NO: 32; PRT;->
AVQLVESGGGLVQPGNSLRLSCAASGFTFRSFGMSWVRQAPGKEPEWVSS
ISGSGSDTLYADSVKGRFTISRDNAKTTLYLQMNSLKPEDTAVYYCTIGG
SLSRSSQGTQVTVSS
<ALB-8 (humanized ALB-1), SEQ ID NO: 33; PRT;->
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTLVTVSS
<PMP 6A8 (ALB-2), SEQ ID NO: 34; PRT;->
AVQLVESGGGLVQGGGSLRLACAASERIFDLNLMGWYRQGPGNERELVAT
CITVGDSTNYADSVKGRFTISMDYTKQTVYLHMNSLRPEDTGLYYCKIRR
TWHSELWGQGTQVTVSS

Generally, any derivatives and/or polypeptides of the invention with increased half-life (for example pegylated Nanobodies or polypeptides of the invention, multispecific Nanobodies directed against EGFR or IGF-IR, respectively, and (human) serum albumin, or Nanobodies fused to an Fc portion, all as described herein) have a half-life that is at least 1.5 times, preferably at least 2 times, such as at least 5 times, for example at least 10 times or more than 20 times, the half-life of the corresponding Nanobody of the invention.

Also, any derivatives or polypeptides of the invention with an increase half-life preferably have a half-life of more than 1 hour, preferably more than 2 hours, more preferably of more than 6 hours, such as of more than 12 hours, and for example of about one day, two days, one week, two weeks or three weeks, and preferably no more than 2 months, although the latter may be less critical.

Half-life can generally be defined as the time taken for the serum concentration of the polypeptide to be reduce by 50%, in vivo, for example due to degradation of the ligand and/or clearance or sequestration of the ligand by natural mechanisms. Methods for pharmacokinetic analysis and determination of half-life are familiar to those skilled in the art. Details may be found in Kenneth, A et al: Chemical Stability of Pharmaceuticals: A Handbook for Pharmacists and in Peters et al, Pharmacokinete analysis: A Practical Approach (1996).

Reference is also made to “Pharmacokinetics”, M Gibaldi & D Perron, published by Marcel Dekker, 2 nd Rev. ex edition (1982).

According to one aspect of the invention the polypeptides are capable of binding to one or more molecules which can increase the half-life of the polypeptide in vivo.

The polypeptides of the invention are stabilised in vivo and their half-life increased by binding to molecules which resist degradation and/or clearance or sequestration. Typically, such molecules are naturally occurring proteins which themselves have a long half-life in vivo.

Another preferred, but non-limiting example of a multispecific polypeptide of the invention comprises at least one Nanobody of the invention and at least one Nanobody that directs the polypeptide of the invention towards, and/or that allows the polypeptide of the invention to penetrate or to enter into specific organs, tissues, cells, or parts or compartments of cells, and/or that allows the Nanobody to penetrate or cross a biological barrier such as a cell membrane, a cell layer such as a layer of epithelial cells, a tumor including solid tumors, or the blood-brain-barrier. Examples of such Nanobodies include Nanobodies that are directed towards specific cell-surface proteins, markers or epitopes of the desired organ, tissue or cell (for example cell-surface markers associated with tumor cells), and the single-domain brain targeting antibody fragments described in WO 02/057445, of which FC44 (SEQ ID NO 35) and FC5 (SEQ ID NO: 36) are preferred examples.

TABLE A-10
Sequence listing of FC44 and FC5

<Name, SEQ ID #; PRT (protein); ->
Sequence
<FC44, SEQ ID NO: 35; PRT;>
EVQLQASGGGLVQAGGSLRLSCSASVRTFSIYAMGWFRQAPGKEREFVAG
INRSGDVTKYADFVKGRFSISRDNAKNMVYLQMNSLKPEDTALYYCAATW
AYDTVGALTSGYNFWGQGTQVTVSS
<FC5, SEQ ID NO: 36; PRT;->
EVQLQASGGGLVQAGGSLRLSCAASGFKITHYTMGWFRQAPGKEREFVSR
ITWGGDNTFYSNSVKGRFTISRDNAKNTVYLQMNSLKPEDTADYYCAAGS
TSTATPLRVDYWGKGTQVTVSS

In the polypeptides of the invention, the one or more Nanobodies and the one or more polypeptides may be directly linked to each other (as for example described in WO 99/23221) and/or may be linked to each other via one or more suitable spacers or linkers, or any combination thereof.

Suitable spacers or linkers for use in multivalent and multispecific polypeptides will be clear to the skilled person, and may generally be any linker or spacer used in the art to link amino acid sequences. Preferably, said linker or spacer is suitable for use in constructing proteins or polypeptides that are intended for pharmaceutical use.

Some particularly preferred spacers include the spacers and linkers that are used in the art to link antibody fragments or antibody domains. These include the linkers mentioned in the general background art cited above, as well as for example linkers that are used in the art to construct diabodies or ScFv fragments (in this respect, however, its should be noted that, whereas in diabodies and in ScFv fragments, the linker sequence used should have a length, a degree of flexibility and other properties that allow the pertinent V_{H} and V_{L} domains to come together to form the complete antigen-binding site, there is no particular limitation on the length or the flexibility of the linker used in the polypeptide of the invention, since each Nanobody by itself forms a complete antigen-binding site).

For example, a linker may be a suitable amino acid sequence, and in particular amino acid sequences of between 1 and 50, preferably between 1 and 30, such as between 1 and 10 amino acid residues. Some preferred examples of such amino acid sequences include gly-ser linkers, for example of the type (glyₓser_{y})_{z}, such as (for example (gly₄ser)₃ or (gly₃ser₂)₃, as described in WO 99/42077, hinge-like regions such as the hinge regions of naturally occurring heavy chain antibodies or similar sequences (such as described in WO 94/04678).

Some other particularly preferred linkers are poly-alanine (such as AAA), as well as the linkers mentioned in Table A-11, of which AAA, GS-7 and GS-9 are particularly preferred.

TABLE A-11
Sequence listing of linkers

<Name, SEQ ID #; PRT (protein); ->
Sequence

< GS30, SEQ ID NO: 37; PRT;->
GGGGSGGGGSGGGGSGGGGSGGGGSGGGGS
< GS15, SEQ ID NO: 38; PRT;->
GGGGSGGGGSGGGGS
< GS9, SEQ ID NO: 39; PRT;->
GGGGSGGGS
< GS7, SEQ ID NO: 40; PRT;->
SGGSGGS
< Llama upper long hinge region, SEQ ID NO: 41;
PRT;->
EPKTPKPQPAAA

Other suitable linkers generally comprise organic compounds or polymers, in particular those suitable for use in proteins for pharmaceutical use. For instance, poly(ethyleneglycol) moieties have been used to link antibody domains, see for example WO 04/081026.

It is encompassed within the scope of the invention that the length, the degree of flexibility and/or other properties of the linker(s) used (although not critical, as it usually is for linkers used in ScFv fragments) may have some influence on the properties of the final polypeptide of the invention, including but not limited to the affinity, specificity or avidity for EGFR or IGF-IR, respectively, or against the one or more of the other antigens. Based on the disclosure herein, the skilled person will be able to determine the optimal linker(s) for use in a specific polypeptide of the invention, optionally after some limited routine experiments.

For example, in multivalent polypeptides of the invention that comprise Nanobodies directed against a multimeric antigen (such as a multimeric receptor or other protein), the length and flexibility of the linker are preferably such that it allows each Nanobody of the invention present in the polypeptide to bind to the antigenic determinant on each of the subunits of the multimer. Similarly, in a multispecific polypeptide of the invention that comprises Nanobodies directed against two or more different antigenic determinants on the same antigen (for example against different epitopes of an antigen and/or against different subunits of a multimeric receptor, channel or protein), the length and flexibility of the linker are preferably such that it allows each Nanobody to bind to its intended antigenic determinant. Again, based on the disclosure herein, the skilled person will be able to determine the optimal linker(s) for use in a specific polypeptide of the invention, optionally after some limited routine experiments.

It is also within the scope of the invention that the linker(s) used confer one or more other favourable properties or functionality to the polypeptides of the invention, and/or provide one or more sites for the formation of derivatives and/or for the attachment of functional groups (e.g. as described herein for the derivatives of the Nanobodies of the invention). For example, linkers containing one or more charged amino acid residues (see Table A-2 above) can provide improved hydrophilic properties, whereas linkers that form or contain small epitopes or tags can be used for the purposes of detection, identification and/or purification. Again, based on the disclosure herein, the skilled person will be able to determine the optimal linkers for use in a specific polypeptide of the invention, optionally after some limited routine experiments.

Finally, when two or more linkers are used in the polypeptides of the invention, these linkers may be the same or different. Again, based on the disclosure herein, the skilled person will be able to determine the optimal linkers for use in a specific polypeptide of the invention, optionally after some limited routine experiments.

Usually, for easy of expression and production, a polypeptide of the invention will be a linear polypeptide. However, the invention in its broadest sense is not limited thererto. For example, when a polypeptide of the invention comprises three of more Nanobodies, it is possible to link them by use of a linker with three or more “arms”, of which each “arm” being linked to a Nanobody, so as to provide a “star-shaped” construct. It is also possible, although usually less preferred, to use circular constructs.

The invention also comprises derivatives of the polypeptides of the invention, which may be essentially analogous to the derivatives of the Nanobodies of the invention, i.e. as described herein.

The invention also comprises proteins or polypeptides that “essentially consist” of a polypeptide of the invention (in which the wording “essentially consist of” has essentially the same meaning as indicated hereinabove).

According to one embodiment of the invention, the polypeptide of the invention is in essentially isolated from, as defined herein.

The Nanobodies, polypeptides and nucleic acids of the invention can be prepared in a manner known per se, as will be clear to the skilled person from the further description herein. For example, the Nanobodies and polypetides of the invention can be prepared in any manner known per se for the preparation of antibodies and in particular for the preparation of antibody fragments (including but not limited to (single) domain antibodies and ScFv fragments). Some preferred, but non-limiting methods for preparing the Nanobodies, polypeptides and nucleic acids include the methods and techniques described herein.

As will be clear to the skilled person, one particularly useful method for preparing a Nanobody and/or a polypeptide of the invention generally comprises the steps of:
the expression, in a suitable host cell or host organism (also referred to herein as a “host of the invention”) or in another suitable expression system of a nucleic acid that encodes said Nanobody or polypeptide of the invention (also referred to herein as a “nucleic acid of the invention”), optionally followed by:
isolating and/or purifying the Nanobody or polypeptide of the invention thus obtained.

In particular, such a method may comprise the steps of:
cultivating and/or maintaining a host of the invention under conditions that are such that said host of the invention expresses and/or produces at least one Nanobody and/or polypeptide of the invention; optionally followed by:
isolating and/or purifying the Nanobody or polypeptide of the invention thus obtained.

A nucleic acid of the invention can be in the form of single or double stranded DNA or RNA, and is preferably in the form of double stranded DNA. For example, the nucleotide sequences of the invention may be genomic DNA, cDNA or synthetic DNA (such as DNA with a codon usage that has been specifically adapted for expression in the intended host cell or host organism).

According to one embodiment of the invention, the nucleic acid of the invention is in essentially isolated from, as defined herein.

The nucleic acid of the invention may also be in the form of, be present in and/or be part of a vector, such as for example a plasmid, cosmid or YAC, which again may be in essentially isolated form.

The nucleic acids of the invention can be prepared or obtained in a manner known per se, based on the information on the amino acid sequences for the polypeptides of the invention given herein, and/or can be isolated from a suitable natural source. To provide analogs, nucleotide sequences encoding naturally occurring V_{HH} domains can for example be subjected to site-directed mutagenesis, so at to provide a nucleic acid of the invention encoding said analog. Also, as will be clear to the skilled person, to prepare a nucleic acid of the invention, also several nucleotide sequences, such as at least one nucleotide sequence encoding a Nanobody and for example nucleic acids encoding one or more linkers can be -linked-together in a suitable manner.

Techniques for generating the nucleic acids of the invention will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers, using for example a sequence of a naturally occurring GPCR as a template. These and other techniques will be clear to the skilled person, and reference is again made to the standard handbooks, such as Sambrook et al. and Ausubel et al., mentioned above, as well as the Examples below.

The nucleic acid of the invention may also be in the form of, be present in and/or be part of a genetic construct, as will be clear to the person skilled in the art. Such genetic constructs generally comprise at least one nucleic acid of the invention that is optionally linked to one or more elements of genetic constructs known per se, such as for example one or more suitable regulatory elements (such as a suitable promoter(s), enhancer(s), terminator(s), etc.) and the further elements of genetic constructs referred to herein. Such genetic constructs comprising at least one nucleic acid of the invention will also be referred to herein as “genetic constructs of the invention”.

The genetic constructs of the invention may be DNA or RNA, and are preferably double-stranded DNA. The genetic constructs of the invention may also be in a form suitable for transformation of the intended host cell or host organism, in a form suitable for integration into the genomic DNA of the intended host cell or in a form suitable for independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell, host organism and/or expression system).

In a preferred but non-limiting embodiment, a genetic construct of the invention comprises
a) at least one nucleic acid of the invention; operably connected to
b) one or more regulatory elements, such as a promoter and optionally a suitable terminator;
   and optionally also
c) one or more further elements of genetic constructs known per se; in which the terms “regulatory element”, “promoter”, “terminator” and “operably connected” have their usual meaning in the art (as further described herein); and in which said “further elements” present in the genetic constructs may for example be 3′- or 5′-UTR sequences, leader sequences, selection markers, expression markers/reporter genes, and/or elements that may facilitate or increase (the efficiency of) transformation or integration. These and other suitable elements for such genetic constructs will be clear to the skilled person, and may for instance depend upon the type of construct used, the intended host cell or host organism; the manner in which the nucleotide sequences of the invention of interest are to be expressed (e.g. via constitutive, transient or inducible expression); and/or the transformation technique to be used. For example, regulatory sequences, promoters and terminators known per se for the expression and production of antibodies and antibody fragments (including but not limited to (single) domain antibodies and ScFv fragments) may be used in an essentially analogous manner.

Preferably, in the genetic constructs of the invention, said at least one nucleic acid of the invention and said regulatory elements, and optionally said one or more further elements, are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor). Generally, when two nucleotide sequences are operably linked, they will be in the same orientation and usually also in the same reading frame. They will usually also be essentially contiguous, although this may also not be required.

Preferably, the regulatory and further elements of the genetic constructs of the invention are such that they are capable of providing their intended biological function in the intended host cell or host organism.

For instance, a promoter, enhancer or terminator should be “operable” in the intended host cell or host organism, by which is meant that (for example) said promoter should be capable of initiating or otherwise controlling/regulating the transcription and/or the expression of a nucleotide sequence—e.g. a coding sequence—to which it is operably linked (as defined herein).

Some particularly preferred promoters include, but are not limited to, promoters known per se for the expression in the host cells mentioned herein; and in particular promoters for the expression in the bacterial cells, such as those mentioned herein and/or those used in the Examples.

A selection marker should be such that it allows—i.e. under appropriate selection conditions—host cells and/or host organisms that have been (successfully) transformed with the nucleotide sequence of the invention to be distinguished from host cells/organisms that have not been (successfully) transformed. Some preferred, but non-limiting examples of such markers are genes that provide resistance against antibiotics (such as kanamycin or ampicillin), genes that provide for temperature resistance, or genes that allow the host cell or host organism to be maintained in the absence of certain factors, compounds and/or (food) components in the medium that are essential for survival of the non-transformed cells or organisms.

A leader sequence should be such that—in the intended host cell or host organism—it allows for the desired post-translational modifications and/or such that it directs the transcribed mRNA to a desired part or organelle of a cell. A leader sequence may also allow for secretion of the expression product from said cell. As such, the leader sequence may be any pro-, pre-, or prepro-sequence operable in the host cell or host organism. Leader sequences may not be required for expression in a bacterial cell. For example, leader sequences known per se for the expression and production of antibodies and antibody fragments (including but not limited to single domain antibodies and ScFv fragments) may be used in an essentially analogous manner.

An expression marker or reporter gene should be such that—in the host cell or host organism—it allows for detection of the expression of (a gene or nucleotide sequence present on) the genetic construct. An expression marker may optionally also allow for the localisation of the expressed product, e.g. in a specific part or organelle of a cell and/or in (a) specific cell(s), tissue(s), organ(s) or part(s) of a multicellular organism. Such reporter genes may also be expressed as a protein fusion with the amino acid sequence of the invention. Some preferred, but non-limiting examples include fluorescent proteins such as GFP.

Some preferred, but non-limiting examples of suitable promoters, terminator and further elements include those that can be used for the expression in the host cells mentioned herein; and in particular those that are suitable for expression bacterial cells, such as those mentioned herein and/or those used in the Examples below. For some (further) non-limiting examples of the promoters, selection markers, leader sequences, expression markers and further elements that may be present/used in the genetic constructs of the invention—such as terminators, transcriptional and/or translational enhancers and/or integration factors—reference is made to the general handbooks such as Sambrook et al. and Ausubel et al. mentioned above, as well as to the examples that are given in WO 95/07463, WO 96/23810, WO 95/07463, WO 95/21191, WO 97/11094, WO 97/42320, WO 98/06737, WO 98/21355, U.S. Pat. No. 6,207,410, U.S. Pat. No. 5,693,492 and EP 1 085 089. Other examples will be clear to the skilled person. Reference is also made to the general background art cited above and the further references cited herein.

The genetic constructs of the invention may generally be provided by suitably linking the nucleotide sequence(s) of the invention to the one or more further elements described above, for example using the techniques described in the general handbooks such as Sambrook et al. and Ausubel et al., mentioned above.

Often, the genetic constructs of the invention will be obtained by inserting a nucleotide sequence of the invention in a suitable (expression) vector known per se. Some preferred, but non-limiting examples of suitable expression vectors are those used in the Examples below, as well as those mentioned herein.

The nucleic acids of the invention and/or the genetic constructs of the invention may be used to transform a host cell or host organism, i.e. for expression and/or production of the Nanobody or polypeptide of the invention. Suitable hosts or host cells will be clear to the skilled person, and may for example be any suitable fungal, prokaryotic or eukaryotic cell or cell line or any suitable fungal, prokaryotic or eukaryotic organism, for example:
a bacterial strain, including but not limited to gram-negative strains such as strains of Escherichia coli; of Proteus, for example of Proteus mirabilis; of Pseudomonas, for example of Pseudomonas fluorescens; and gram-positive strains such as strains of Bacillus, for example of Bacillus subtilis or of Bacillus brevis; of Streptomyces, for example of Streptomyces lividans; of Staphylococcus, for example of Staphylococcus carnosus; and of Lactococcus, for example of Lactococcus lactis;
a fungal cell, including but not limited to cells from species of Trichoderma, for example from Trichoderma reesei; of Neurospora, for example from Neurospora crassa; of Sordaria, for example from Sordaria macrospora; of Aspergillus, for example from Aspergillus niger or from Aspergillus sojae; or from other filamentous fungi;
a yeast cell, including but not limited to cells from species of Saccharomyces, for example of Saccharomyces cerevisiae; of Schizosaccharomyces; for example of Schizosaccharomyces pombe; of Pichia, for example of Pichia pastoris or of Pichia methanolica; of Hansenula, for example of Hansenula polymorpha; of Kluyveromyces, for example of Kluyveromyces lactis; of Arxula, for example of Arxula adeninivorans; of Yarrowia, for example of Yarrowia lipolytica;
an amphibian cell or cell line, such as Xenopus oocytes;
an insect-derived cell or cell line, such as cells/cell lines derived from lepidoptera, including but not limited to Spodoptera SF9 and Sf21 cells or cells/cell lines derived from Drosophila, such as Schneider and Kc cells;
a plant or plant cell, for example in tobacco plants; and/or
a mammalian cell or cell line, for example derived a cell or cell line derived from a human, from the mammals including but not limited to CHO-cells, BHK-cells (for example BHK-21 cells) and human cells or cell lines such as HeLa, COS (for example COS-7) and PER.C6 cells;
   as well as all other hosts or host cells known per se for the expression and production of antibodies and antibody fragments (including but not limited to (single) domain antibodies and ScFv fragments), which will be clear to the skilled person. Reference is also made to the general background art cited hereinabove, as well as to for example WO 94/29457; WO 96/34103; WO 99/42077; Frenken et al., (1998), supra; Riechmann and Muyldermans, (1999), supra; van der Linden, (2000), supra; Thomassen et al., (2002), supra; Joosten et al., (2003), supra; Joosten et al., (2005), supra; and the further references cited herein.

The Nanobodies and polypeptides of the invention can also be introduced and expressed in one or more cells, tissues or organs of a multicellular organism, for example for prophylactic and/or therapeutic purposes (e.g. as a gene therapy). For this purpose, the nucleotide sequences of the invention may be introduced into the cells or tissues in any suitable way, for example as such (e.g. using liposomes) or after they have been inserted into a suitable gene therapy vector (for example derived from retroviruses such as adenovirus, or parvoviruses such as adeno-associated virus). As will also be clear to the skilled person, such gene therapy may be performed in vivo and/or in situ in the body of a patient by administering a nucleic acid of the invention or a suitable gene therapy vector encoding the same to the patient or to specific cells or a specific tissue or organ of the patient; or suitable cells (often taken from the body of the patient to be treated, such as explanted lymphocytes, bone marrow aspirates or tissue biopsies) may be treated in vitro with a nucleotide sequence of the invention and then be suitably (re-)introduced into the body of the patient. All this can be performed using gene therapy vectors, techniques and delivery systems which are well known to the skilled person, for Culver, K. W., “Gene Therapy”, 1994, p. xii, Mary Ann Liebert, Inc., Publishers, New York, N.Y). Giordano, Nature F Medicine 2 (1996), 534-539; Schaper, Circ. Res. 79 (1996), 911-919; Anderson, Science 256 (1992), 808-813; Verma, Nature 389 (1994), 239; Isner, Lancet 348 (1996), 370-374; Muhlhauser, Circ. Res. 77 (1995), 1077-1086; Onodera, Blood 91; (1998), 30-36; Verma, Gene Ther. 5 (1998), 692-699; Nabel, Ann. N.Y. Acad. Sci.: 811 (1997), 289-292; Verzeletti, Hum. Gene Ther. 9 (1998), 2243-51; Wang, Nature Medicine 2 (1996), 714-716; WO 94/29469; WO 97/00957, U.S. Pat. No. 5,580,859; 1 U.S. Pat. No. 55,895,466; or Schaper, Current Opinion in Biotechnology 7 (1996), 635-640. For example, in situ expression of ScFv fragments (Afanasieva et al., Gene Ther., 10, 1850-1859 (2003)) and of diabodies (Blanco et al., J. Immunol, 171, 1070-1077 (2003)) has been described in the art.

For expression of the Nanobodies in a cell, they may also be expressed as so-called “intrabodies”, as for example described in WO 94/02610, WO 95/22618 and U.S. Pat. No. 6,004,940; WO 03/014960; in Cattaneo, A. & Biocca, S. (1997) Intracellular Antibodies: Development and Applications. Landes and Springer-Verlag; and in Kontermann, Methods 34, (2004), 163-170.

For production, the Nanobodies and polypeptides of the invention can for example also be produced in the milk of transgenic mammals, for example in the milk of rabbits, cows, goats or sheep (see for example U.S. Pat. No. 5,741,957, U.S. Pat. No. 5,304,489 and U.S. Pat. No. 5,849,992 for general techniques for introducing transgenes into mammals), in plants or parts of plants including but not limited to their leaves, flowers, fruits, seed, roots or turbers (for example in tobacco, maize, soybean or alfalfa) or in for example pupae of the silkworm Bombix mori.

Furthermore, the Nanobodies and polypeptides of the invention can also be expressed and/or produced in cell-free expression systems, and suitable examples of such systems will be clear to the skilled person. Some preferred, but non-limiting examples include expression in the wheat germ system; in rabbit reticulocyte lysates; or in the E. coli Zubay system.

As mentioned above, -one of the advantages of the use of Nanobodies is that the polypeptides based thereon can be prepared through expression in a suitable bacterial system, and suitable bacterial expression systems, vectors, host cells, regulatory elements, etc., will be clear to the skilled person, for example from the references cited above. It should however be noted that the invention in its broadest sense is not limited to expression in bacterial systems.

Preferably, in the invention, an (in vivo or in vitro) expression system, such as a bacterial expression system, is used that provides the polypeptides of the invention in a form that is suitable for pharmaceutical use, and such expression systems will again be clear to the skilled person. As also will be clear to the skilled person, polypeptides of the invention suitable for pharmaceutical use can be prepared using techniques for peptide synthesis.

For production on industrial scale, preferred heterologous hosts for the (industrial) production of Nanobodies or Nanobody-containing protein therapeutics include strains of E. coli, Pichia pastoris, S. cerevisiae that are suitable for large scale expression/production/fermentation, and in particular for large scale pharmaceutical expression/production/fermentation. Suitable examples of such strains will be clear to the skilled person. Such strains and production/expression systems are also made available by companies such as Biovitrum (Uppsala, Sweden).

Alternatively, mammalian cell lines, in particular Chinese hamster ovary (CHO) cells, can be used for large scale expression/production/fermentation, and in particular for large scale pharmaceutical expression/production/fermentation. Again, such expression/production systems are also made available by some of the companies mentioned above.

The choice of the specific expression system would depend in part on the requirement for certain post-translational modifications, more specifically glycosylation. The production of a Nanobody-containing recombinant protein for which glycosylation is desired or required would necessitate the use of mammalian expression hosts that have the ability to glycosylate the expressed protein. In this respect, it will be clear to the skilled person that the glycosylation pattern obtained (i.e. the kind, number and position of residues attached) will depend on the cell or cell line that is used for the expression. Preferably, either a human cell or cell line is used (i.e. leading to a protein that essentially has a human glycosylation pattern) or another mammalian cell line is used that can provide a glycosylation pattern that is essentially and/or functionally the same as human glycosylation or at least mimics human glycosylation. Generally, prokaryotic hosts such as E. coli do not have the ability to glycosylate proteins, and the use of lower eukaryotes such as yeast usually leads to a glycosylation pattern that differs from human glycosylation. Nevertheless, it should be understood that all the foregoing host cells and expression systems can be used in the invention, depending on the desired Nanobody or protein to be obtained.

Thus, according to one non-limiting embodiment of the invention, the Nanobody or polypeptide of the invention is glycosylated. According to another non-limiting embodiment of the invention, the Nanobody or polypeptide of the invention is non-glycosylated.

According to one preferred, but non-limiting embodiment of the invention, the Nanobody or polypeptide of the invention is produced in a bacterial cell, in particular a bacterial cell suitable for large scale pharmaceutical production, such as cells of the strains mentioned above.

According to another preferred, but non-limiting embodiment of the invention, the Nanobody or polypeptide of the invention is produced in a yeast cell, in particular a yeast cell suitable for large scale pharmaceutical production, such as cells of the species mentioned above.

According to yet another preferred, but non-limiting embodiment of the invention, the Nanobody or polypeptide of the invention is produced in a mammalian cell, in particular in a human cell or in a cell of a human cell line, and more in particular in a human cell or in a cell of a human cell line that is suitable for large scale pharmaceutical production, such as the cell lines mentioned hereinabove.

When expression in a host cell is used to produce the Nanobodies and the proteins of the invention, the Nanobodies and proteins of the invention can be produced either intracellullarly (e.g. in the cytosol, in the periplasma or in inclusion bodies) and then isolated from the host cells and optionally further purified; or can be produced extracellularly (e.g. in the medium in which the host cells are cultured) and then isolated from the culture medium and optionally further purified. When eukaryotic hosts cells are used, extracellular production is usually preferred since this considerably facilitates the further isolation and downstream processing of the Nanobodies and proteins obtained. Bacterial cells such as the strains of E. coli mentioned above normally do not secrete proteins extracellularly, except for a few classes of proteins such as toxins and hemolysin, and secretory production in E. coli refers to the translocation of proteins across the inner membrane to the periplasmic space. Periplasmic production provides several advantages over cytosolic production. For example, the N-terminal amino acid sequence of the secreted product can be identical to the natural gene product after cleavage of the secretion signal sequence by a specific signal peptidase. Also, there appears to be much less protease activity in the periplasm than in the cytoplasm. In addition, protein purification is simpler due to fewer contaminating proteins in the periplasm.

Another advantage is that correct disulfide bonds may form because the periplasm provides a more oxidative environment than the cytoplasm. Proteins overexpressed in E. coli are often found in insoluble aggregates, so-called inclusion bodies. These inclusion bodies may be located in the cytosol or in the periplasm; the recovery of biologically active proteins from these inclusion bodies requires a denaturation/refolding process. Many recombinant proteins, including therapeutic proteins, are recovered from inclusion bodies. Alternatively, as will be clear to the skilled person, recombinant strains of bacteria that have been genetically modified so as to secrete a desired protein, and in particular a Nanobody or a polypeptide of the invention, can be used.

Thus, according to one non-limiting embodiment of the invention, the Nanobody or polypeptide of the invention is a Nanobody or polypeptide that has been produced intracellularly and that has been isolated from the host cell, and in particular from a bacterial cell or from an inclusion body in a bacterial cell. According to another non-limiting embodiment of the invention, the Nanobody or polypeptide of the invention is a Nanobody or polypeptide that has been produced extracellularly, and that has been isolated from the medium in which the host cell is cultivated.

Some preferred, but non-limiting promoters for use with these host cells include,
for expression in E. coli: lac promoter (and derivatives thereof such as the lacUV5 promoter); arabinose promoter; left-(PL) and rightward (PR) promoter of phage lambda; promoter of the trp operon; hybrid lac/trp promoters (tac and trc); T7-promoter (more specifically that of T7-phage gene 10) and other T-phage promoters; promoter of the Tn10 tetracycline resistance gene; engineered variants of the above promoters that include one or more copies of an extraneous regulatory operator sequence;
for expression in S. cerevisiae: constitutive: ADH1 (alcohol dehydrogenase 1), ENO (enolase), CYC1 (cytochrome c iso-1), GAPDH (glyceraldehydes-3-phosphate dehydrogenase); PGK1 (phosphoglycerate kinase), PYKI (pyruvate kinase); regulated: GAL1,10,7 (galactose metabolic enzymes), ADH2 (alcohol dehydrogenase 2), PHO5 (acid phosphatase), CUP1 (copper metallothionein); heterologous: CaMV (cauliflower mosaic virus 35S promoter);
for expression in Pichia pastoris: the AOX1 promoter (alcohol oxidase I)
for expression in mammalian cells: human cytomegalovirus (hCMV) immediate early enhancer/promoter; human cytomegalovirus (hCMV) immediate early promoter variant that contains two tetracycline operator sequences such that the promoter can be regulated by the Tet repressor; Herpes Simplex Virus thymidine kinase (TK) promoter; Rous Sarcoma Virus long terminal repeat (RSV LTR) enhancer/promoter; elongation factor 1α (hEF-1α) promoter from human, chimpanzee, mouse or rat; the SV40 early promoter; HIV-1 long terminal repeat promoter; β-actin promoter;

Some preferred, but non-limiting vectors for use with these host cells include:
vectors for expression in mammalian cells: pMAMneo (Clontech), pcDNA3 (Invitrogen), pMClneo (Stratagene), pSG5 (Stratagene), EBO-pSV2-neo (ATCC 37593), pBPV-1 (8-2) (ATCC 37110), pdBPV-MMTneo (342-12) (ATCC 37224), pRSVgpt (ATCC37199), pRSVneo (ATCC37198), pSV2-dhfr (ATCC 37146), pUCTag (ATCC 37460) and 1ZD35 (ATCC 37565), as well as viral-based expression systems, such as those based on adenovirus;
vectors for expression in bacterials cells: pET vectors (Novagen) and pQE vectors (Qiagen);
vectors for expression in yeast or other fungal cells: pYES2 (Invitrogen) and Pichia expression vectors (Invitrogen);
vectors for expression in insect cells: pBlueBacII (Invitrogen) and other baculovirus vectors
vectors for expression in plants or plant cells: for example vectors based on cauliflower mosaic virus or tobacco mosaic virus, suitable strains of Agrobacterium, or Ti-plasmid based vectors.

Some preferred, but non-limiting secretory sequences for use with these host cells include:
for use in bacterial cells such as E. coli: PelB, Bla, OmpA, OmpC, OmpF, OmpT, SthI, PhoA, PhoE, MalE, Lpp, LamB, and the like; TAT signal peptide, hemolysin C-terminal secretion signal
for use in yeast: α-mating factor prepro-sequence, phosphatase (pho1), invertase (Suc), etc.;
for use in mammalian cells: indigenous signal in case the target protein is of eukaryotic origin; murine Ig κ-chain V-J2-C signal peptide; etc.

Suitable techniques for transforming a host or host cell of the invention will be clear to the skilled person and may depend on the intended host cell/host organism and the genetic construct to be used. Reference is again made to the handbooks and patent applications mentioned above.

After transformation, a step for detecting and selecting those host cells or host organisms that have been successfully transformed with the nucleotide sequence/genetic construct of the invention may be performed. This may for instance be a selection step based on a selectable marker present in the genetic construct of the invention or a step involving the detection of the amino acid sequence of the invention, e.g. using specific antibodies.

The transformed host cell (which may be in the form or a stable cell line) or host organisms (which may be in the form of a stable mutant line or strain) form further aspects of the present invention.

Preferably, these host cells or host organisms are such that they express, or are (at least) capable of expressing (e.g. under suitable conditions), an amino acid sequence of the invention (and in case of a host organism: in at least one cell; part, tissue or organ thereof). The invention also includes further generations, progeny and/or offspring of the host cell or host organism of the invention, that may for instance be obtained by cell division or by sexual or asexual reproduction.

To produce/obtain expression of the amino acid sequences of the invention, the transformed host cell or transformed host organism may generally be kept, maintained and/or cultured under conditions such that the (desired) amino acid sequence of the invention is expressed/produced. Suitable conditions will be clear to the skilled person and will usually depend upon the host cell/host organism used, as well as on the regulatory elements that control the expression of the (relevant) nucleotide sequence of the invention. Again, reference is made to the handbooks and patent applications mentioned above in the paragraphs on the genetic constructs of the invention.

Generally, suitable conditions may include the use of a suitable medium, the presence of a suitable source of food and/or suitable nutrients, the use of a suitable temperature, and optionally the presence of a suitable inducing factor or compound (e.g. when the nucleotide sequences of the invention are under the control of an inducible promoter); all of which may be selected by the skilled person. Again, under such conditions, the amino acid sequences of the invention may be expressed in a constitutive manner, in a transient manner, or only when suitably induced.

It will also be clear to the skilled person that the amino acid sequence of the invention may (first) be generated in an immature form (as mentioned above), which may then be subjected to post-translational modification, depending on the host cell/host organism used. Also, the amino acid sequence of the invention may be glycosylated, again depending on the host cell/host organism used.

The amino acid sequence of the invention may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g. using a specific, cleavable amino acid sequence fused with the amino acid sequence of the invention) and/or preparative immunological techniques (i.e. using antibodies against the amino acid sequence to be isolated).

Generally, for pharmaceutical use, the polypeptides of the invention may be formulated as a pharmaceutical preparation comprising at least one polypeptide of the invention and at least one pharmaceutically acceptable carrier, diluent or excipient and/or adjuvant, and optionally one or more further pharmaceutically active polypeptides and/or compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers for use in the preparation thereof, will be clear to the skilled person, and are further described herein.

Thus, in a further aspect, the invention relates to a pharmaceutical composition that contains at least one Nanobody of the invention or at least one polypeptide of the invention and at least one suitable carrier, diluent or excipient (i.e. suitable for pharmaceutical use), and optionally one or more further active substances.

Generally, the Nanobodies and polypeptides of the invention can be formulated and administered in any suitable manner known per se, for which reference is for example made to the general background art cited above (and in particular to WO 04/041862, WO 04/041863, WO 04/041865 and WO 04/041867) as well as to the standard handbooks, such as Remington's Pharmaceutical Sciences, 18^{th} Ed., Mack Publishing Company, USA (1990) or Remington, the Science and Practice of Pharmacy, 21th Edition, Lippincott Williams and Wilkins (2005).

For example, the Nanobodies and polypeptides of the inventions may be formulated and administered in any manner known per se for conventional antibodies and antibody fragments (including ScFv's and diabodies) and other pharmaceutically active proteins. Such formulations and methods for preparing the same will be clear to the skilled person, and for example include preparations suitable for parenteral administration (for example intravenous, intraperitoneal, subcutaneous, intramuscular, intraluminal, intra-arterial or intrathecal administration) or for topical (i.e. transdermal or intradermal) administration.

Preparations for parenteral administration may for example be sterile solutions, suspensions, dispersions or emulsions that are suitable for infusion or injection. Suitable carriers or diluents for such preparations for example include, without limitation, sterile water and aqueous buffers and solutions such as physiological phosphate-buffered saline, Ringer's solutions, dextrose solution, and Hank's solution; water oils; glycerol; ethanol; glycols such as propylene glycol or as well as mineral oils, animal oils and vegetable oils, for example peanut oil, soybean oil, as well as suitable mixtures thereof. Usually, aqueous solutions or suspensions will be preferred.

The present invention further relates to the use of the Nanobodies, polypeptides and compositions described herein in the diagnosis, treatment or prophylaxis of cancer. The present invention provides single domain antibodies, more precisely Nanobodies, directed to tumor-specific or tumor-associated antigens such as EGFR (EGFR) and insulin growth factor receptor (IGF-IR). The present invention further relates to their use in diagnosis and therapy.

Such antibodies may have a framework sequence with high homology to the human framework sequences. Compositions comprising antibodies to IGF-IR and EGFR alone or in combination with other drugs are described.

EGFR is part of the ERBB receptor family, which has four closely related members: EGFR (ErbB-1), HER2 (ErbB-2 or Neu), HER3 (ErbB-3) and HER4 (ErbB-4). Each of these members consist of an extracellular ligand-binding domain, a transmembrane domain and an intracellular tyrosine kinase domain (Yarden et al. 2001, Nature Rev. Mol. Cell. Biol. 2, 127-137).

The first step in the mitogenic stimulation of epidermal cells is the specific binding of ligands such as epidermal growth factor (EGF) or transforming growth factor alpha (TGFα) to a membrane glycoprotein known as the EGFR (EGF receptor). (Carpenter et al. 1979, Epidermal Growth Factor, Annual Review Biochem., Vol. 48, 193-216). The mature EGF receptor is composed of 1,186 amino acids which are divided into an extracellular portion of 621 residues and a cytoplasmic portion of 542 residues connected by a single hydrophobic transmembrane segment of 23 residues. (Ulhrich et al. 1986, Nature, Vol. 309, 418-425).

The external portion of the EGF receptor can be subdivided into four domains. It has been demonstrated that domain I and III, flanked by two cysteine rich domains, are likely to contain the EGF binding site of the receptor. (Ogiso et al. 2002. Crystal structure of the complex of human epidermal growth factor and receptor extracellular domains. Cell 110, 775-787; Garrett et al. 2002. Crystal structure of a truncated EGFR extracellular domain bound to transforming growth factor alpha. Cell 110, 763-773.). Without binding of ligand to the receptor, intramolecular domain II-domain IV interactions maintain the receptor in an inactive tethered state (Ferguson K M, Berger M B, Mendrola J M, Cho H S, Leahy D J, Lemmon M A 2003. EGF activates its receptor by removing interactions that autoinhibit ectodomain dimerization. Mol. Cell. 11:507-17).

As a consequence of monovalent ligand binding to domain I and III of EGFR, domain II becomes exposed and makes intermolecular contact with domain II of a neighboring receptor resulting in receptor dimerization. The dimerization state of the receptor is required to activate the tyrosine kinase in the cytoplasmic domain. This leads to transphosphorylation of tyrosine residus in the intracellular domain and the initiation of a myriad of signal transduction cascades resulting in DNA synthesis and eventually in cell proliferation and differentiation. EGFR activation initiates a cascade of events leading to the assembly of a number of adaptor proteins in a structure known as a clathrin coated pit, which, after invagination of the plasma membrane and budding gives rise to a vesicle termed clathrin coated vesicle (CCV).

Subsequently, the activated receptor is sorted into endosomes and finally to lysosomes for proteolitic degradation, resulting in receptor downregulation or sequestration (Vieira A V, Lamaze C, Schmid S L, 1996. Control of EGF receptor signaling by clathrin-mediated endocytosis. Science 274:2086-2089). In normal cells, this feedback mechanism controls abberant receptor signaling.

Abberant activation of EGFR has been implicated in processes involved in tumor growth and progression, including tumor cell proliferation, angiogenesis, metastasis, inhibition of apoptosis and resistance to radio- or chemotherapy (Grünwald V, Hidalgo M 2003. Developing inhibitors of the EGFR for cancer treatment. J. Natl. Cancer Inst. 95:851-867; and references herein). The dysregulation of EGF receptor signaling is a consequence of i) ligand or receptor overexpression (for example after gene duplication) or ii) constitutive receptor signaling by the formation of heterodimers or EGFR mutant forms such as EGFRvIII. As an example the heterodimer EGFR-HER2 is characterized by a more sustained signalling activity compared to the EGFR homodimer (Arteaga CL 2001. The EGFR: from mutant oncogene in nonhuman cancers to therapeutic target in human neoplasia. J. Clin. Oncol. 19:32 S-40S).

EGFR is expressed in a wide variety of tumors of epithelial origin, including >40% of NSCLC (none-small-cell-lung cancer), >95% of head and neck cancer, >30% of pancreatic cancer, >90% of renal carcinoma, >35% of ovarian cancer, >40% of glioma and >31% of bladder cancer (Salomon et al. 1995. Crit. Review Oncol. Hematol, 19, 183-232). Breast cancer cells exhibit a positive correlation between EGF receptor density and tumor size and a negative correlation with the extent of differentiation. (Sainsbury et al. 1985, EGFRs and Oestrogen Receptors in Human Breast Cancer, Lancet, Vol. 1, 364-366; Sainsbury et al. 1985, Presence of EGFR as an Indicator of Poor Prognosis in Patients with Breast Cancer, J. Clin. Path., Vol. 38, 1225-1228; Sainsbury et al. 1987. Epidermal-Growth-Factor Receptor Status as Predictor of Early Recurrence and Death From Breast Cancer, Lancet, Vol. 1, 1398-1400). Since high levels of EGFR expression are correlated to disease progression, increased metastasis and poor prognosis, this provides a strong rationale for developing effective EGFR targeting antibodies for the treatment of various solid tumors.

As synovial fibroblasts and keratinocytes are cell types that also express EGF receptor, these cells are candidate target cells for treatment of inflammatory arthritis and psoriasis, respectively. EGFR has also been implicated in several other diseases, such as inflammatory arthritis (U.S. Pat. No. 5,906,820, U.S. Pat. No. 5,614,488), laryngeal papillomas (Johnston D, Hall H, DiLorenzo TP, Steinberg B M 1999. Elevation of the EGFR and dependent signaling in human papillomavirus-infected laryngeal papillomas. Cancer Res. 59:968-74.) and hypersecretion of mucus in the lungs (Barnes P J, Hansel T T 2003. Prospects for new drugs for chronic obstructive pulmonary disease. Lancet 364:985-996; U.S. Pat. No. 6,566,324 and U.S. Pat. No. 6,551,989).

The identification of MAbs that inhibit EGFR is an approach used in clinical development to target aberrant signalling of EGFR in malignant neoplasia. Examples of such EGFR targeting antibodies are IMC-C225 (Erbitux, Imclone), EMD72000 (Merck Darmstadt), ABX-EGF (Abgenix), h-R3 (theraCIM, YM Biosciences) and Humax-EGFR (Genmab). The mechanism of action of these antibodies relies on the inhibition with ligand binding to the receptor and subsequent inhibition of receptor transphosphorylation and the downstream signaling cascade. Mab 225 (of which Erbitux is the chimeric derivative), the 225-derived F(ab′)₂ fragment are able to induce EGFR internalization and modest receptor sequestration but only after sustained incubation with EGFR expressing cells. The monovalent 225-derived Fab′ fragment however only induces receptor downregulation after preincubation with a rabbit anti-mouse antibody (Fan Z, Mendelsohn J, Masui H, Kumar R 1993. Regulation of EGFR in NIH3T3/Her-14 cells by antireceptor monoclonal antibodies. J. Biol. Chem. 268:21073-21079; Fan Z, Lu Y, Wu X, Mendelsohn J 1994. Antibody-induced EGFR dimerization mediates inhibition of autocrine proliferation of A431 squamous carcinoma cells. J. Biol. Chem. 269:27595-27602). These antibodies show an antitumoral activity against a broad panel of human tumor xenografts (reviewed in Grünwald V, Hidalgo M 2003. Developing inhibitors of the EGFR for cancer treatment. J. Natl. Cancer Inst. 95:851-867).

The primary goal in treating tumors is to kill all the cells of the tumor. A therapeutic agent that kills the cell is defined as cytotoxic. A therapeutic agent that prevents the cells to replicate rather than killing them is defined as cytostatic. The known antibody-based therapeutics which bind to the EGF receptor merely prevent the cells from replicating and thus such conventional antibodies act as a cytostatic agent (EP 667165, EP 359282, U.S. Pat. No. 5,844,093). Yet none of these antibodies nor the presently available small molecule drugs are completely effective for the treatment of cancer, and most are limited by severe toxicity. In addition, it is extremely difficult and a lengthy process to develop a new chemical entity (NCE) with sufficient potency and selectivity to such target sequence. Antibodies offer significant potential as drugs because they have exquisite specificity to their target and a low inherent toxicity. Additionally, the development time can be reduced considerably when compared to the development of new chemical entities (NCE's).

The use of antibodies derived from sources such as mouse, sheep, goat, rabbit etc., and humanized derivatives thereof as a treatment for cancer, requiring a cytostatic or cytotoxic effect, is problematic for several reasons. Since conventional antibodies are not stable at room temperature, they can not leave the required continuous cold chain (from production to patient treatment) adding extra costs to its development and use in therapy. Refrigeration is sometimes not feasible in developing countries. Furthermore, the manufacture or small-scale production of said antibodies is expensive because the mammalian cellular systems necessary for the expression of intact and functional antibodies require high levels of support in terms of time and equipment, and yields are very low. Conventional antibodies often show functional antigen binding activity in a limited pH window. Hence they are unsuitable for applications in environments with extreme pH conditions such as the digestive tract. Additionally, conventional antibodies are unstable at low or high pH and thus not suitable for oral administration. Furthermore, conventional antibodies are unsuitable for use in assays or kits performed at temperatures outside biologically active-temperature ranges (e.g. 37±20° C.) since the binding activity depends on temperature.

Polypeptide therapeutics and in particular antibody-based therapeutics have significant potential as drugs because they have exquisite specificity to their target and a low inherent toxicity. However, it is known by the skilled addressee that an antibody which has been obtained for a therapeutically useful target requires additional modification in order to prepare it for human therapy, so as to avoid an unwanted immunological reaction in a human individual upon administration. The modification process is commonly termed “humanization”. It is known by the skilled artisan that antibodies raised in species, other than in humans, require humanization to render the antibody therapeutically useful in humans ((1) CDR grafting: Protein Design Labs: U.S. Pat. No. 6,180,370, U.S. Pat. No. 5,693,761; Genentech U.S. Pat. No. 6,054,297; Celltech: 460167, EP 626390, U.S. Pat. No. 5,859,205; (2) Veneering: Xoma: U.S. Pat. No. 5,869,619, U.S. Pat. No. 5,766,886, U.S. Pat. No. 5,821,123). There is a need for a method for producing antibodies which avoids the requirement for substantial humanization, or which completely obviates the need for humanization. There is a need for a new class of antibodies which have defined framework regions or amino acid residues and which can be administered to a human subject without the requirement for substantial humanization, or the need for humanization at all.

Another important drawback of conventional antibodies is that they are complex, large molecules and therefore relatively unstable, and they are sensitive to breakdown by proteases. This means that conventional antibody drugs cannot be administered orally, sublingually, topically, nasally, vaginally, rectally or by inhalation because they are not resistant to the low pH at these sites, the action of proteases at these sites and in the blood and/or because of their large size. They have to be administered by injection (intravenously, subcutaneously, etc.) to overcome some of these problems. Administration by injection requires specialist training in order to use a hypodermic syringe or needle correctly and safely. It further requires sterile equipment, a liquid formulation of the therapeutic polypeptide, vial packing of said polypeptide in a sterile and stable form and, of the subject, a suitable site for entry of the needle. Furthermore, subjects commonly experience physical and psychological stress prior to and upon receiving an injection. Therefore, there is need for a method for the delivery of therapeutic polypeptides which avoids the need for injection which is not only cost- and time-saving, but which would also be more convenient and more comfortable for the subject.

Solid tumors consist of densely packed, highly proliferating cells. For the treatment of solid tumors, it is essential that the therapeutic antibody penetrates into the deepest layers of the tumor resulting in a rapid and homogeneous distribution to avoid tumor relapse. When comparing intact conventional antibodies (150 kDa) with derived immunoglobulin formats such as F(ab′)₂, Fab′ and scFv fragments (the smallest antigen binding unit formed by the genetic fusion of gene segments coding for the variable domain of the heavy and light chain separated by a short linker seqiuence), it was shown that scFvs showed the fastest and most homogeneous distribution in the tumor mass (Yokota T, Milenic D E, Whitlow M, Schlom J 1992. Rapid tumor penetration of a single-chain Fv and comparison with other immunoglobulin forms).

These and other objectives are accomplished with the use of the antibodies of the invention.

Nanobodies described in the invention which are derived from heavy chain antibodies from Camelidae, are known to have cavity-binding propensity (WO97/49805; Lauwereys et al, EMBO J. 17, 5312, 1998). Therefore, such Nanobodies are inherently suited to recognize ligand-binding domains on the receptor or to bind epitopes that are less accessible to conventional antibodies and may therefore operate via a different mechanism of action to yield a cytotoxic effect on tumour cells.

Heavy chain antibodies are devoid of light chains. Hence, its antigen binding domain is formed by a single domain, the V_{HH} (approximately 15 kDa), contrary to the antigen binding domain of conventional antibodies which requires functional folding of the variable domains of a heavy and a light chain. Only a single gene segment is required for expression of a functional V_{HH}, making these V_{HH}s suitable as building blocks (Conrath K E, Lauwereys M, Wyns L, Muyldermans S 2001. Camel single-domain antibodies as modular building units in bispecific and bivalent antibody constructs. J. Biol. Chem. 276:7346-7350). Such heavy chain antibody fragments (or multimeric derivatives) can easily be produced ‘en-masse’ in fermentors using cheap expression systems compared to mammalian cell culture fermentation, such as yeast, E. coli or other microorganisms (EP 0 698 097).

Furthermore, the Camelidae Nanobodies have been shown to have unexpectedly high thermal stability with Tₘs in the range of 60° C. to 80° C. and resist harsh conditions, such as extreme pH and denaturing reagents (Ewert S et al, Biochemistry 2002 Mar. 19; 41(11):3628-36; Pérez et al, Biochemistry, 2001, 40: 74-83), so making them suitable for delivery by oral administration. This allows, among others, for better labeling efficiency compared to labeling of conventional antibodies and their derivatives (e.g. scFv). As such, higher specific activities can be obtained resulting in superior imaging results or therapeutic efficacy. In addition, the Camelidae Nanobodies have increased shelf-life (Perez et al, Biochemistry, 40, 74, 2001). As V_{HH}s are the smallest antigen binding domains (15 kDa) they are extremely suitable for optimal distribution in solid tumor masses. Since V_{HH}s originate from naturally occurring heavy chain antibodies which already underwent an in vivo maturation, no time consuming and labor intensive in vitro affinity maturation is required anymore. It is an aim of the present invention to provide polypeptides comprising one or more single domain antibodies (and in particular Nanobodies) which bind to tumor-specific or tumor-associated antigens such as EGFR (EGFR) and insulin growth factor receptor (IGF-IR), homologues of said polypeptides and functional portions. Said polypeptides can i) inhibit binding of the natural ligand to the receptor and/or, ii) induce receptor downregulation, and/or iii) prevent homo- and heterodimerization of the receptor and/or iv) induce apoptosis in human cells, thereby modifying the biological activity of EGFR upon binding, v) detect tumors expressing IGF-IR and/or EGFR by using labeled polypeptides, vi) have a cytotoxic effect on the cell due to its binding to the tumor antigen. Such polypeptides might bind into the ligand-binding groove of EGFR, or might not bind in the ligand binding groove. Such polypeptides are single domain antibodies.

It is a further aim of the invention to provide Nanobodies or polypeptides joined to therapeutic compounds such as anti-tumor agents, or joined to imaging agents suitable for visualization in MRI or CAT-scans.

One embodiment of the present invention is an anti-EGFR or anti-IGF-IR polypeptide comprising at least one single domain antibody directed against EGRF or IGF-IR respectively.

Another embodiment of the present invention is an anti-EGFR or anti-IGF-IR polypeptide as described above wherein at least one single domain antibody is a heavy chain variable domain.

Another embodiment of the present invention is an anti-EGFR or anti-IGF-IR polypeptide as described above wherein at least one single domain antibody is a V_{HH} or Nanobody.

Another embodiment of the present invention is an anti-EGFR or anti-IGF-IR polypeptide as described above wherein at least one single domain antibody is a V_{HH} in which one or more amino acid residues have been substituted without substantially altering the antigen binding capacity.

Another embodiment of the present invention is an anti-EGFR or anti-IGF-IR polypeptide as described above wherein at least one single domain antibody is a VH wherein one or more amino acid residues have been substituted, and in particular Camelized, without substantially altering the antigen binding capacity.

Another embodiment of the present invention is an anti-EGFR or anti-IGF-IR polypeptide as described above wherein at least one single domain antibody is a humanized V_{HH}.

Another embodiment of the present invention is an anti-EGFR or anti-IGF-IR polypeptide as described above wherein said single domain antibody is an homologous sequence, a functional portion, or a functional portion of an homologous sequence of the full length single domain antibody.

Another embodiment of the present invention is an anti-EGFR or anti-IGF-IR polypeptide as described above further comprising at least one covalently attached or recombinantly fused substance directed to improving the half-life.

Another embodiment of the present invention is an anti-EGFR or anti-IGF-IR polypeptide as described above wherein said substance directed to improving the half-life is any of polyethylene glycol, serum protein.

Another embodiment of the present invention is an anti-EGFR or anti-IGF-IR polypeptide as described above wherein said substance is a single domain antibody directed against a serum protein.

Another embodiment of the present invention is an anti-EGFR or anti-IGF-IR polypeptide as described above wherein said serum protein is any of serum albumin, serum immunoglobulins, thyroxine-binding protein, transferrin, or fibrinogen.

Another embodiment of the present invention is an anti-EGFR or anti-IGF-IR polypeptide as described above wherein said serum protein is human.

Another embodiment of the present invention is an anti-EGFR or anti-IGF-IR polypeptide as described above wherein said single domain antibody is an homologous sequence, a functional portion, or a functional portion of an homologous sequence of the full length single domain antibody.

Another embodiment of the present invention is an anti-EGFR or anti-IGF-IR polypeptide as described above wherein said polypeptide is an homologous sequence, a functional portion, or a functional portion of an homologous sequence of the full length single domain antibody.

Another embodiment of the present invention is an anti-EGFR or anti-IGF-IR polypeptide as described above, wherein the number of single domain antibodies directed against EGFR or IGF-IR is at least two.

Another embodiment of the present invention is an anti-EGFR or anti-IGF-IR polypeptide as described above, wherein said at least two single domain antibodies are joined head to tail in the absence of a linker sequence.

Another embodiment of the present invention is an anti-EGFR or anti-IGF-IR polypeptide as described above, wherein at least one single domain antibody is capable of binding to EGFR, internalising the receptor and but not co-localising with transfenin.

Another embodiment of the present invention is an anti-EGFR or anti-IGF-IR polypeptide as described above further comprising one or more radioisotopes.

Another embodiment of the present invention is an anti-EGFR or anti-IGF-IR polypeptide as described above wherein at least one isotope is any of ¹⁸⁸Re, ¹³¹I or ²¹¹At.

Another embodiment of the present invention is an anti-EGFR or anti-IGF-IR polypeptide as described above further comprising one or more anti-tumour agents.

Another embodiment of the present invention is an anti-EGFR or anti-IGF-IR polypeptide as described above wherein at least one anti-tumour agent is any of antracyclines, methotraxate, vindesine, cis-platinum, ricin, calicheamicin and cytokine.

Another embodiment of the present invention is an anti-EGFR or anti-IGF-IR polypeptide as described above further comprising one or more enzymes capable of activating a prodrug.

Another embodiment of the present invention is a nucleic acid encoding an anti-EGFR or anti-IGF-IR polypeptide as described above.

Another embodiment of the present invention is an anti-EGFR or anti-IGF-IR polypeptide as described above, or a nucleic acid encoding said polypeptide for treating and/or preventing and/or alleviating disorders relating to inflammatory processes.

Another embodiment of the present invention is an anti-EGFR or anti-IGF-IR polypeptide as described above, or a nucleic acid encoding said polypeptide for treating and/or preventing and/or alleviating disorders relating to cancer.

Another embodiment of the present invention is a use of an anti-EGFR or anti-IGF-IR polypeptide as described above, or a nucleic acid encoding said polypeptide for the preparation of a medicament for treating and/or preventing and/or alleviating disorders relating to cancer.

Another embodiment of the present invention is an anti-EGFR polypeptide, anti-IGF-IR polypeptide or nucleic acid as described above wherein said cancer is selected from the group consisting of cancer of the breast, ovary, testis, lung, colon, rectum, pancreas, liver, central nervous system, head and neck, kidney, bone, blood and lymphatic system.

Another embodiment of the present invention is a composition comprising an anti-EGFR or anti-IGF-IR polypeptide as described above or a nucleic acid encoding said polypeptide and a suitable pharmaceutical vehicle.

Another embodiment of the present invention is a method of diagnosing a disorder characterised by the abberant signaling of EGFR or the presence of IGF-IR comprising:

(a) contacting a sample with an anti-EGFR or anti-IGF-IR polypeptide as described above,
(b) detecting binding of said polypeptide to said sample, and
(c) comparing the binding detected in step (b) with a standard, wherein a difference in binding relative to said sample is diagnostic of a disorder characterised by the aberrent signalling of EGFR or the presence of IGF-IR respectively.

Another embodiment of the present invention is a kit for screening for a disorder cited above, using a method as described above.

Another embodiment of the present invention is a kit for screening for a disorder cited above comprising an isolated anti-EGFR or anti-IGF-IR polypeptide as described above.

Another embodiment of the present invention is a use of an anti-EGFR or anti-IGF-IR polypeptide as described above for inhibiting the interaction between EGF and one or more EGF receptors or IGF-IR and one or more IGF-IR receptors respectively.

Another embodiment of the present invention is a method of producing an anti-EGFR or anti-IGF-IR polypeptide as described above comprising:

(a) culturing host cells comprising nucleic acid capable of encoding an anti-EGFR or anti-IGF-IR polypeptide as described above, under conditions allowing the expression of the polypeptide, and,
(b) recovering the produced polypeptide from the culture.

Another embodiment of the present invention is a method as described above, wherein said host cells are bacteria or yeast.

Another embodiment of the present invention is a kit for screening for cancer comprising an anti-EGFR or anti-IGF-IR polypeptide as described above.

Another embodiment of the present invention is an anti-EGFR or anti-IGF-IR polypeptide as described above further comprising one or more imaging agents.

Another embodiment of the present invention is an anti-EGFR or anti-IGF-IR polypeptide as described above wherein said imaging agents are any of radiolabels, enzyme labels, magnetic resonance paramagnetic chelates, and/or optical dyes.

Another embodiment of the present invention is a use of an anti-EGFR or anti-IGF-IR polypeptide as described above for imaging.

Another embodiment of the present invention is a method for imaging EGFR or IGF-IR targets comprising adminstering an anti-EGFR or anti-IGF-IR polypeptide as described above.

Use of an anti-EGFR or anti-IGF-IR polypeptide as described above for the preparation of a composition, more preferably a diagnostic composition for imaging.

Another embodiment of the present invention is a method as described above, wherein the number of anti-EGFR or anti-IGF-IR single domain antibodies is at least two.

Another embodiment of the present invention is a method as described above, wherein said polypeptides are administered to a subject in microparticles, ultrasound bubbles, microspheres, emulsions or liposomes.

Another embodiment of the present invention is a method for increasing the residence time of an anti-EGFR or anti-IGF-IR polypeptide on their respective receptor comprising fusing said polypeptide to one one or more single domain antibodies directed against serum protein.

Another embodiment of the present invention is a method as described above wherein said anti-EGFR or anti-IGF-IR polypeptide is a polypeptide as described above.

Another embodiment of the present invention is a method of identifying an agent that modulates the binding of an anti-EGFR or anti-IGF-IR polypeptide as described above to EGFR or IGF-IR comprising:

(a) contacting a polypeptide as described above with a target that is EGFR or IGF-IR, or a fragment thereof, in the presence and absence of a candidate modulator under conditions permitting binding between said polypeptide and corresponding target, and
(b) measuring the binding between the polypeptide and target of step (a), wherein a decrease in binding in the presence of said candidate modulator, relative to the binding in the absence of said candidate modulator identified said candidate modulator as an agent that modulates the binding of an anti-EGFR or anti-IGF-IR polypeptide as described above and EGFR or IGF-IR respectively.

Another embodiment of the present invention is a method of identifying an agent that modulates EGFR- or IGF-IR-mediated disorders through the binding of an anti-EGFR or anti-IGF-IR polypeptide or anti-IGF-IR polypeptide respectively as described above to EGFR or IGF-IR comprising:

(a) contacting an anti-EGFR or anti-IGF-IR polypeptide as described above with a target that is EGFR or IGF-IR, or a fragment thereof, in the presence and absence of a candidate modulator under conditions permitting binding between said polypeptide and corresponding target, and
(b) measuring the binding between the polypeptide and target of step (a), wherein a decrease in binding in the presence of said candidate modulator, relative to the binding in the absence of said candidate modulator identified said candidate modulator as an agent that modulates EGFR- or IGF-IR-mediated disorders.

Another embodiment of the present invention is a method of identifying an agent that modulates the binding of Epidermal Growth Factor to its receptor through the binding of an anti-EGFR or anti-IGF-IR polypeptide as described above to EGFR comprising:

(a) contacting an anti-EGFR or anti-IGF-IR polypeptide as described above with a target that is EGFR, or a fragment thereof, or homologous sequence thereof, in the presence and absence of a candidate modulator under conditions permitting binding between said polypeptide and target, and
(b) measuring the binding between the polypeptide and target of step (a), wherein a decrease in binding in the presence of said candidate modulator, relative to the binding in the absence of said candidate modulator identified said candidate modulator as an agent that modulates the binding of EGFR natural ligand.

Another embodiment of the present invention is a kit for screening for agents that modulate EGFR -mediated or anti-IGF-IR-mediated disorders comprising an anti-EGFR or anti-IGF-IR polypeptide as described above and EGFR or IGF-IR respectively, or a fragment thereof.

Another embodiment of the present invention is an unknown agent that modulates the binding of the polypeptides as described above to EGFR or IGF-IR, identified according to the method as described above.

Another embodiment of the present invention is an unknown agent that modulates EGFR-mediated or IGF-IR-mediated disorders, identified according to the methods as described above.

Another embodiment of the present invention is an unknown agent as described above wherein said disorders are one or more of cancer, rheumatoid arthritis, psoriasis, or hypersecretion of mucus in the lung.

Another embodiment of the present invention is an anti-EGFR or anti-IGF-IR polypeptide as described above for treating and/or preventing and/or alleviating disorders susceptible to modulation by the delivery of an EGFR or IGF-IR antagonist that is able to pass through the gastric environment without being inactivated.

Another embodiment of the present invention is a use of anti-EGFR or anti-IGF-IR polypeptide as described above for the preparation of a medicament for treating, preventing and/or alleviating the symptoms of disorders susceptible to modulation by the delivery of an EGFR or IGF-IR antagonist that is able to pass through the gastric environment without being inactivated.

Another embodiment of the present invention is a polypeptide as described above for treating and/or preventing and/or alleviating the symptoms of disorders susceptible to modulation by the delivery of an EGFR or IGF-IR antagonist to the vaginal and/or rectal tract without inactivation.

Another embodiment of the present invention is a use of a polypeptide as described above for the preparation of a medicament for treating, preventing and/or alleviating the symptoms of disorders susceptible to modulation by the delivery of an EGFR or IGF-IR antagonist to the vaginal and/or rectal tract without inactivation.

Another embodiment of the present invention is a polypeptide as described above for treating and/or preventing and/or alleviating disorders susceptible to modulation by the delivery of an EGFR or IGF-IR antagonist to the upper respiratory tract and lung without inactivation.

Another embodiment of the present invention is a use of a polypeptide as described above for the preparation of a medicament for treating, preventing and/or alleviating the symptoms of disorders requiring the delivery of a therapeutic compound to the upper respiratory tract and lung.

Another embodiment of the present invention is a polypeptide as described above for treating and/or preventing and/or alleviating disorders susceptible to modulation by the delivery of an EGFR or IGF-IR antagonist to the intestinal mucosa without inactivation, wherein said disorder increases the permeability of the intestinal mucosa.

Another embodiment of the present invention is a use of a polypeptide as described above for the preparation of a medicament for treating, preventing and/or alleviating the symptoms of disorders susceptible to modulation by the delivery of an EGFR or IGF-IR antagonist without inactivation, wherein said disorder increases the permeability of the intestinal mucosa.

Another embodiment of the present invention is a polypeptide as described above for treating and/or preventing and/or alleviating disorders susceptible to modulation by the delivery of an EGFR or IGF-IR antagonist to the tissues beneath the tongue without inactivation.

Another embodiment of the present invention is a use of a polypeptide as described above for the preparation of a medicament for treating, preventing and/or alleviating the symptoms of disorders susceptible to modulation by the delivery of an EGFR or IGF-IR antagonist to the tissues beneath the tongue without inactivation.

Another embodiment of the present invention is a polypeptide as described above for treating and/or preventing and/or alleviating disorders susceptible to modulation by the delivery of an EGFR or IGF-IR antagonist through the skin without inactivation.

Another embodiment of the present invention is a use of a polypeptide as described above for the preparation of a medicament for treating, preventing and/or alleviating the symptoms of disorders susceptible to modulation by the delivery of an EGFR or IGF-IR antagonist through the skin without inactivation.

Another embodiment of the present invention is a polypeptide, nucleic acid or agent as described above, use of a polypeptide, nucleic acid or agent as described above, a polypeptide as described above, use of a polypeptide as described above wherein said disorders are cancer, rheumatoid arthritis, psoriasis, or hypersecretion of mucus in the lung.

Another embodiment of the present invention is a use of a polypeptide as described above for inhibiting the interaction between EGF and one or more EGFR. Another embodiment of the present invention is a therapeutic composition comprising:

(a) a V_{HH} which inhibits the growth of human tumor cells by said V_{HH} binding to EGFR or IGF-IR of said tumour cell, and
(b) an anti-neoplastic agent.

Another embodiment of the present invention is a therapeutic composition as described above for separate administration of the components.

The present invention relates to an anti-EGFR (EGFR) polypeptide, comprising at least one single domain antibody which is directed towards EGFR. The present invention also relates to an anti-IGF-IR (IGF-IR) polypeptide, comprising at least one single domain antibody which is directed towards IGF-IR. The invention also relates to nucleic acids capable of encoding said polypeptides.

The EGFR is overexpressed on the surface of many cancer cells, and this overexpression is associated with bad prognosis, progression, shortened survival and resistance to chemotherapy or hormone therapy. Also it has been proven that EGFR overexpression alters cell cycle regulation (increasing proliferation on tumor cell), blocks apoptosis and promotes angiogenesis. In this way the blockade of the EGFR associated signal transduction pathway in tumor cells should result in: (i) Inhibition of tumor cell proliferation, (ii) cell cycle arrest, (iii) induction of apoptosis and (iv) inhibition of angiogenesis.

The pharmaceutical compounds of the present invention block the binding of the EGF to the receptor, inhibit activation of receptor tyrosine kinase (inhibit tyrosine phosphorylation) induced by EGF binding, stimulate receptor internalization, inhibit cell proliferation induced by EGF on in vitro cell culture, have significant pro-apoptotic effect, and have a potent anti-angiogenic activity causing down regulation of the VEGF production and diminishing the number of microvessel counts. These effects are further amplified when the pharmaceutical compound is combined with other anti-cancer treatments.

One embodiment of the present invention relates to a pharmaceutical composition comprising at least one polypeptide of the invention and at least a pharmaceutical acceptable carrier, diluent or excipients.

According to one preferred, but non-limiting embodiment, said pharmaceutical composition is suitable for oral administration.

According to one aspect of the invention, Nanobodies are polypeptides which are derived from heavy chain antibodies and whose framework regions and complementary determining regions are part of a single domain polypeptide. Examples of such heavy chain antibodies include, but are not limited to, naturally occurring immunoglobulins devoid of light chains. Such immunoglobulins are disclosed in WO 94/04678 for example.

The antigen-binding site of this unusual class of heavy chain antibodies has a unique structure that comprises a single variable domain. For clarity reasons, the variable domain derived from a heavy chain antibody naturally devoid of light chain is known herein as a V_{HH} or V_{HH} domain or nanobody. Such a V_{HH} domain peptide can be derived from antibodies raised in Camelidae species, for example in camel, dromedary, llama, alpaca and guanaco.

Other species besides Camelidae (e.g. shark, pufferfish) may produce functional antigen-binding heavy chain antibodies naturally devoid of light chain. V_{HH} domains derived from such heavy chain antibodies are within the scope of the invention.

Camelidae antibodies express a unique, extensive repertoire of functional heavy chain antibodies that lack light chains. The V_{HH} molecules derived from Camelidae antibodies are the smallest intact antigen-binding domains known (approximately 15 kDa, or 10 times smaller than conventional IgG) and hence are well suited towards delivery to dense tissues and for accessing the limited space between macromolecules.

Other examples of Nanobodies include Nanobodies derived from VH domains of conventional four chain antibodies which have been modified by substituting one or more amino acid residues with Camelidae-specific residues (the so-called camelisation of heavy chain antibodies, WO 94/04678). Such positions may preferentially occur at the VH-VL interface and at the so-called Camelidae hallmark residues (WO 94/04678), comprising positions 37, 44, 45, 47, 103 and 108.

Nanobodies correspond to small, robust and efficient recognition units formed by a single immunoglobulin (Ig) domain.

The anti-EGFR or anti-IGF-IR polypeptides as disclosed herein and their derivatives not only possess the advantageous characteristics of conventional antibodies, such as low toxicity and high selectivity, but they also exhibit additional properties. They are more soluble; as such they may be stored and/or administered in higher concentrations compared with conventional antibodies.

Conventional antibodies are not stable at room temperature, and have to be refrigerated for preparation and storage, requiring necessary refrigerated laboratory equipment, storage and transport, which contribute towards time and expense. The anti-EGFR or anti-IGF-IR polypeptides of the present invention are stable at room temperature, as such they may be prepared, stored and/or transported without the use of refrigeration equipment, conveying a cost, time and environmental savings. Furthermore, conventional antibodies are unsuitable for use in assays or kits performed at temperatures outside biologically active-temperature ranges (e.g. 37±20° C.).

Other advantageous characteristics of the anti-EGFR or anti-IGF-IR polypeptides as disclosed herein as compared to conventional antibodies include modulation of half-life in the circulation which may be modulated according to the invention by, for example, albumin-coupling, or by coupling to one or more Nanobodies directed against a serum protein such as, for example, serum albumin. One aspect of the invention is a bispecific anti-EGFR polypeptide, with one specificity against a serum protein such as serum albumin and the other against the target as disclosed in WO04/041865 and incorporated herein by reference. Other means to enhance half life include coupling a polypeptide of the present invention to Fc, or to other Nanobodies directed against EGFR (i.e. creating multivalent Nanobodies—bivalent, trivalent, etc.) or coupling to polyethylene glycol. A controllable half-life is desirable for modulating dosage with immediate effect.

Conventional antibodies are unsuitable for use in environments outside the usual physiological pH range. They are unstable at low or high pH and hence are not suitable for oral administration. Camelidae antibodies resist harsh conditions, such as extreme pH, denaturing reagents and high temperatures, so making the anti-EGFR or anti-IGF-IR polypeptides as disclosed herein suitable for delivery by oral administration. Camelidae antibodies are resistant to the action of proteases which is less the case for conventional antibodies.

The yields of expression of conventional antibodies are very low and the method of production is very labor intensive. Furthermore, the manufacture or small-scale production of said antibodies is expensive because the mammalian cellular systems necessary for the expression of intact and active antibodies require high levels of support in terms of time and equipment, and yields are very low. The anti-EGFR or anti-IGF-IR polypeptides of the present invention may be cost-effectively produced through fermentation in convenient recombinant host organisms such as Escherichia coli and yeast; unlike conventional antibodies which also require expensive mammalian cell culture facilities, achievable levels of expression are high. Examples of yields of the polypeptides of the present invention are 1 to 10 mg/ml (E. coli) and up to 1 g/l (yeast).

The anti-EGFR or anti-IGF-IR polypeptides of the present invention exhibit high binding affinity for a broad range of different antigen types, and ability to bind to epitopes not recognised by conventional antibodies; for example they display long CDR3 loops with the potential to penetrate into cavities.

The anti-EGFR or anti-IGF-IR polypeptides of the present invention exhibit a straightforward generation of bi- or multi-functional molecules by (head-to-tail) fusion as disclosed in WO96/34103 (incorporated herein by reference).

Through their small size, the anti-EGFR or anti-IGF-IR polypeptides of the present invention allow better tissue penetration and ability to reach all parts of the body than conventional antibodies.

Llama single-domain antibodies can transmigrate across human blood-brain barrier. In one embodiment of the invention the anti-EGFR or anti-IGF-IR polypeptides can penetrate the blood-brain-barrier. In another embodiment of the invention the anti-EGFR or anti-IGF-IR polypeptides may not penetrate the blood-brain barrier.

The anti-EGFR or anti-IGF-IR polypeptides as disclosed herein are less immunogenic than conventional antibodies. A subclass of Camelidae antibodies has been discovered which displays 95% amino acid sequence homology to human VH framework regions. This suggests that immunogenicity upon administration in human patients can be anticipated to be minor or even non-existent. Alternatively, if so required, humanization of Nanobodies surprisingly requires only a few residues that need to be substituted.

According to one preferred, but non-limiting embodiment, a polypeptide of the invention has an iso-electrical point between 4 and 11. Preferably, a polypeptide of the invention has an iso-electrical point between 5 and 10.

According to one preferred, but non-limiting embodiment, the polypeptides of the invention comprise two amino acid chains (herein called “heavy chains”) which are covalently linked.

The heavy chains of the invention are preferably linked via a disulfide bond. More preferably, the heavy chains of the invention are linked via cysteine residues forming a disulfide bond.

According to one preferred, but non-limiting embodiment, the heavy chains of the invention have an approximate molecular weight of between 35 kDa and 50 kDa. The molecular weight is determined as described in Hamers-Casterman et al. (Nature 1993).

Preferably, the heavy chains of the invention have a molecular weight of between 40 kDa and 50 kDa.

More preferably, the heavy chains of the invention have a molecular weight of between 41 kDa and 49 kDa, 42 kDa and 48 kDa, 43 kDa and 47 kDa, or 44 kDa and 46 kDa. Most preferably, the heavy chains of the invention have a molecular weight of between 43 kDa and 46 kDa.

According to one preferred, but non-limiting embodiment, the heavy chains of the invention have a molecular weight of 43 kDa.

According to another preferred, but non-limiting embodiment, the heavy chains of the invention have a molecular weight of 46 kDa.

Single domain antibodies are antibodies whose complementary determining regions are part of a single domain polypeptide. Examples include, but are not limited to, heavy or light chain antibodies, antibodies naturally devoid of light chains, single domain antibodies derived from conventional 4-chain antibodies, VH domain from conventional antibodies, the respective human germline sequences encoding parts of these, engineered antibodies and single domain scaffolds other than those derived from antibodies. Single domain antibodies may be any of the art, or any future single domain antibodies. Single domain antibodies may be derived from any species including, but not limited to mouse, human, camel, llama, shark, pufferfish, goat, rabbit, bovine. According to one aspect of the invention, a single domain antibody as used herein is a naturally occurring immunoglobulin devoid of light chains. Such single domain antibodies are disclosed in WO 94/04678 for example. For clarity reasons, this variable domain derived from a heavy chain antibody naturally devoid of light chain is known herein as a V_{HH} or Nanobody to distinguish it from the conventional VH of four chain immunoglobulins. Such a V_{HH} molecule can be derived from antibodies raised in Camelidae species, for example in camel, dromedary, llama, vicufia, alpaca and guanaco. Other species besides Camelidae (e.g. shark, pufferfish) may produce functional antigen-binding heavy chain antibodies naturally devoid of light chain; and V_{HH}s derived from such heavy chain antibodies are within the scope of the invention.

Nanobodies, according to the present invention, are heavy chain variable domains derived from immunoglobulins naturally devoid of light chains such as those derived from Camelidae as described in WO 94/04678 (and referred to hereinafter as V_{HH} domains or Nanobodies). V_{HH} molecules are about 10× smaller than IgG molecules. They are single polypeptides and very stable, resisting extreme pH and temperature conditions. Furthermore, expression of V_{HH}s in microbial hosts produce high yields, properly folded functional V_{HH}s. Expression in mammalian hosts can therefore be avoided.

Moreover, they are resistant to the action of proteases which is less the case for conventional antibodies. V_{HH}s of the invention are resistant to digestion by proteases of the digestive tract, and generally more so than conventional antibodies. This would allow such V_{HH}s to be used therapeutically via oral intake. Furthermore, the V_{HH}s of the invention have been shown to be stable over several months when incubated in human serum at 37° C.

In addition, antibodies generated in Camelids will recognise epitopes other than those recognised by antibodies generated in vitro through the use of antibody libraries or via immunization of mammals other than Camelids (WO 97/49805). As such, anti EGFR and anti-IGF-IR V_{HH}'S may interact more efficiently or induce a unique biological effect when binding to EGFR or IGF-IR respectively than conventional antibodies, thereby blocking its interaction with the EGFR ligand(s) or block the activity of the IGF-IR antigen more efficiently on their respective receptors. Since V_{HH}'s are known to bind into ‘unusual’ epitopes such as cavities or grooves (WO 97/49805), the affinity of such V_{HH}'S may be more suitable for therapeutic treatment.

Several EGFR targeting antibodies such as IMC-C225, ABX-EGF, Humax-EGFR, hR3 and EMD72000 have been described that show a cytostatic effect on human carcinoma cells. The biological mechanism thereof is explained by the capacity of these EGFR targeting antibodies to inhibit binding of ligand to the ectodomain of the EGF receptor, thus preventing receptor-mediated downstream signaling required for cell proliferation. One of the commercially available EGFR antibodies showing a cytostatic effect is Erbitux (the chimeric version of a mouse monoclonal antibody 225) which was recently approved for treatment of colorectal cancer in a combination therapy with irinotecan. One of the machineries of the cell to reduce EGF receptor-mediated signaling is the mechanism of receptor sequestration or down-regulation. After binding of a ligand to the receptor, a cell can down-regulate receptor signaling by internalization of the ligand-receptor complex resulting in the degradation of the ligand-receptor complex in the lysosomes.

The inventors have surprisingly found that certain anti-EGFR Nanobodies are able to compete with EGF, TGFα and/or Erbitux. The new anti-EGFR Nanobodies are functionally classified into 3 Types of ligand competing Nanobodies. Non-limiting examples of the different classes of Nanobodies are as follows:
Type A Nanobody (binds to ectodomain EGFR, competes with the EGF, TGFα but not with the Erbitux binding sites on EGFR. Examples of such Nanobodies are 27-10-E8 (SEQ ID NO: 80; family I) and PMP7A5 (SEQ ID NO: 84; family III).
Type B Nanobody (binds to ectodomain EGFR, competes with the EGF, Erbitux and TGFα-binding sites on EGFR. Examples of such Nanobodies are PMP7D12 and PMP7C12 (SEQ ID NO's: 81-82; family II).
Type C Nanobody (binds to ectodomain EGFR, competes with the TGFα but not the EGF or Erbitux-binding sites on EGFR downregulation. An example of such a Nanobody is PMP8C7 (SEQ ID NO: 90; family XIII).

Accordingly, an embodiment of the invention is an anti-EGFR polypeptide comprising a single domain antibody or Nanobody specifically binding to the ectodomain of EGFR and competing with the EGF and TGFα binding site on EGFR, but not with the Erbitux binding site on EGFR. Another embodiment of the invention is an anti-EGFR polypeptide comprising a single domain antibody or Nanobody specifically binding the ectodomain of EGFR and competing with the EGF, TGFα and Erbitux binding site on EGFR. In yet another embodiment of the invention, the anti-EGFR polypeptide comprises a single domain antibody or Nanobody specifically binding the ectodomain of EGFR and competing with the TGFα binding site on EGFR but not with the EGF or Erbitux binding site on EGFR

One aspect of the invention is an anti-EGFR or anti-IGF-IR polypeptide comprising at least one anti-EGFR Nanobody. It is an aspect of the invention that such a polypeptide may comprise additional components. Such components may be polypeptide sequences, for example, one or more anti-EGFR Nanobodies, one or more anti-IGF-IR Nanobodies , one or more anti-serum albumin Nanobodies. Other fusion proteins are within the scope of the invention, and may include, for example, fusions with carrier polypeptides, signaling molecules, tags, and enzymes. Other components may include, for example, radiolabels, organic dyes, fluorescent compounds.

One embodiment of the present invention is an anti-EGFR or anti-IGF-IR polypeptide consisting of a sequence corresponding to that of a Camelidae V_{HH} directed towards EGFR or IGF-IR respectively, or a closely related family member.

A single domain antibody of the present invention is directed against EGFR, IGF-IR or a closely related family member.

Another embodiment of the present invention is a multivalent anti-EGFR or anti-IGF-IR polypeptide as disclosed herein comprising at least two single domain antibodies directed against EGFR. Another embodiment of the present invention is a multivalent anti-IGF-IR polypeptide as disclosed herein comprising at least two single domain antibodies directed against IGF-IR. Such multivalent anti-EGFR or anti-IGF-IR polypeptides have the advantage of unusually high functional affinity for the target, displaying much higher than expected inhibitory properties compared to their monovalent counterparts.

The multivalent anti-EGFR or anti-IGF-IR polypeptides have functional-affinities that are several orders of magnitude higher than the monovalent parent anti-EGFR or anti-IGF-IR polypeptides respectively. The inventors have found that the functional affinities of these multivalent polypeptides are much higher than those reported in the prior art for bivalent and multivalent antibodies. Surprisingly, anti-EGFR or anti-IGF-IR polypeptides of the present invention comprising single domain antibodies linked to each other directly or via a short linker sequence show the high functional affinities expected theoretically with multivalent conventional four-chain antibodies.

The inventors have found that such large increased functional activities can be detected preferably with antigens composed of multidomain and multimeric proteins, either in straight binding assays or in functional assays, e.g. cytotoxicity assays.

The inventors have also found that multivalent polypeptides have increased residence times and affinity/avidity towards their respective IGF-IR and/or EGFR targets. These multimeric polypeptides can be altered to provide IGF-IR or EGFR-specific imaging agents by radiolabeling, enzymatic labeling, or labeling with MR paramagnetic chelates or incorporated in microparticles, ultrasound bubbles, microspheres, emulsions, or liposomes; or wherein the binding moieties are conjugated with optical dyes.

A multivalent anti-EGFR or anti-IGF-IR polypeptide as used herein refers to a polypeptide comprising two or more anti-EGFR single domain antibodies which have been covalently linked. A multivalent anti-IGF-IR polypeptide as used herein refers to a polypeptide comprising two or more anti-IGF-IR single domain antibodies which have been covalently linked. The anti-EGFR or anti-IGF-IR single domain antibodies may be identical in sequence or may be different in sequence, but are directed against the same target or the same antigens or epitopes thereof. Alternatively, such multivalent constructs may be directed to different epitopes of the same target.

Depending on the number of anti-EGFR or anti-IGF-IR single domain antibodies linked, a multivalent anti-EGFR or anti-IGF-IR polypeptide may be bivalent (two anti-EGFR single domain antibodies or two anti-IGF-IR single domain antibodies), trivalent (3 anti-EGFR or anti-IGF-IR single domain antibodies), tetravalent (4 anti-EGFR or anti-IGF-IR single domain antibodies) or higher valency molecules.

According to another aspect of the invention, an anti-EGFR or anti-IGF-IR polypeptide may comprise at least two anti-EGFR Nanobodies. It is an aspect of the invention that such a polypeptide may comprise additional components as described above.

Examples of an anti-EGFR or anti-IGF-IR polypeptide of the invention comprising two anti-EGFR Nanobodies or two anti-IGF-IR Nanobodies are the polypeptides described below in Table 4 (SEQ ID NO's: 122-133 and 141-143) and Table 6 (SEQ ID NO's: 134-135) respectively.

According to a further aspect of the invention, an anti-EGFR or anti-IGF-IR polypeptide of the invention may comprise 1, 2, 3, 4, 5, 6, 7, 8, 9, 10, 11, 12, 13, 14, 15 or more than 15 Nanobodies directed against EGFR or IGF-IR respectively.

According to an aspect of the invention, an anti-EGFR or anti-IGF-IR polypeptide of the invention may comprise at least two identical or non identical anti-EGFR Nanobody sequences. It may be an aspect of the invention that at least two of the aforementioned sequences do not have equal affinity for EGFR, so forming an anti-EGFR or anti-IGF-IR polypeptide combining weak and high affinity binding sequences.

Examples of bivalent anti-EGFR or anti-IGF-IR polypeptides of the invention, comprising two identical Nanobodies directed against EGFR or IGF-IR respectively, include sequences given below in Table 4 (SEQ IS NO's: 122-133) and Table 6 (SEQ ID NO's: 134-135) respectively.

As described below, Nanobodies may be combined with or without linker sequences.

Methods of constructing bivalent polypeptides are known in the art (e.g. US 2003/0088074), and are also described below.

It may be desirable to modify the anti-EGFR or anti-IGF-IR polypeptide of the invention with respect to effector function so as to enhance its therapeutic efficacy. For example, Nanobody-fusions with certain Fc domains may be advantageous, especially with Fc domains of human origin.

The present invention also relates to the finding that an anti-EGFR or anti-IGF-IR polypeptide as disclosed herein further comprising one or more Nanobodies each directed against a serum protein of a subject, surprisingly has significantly prolonged half-life in the circulation of said subject compared with the half-life of the anti-EGFR or anti-IGF-IR Nanobody when not part of said polypeptide. Furthermore, said anti-EGFR or anti-IGF-IR polypeptides were found to exhibit the same favourable properties of Nanobodies as described above, such as, for example, high stability remaining intact in mice, extreme pH resistance, high temperature stability and high target specificity and affinity.

Thus, an anti-EGFR or anti-IGF-IR polypeptide as disclosed herein comprising one or more Nanobodies directed against EGFR or IGF-IR respectively, and one or more Nanobodies with specificity to a serum protein is much more efficient than a polypeptide only targeting EGFR or IGF-IR respectively.

Examples of bispecific anti-EGFR polypeptides e.g. comprising one Nanobody against EGFR and one Nanobody against serum albumin may be essentially as described in WO 05/044858 and WO 04/041867.

The serum protein may be any suitable protein found in the serum of a subject, or fragment thereof. In one aspect of the invention, the serum protein is any of serum albumin, serum immunoglobulins, thyroxine-binding protein, transferrin or fibrinogen. The subject may be, for example, rabbit, goat, mice, rat, cow, calve, camel, llama, monkey, donkey, guinea pig, chicken, sheep, dog, cat, horse, and preferably human. Depending on the intended use such as the required half-life for effective treatment and/or compartmentalization of the target antigen, the Nanobody partner can be directed to one of the above serum proteins.

According to one aspect of the invention, the number of Nanobodies directed against a serum protein in an anti-EGFR or anti-IGF-IR polypeptide of the invention is 1, 2, 3, 4, 5, 6, 7, 8, 9, 10, 11, 12, 13, 14, 15 ormorethan 15.

Another aspect of the invention is an anti-EGFR or anti-IGF-IR polypeptide further comprising at least one substance, covalently Ooined) or non-covalently bound, directed to improving the half-life of the polypeptide in vivo. Examples of substances which improve the half-lives are known in the art and include, for example, polyethylene glycol and serum albumin.

Methods for joining Nanobodies and other substances to form bi- and multi-specific polypeptides are known to the skilled person, and described below.

Polypeptides of the invention not modified according to the present invention to increase-half life, have the characteristic of rapid clearance from the body. Conversely, bispecific polypeptides comprising one or more Nanobodies directed against EGFR and one or more anti-serum protein Nanobodies are able to circulate in the subject's serum for several days, reducing the frequency of treatment, increasing the persistence times of the functional activity in the body, reducing the inconvenience to the subject and resulting in a decreased cost of treatment. The same advantageous characteristics are observable for polypeptides of the present invention comprising other substances aimed at improving the half life.

Furthermore, it is an aspect of the invention that the half-life of the anti-EGFR or anti-IGF-IR polypeptides disclosed herein may be controlled by the number of anti-serum protein Nanobodies present in the polypeptide. A controllable half-life is desirable in several circumstances, for example, in the application of a timed dose of a therapeutic anti-EGFR polypeptide.

Methods for pharmacokinetic analysis and determination of half-life are familiar to those skilled in the art. Details may be found in Kenneth, A et al: Chemical Stability of Pharmaceuticals: A Handbook for Pharmacists and in Peters et al, Pharmacokinete analysis: A Practical Approach (1996). Reference is also made to “Pharmacokinetics”, M Gibaldi & D Perron, published by Marcel Dekker, 2 nd Rev. ex edition (1982).

According to one aspect of the invention the polypeptides are capable of binding to one or more molecules which can increase the half-life of the polypeptide in vivo.

Half-life is the time taken for the serum concentration of the polypeptide to reduce by 50%, in vivo, for example due to degradation of the ligand and/or clearance or sequestration of the ligand by natural mechanisms. The polypeptides of the invention are stabilised in vivo and their half-life increased by binding to molecules which resist degradation and/or clearance or sequestration. Typically, such molecules are naturally occurring proteins which themselves have a long half-life in vivo.

The half-life of a polypeptide of the invention is increased if its functional activity persists, in vivo, for a longer period than a similar polypeptide which is not specific for the half-life increasing molecule. Thus, a polypeptide of the invention specific for HSA and a target molecule is compared with the same polypeptide wherein the specificity for HSA is not present, that it does not bind HSA but binds another molecule. For example, it may bind a second epitope on the target molecule. Typically, the half-life is increased by 10%, 20%, 30%, 40%, 50% or more. Increases in the range of 2×, 3×, 4×, 5×, 10×, 20×, 30×, 40×, 50× or more of the half-life are possible. Alternatively, or in addition, increases in the range of up to 30×, 40×, 50×, 60×, 70×, 80×, 90×, 100×, 150× of the half-life are possible.

Typically, molecules which can increase the half-life of the polypeptide in vivo are polypeptides which occur naturally in vivo and which resist degradation or removal by endogenous mechanisms which remove unwanted material from the organism. For example, the molecule which increases the half-life of the organism may be selected from the following:
Proteins from the extracellular matrix; for example collagen, laminins, integrins and fibronectin. Collagens are the major proteins of the extracellular matrix. About 15 types of collagen molecules are currently known, found in different parts of the body, e.g. type I collagen (accounting for 90% of body collagen) found in bone, skin, tendon, ligaments, cornea, internal organs or type II collagen found in cartilage, invertebral disc, notochord, vitreous humour of the eye;
Proteins found in blood, including: Plasma proteins such as fibrin, alpha-2 macroglobulin, serum albumin, fibrinogen A, fibrinogen B, serum amyloid protein A, heptaglobin, profilin, ubiquitin, uteroglobulin and beta-2-microglobulin;
Enzymes and inhibitors such as plasminogen, lysozyme, cystatin C, alpha-1-antitrypsin and pancreatic trypsin inhibitor. Plasminogen is the inactive precursor of the trypsin-like serine protease plasmin. It is normally found circulating through the blood stream. When plasminogen becomes activated and is converted to plasmin, it unfolds a potent enzymatic domain that dissolves the fibrinogen fibers that entangle the blood cells in a blood clot. This is called fibrinolysis.
Immune system proteins, such as IgE, IgG, IgM.
Transport proteins such as retinol binding protein, alpha-1 microglobulin.
Defensins such as beta-defensin 1, Neutrophil defensins 1, 2 and 3.
Proteins found at the blood brain barrier or in neural tissues, such as melanocortin receptor, myelin, ascorbate transporter.
Transferrin receptor specific ligand-neuropharmaceutical agent fusion proteins (see U.S. Pat. No. 5,977,307);
brain capillary endothelial cell receptor, transferrin, transferrin receptor, insulin, insulinlike growth factor 1 (IGF 1) receptor, insulin-like growth factor 2 (IGF 2) receptor, insulin receptor.
Proteins localised to the kidney, such as polycystin, type IV collagen, organic anion transporter KI, Heymann's antigen.
Proteins localised to the liver, for example alcohol dehydrogenase, G250.
Blood coagulation factor X, Alpha I antitrypsin, HNF 1 alpha.
Proteins localised to the lung, such as secretory component (binds IgA).
Proteins localised to the heart, for example HSP 27. This is associated with dilated cardiomyopathy.
Proteins localised to the skin, for example keratin.
Bone specific proteins, such as bone morphogenic proteins (BMPs), which are a subset of the transforming growth factor beta superfamily that demonstrate osteogenic activity. Examples include BMP-2, -4, -5, -6, -7 (also referred to as osteogenic protein (OP-1) and -8 (OP-2)).
Tumour specific proteins, including human trophoblast antigen, herceptin receptor, oestrogen receptor, cathepsins e.g., cathepsin B (found in liver and spleen).
Disease-specific proteins, such as antigens expressed only on activated T-cells: including LAG-3 (lymphocyte activation gene), osteoprotegerin ligand (OPGL), OX40 (a member of the TNF receptor family, expressed on activated T cells and the only costimulatory T cell molecule known to be specifically up-regulated in human T cell leukaemia virus type-I (HTLV-I)-producing cells); Metalloproteases (associated with arthritis/cancers), including CG6512 Drosophila, human paraplegin, human FtsH, human AFG3L2, murine ftsH; angiogenic growth factors, including acidic fibroblast growth factor (FGF-1), basic fibroblast growth factor (FGF-2), Vascular endothelial growth factor/vascular permeability factor (VEGF/VPF), transforming growth factor-a (TGF a), tumor necrosis factor-alpha (TNF-alpha), angiogenin, interleukin-3 (IL-3), interleukin-8 (IL-8), plateletderived endothelial growth factor (PD-ECGF), placental growth factor (P1GF), midkine platelet-derived growth factor-BB (PDGF), fractalkine.
Stress proteins (heat shock proteins): HSPs are normally found intracellularly. When they are found extracellularly, it is an indicator that a cell has died and spilled out its contents. This unprogrammed cell death (necrosis) only occurs when as a result of trauma, disease or injury and therefore in vivo, extracellular HSPs trigger a response from the immune system that will fight infection and disease. A dual specific which binds to extracellular HSP can be localised to a disease site.
Proteins involved in Fc transport: Brambell receptor (also known as FcRB). This Fc receptor has two functions, both of which are potentially useful for delivery. The functions are: the transport of IgG from mother to child across the placenta, and the protection of IgG from degradation thereby prolonging its serum half life of IgG. It is thought that the receptor recycles IgG from endosome.

Polypeptides according to the invention may be designed to be specific for the above targets without requiring any increase in or increasing half life in vivo. For example, polypeptides according to the invention can be specific for targets selected from the foregoing which are tissue-specific, thereby enabling tissue-specific targeting of the polypeptide, irrespective of any increase in half-life, although this may result. Moreover, where the polypeptide targets kidney or liver, this may redirect the polypeptide to an alternative clearance pathway in vivo (for example, the polypeptide may be directed away from liver clearance to kidney clearance).

Another embodiment of the present invention is an anti-EGFR or anti-IGF-IR polypeptide as described herein in which one or more Nanobodies is humanized. The humanized Nanobody may be an anti-EGFR Nanobody, an anti-IGF-IR Nanobody, an anti-serum albumin, another Nanobody useful according to the invention, or a combination of these.

One embodiment of the invention, is an anti-EGFR or anti-IGF-IR polypeptide comprising one or more humanized anti-EGFR or anti-IGF-IR Nanobodies and one or more humanized anti-human serum albumin Nanobodies.

By humanized is meant mutated so that potential immunogenicity upon administration in human patients is minor or nonexistent. Humanizing a polypeptide, according to the present invention, may comprise a step of replacing one or more of the non-human immunoglobulin amino acids by their human counterpart as found in a human consensus sequence or human germline gene sequence, without that polypeptide losing its typical character, i.e. the humanization does not significantly affect the antigen binding capacity of the resulting polypeptide.

According to one aspect of the invention, a humanized Nanobody is defined as a Nanobody having at least 50% homology (e.g. 55%, 60%, 65%, 70%, 75%, 80%, 85%, 90%, 95%, 98%, 100%) to the human framework region.

The inventors have determined the amino acid residues of a Nanobody which may be modified without diminishing the native affinity, in order to reduce its immunogenicity with respect to a heterologous species.

The inventors have also found that humanization of Nanobody polypeptides requires the introduction and mutagenesis of only a limited number of amino acids in a single polypeptide chain without dramatic loss of binding and/or inhibition activity. This is in contrast to humanization of scFv, Fab, (Fab)₂ and IgG, which requires the introduction of amino acid changes in two chains, the light and the heavy chain, and the preservation of the assembly of both chains.

The inventors have surprisingly found that Nanobodies of the invention comprising framework sequences highly homologous to human germline sequences such as DP29, DP47 and DP51 are highly effective. They occur naturally in some species such as those of the Camelidae. Such nanobodies are characterised in that they carry an amino acid from the group consisting of glycine, alanine, valine, leucine, isoleucine, proline, phenylalanine, tyrosine, tryptophan, methionine, serine, threonine, asparagine, or glutamine at position 45, such as, for example, L45. In addition, they may carry the human germline ‘J’ tryptophan at position 103, according to the Kabat numbering. Camelidae V_{HH} of this class, or other mutated Nanobodies which carry one or more framework sequences of this class are within the scope of the present invention.

As such, Nanobodies belonging to the class mentioned above, or Nanobodies carrying mutations of this class show a high amino acid sequence homology to human VH framework regions and polypeptides of the invention comprising these might be administered to a human directly without expectation of an unwanted immune response therefrom, and without the burden of further humanization. The invention also relates to nucleic acids capable of encoding said polypeptides.

A humanization technique may be performed by a method comprising the replacement of any of the Nanobody residues with the corresponding framework 1, 2 and 3 (FR1, FR2 and FR3) residues of germline VH genes (such as DP 47, DP 29 and DP 51) either alone or in combination.

According to one aspect of the present invention, humanization of Nanobodies is performed by substituting in said Nanobodies one or more of the amino acids at the positions described below, with the corresponding amino acids from the framework of germline VH genes, the numbering in accordance with the Kabat numbering:

According to an aspect of the invention, a framework region of the Nanobody which is unsubstituted remains as the original Nanobody framework.

According to one aspect of the invention, the residues of one or more of FR1, FR2 and FR3 are substituted according to the above scheme.

According to one aspect of the invention, at least 1, 2, 3 or all the residues of FR1 are substituted according to the above scheme.

According to one aspect of the invention, at least 1, 2, 3 or all the residues of FR2 are substituted according to the above scheme.

According to one aspect of the invention, at least 1, 2, 3, 4, 5, 6 or all the residues of FR3 are substituted according to the above scheme.

According to one aspect of the invention, at least 1, 2, 3 or all the residues of FR4 are substituted according to the above scheme.

In another embodiment of the invention, a humanized Nanobody is obtained by grafting all or part of the Nanobody CDR regions onto the germline human VH framework scaffold.

According to one aspect of the present invention, humanization of a Nanobody is performed by substituting one or more of CDR1, CDR2 and CDR3 of said Nanobody onto the germline human VH framework scaffold. Examples of suitable framework scaffold include those of DP47, DP29 and DP51.

The Nanobodies of the invention obtained according to the above mentioned humanization methods are part of the present invention.

Conventional four chain antibodies directed against EGFR or IGF-IR may be camelized, i.e. mutated such that the light chains are removed and one or more amino acid residues are substituted with Camelidae-specific residues (see for example, WO 94/04678 which is incorporated herein by reference). Such positions may preferentially occur at the VH-VL interface and at the so-called Camelidae hallmark residues, comprising positions 37, 44, 45, 47, 103 and 108. Such camelized antibodies are Nanobodies according to the invention. Polypeptides wherein at least one Nanobody is a VH wherein one or more amino acid residues have been partially substituted by specific sequences or amino acid residues of Nanobodies are Nanobodies according to the invention.

The Nanobodies as described above may be joined to form any of the anti-EGFR or anti-IGF-IR polypeptides disclosed herein comprising more than one Nanobody using methods known in the art. For example, they may be fused by chemical cross-linking by reacting amino acid residues with an organic derivatising agent such as described by Blättler et al (Biochemistry 24, 1517-1524; EP294703). Alternatively, the Nanobodies may be fused genetically at the DNA level i.e. a polynucleotide formed which encodes the complete anti-EGFR or anti-IGF-IR polypeptide comprising one or more anti-EGFR or anti-IGF-IR Nanobodies and optionally one or more anti-serum protein Nanobodies. A method for producing bivalent or multivalent Nanobodies is disclosed in PCT patent application WO 96/34103.

According to another aspect of the invention, Nanobodies can be linked to each other either directly or via a linker sequence. Such constructs are difficult to produce with conventional antibodies where due to steric hindrance of the bulky subunits, functionality will be lost or greatly diminished. As seen with the Nanobodies of the invention functionality is increased considerably when they are joined together, compared to the monovalent anti-EGFR or anti-IGF-IR polypeptide.

According to one aspect of the present invention, the Nanobodies are linked to each other directly, without use of a linker. Contrary to joining bulky conventional antibodies where a linker sequence is needed to retain binding activity in the two subunits, polypeptides of the invention can be linked directly thereby avoiding potential problems of the linker sequence, such as antigenicity when administered to a human subject, or instability of the linker sequence leading to dissociation of the subunits.

According to another aspect of the present invention, the Nanobodies are linked to each other via a peptide linker sequence. Such a linker sequence may be a naturally occurring sequence or a non-naturally occurring sequence. The linker sequence is expected to be non-immunogenic in the subject to which the anti-EGFR or anti-IGF-IR polypeptide is administered. The linker sequence may provide sufficient flexibility to the multivalent anti-EGFR or anti-IGF-IR polypeptide, at the same time being resistant to proteolytic degradation. A non-limiting example of a linker sequence is one that can be derived from the hinge region of Nanobodies as described in WO 96/34103. Another example is the linker sequence 3a (Ala-Ala-Ala).

Alternative linker sequences constructed by the inventors for fusion of bispecific and bivalent anti-EGFR or anti-IGF-IR polypeptides are listed in pending international application WO 06/040153. One linker sequence is the llama upper long hinge region. The other linkers are Gly/Ser linkers of different length. It is obvious to the person skilled in the art that said sequence linkers can be used to fuse any two monovalent sequences of this invention. According to an aspect of the invention an anti-EGFR or anti-IGF-IR polypeptide may be a homologous sequence of a full-length anti-EGFR or anti-IGF-IR polypeptide. According to another aspect of the invention, an anti-EGFR or anti-IGF-IR polypeptide may be a functional portion of a full-length anti-EGFR or anti-IGF-IR polypeptide. According to another aspect of the invention, an anti-EGFR or anti-IGF-IR polypeptide may be a functional portion of a homologous sequence of a full-length anti-EGFR or anti-IGF-IR polypeptide. According to an aspect of the invention an anti-EGFR or anti-IGF-IR polypeptide may comprise a sequence of an anti-EGFR or anti-IGF-IR polypeptide.

According to an aspect of the invention a Nanobody used to form an anti-EGFR or anti-IGF-IR polypeptide may be a complete Nanobody or a homologous sequence thereof. According to another aspect of the invention, a Nanobody used to form an anti-EGFR or anti-IGF-IR polypeptide may be a functional portion of a complete Nanobody. According to another aspect of the invention, a Nanobody used to form an anti-EGFR or anti-IGF-IR polypeptide may be a homologous sequence of a complete Nanobody. According to another aspect of the invention, a Nanobody used to form an anti-EGFR or anti-IGF-IR polypeptide may be a functional portion of a homologous sequence of a complete Nanobody.

As used herein, a homologous sequence of the present invention may comprise additions, deletions or substitutions of one or more amino acids, which do not substantially alter the functional characteristics of the polypeptides of the invention. The number of amino acid deletions or substitutions is preferably up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

A homologous sequence according to the present invention may be an anti-EGFR or anti-IGF-IR polypeptide modified by the addition, deletion or substitution of amino acids, said modification not substantially altering the functional characteristics compared with the unmodified polypeptide.

A homologous sequence according to the present invention may be a sequence which exists in other Camelidae species such as, for example, camel, dromedary, llama, alpaca, guanaco etc.

Where homologous sequence indicates sequence identity, it means a sequence which presents a high sequence identity (more than 70%, 75%, 80%, 85%, 90%, 95% or 98% sequence identity) with the parent sequence and is preferably characterised by similar properties of the parent sequence, namely binding to the same target.

A homologous nucleotide sequence according to the present invention may refer to nucleotide sequences of more than 50, 100, 200, 300, 400, 500, 600, 800 or 1000 nucleotides able to hybridize to the reverse-complement of the nucleotide sequence capable of encoding the parent sequence, under stringent hybridisation conditions (such as the ones described by Sambrook et al., Molecular Cloning, Laboratory Manuel, Cold Spring, Harbor Laboratory press, New York).

As used herein, a functional portion refers to a sequence of a Nanobody that is of sufficient size such that the interaction of interest is maintained with affinity of 1×10⁻⁶ M or better.

Alternatively, a functional portion comprises a partial deletion of the complete amino acid sequence and still maintains the binding site(s) and protein domain(s) necessary for the binding of and interaction with the target.

Alternatively a functional portion of a Nanobody of the invention comprises a partial deletion of the complete amino acid sequence and still maintains the binding site(s) and protein domain(s) necessary for the binding of and interaction with the target.

Alternatively a functional portion is a polypeptide which comprises a partial deletion of the complete amino acid sequence and which still maintains the binding site(s) and protein domain(s) necessary for the inhibition of binding of EGFR to another EGFR.

Alternatively a functional portion is a polypeptide which comprises a partial deletion of the complete amino acid sequence and which still maintains the binding site(s) and protein domain(s) necessary for the binding of and interaction with EGFR. Alternatively a functional portion comprises a partial deletion of the complete amino acid sequence of a polypeptide and which still maintains the binding site(s) and protein domain(s) necessary for the binding of and interaction with the antigen against which it was raised. It includes, but is not limited to Nanobodies.

As used herein, a functional portion refers to less than 100% of the complete sequence (e.g., 99%, 90%, 80%, 70%, 60%, 50%, 40%, 30%, 20%, 10%, 5%, 1% etc.), but comprises 5 or more amino acids or 15 or more nucleotides.

A homologous sequence of the present invention may include an anti-EGFR or anti-IGF-IR polypeptide which has been humanized. The humanization of antibodies of the new class of Nanobodies would further reduce the possibility of unwanted immunological reaction in a human individual upon administration.

Yet other examples of Nanobodies include “functional fragments”, meaning fragments that are functional in antigen binding (as described in WO03/035694). Such fragments comprise active antigen binding regions. Such fragments may be fragments of functional Nanobodies as described above, fragments of molecules that behave like functional Nanobodies, fragments of functionalized antibodies, or fragments of Nanobodies derived from conventional four chain antibodies which have been modified by substituting one or more amino acid residues with Camelidae-specific residues.

“Functional” in reference to a heavy chain antibody, a Nanobody, a VH domain or fragments thereof means that the same retains a significant binding (dissociation constant in the micromolar range or better) to its epitope, compared with its binding in vivo, and that it shows no or limited aggregation (soluble and non-aggregated above 1 mg/ml), so allowing the use of the antibody as a binder.

“Functionalized” in reference to a heavy chain antibody, a Nanobody or fragments thereof means to render said heavy chain antibody, Nanobody or fragments thereof functional. By “fragments thereof” as used in the sense of functional fragments, is meant a portion corresponding to more than 95% of the sequence, more than 90% of the sequence, more than 85% of the sequence, more than 80% of the sequence, more than 75% of the sequence, more than 70% of the sequence, more than 65% of the sequence, more than 60% of the sequence, more than 55% of the sequence, or more than 50% of the sequence.

According to the invention, a target is any of EGFR, IGF-IR or serum protein. Said targets are mammalian, and are derived from species such as rabbits, goats, mice, rats, cows, calves, camels, llamas, monkeys, donkeys, guinea pigs, chickens, sheep, dogs, cats, horses, and preferably humans.

Targets as mentioned herein such as EGFR, IGF-IR and serum proteins (e.g. serum albumin, serum immunoglobulins, thyroxine-binding protein, transferrin, fibrinogen) may be fragments of said targets. Thus a target is also a fragment of said target, capable of eliciting an immune response. A target is also a fragment of said target, capable of binding to a Nanobody raised against the full length target.

A Nanobody directed against a target preferably means a Nanobody that it is capable of binding to its target with an affinity of better than 10⁻⁶ M.

EGFR is to be understood as full-length EGFR or any fragment of EGFR. It is also expected that the Nanobodies and polypeptides of the invention will generally bind to all naturally occurring or synthetic analogs, variants, mutants, alleles, parts and fragments of EGFR, or at least to those analogs, variants, mutants, alleles, parts and fragments of EGFR that contain one or more antigenic determinants or epitopes that are essentially the same as the antigenic determinant(s) or epitope(s) to which the Nanobodies and polypeptides of the invention bind in EGFR (e.g. in wild-type EGFR).

IGF-IR (also called IGF-IR) is to be understood as full-length IGF-IR or any fragment of IGF-IR. It is also expected that the Nanobodies and polypeptides of the invention will generally bind to all naturally occurring or synthetic analogs, variants, mutants, alleles, parts and fragments of IGF-IR, or at least to those analogs, variants, mutants, alleles, parts and fragments of IGF-IR that contain one or more antigenic determinants or epitopes that are essentially the same as the antigenic determinant(s) or epitope(s) to which the Nanobodies and polypeptides of the invention bind in IGF-IR (e.g. in wild-type IGF-IR).

A fragment as used herein refers to less than 100% of the sequence (e.g., 99%, 90%, 80%, 70%, 60%, 50%, 40%, 30%, 20%, 10% etc.), but comprising 5, 6, 7, 8, 9, 10, 12, 13, 14, 15, 16, 17, 18, 19, 20, 21, 22, 23, 24, 25 or more amino acids. A fragment is preferably of sufficient length such that the interaction of interest is maintained with affinity of 1×10⁻⁶ M or better.

A fragment as used herein also refers to optional insertions, deletions and substitutions of one or more amino acids which do not substantially alter the ability of the target to bind to a Nanobody raised against the wild-type target. The number of amino acid insertions deletions or substitutions is preferably up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 amino acids.

One embodiment of the present invention relates to a polypeptide comprising at least one Nanobody wherein one or more amino acid residues have been substituted without substantially altering the antigen binding capacity.

Targets as mentioned herein such as EGFR, IGF-IR and serum proteins may be a sequence which exists in any species including, but not limited to mouse, human, camel, llama, shark, pufferfish, goat, rabbit, bovine.

A target may be a homologous sequence of a complete target. A target may be a fragment of a homologous sequence of a complete target.

The skilled person will recognise that the anti-EGFR and anti-IGF-IR polypeptides of the present invention may be modified, and such modifications are within the scope of the invention. For example, the polypeptides may be used as drug carriers, in which case they may be fused to a therapeutically active agent, or their solubility properties may be altered by fusion to ionic/bipolar groups, or they may be used in imaging by fusion to an appropriate imaging marker, or they may comprise modified amino acids etc. They may be also be prepared as salts. Such modifications which retain essentially the binding to EGFR and/or IGF-IR are within the scope of the invention.

According to an aspect of the invention, the anti-EGFR or anti-IGF-IR polypeptides can be used for oral administration. Conventional antibody-based therapeutics have significant potential as drugs because they have exquisite specificity to their target and a low inherent toxicity, however, they have one important drawback: they are relatively unstable, and are sensitive to breakdown by proteases. This means that conventional antibody drugs cannot be administered orally, sublingually, topically, nasally, vaginally, rectally or by inhalation because they are not resistant to the low pH at these sites, the action of proteases at these sites and in the blood and/or because of their large size. They have to be administered by injection (intravenously, subcutaneously, etc.) to overcome some of these problems. Administration by injection requires specialist training in order to use a hypodermic syringe or needle correctly and safely. It further requires sterile equipment, a liquid formulation of the therapeutic polypeptide, vial packing of said polypeptide in a sterile and stable form and, of the subject, a suitable site for entry of the needle. Furthermore, subjects commonly experience physical and psychological stress prior to and upon receiving an injection. Nevertheless, the polypeptides of the invention may be used for administration through injection.

An aspect of the present invention overcomes these problems of the prior art, by providing the anti-EGFR or anti-IGF-IR polypeptides of the present invention. Said polypeptides are sufficiently small, resistant and stable to be delivered orally, sublingually, topically, nasally, vaginally, rectally or by inhalation substantial without loss of activity. The polypeptides of the present invention avoid the need for injections, are not only cost/time savings, but are also more convenient and more comfortable for the subject.

One embodiment of the present invention is an anti-EGFR or anti-IGF-IR polypeptide as disclosed herein for use in treating, preventing and/or alleviating the symptoms of disorders susceptible to modulation by a substance that controls EGFR or IGF-IR, respectively, which is able to pass through the gastric environment without the substance being inactivated.

As known by persons skilled in the art, once in possession of said polypeptide, formulation technology may be applied to release a maximum amount of polypeptide in the right location (in the stomach, in the colon, etc.). This method of delivery is important for treating, preventing and/or alleviating the symptoms of disorders whose targets are located in the gut system.

An aspect of the invention is a method for treating, preventing and/or alleviating the symptoms of a disorder susceptible to modulation by a substance that controls EGFR or IGF-IR, respectively, which is able to pass through the gastric environment without being inactivated, by orally administering to a subject an anti-EGFR or anti-IGF-IR polypeptide as disclosed herein.

Another embodiment of the present invention is a use of an anti-EGFR or anti-IGF-IR polypeptide as disclosed herein for the preparation of a medicament for treating, preventing and/or alleviating the symptoms of disorders susceptible to modulation by a substance that controls EGFR or IGF-IR, respectively, which is able to pass through the gastric environment without being inactivated.

An aspect of the invention is a method for delivering a substance that controls EGFR or IGF-IR, respectively, to the gut system without said substance being inactivated, by orally administering to a subject an anti-EGFR or anti-IGF-IR polypeptide as disclosed herein.

An aspect of the invention is a method for delivering a substance that controls EGFR or IGF-IR, respectively, to the bloodstream of a subject without the substance being inactivated, by orally administering to a subject an anti-EGFR or anti-IGF-IR polypeptide as disclosed herein.

Another embodiment of the present invention is an Nanobody or polypeptide as disclosed herein for use in treating, preventing and/or alleviating the symptoms or disorders susceptible to modulation by a Nanobody or polypeptide of the invention delivered to the vaginal and/or rectal tract.

Examples of disorders are any that cause inflammation, including, but not limited to rheumatoid arthritis, Crohn's disease, ulcerative colitis, inflammatory bowl syndrome, and multiple sclerosis. In a non-limiting example, a formulation according to the invention comprises an Nanobody or polypeptide as disclosed herein, in the form of a gel, cream, suppository, film, or in the form of a sponge or as a vaginal ring that slowly releases the active ingredient over time (such formulations are described in EP 707473, EP 684814, U.S. Pat. No. 5,629,001).

An aspect of the invention is a method for treating, preventing and/or alleviating the symptoms of disorders susceptible to modulation by a Nanobody or polypeptide as described herein delivered to the vaginal and/or rectal tract, by vaginally and/or rectally administering to a subject a Nanobody or polypeptide as disclosed herein.

Another embodiment of the present invention is a use of a Nanobody or polypeptide as disclosed herein for the preparation of a medicament for treating, preventing and/or alleviating the symptoms of disorders susceptible to modulation by a Nanobody or polypeptide as described herein delivered to the vaginal and/or rectal tract.

An aspect of the invention is a method for delivering a Nanobody or polypeptide as described herein to the vaginal and/or rectal tract without said substance being inactivated, by administering to the vaginal and/or rectal tract of a subject an Nanobody or polypeptide as disclosed herein.

An aspect of the invention is a method for delivering a Nanobody or polypeptide as described herein to the bloodstream of a subject without said substance being inactivated, by administering to the vaginal and/or rectal tract of a subject an Nanobody or polypeptide as disclosed herein.

Another embodiment of the present invention is an anti-EGFR or anti-IGF-IR polypeptide as disclosed herein, for use in treating, preventing and/or alleviating the symptoms of disorders susceptible to modulation by a substance that controls EGFR or IGF-IR, respectively, delivered to the nose, upper respiratory tract and/or lung.

In a non-limiting example, a formulation according to the invention, comprises an anti-EGFR or anti-IGF-IR polypeptide as disclosed herein in the form of a nasal spray (e.g. an aerosol) or inhaler. Since the polypeptide is small, it can reach its target much more effectively than therapeutic IgG molecules.

An aspect of the invention is a method for treating, preventing and/or alleviating the symptoms of disorders susceptible to modulation by a substance that controls EGFR or IGF-IR, respectively, delivered to the upper respiratory tract and lung, by administering to a subject an anti-EGFR or anti-IGF-IR polypeptide as disclosed herein, by inhalation through the mouth or nose.

Another embodiment of the present invention is a use of an anti-EGFR or anti-IGF-IR polypeptide as disclosed herein for the preparation of a medicament for treating, preventing and/or alleviating the symptoms of disorders susceptible to modulation by a substance that controls EGFR or IGF-IR, respectively, delivered to the nose, upper respiratory tract and/or lung, without said polypeptide being inactivated.

An aspect of the invention is a method for delivering a substance that controls EGFR or IGF-IR, respectively, to the nose, upper respiratory tract and lung without inactivation, by administering to the nose, upper respiratory tract and/or lung of a subject an anti-EGFR or anti-IGF-IR polypeptide as disclosed herein.

An aspect of the invention is a method for delivering a substance that controls EGFR or IGF-IR, respectively, to the bloodstream of a subject without inactivation by administering to the nose, upper respiratory tract and/or lung of a subject an anti-EGFR or anti-IGF-IR polypeptide as disclosed herein.

One embodiment of the present invention is an anti-EGFR or anti-IGF-IR polypeptide as disclosed herein for use in treating, preventing and/or alleviating the symptoms of disorders susceptible to modulation by a substance that controls EGFR or IGF-IR, respectively, which is able pass through the tissues beneath the tongue effectively. A formulation of said polypeptide as disclosed herein, for example, a tablet, spray, drop is placed under the tongue and adsorbed through the mucus membranes into the capillary network under the tongue. An aspect of the invention is a method for treating, preventing and/or alleviating the symptoms of disorders susceptible to modulation by a substance that controls EGFR or IGF-IR, respectively, which is able pass through the tissues beneath the tongue effectively, by sublingually administering to a subject an anti-EGFR or anti-IGF-IR polypeptide as disclosed herein.

Another embodiment of the present invention is a use of an anti-EGFR or anti-IGF-IR polypeptide as disclosed herein for the preparation of a medicament for treating, preventing and/or alleviating the symptoms of disorders susceptible to modulation by a substance that controls EGFR or IGF-IR, respectively, which is able to pass through the tissues beneath the tongue.

An aspect of the invention is a method for delivering a substance that controls EGFR or IGF-IR, respectively, to the tissues beneath the tongue without being inactivated, by administering sublingually to a subject an anti-EGFR or anti-IGF-IR polypeptide as disclosed herein.

An aspect of the invention is a method for delivering a substance that controls EGFR or IGF-IR, respectively, to the bloodstream of a subject without being inactivated, by administering orally to a subject an anti-EGFR or anti-IGF-IR polypeptide as disclosed herein.

One embodiment of the present invention is an Nanobody or polypeptide as disclosed herein for use in treating, preventing and/or alleviating the symptoms of disorders susceptible to modulation by a Nanobody or polypeptide as described herein which is able to pass through the skin effectively.

Examples of disorders are any that cause inflammation, including, but not limited to rheumatoid arthritis, psoriasis, Crohn's disease, ulcerative colitis, inflammatory bowl syndrome, and multiple sclerosis. A formulation of said polypeptide construct, for example, a cream, film, spray, drop, patch, is placed on the skin and passes through.

An aspect of the invention is a method for treating, preventing and/or alleviating the symptoms of disorders susceptible to modulation by a Nanobody or polypeptide as described herein which is able to pass through the skin effectively, by topically administering to a subject an Nanobody or polypeptide as disclosed herein.

Another embodiment of the present invention is a use of an Nanobody or polypeptide as disclosed herein for the preparation of a medicament for treating, preventing and/or alleviating the symptoms of disorders susceptible to modulation by a Nanobody or polypeptide as described herein which is able to pass through the skin effectively.

An aspect of the invention is a method for delivering a Nanobody or polypeptide as described herein to the skin without being inactivated, by administering topically to a subject an Nanobody or polypeptide as disclosed herein.

An aspect of the invention is a method for delivering a Nanobody or polypeptide as described herein to the bloodstream of a subject, by administering topically to a subject an Nanobody or polypeptide as disclosed herein.

One embodiment of the present invention is an anti-EGFR or anti-IGF-IR polypeptide as disclosed herein for use in treating, preventing and/or—alleviating the symptoms of disorders susceptible to modulation by a substance that controls EGFR or IGF-IR, respectively, delivered to the intestinal mucosa, wherein said disorder increases the permeability of the intestinal mucosa. Because of its small size, an anti-EGFR or anti-IGF-IR polypeptide as disclosed herein can pass through the intestinal mucosa and reach the bloodstream more efficiently in subjects suffering from disorders which cause an increase in the permeability of the intestinal mucosa.

An aspect of the invention is a method for treating, preventing and/or alleviating the symptoms of disorders susceptible to modulation by a substance that controls EGFR or IGF-IR, respectively, delivered to the intestinal mucosa, wherein said disorder increases the permeability of the intestinal mucosa, by orally administering to a subject an anti-EGFR or anti-IGF-IR polypeptide as disclosed herein.

This process can be even further enhanced by an additional aspect of the present invention—the use of active transport carriers. In this aspect of the invention, a Nanobody is fused to a carrier that enhances the transfer through the intestinal wall into the bloodstream. In a non-limiting example, this “carrier” is a second a Nanobody which is fused to the therapeutic a Nanobody. Such fusion polypeptides are made using methods known in the art. The “carrier” Nanobody binds specifically to a receptor on the intestinal wall which induces an active transfer through the wall.

Another embodiment of the present invention is a use of an anti-EGFR or anti-IGF-IR polypeptide as disclosed herein for the preparation of a medicament for treating, preventing and/or alleviating the symptoms of disorders susceptible to modulation by a substance that controls EGFR or IGF-IR, respectively, delivered to the intestinal mucosa, wherein said disorder increases the permeability of the intestinal mucosa.

An aspect of the invention is a method for delivering a substance that controls EGFR or IGF-IR, respectively, to the intestinal mucosa without being inactivated, by administering orally to a subject an anti-EGFR or anti-IGF-IR polypeptide of the invention.

An aspect of the invention is a method for delivering a substance that controls EGFR or IGF-IR, respectively, to the bloodstream of a subject without being inactivated, by administering orally to a subject an anti-EGFR or anti-IGF-IR polypeptide of the invention. This process can be even further enhanced by an additional aspect of the present invention—the use of active transport carriers. In this aspect of the invention, an anti-EGFR or anti-IGF-IR polypeptide as described herein is fused to a carrier that enhances the transfer through the intestinal wall into the bloodstream. In a non-limiting example, this “carrier” is a Nanobody which is fused to said polypeptide. Such fusion polypeptides can be made using methods known in the art. The “carrier” Nanobody binds specifically to a receptor on the intestinal wall which induces an active transfer through the wall.

In another embodiment of the present invention, an anti-EGFR or anti-IGF-IR polypeptide as disclosed herein further comprises a carrier Nanobody (e.g. Nanobody) which acts as an active transport carrier for transport of said polypeptide via the lung lumen to the blood.

An anti-EGFR or anti-IGF-IR polypeptide may further comprise a carrier that binds specifically to a receptor present on the mucosal surface (bronchial epithelial cells) resulting in the active transport of the polypeptide from the lung lumen to the blood. The carrier Nanobody may be fused to the polypeptide. Such fusion polypeptidescan be made using methods known in the art and are describe herein. The “carrier” Nanobody binds specifically to a receptor on the mucosal surface which induces an active transfer through the surface. Another aspect of the present invention is a method to determine which Nanobodies (e.g. Nanobodies) are actively transported into the bloodstream upon nasal administration. Similarly, a naïve or immune Nanobody phage library can be administered nasally, and after different time points after administration, blood or organs can be isolated to rescue phages that have been actively transported to the bloodstream. A non-limiting example of a receptor for active transport from the lung lumen to the bloodstream is the Fc receptor N (FcRn). One aspect of the invention includes the Nanobodies identified by the method. Such Nanobodies can then be used as a carrier Nanobody for the delivery of a therapeutic Nanobody to the corresponding target in the bloodstream upon nasal administration.

The polypeptides of the invention may be used as the following administrations: as repeat dose administration in combination with conventional chemotherapeutic and radiation therapies; as intracavitary administration through stereotactic surgery, such as the treatment of brain cancers; or as a diagnostic to identify patients whose tumors over-express EGFR.

One embodiment of the present invention is an anti-EGFR or anti-IGF-IR polypeptide as disclosed herein, or a nucleic acid capable of encoding said polypeptide for use in treating, preventing and/or alleviating the symptoms of disorders relating to inflammatory processes, or cancer.

Another embodiment of the present invention is an anti-IGF-IR polypeptide as disclosed herein, or a nucleic acid capable of encoding said polypeptide for use in treating, preventing and/or alleviating the symptoms of disorders relating to cancer.

Another embodiment of the present invention is a use of an anti-EGFR or anti-IGF-IR polypeptide as disclosed herein, or a nucleic acid capable of encoding said polypeptide for the preparation of a medicament for treating a disorder relating to inflammatory processes or cancer.

EGFR is involved in inflammatory processes, and the blocking of EGFR action can have an anti-inflammatory effect, which is highly desirable in certain disease states such as, for example, inflammatory arthritis or psoriasis. Furthermore, blocking of the EGFR and IGF-IR can inhibit the growth of human tumors, therefore, the anti-EGFR or anti-IGF-IR polypeptides of the invention can have a cytostatic or cytotoxic effect on tumors. Our Examples demonstrate V_{HH}s according to the invention which bind EGFR and moreover, block ligand binding to the EGFR, prevent (hetero) dimerization of the receptor and/or induce apoptosis.

The polypeptides and method of the present invention are applicable to the treatment and diagnosis of epithelial cancers, such as lung, liver, central nervous system, bone, blood and lymphatic system, colon, breast, prostate, rectum, bladder, head and neck, ovarian, testis, pancreatic, testis, kidney, and squamos cell carcinoma. This listing of human cancers is intended to be exemplary rather than inclusive.

The anti-EGFR or anti-IGF-IR polypeptides and methods of the present invention are also applicable to other diseases associated with (the over-expression) of EGFR and/or IGF-IR. Examples of such diseases and disorders will be clear to the skilled person.

The present invention provides a therapeutic composition comprising an anti-EGFR or anti-IGF-IR polypeptide which inhibits or kills human tumor cells by said V_{HH} binding to the human EGFR or IGF-IR respectively of said tumor cells either alone or in combination with anti-neoplastic or chemotherapeutic agents. Anti-neoplastic or chemotherapeutic agents such as doxorubicin and cisplatin are well known in the art. Therapeutic compositions containing both anti-EGFR and anti-IGF-IR single domain antibodies are also within the scope of the invention.

The present invention also provides diagnostic methods which utilize an anti-EGFR or anti-IGF-IR polypeptide as disclosed here for detection of the corresponding conditions.

Diagnostic methods are well known in the art and include screening assays and imaging techniques (discussed further below).

Another embodiment of the present invention is a method of diagnosing a disorder characterised by the aberrant signaling of EGFR comprising:

(a) contacting a sample with an anti-EGFR or anti-IGF-IR polypeptide as described above,
(b) detecting binding of said polypeptide to said sample, and
(c) comparing the binding detected in step (b) with a standard, wherein a difference in binding relative to said sample is diagnostic of a disorder characterized by aberrant signaling of EGFR.

Another embodiment of the present invention is a method of diagnosing a disorder characterised by the presence of IGF-IR comprising:

(a) contacting a sample with an anti-IGF-IR polypeptide as described above,
(b) detecting binding of said polypeptide to said sample, and
(c) comparing the binding detected in step (b) with a standard, wherein a difference in binding relative to said sample is diagnostic of a disorder characterized by the presence of IGF-IR.

In one aspect of the invention, one can use an anti-EGFR or anti-IGF-IR polypeptide as disclosed herein, in order to screen for agents that modulate the binding of said polypeptide to EGFR or IGF-IR respectively. When identified in an assay that measures binding or said polypeptide displacement alone, agents may to be subjected to functional testing to determine whether they would modulate the action of the EGFR or IGF-IR in vivo.

In general, “therapeutically effective amount”, “therapeutically effective dose” and “effective amount” means the amount needed to achieve the desired result or results (modulating EGFR or IGF-IR binding; treating or preventing cancer or inflammation). One of ordinary skill in the art will recognize that the potency and, therefore, an “effective amount” can vary for the various compounds that modulate EGFR or IGF-IR binding used in the invention. One skilled in the art can readily assess the potency of the compound.

As used herein, the term “compound” refers to an anti-EGFR or anti-IGF-IR Nanobody or polypeptide of the present invention, or a nucleic acid capable of encoding said polypeptide or an agent identified according to the screening method described herein or said polypeptide comprising one or more derivatized amino acids.

By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained.

The polypeptides of a human-like class of V_{HH}'s as disclosed herein are useful for treating or preventing conditions in a subject and comprises administering a therapeutically effective amount of a compound or composition.

The polypeptides of the present invention are useful for treating or preventing conditions relating to cancer, rheumatoid arthritis and psoriasis in a subject and comprises administering a therapeutically effective amount of a compound or composition that binds EGFR or IGF-IR or both.

The anti-EGFR or anti-IGF-IR polypeptides as disclosed herein are useful for treating or preventing conditions relating to cancer, rheumatoid arthritis and psoriasis in a subject and comprise administering a therapeutically effective amount of a compound combination with another, such as, for example, doxorubicin.

The present invention is not limited to the administration of formulations comprising a single compound of the invention. It is within the scope of the invention to provide combination treatments wherein a formulation is administered to a patient in need thereof that comprises more than one compound of the invention.

Conditions mediated by EGFR or/and IGF-IR include, but are not limited to cancer, rheumatoid arthritis and psoriasis.

A compound useful in the present invention can be formulated as pharmaceutical compositions and administered to a mammalian host, such as a human patient or a domestic animal in a variety of forms adapted to the chosen route of administration, i.e., orally or parenterally, intranassally by inhalation, intravenous, intramuscular, topical or subcutaneous routes. A pharmaceutical composition of the present invention may comprise a compound and a suitable pharmaceutical vehicle as listed below.

A compound of the present invention can also be administered using gene therapy methods of delivery. See, e.g., U.S. Pat. No. 5,399,346, which is incorporated by reference in its entirety. Using a gene therapy method of delivery, primary cells transfected with the gene for the compound of the present invention can additionally be transfected with tissue specific promoters to target specific organs, tissue, grafts, tumors, or cells and can additionally be transfected with signal and stabilization sequences for subcellularly localized expression.

Thus, the present compound may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol.

Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by -various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compound may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, hydroxyalkyls or glycols or water-alcohol/glycol blends, in which the present compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compound to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compound can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician. Also the dosage of the compound varies depending on the target cell, tumor, tissue, graft, or organ.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

An administration regimen could include long-term, daily treatment. By “long-term” is meant at least two weeks and preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed. 4), Mack Publishing Co., Easton, Pa. The dosage can also be adjusted by the individual physician in the event of any complication.

The pharmaceutical compounds of the present invention may be administered in combination with conventional chemotherapeutic and radiation therapies.

A high throughput screening kit according to the invention comprises all the necessary means and media for performing the detection of an agent that modulates EGFR/ligand or IGF-IR/ligand interactions by interacting with EGFR or IGF-IR respectively, or fragment thereof in the presence of a polypeptide, preferably at a concentration in the range of 1 μM to 1 mM.

The kit comprises the following. Recombinant cells of the invention, comprising and expressing the nucleotide sequence encoding EGFR, or fragment thereof, which are grown according to the kit on a solid support, such as a microtiter plate, more preferably a 96 well microtiter plate, according to methods well known to the person skilled in the art especially as described in WO 00/02045. Alternatively EGFR, or fragment thereof is supplied in a purified form to be immobilized on, for example, a 96 well microtiter plate by the person skilled in the art. Alternatively EGFR, or fragment thereof is supplied in the kit pre-immobilized on, for example, a 96 well microtiter plate. The EGFR may be whole EGFR or a fragment thereof. Similar kits for IGF-IR can be developed.

Modulator agents according to the invention, at concentrations from about 1 μM to 1 mM or more, are added to defined wells in the presence of an appropriate concentration of anti-EGFR or anti-IGF-IR polypeptide, an homologous sequence thereof, a functional portion thereof or a functional portion of an homologous sequence thereof, said concentration of said polypeptide preferably in the range of 1 μM to 1 mM. Kits may contain one or more anti-EGFR or anti-IGF-IR polypeptides as described herein.

The inventors have found that the anti-EGFR or anti-IGF-IR polypeptides of the present invention, when labeled with suitable imaging agents, provide good markers for in vivo imaging. Good specificity without abolishing the high anti-IGF-IR affinity of IGF-IR Nanobody was obtained with superior tumor localization in vivo. Using the radionucleotide ^{99m}Tc as a labeling agent having a short half-life of 6 hours, in combination with the rapidly cleared Nanobodies of the invention, resulted in low background radiation and consequently in superior imaging results compared to the currently available alternatives with conventional antibodies.

One aspect of the present invention is an anti-EGFR or anti-IGF-IR polypeptide as disclosed herein further comprising one or more imaging agents. Imaging agents are any suitable for in vivo use, including, but not limited to ^{99m}Tc, ¹¹¹Indium, ¹²³Iodine. Other imaging agents suitable for magnetic resonance imaging include paramagnetic compounds, MR paramagnetic chelates. Other imaging agents include optical dyes.

Another aspect of the present invention is a use of an anti-EGFR or anti-IGF-IR polypeptide further comprising one or more imaging agents, for in vivo imaging. The anti-EGFR or anti-IGF-IR polypeptides may be labeled with imaging agents using methods known in the art.

It is an aspect of the invention that the labelled polypeptides are incorporated in microparticles, ultrasound bubbles, microspheres, emulsions, or liposomes. Such preparations allow for a more efficient delivery.

Anti-EGFR or anti-IGF-IR polypeptides of the invention may be used to direct a dose of radiotherapy treatment directly to a tumor. The polypeptides are labelled with one or more radioisotopes which cause damage or destruction to the tumor. Examples of suitable radioisotopes include, but are not limited to ¹⁸⁸Re, ¹³¹I and ²¹¹At. These isotopes may be attached to the polypeptide using conventional techniques or to tags (e.g. His-tag) fused to the polypeptide of the invention.

The polypeptides of the invention can also be linked to one or more anti-tumor agents, which cause destruction or damage to the tumor upon anti-EGFR or anti-IGF-IR polypeptide binding to EGFR or IGF-IR respectively. Suitable chemotherapeutic agents are known to those skilled in the art and include anthracyclines, methotraxate, vindesine, cis-platinum, ricin and calicheamicin.

The anti-tumor agent which is attached to the polypeptide of the invention may also be an enzyme which activates a prodrug. This allows activation of an inactive prodrug to its active cytotoxic form. The anti-tumor agent conjugated to the polypeptide of the invention may also be a cytokine such as interleukin-2, interleukin-4 or tumor necrosis factor alpha.

It is an aspect of the invention that the therapeutically labelled polypeptides are incorporated in microparticles, ultrasound bubbles, microspheres, emulsions, or liposomes. Such preparations allow for a more efficient delivery of the labelled polypeptides.

In a further aspect, the present invention provides one or more nucleic acid molecules encoding a Nanobody as herein defined.

The multivalent or multispecific Nanobody may be encoded on a single nucleic acid molecule; alternatively, each Nanobody may be encoded by a separate nucleic acid molecule.

Where the multivalent or multispecific Nanobody is encoded by a single nucleic acid molecule, the Nanobodies forming part of it may be expressed as a fusion polypeptide, in the manner of a scFv molecule, or may be separately expressed and subsequently linked together, for example using chemical linking agents. Multivalent or multispecific Nanobodies expressed from separate nucleic acids will be linked together by appropriate means.

The nucleic acid may further encode a signal sequence for export of the polypeptides from a host cell upon expression and may be fused with a surface component of a filamentous bacteriophage particle (or other component of a selection display system) upon expression.

In a further aspect the present invention provides a vector comprising nucleic acid encoding a polypeptide according to the present invention.

In a yet further aspect, the present invention provides a host cell transfected with a vector encoding a polypeptide according to the present invention.

Expression from such a vector may be configured to produce, for example on the surface of a bacteriophage particle, Nanobodies for selection. This allows selection of displayed Nanobodies and thus selection of polypeptides using the method of the present invention.

The present invention further provides a kit comprising at least a polypeptide according to the present invention.

A cell that is useful according to the invention are any bacterial cells such as for example E. coli, yeast cells such as for example S. cerevisiae and P. pastoris, insect cells, mammalian cells or molds comprising those belonging to the genera Aspergillus or Trichoderma.

A cell that is useful according to the invention can be any cell into which a nucleic acid sequence encoding a Nanobody or polypeptide according to the invention can be introduced such that the polypeptide is expressed at natural levels or above natural levels, as defined herein. Preferably a polypeptide of the invention that is expressed in a cell exhibits normal or near normal pharmacology, as defined herein.

According to a preferred embodiment of the present invention, a cell is selected from the group consisting of COS7-cells, a CHO cell, a LM (TK−) cell, a NIH-3T3 cell, HEK-293 cell, K-562 cell or a 1321N1 astrocytoma cell but also other transfectable cell lines.

Finally, although the use of the Nanobodies of the invention (as defined herein) and of the polypeptides of the invention is much preferred, it will be clear that on the basis of the description herein, the skilled person will also be able to design and/or generate, in an analogous manner, other (single) domain antibodies against EGFR or IGF-IR, respectively, as well as polypeptides comprising such (single) domain antibodies (in which the terms “domain antibody” and “single domain antibody” have their usual meaning in the art, see for example the prior art referred to herein).

Thus, one further aspect of the invention relates to domain antibodies or single domain antibodies against EGFR or IGF-IR, respectively, and to polypeptides that comprise at least one such (single) domain antibody and/or that essentially consist of such a (single) domain antibody.

In particular, such a (single) domain antibody against EGFR or IGF-IR, respectively, may comprise 3 CDR's, in which said CDR's are as defined above for the Nanobodies of the invention. For example, such (single) domain antibodies may be the single domain antibodies known as “dAb's”, which are for example as described by Ward et al, supra, but which have CDR's that are as defined above for the Nanobodies of the invention. However, as mentioned above, the use of such “dAb's” will usually have several disadvantages compared to the use of the corresponding Nanobodies of the invention. Thus, any (single) domain antibodies against EGFR or IGF-IR, respectively, according to this aspect of the invention will preferably have framework regions that provide these (single) domain antibodies against EGFR- or IGF-IR, respectively, with properties that make them substantially equivalent to the Nanobodies of the invention.

This aspect of the invention also encompasses nucleic acids that encode such (single) domain antibodies and/or polypeptides, compositions that comprise such (single) domain antibodies, polypeptides or nucleic acids, host cells that (can) express such (single) domain antibodies or polypeptides, and methods for preparing and using such (single) domain antibodies, polypeptides or nucleic acids, which may be essentially analogous to the polypeptides, nucleic acids, compositions, host cells, methods and uses described above for the Nanobodies of the invention.

Furthermore, it will also be clear to the skilled person that it may be possible to “graft” one or more of the CDR's mentioned above for the Nanobodies of the invention onto other “scaffolds”, including but not limited to human scaffolds or non-immunoglobulin scaffolds. Suitable scaffolds and techniques for such CDR grafting will be clear to the skilled person and are well known in the art, see for example U.S. Pat. No. 6,180,370, WO 01/27160, EP 0 605 522, EP 0 460 167, U.S. Pat. No. 6,054,297, Nicaise et al., Protein Science (2004), 13:1882-1891; Ewert et al., Methods, 2004 October; 34(2):184-199; Kettleborough et al., Protein Eng. 1991 October; 4(7): 773-783; O'Brien and Jones, Methods Mol. Biol. 2003:207:81-100; and Skerra, J. Mol. Recognit. 2000:13:167-187, and Saerens et al., J. Mol. Biol. 2005 Sep. 23; 352(3):597-607, and the further references cited therein. For example, techniques known per se for grafting mouse or rat CDR's onto human frameworks and scaffolds can be used in an analogous manner to provide chimeric proteins comprising one or more of the CDR's of the Nanobodies of the invention and one or human framework regions or sequences.

Thus, in another embodiment, the invention comprises a chimeric polypeptide comprising at least one CDR sequence chosen from the group consisting of CDR1 sequences, CDR2 sequences and CDR3 sequences mentioned herein for the Nanobodies of the invention. Preferably, such a chimeric polypeptide comprises at least one CDR sequence chosen from the group consisting of the CDR3 sequences mentioned herein for the Nanobodies of the invention, and optionally also at least one CDR sequence chosen from the group consisting of the CDR1 sequences and CDR2 sequences mentioned herein for the Nanobodies of the invention. For example, such a chimeric polypeptide may comprise one CDR sequence chosen from the group consisting of the CDR3 sequences mentioned herein for the Nanobodies of the invention, one CDR sequence chosen from the group consisting of the CDR1 sequences mentioned herein for the Nanobodies of the invention and one CDR sequence chosen from the group consisting of the CDR2 sequences mentioned herein for the Nanobodies of the invention. The combinations of CDR's that are mentioned herein as being preferred for the Nanobodies of the invention will usually also be preferred for these chimeric polypeptides.

In said chimeric polypeptides, the CDR's may be linked to further amino acid sequences sequences and/or may be linked to each other via amino acid sequences, in which said amino acid sequences are preferably framework sequences or are amino acid sequences that act as framework sequences, or together form a scaffold for presenting the CDR's. Reference is again made to the prior art mentioned in the last paragraph. According to one preferred embodiment, the amino acid sequences are human framework sequences, for example V_{H}3 framework sequences. However, non-human, synthetic, semi-synthetic or non-immunoglobulin framework sequences may also be used. Preferably, the framework sequences used are such that (1) the chimeric polypeptide is capable of binding EGFR or IGF-IR, respectively, i.e. with an affinity that is at least 1%, preferably at least 5%, more preferably at least 10%, such as at least 25% and up to 50% or 90% or more of the affinity of the corresponding Nanobody of the invention; (2) the chimeric polypeptide is suitable for pharmaceutical use; and (3) the chimeric polypeptide is preferably essentially non-immunogenic under the intended conditions for pharmaceutical use (i.e. indication, mode of administration, dosis and treatment regimen) thereof (which may be essentially analogous to the conditions described herein for the use of the Nanobodies of the invention).

According to one non-limiting embodiment, the chimeric polypeptide comprises at least two CDR sequences (as mentioned above) linked via at least one framework sequence, in which preferably at least one of the two CDR sequences is a CDR3 sequence, with the other CDR sequence being a CDR1 or CDR2 sequence. According to a preferred, but non-limiting embodiment, the chimeric polypeptide comprises at least three CDR sequences (as mentioned above) linked at least two framework sequences, in which preferably at least one of the three CDR sequences is a CDR3 sequence, with the other two CDR sequences being CDR1 or CDR2 sequences, and preferably being one CDR1 sequence and one CDR2 sequence. According to one specifically preferred, but non-limiting embodiment, the chimeric polypeptides have the structure FR1′-CDR1-FR2′-CDR2-FR3′-CDR3-FR4′, in which CDR1, CDR2 and CDR3 are as defined herein for the CDR's of the Nanobodies of the invention, and FR1′, FR2′, FR3′ and FR4′ are framework sequences. FR1′, FR2′, FR3′ and FR4′ may in particular be Framework 1, Framework 2, Framework 3 and Framework 4 sequences, respectively, of a human antibody (such as V_{H}3 sequences) and/or parts or fragments of such Framework sequences. It is also possible to use parts or fragments of a chimeric polypeptide with the structure FR1′-CDR1-FR2′-CDR2-FR3′-CDR3-FR4′. Preferably, such parts or fragments are such that they meet the criteria set out in the preceding paragraph.

The invention also relates to proteins and polypeptides comprising and/or essentially consisting of such chimeric polypeptides, to nucleic acids encoding such proteins or polypeptides; to methods for preparing such proteins and polypeptides; to host cells expressing or capable of expressing such proteins or polypeptides; to compositions, and in particular to pharmaceutical compositions, that comprise such proteins or polypeptides, nucleic acids or host cells; and to uses of such proteins or polypeptides, such nucleic acids, such host cells and/or such compositions, in particular for prophylactic, therapeutic or diagnostic purposes, such as the prophylactic, therapeutic or diagnostic purposes mentioned herein. For example, such proteins, polypeptides, nucleic acids, methods, host cells, compositions and uses may be analogous to the proteins, polypeptides, nucleic acids, methods, host cells, compositions and use described herein for the Nanobodies of the invention.

It should also be noted that, when the Nanobodies of the inventions contain one or more other CDR sequences than the preferred CDR sequences mentioned above, these CDR sequences can be obtained in any manner known per se, for example from Nanobodies (preferred), V_{H} domains from conventional antibodies (and in particular from human antibodies), heavy chain antibodies, conventional 4-chain antibodies (such as conventional human 4-chain antibodies) or other immunoglobulin sequences directed against EGFR or IGF-IR, respectively. Such immunoglobulin sequences directed against EGFR or IGF-IR, respectively, can be generated in any manner known per se, as will be clear to the skilled person, i.e. by immunization with EGFR or IGF-IR, respectively, or by screening a suitable library of immunoglobulin sequences with EGFR or IGF-IR, respectively, or any suitable combination thereof. Optionally, this may be followed by techniques such as random or site-directed mutagenesis and/or other techniques for affinity maturation known per se. Suitable techniques for generating such immunoglobulin sequences will be clear to the skilled person, and for example include the screening techniques reviewed by Hoogenboom, Nature Biotechnology, 23, 9, 1105-1116 (2005). Other techniques for generating immunoglobulins against a specified target include for example the Nanoclone technology (as for example described in the non-prepublished International Application PCT/EP2005/011819), so-called SLAM technology (as for example described in the European patent application 0 542 810), the use of transgenic mice expressing human immunoglobulins or the well-known hybridoma techniques (see for example Larrick et al, Biotechnology, Vol. 7, 1989, p. 934). All these techniques can be used to generate immunoglobulins against EGFR or IGF-IR, respectively, and the CDR's of such immunoglobulins can be used in the Nanobodies of the invention, i.e. as outlined above. For example, the sequence of such a CDR can be determined, synthesized and/or isolated, and inserted into the sequence of a Nanobody of the invention (e.g. so as to replace the corresponding native CDR), all using techniques known per se such as those described herein, or Nanobodies of the invention containing such CDR's (or nucleic acids encoding the same) can be synthesized de novo, again using the techniques mentioned herein.

### FIGURE LEGENDS

FIG. 1: Inhibition by the Nanobodies of the invention of the interaction of EGF with EGFR harboring A431 vesicles.

FIG. 2: Inhibition by the Nanobodies of the invention of the interaction of TGFα with EGFR harboring A431 vesicles.

FIG. 3: Inhibition of EGF binding to EGFR-Fcγ1.

FIG. 4: Inhibition of Fab_{Erbitrux} binding to EGFR-Fcγ1.

FIG. 5: Binding of EGFR Nanobodies to A431 tumor cells expressing EGFR.

FIG. 6: Modulation of EGFR phosphorylation status on HER14, following incubation with EGF (lane 1), PMP7C12 (lane 3), PMP7D12 (lane 4) and PMP9C1 (lane 5). Background EGFR phosphorylation in absence of exogenous ligand is shown in lane 2. The references to the detection antibodies for Western blot development are described in the text.

FIG. 7: Inhibition of EGF mediated EGFR signaling on HER14 cells.

FIG. 8: Nanobody mediated inhibition of tumor cell proliferation. The concentration dependent inhibition of cell proliferation is represented as the relative number of cells compared to the number of cells in absence of Nanobody or antibody.

FIG. 9: Nanobody mediated degradation of EGFR expressed on Hela cells.

FIG. 10: Sandwich ELISA showing simultaneous binding of Nanobody to EGFR harboring A431 vesicles and serum albumin.

FIG. 11: Inhibition by the Nanobodies or polypeptides of the invention of EGF binding to EGFR harboring A431 vesicles.

FIG. 12: Inhibition of A431 tumor cell proliferation by trivalent bispecific Nanobodies. The concentration dependent inhibition of cell proliferation is represented as the relative number of cells compared to the number of cells in absence of Nanobody or antibody.

FIG. 13: Effect of albumin interaction on A431 binding.

FIG. 14: Inhibition by the Nanobodies of the invention of IGF-I binding to rhIGF-IR.

### EXAMPLES

### Example 1

### Induction of an AEGFR Heavy-Chain Dependent Immune Response in Llama

The induction of a heavy-chain dependent immune response in four llamas by injecting intact human epidermoid carcinoma cells (A431; ATCC CRL 1555; Giard D J, Aaronson S A, Todaro G J, Arnstein P, Kersey J H, Dosik H, Parks W P 1973. In vitro cultivation of human tumors: establishment of cell lines derived from a series of solid tumors. J. Natl. Cancer Inst. 51:1417-1423), A431 derived membrane vesicles (prepared according to Cohen S, Ushiro H, Stoscheck C, Chinkers M, 1982. A native 170,000 epidermal growth factor receptor-kinase complex from shed plasma membrane vesicles. J. Biol. Chem. 257:1523-31) or purified EGFR (Sigma) is described in patent WO 05/044858. EGFR (Sigma) is prepared from EGFR overexpressing tumor cells (A431) using an immobilized antibody affinity resin according to Panayotou et al. (Receptor Purification 1990; 1: 289).

### Example 2

### Expression and Purification of EGFR-Fcγ1

A genetic fusion sequence of the N-terminal 618 amino acid residues of the extracellular domain of the EGFR and the sequence coding for human IgG1 hinge-CH2-CH3 (EGFR-Fcγ1) was used. The plasmid allows expression of a 140 kDa EGFR-Fc fusion protein and subsequent transport to the culture supernatant. Chinese Hamster Ovary cell line (CHO) was transiently transfected with the EGFR-Fcγ1 construct and culture supernatant was collected for further processing. EGFR-Fcγ1 fusion was purified from the supernatant following proteinG affinity chromatography. Functionality of EGFR-Fcγ1 was evaluated in ELISA. EGFR specific mAb528 and biotinylated EGF were able to bind significantly to rabbit anti-human IgG captured EGFR-Fcγ1 fusion in ELISA, indicating that the conformation of EGF ligand and mAb528 binding sites on the extracellular EGFR domain are not dramatically affected.

### Example 3

### Generation of Monovalent Fab Fragments of Erbitux

Erbitux is an anti-EGFR therapeutic chimaeric antibody derived from mouse monoclonal mAb225 (Sato J D, Kawamoto T, Le A D, Mendelsohn J, PolikoffJ, Sato G H 1983. Biological effects in vitro of monoclonal antibodies to human epidermal growth factor receptors. Mol. Biol. Med. 1:511-529). Fab fragment from Erbitux was prepared following papain digestion according to Fan and co-authors (Fan Z, Mendelsohn J, Masui H, Kumar R, 1993. Regulation of Epidermal Growth Factor Receptor in NIH3T3/HER14 Cells by Antireceptor Monoclonal Antibodies. J. Biol. Chem. 268:21073-21079). Papain digested Fab fragments were purified from non-digested IgG and Fc fragments via affinity chromatography on a proteinA column and subsequent size exclusion chromatography.

### Example 4

### Biotinylation of EGFR Ectodomain and Receptor Binding Proteins

IMAC-purified, recombinant human EGFR ectodomain encoding amino acid residues 1-614 of EGFR (EGFR-ECD) produced in baculovirus was a kind gift of Dr. K. M. Ferguson (Ferguson K M, Darling P J, Mohan M J, Macatee T L, Lemmon M A, 2000. Extracellular domains drive homo- but not hetero-dimerization of erbB receptors. EMBO J. 19:4632-4643). FabErbitux and EGFR-ECD were biotinylated using the reactive biotin derivative biotin amido hexanoic acid 3-sulfo-N-hydroxy succinimide ester (sodium salt; Sigma) according to the manufacturer's recommendations applying 5- or 3-fold molar excess of biotin reagent, respectively. Non-reacted biotin was removed by dialysis. Both the bivalent antibody Erbitux and its monovalent derivative FabErbitux are able to bind the neutravidin captured biotinylated EGFR-ECD in ELISA, indicating that biotin incorporation does not drastically affect the interaction sites of Erbitux or FabErbitux on EGFR-ECD. Biotinylated EGF and TGFα were obtained from Peprotech or Invitrogen.

### Example 5

### Nanobodies are Able to Inhibit Binding of Ligand to the EGFR

Cloning of the heavy-chain antibody fragment (V_{HH}) repertoire and subsequent isolation of EGFR specific Nanobodies was performed essentially as described in patent WO 05/044858. Periplasmic extracts of individual Nanobodies were screened for EGFR specificity by ELISA on solid phase coated EGFR (Sigma). Following sequence analysis, this resulted in the identification of a panel of 14 EGFR Nanobodies (SEQ ID NO's: 80-93; Table 1) belonging to 13 distinct Nanobody families (Table 2). A Nanobody family is defined as a panel of Nanobodies containing an identical CDR3 or with only a limited number of residue mutations in CDR3.

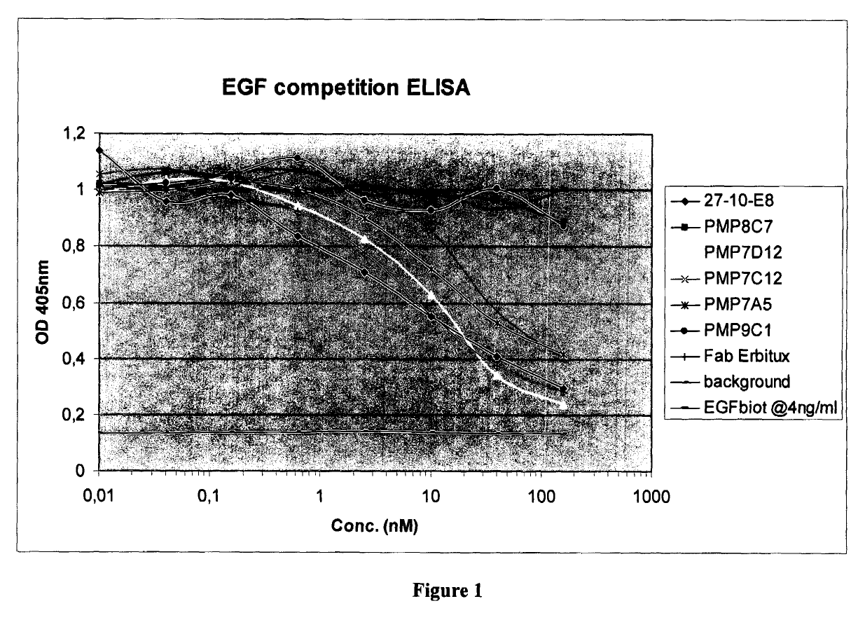

To identify Nanobodies that are able to compete with EGFR interacting proteins (ligands EGF, TGFα or neutralizing anti-EGFR antibodies), several assays were implemented. For a first ELISA based assay, A431 derived vesicles were prepared as described in Example 1 and total protein content was determined spectrophotometrically using the BCA protein assay kit from Pierce following the manufacturer's protocol. A431 vesicles, were immobilized overnight in PBS at 4° C. in Maxisorp microtiter plates (Nunc) at a concentration of 5 μg/ml. Plates were washed with PBS-Tween 0.05% and subsequently blocked with PBS-casein 1% for 2 hours at RT. After washing, equal volumes of 1.6 nM biotinylated EGF (EGFbiot) in presence of a dilution range of IMAC purified soluble Nanobody (range of final concentrations between 160 and 0.009 nM) were simultaneously applied in PBS-casein 0.1% and incubated for 1 hour at room temperature (rt). Receptor bound EGFbiot was detected with extravidine-alkaline phosphatase conjugate (Sigma) followed by a colorimetric reaction using para-nitrophenyl phosphate (Sigma). As a result, eight Nanobody families were identified to compete with EGF binding to EGFR expressing A431 vesicles (families I-VI and XI-XII summarized in Table 2). As an example, the EGF blocking capacity of Nanobodies PMP7A5, PMP7C12, PMP7D12 and 27-10-E8 is shown in FIG. 1. Two Nanobodies however, (PMP9C1 and PMP8C7 belonging to Nanobody families X and XIII, respectively) are not able to compete for the EGF epitope on EGFR in this assay (FIG. 1).

To evaluate EGF blocking capacity, another competition ELISA was performed. A polyclonal anti-human IgG (Dako, cat. nr. A0424) was coated at 1:1000 dilution in PBS overnight at 4° C. The next day, plates were washed three times with 200 ul PBS and blocked with 200 ul 2% BSA in PBS. EGFR-Fcγ1 fusion (Example 2) was captured at 3 μg/ml in 1% BSA in PBS for 1 hour at rt. Plates were washed again and serial dilutions of the different Nanobodies were made in 50 μl BSA in PBS in a separate plate. 50 μl of 20 ng/ml EGFbiot was then added to each well (final concentration of 10 ng/ml) and the mixture of Nanobody and EGFbiot was added to immobilised EGFR-Fcγ1. After incubation for 1.5 hours at rt, plates were washed four times and bound EGF was detected with peroxydase-coupled streptavidin (Jackson Immuno-research Laboratories; at 1:5000 dilution in 1% BSA in PBS) and staining was performed with o-phenylenediamine (OPD)/H₂O₂. Absorbance was read at 490 nm. Following this screening method and subsequent sequencing, Nanobody family IX (type example 27-1-H7) was identified with EGFR blocking capacity (Table 2).

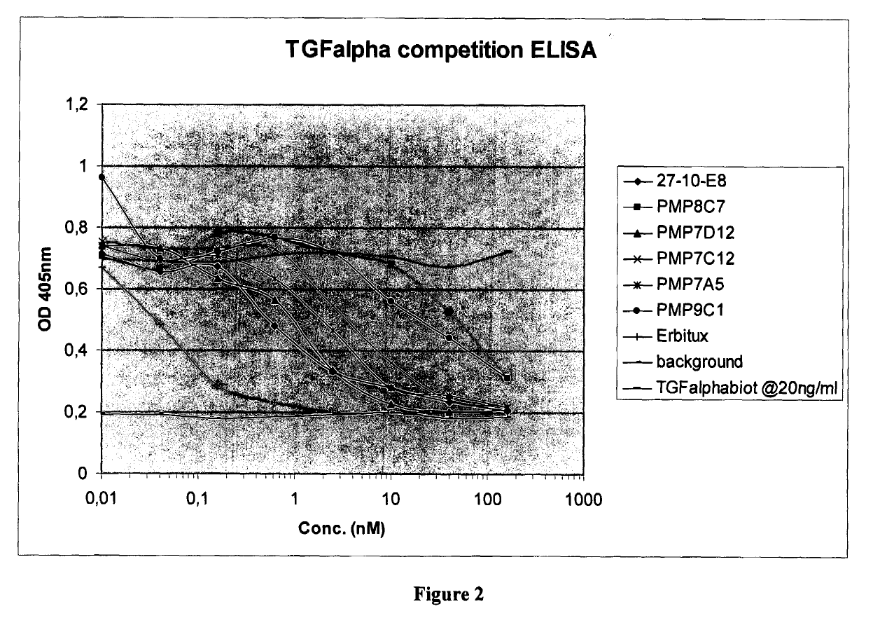

A similar competition ELISA was performed to screen for Nanobodies that are able to inhibit the TGFα interaction with the EGFR. In this competition ELISA, the binding of 4 nM of biotinylated TGFα to A431 vesicles was evaluated in presence of a dilution range of Nanobody (periplasmic extract or IMAC purified). Surprisingly, the previously identified Nanobody families X and XIII, which were not able to block EGF binding to the receptor, do inhibit TGFα binding to EGFR (Table 2; FIG. 2).

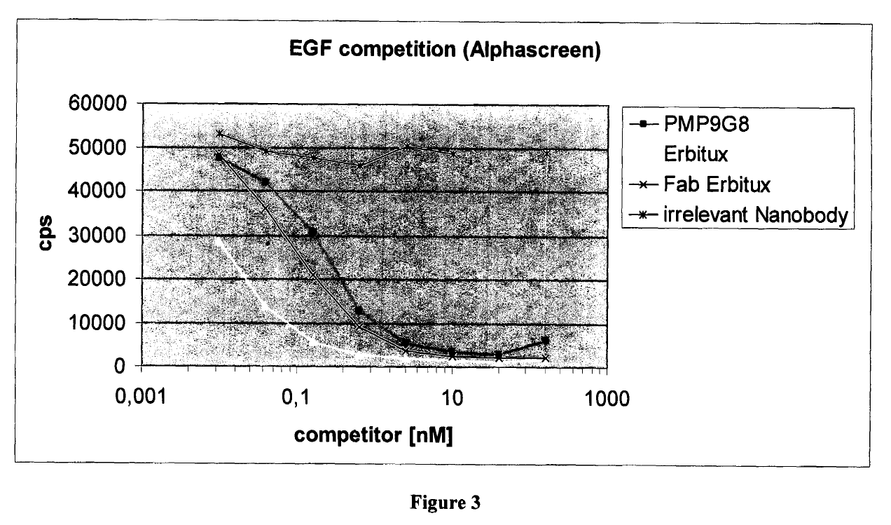

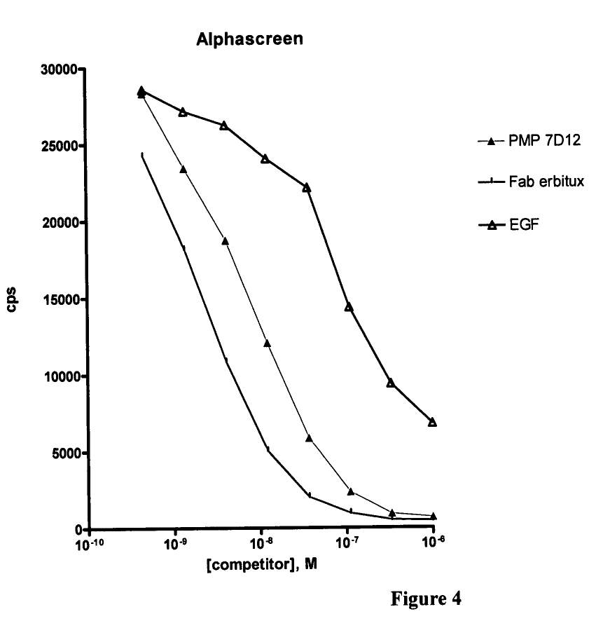

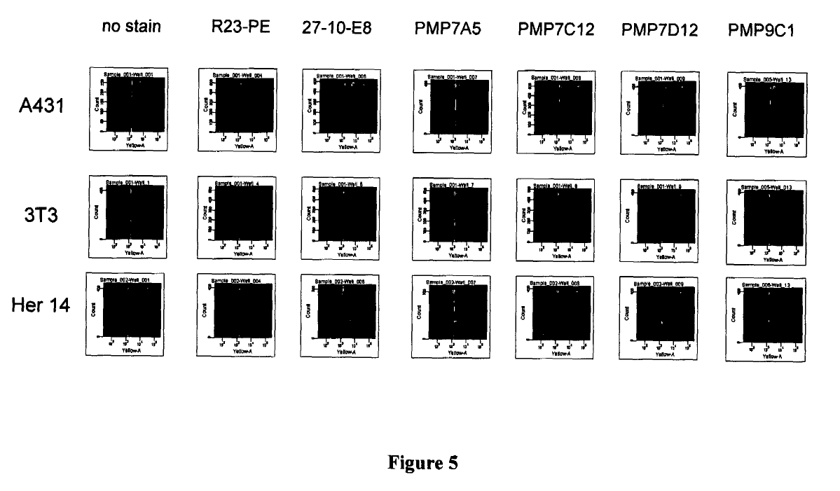

Yet another assay to screen for EGFR competitors was implemented, a so-called Alpha-screen. The Amplified Luminiscent Proximity Homogeneous Assay-screen is a proximity bead-based homogeneous assay which allows quantitative detection of two biologically interacting molecules. When the two interaction partners coupled to the acceptor and donor beads, respectively bring these beads in close vecinity, the transfer of a singlet oxygen molecule, generated following excitation at 680 nm, can result in fluorophore mediated light emission of the acceptor beads at 520-620 nm detected by the Envision (Perkin Elmer). This automated high throughput screening method allows to screen for competitors of the interaction pair in a 384 well format. Streptavidine conjugated donor beads capturing EGFbiot or in an alternative screen biotinylated FabErbitux were used in combination with EGFR-Fcγ1 conjugated acceptor beads prepared according to the manufacturer's (Perkin Elmer) instructions in two alphascreen assays to identify EGF or Erbitux competitors. EGF or FabErbitux were applied at 1 and 0.4 nM, respectively. In a first screen, the inhibition of the EGF -EGFR-Fcγ1 interaction by periplasmic Nanobody extracts was measured for a total of 3000 Nanobodies. Approximately 10 percent of the tested Nanobodies showed inhibition of EGF binding to EGFR-Fcγ1 fusion. Following this screening method and subsequent sequencing, two additional EGF competing Nanobody families (VII and VIII) were identified (Table 2). As an example of the inhibition curves obtained, the concentration dependent EGF competition capacity of Nanobody PMP9G8 (versus Erbitux and Fab_{Erbitux}) is shown in FIG. 3. In a subsequent alphascreen, all EGF competing Nanobodies were screened for their capacity to inhibit the interaction of Fab_{Erbitux} with EGFR-Fc, resulting in the identification of only one Nanobody family (family II; type example PMP7D12) which inhibits both EGF and FabErbitux interaction with the EGFR (FIG. 4).

Summarizing, extensive screening and sequence analysis resulted in the identification of 13 previously unidentified EGFR specific Nanobody families (I-XIII) which are able to block ligand binding to EGFR, compared to the EGFR Nanobodies published in patent WO 05/044858. Out of these 13 Nanobody families, 11 can inhibit EGF binding to EGFR. Surprisingly, the two remaining Nanobody families (X and XIII) are able to block only the TGFα but not EGF interaction with EGFR. An overview of all 13 aEGFR Nanobody families is represented in Table 2.

The new anti-EGFR Nanobodies are functionally classified into 3 Types of ligand competing Nanobodies. Non-limiting examples of the different classes of Nanobodies are as follows:
Type A Nanobody (binds to ectodomain EGFR, competes with the EGF, TGFα but not with the Erbitux binding sites on EGFR. Examples of such Nanobodies are 27-10-E8 (family I) and PMP7A5 (family III).
Type B Nanobody (binds to ectodomain EGFR, competes with the EGF, Erbitux and TGFα-binding sites on EGFR. Examples of such Nanobodies are PMP7D 12 and PMP7C 12 (family II).
Type C Nanobody (binds to ectodomain EGFR, competes with the TGFα but not the EGF or Erbitux-binding sites on EGFR downregulation. An example of such a Nanobody is PMP8C7 (family XIII).

### Example 6

### Screening of Kinetic Off-Rate Constants Via Surface Plasmon Resonance (BIAcore)

Surface plasmon resonance sensor chips (CM5) were coated with purified EGFR (Sigma) according to the manufacturer's instructions at a density of ˜5500 RUs. Regeneration of the chip surface was performed with a three second flow of 50 mM NaOH. Initially, periplasmic Nanobody extracts were screened to determine off-rates. For the most relevant Nanobody families blocking EGF binding and showing highest IC50s in the EGF competition vesicle ELISA (I-III and VI-IX), multiple Nanobody representatives belonging to the same family were screened and the Nanobody showing the lowest off rate is designated as the Nanobody family type example. The off rate of the relevant family type examples was confirmed with IMAC purified Nanobodies and is documented in Table 3.

Out of the 7 Nanobody families tested, five Nanobody family type examples 27-10-E8, PMP7A5, PMP7E12, PMP9G8 and PMP3867 (Table 3) showed an equal or lower off rate compared to Fab_{Erbitux} (k_{d}=1.7E-3 s⁻¹).

### Example 7

### Nanobodies Recognize Cell Surface Expressed EGFR Epitopes on Cells of Distinct Origin

Flow cytometry analysis allows detection of cell surface expressed EGFR. EGFR cell lines used for Nanobody binding assessment were the EGFR transfected mouse fibroblast cell lines HER14 (with an estimated receptor density of approximately 1×10⁵), its parental non transfected mouse fibroblast cell line NIH3T3 and human epidermal carcinoma cell line A431 tumor cells (receptor density of 1.2×10⁶). 1E5 cells (100 μl) were mixed with 1 μg of Nanobody and detection was performed via a polyclonal rabbit anti-Nanobody phycoerythrin (PE) labeled antiserum (R23-PE). All experiments were performed on the FACSarray (BD Biosciences). The type A, B and C Nanobodies (described in Example 5), known to block ligand binding to EGFR, that were tested in flow cytometry are 27-10-E8 (Type A), PMP7D12 (Type B), PMP7A5 (Type A) and PMP9C1 (Type C) (representing Nanobody families I, II, III and X, respectively). Without any exception, all antagonistic Nanobodies tested showed a shift compared to background fluorescence (detection with R23-PE conjugate) with A431 and HER14 but not to non-EGFR expressing NIH3T3, as shown in FIG. 5, indicating that these Nanobodies specifically recognize surface expressed EGFR.

### Example 8

### Nanobodies Inhibit EGF Mediated Receptor Signaling

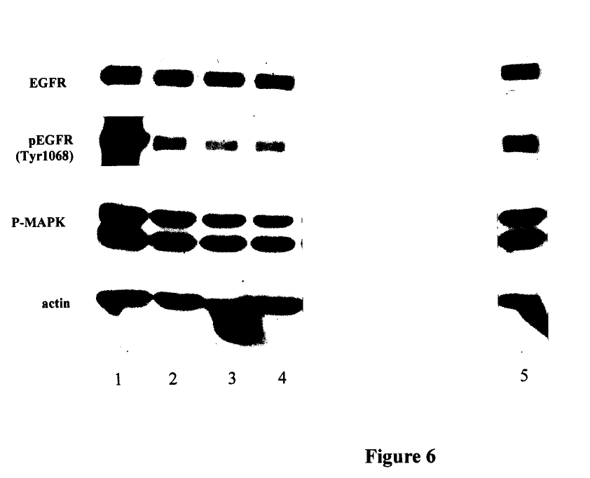

HER14 cells were seeded at 100,000 cells per well in 12-well tissue culture plates in DMEM (Gibco Life Technologies) containing 10% (v/v) serum and grown at 37° C. in 5% CO₂. After 8 hours, cells were washed once with DMEM containing 0.5% (v/v) FCS and serum-starved overnight in the same medium. The day of the assay, medium was refreshed with culture medium supplemented with 2% skimmed milk. Ligand (8 nM) or excess of Nanobody (1 μM) was added at 37° C. After 15 minutes, cells were quickly cooled down on ice and washed twice with ice-cold PBS. Total cell lysates were prepared by scraping the cells off the plate in 50 μl Laemmli protein sample buffer. Proteins were size-separated on 6% (w/v) polyacrylamide gels (20 μl of lysate was loaded per gel on two parallel gels) and subsequently blotted to PVDF membrane (Roche). Blots were stained for total amount of EGFR with a rabbit polyclonal antiserum to the receptor (Santa Cruz Biotechnology) and for phosphorylated receptor using a mouse monoclonal anti-phospho-tyrosine antibody tyr1068 (Cell signalling) followed by the respective peroxidase-conjugated secondary antibodies (donkey anti-rabbit and donkey anti-mouse). As a loading control, parallel blots were stained for presence of actin with a monoclonal anti-actin antibody (ICN Biomedicals Inc). Bound antibody was visualized by enhanced chemoluminescence using Western Lightning™ substrate (Perkin Elmer Life Sciences). The presence of exogenous EGF ligand induced a clear EGFR tyrosine phosphorylation and hence activation of the receptor (FIG. 6, lane 1) while a residual level of receptor phosphorylation was detected in absence of exogenous ligand (FIG. 6, lane 2). Nanobodies PMP7C 12 or PMP7D 12 (family II) do not activate the EGFR expressed on HER14 cells deprived of EGF, as shown by the lack of receptor tyrosine phosphorylation (FIG. 6, lane 3 and 4). Compared to the background receptor phosphorylation level however, Nanobody PMP9C1 (Family X) (FIG. 6, lane 5) showed an increased EGFR phosphorylation status under the conditions applied.

Following EGF induced receptor phosphorylation, downstream receptor mediated activation occurs. One of the downstream effector proteins involved in EGF receptor mediated signaling is the mitogen activated protein kinase (MAPK). After phosphorylation, MAPK triggers events that drive resting cells into cell division (Well A, 1999. Molecules in focus: EGF receptor. Int. J. Biochem. Cell Biol. 31:637-643). Downstream MAPK phosphorylation was evaluated by staining the above described blots with a mouse monoclonal anti-phospho-MAPK antibody (Cell Signalling). A clear induction of MAPK phosphorylation in HER14 is detected following induction by exogenous EGF (FIG. 6, lane 1) and to a lesser extend by PMP9C1 (FIG. 6, lane 5), but not in absence of exogenous EGF or in presence of PMP7C12, PMP7D12 (FIG. 6, lanes 2, 3 and 4, respectively).

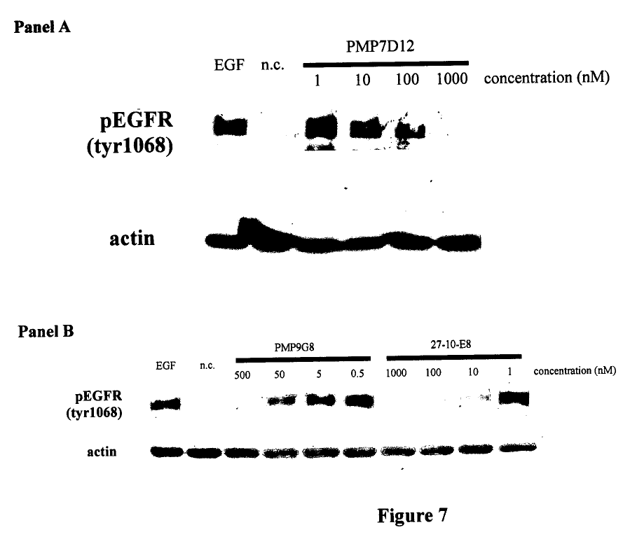

Secondly, the ability of Nanobody to inhibit receptor activation in presence of exogenous ligand was evaluated in a similar setting as described above. In this experiment, Nanobody dilutions (1, 10, 100 or 1000 nM) were added to HER14 cells in presence of 8 nM EGF ligand. Receptor phosphorylation was evaluated by Western blot with a mouse monoclonal that binds the phosphorylated Tyr¹⁰⁶⁸ of the EGFR (Cell Signalling). The addition of exogenous EGF ligand only resulted in a clear phosphorylation of Tyr¹⁰⁶⁸ (FIG. 7A, lane EGF), while a residual level of receptor phosphorylation was still detected even without the addition of exogenous ligand (FIG. 7A, lane n.c.). Nanobody families I (27-10-E8), II (PMP7D12), III (PMP7A5), VII (PMP9G8), VIII (PMP38G7) and X (PMP9C1) were tested in this cellular EGF competition assay. A concentration dependent inhibition of EGF mediated receptor phosphorylation is induced by Nanobody families I, II, III, VII, VIII while not with Nanobody Family X (PMP9C₁; not shown), confirming the EGF competitive capacities of the Nanobody families I, II, III, VII, VIII described in Example 5 (Table 2). FIG. 7 shows the concentration dependent inhibition of EGF mediated receptor phosphorylation by Nanobodies PMP9G8, 27-10-E8 (panel B) and PMP7D12 (panel A).

### Example 9

### Nanobodies Inhibit In Vitro Cell Proliferation of EGFR Expressing Tumor Cells

In a culture treated 96-well plate, 1000 A431 cells/well were seeded in 100 ul bicarbonate buffered DMEM (Gibco)+10% FCS, supplemented with penicillin and streptomycin and incubated for 48 hours at 37° C. in 5% CO₂. Following incubation, one entire 96-well A431 seeded plate was retained as control plate to determine non-inhibited (100%) growth at t=0 via sulforhodamine B staining (SRB), adapted from Sken at al. (Skehan P, Storeng R, Scudiero D, Monks A, McMahon J, Vistica D, Warren J T, Bokesch H, Kenney S, Boyd M R, 1990. New calorimetric cytotoxicity assay for anticancer-drug screening. J. Natl Cancer Inst 82:1107-1112). Subsequently, a dilution series of Nanobody or proliferation modulating control component (0-1000 nM) was applied in 1% FCS containing culture medium without the addition of exogenous ligand. Plates were then incubated at 37° C. in 5% CO₂. After incubation for 72 hours, cell numbers were estimated via a calorimetric assay. Cell proteins were fixed with trichloroacetic acid and subsequently stained with SRB. Unbound dye was removed via washing and protein-bound dye was extracted with 10 mM unbuffered Tris base. The optical density was measured at 564 nm. The relative potency of the Nanobodies tested was verified by comparing the dose-response curves of various Nanobodies to Erbitux (intact or Fab fragment).

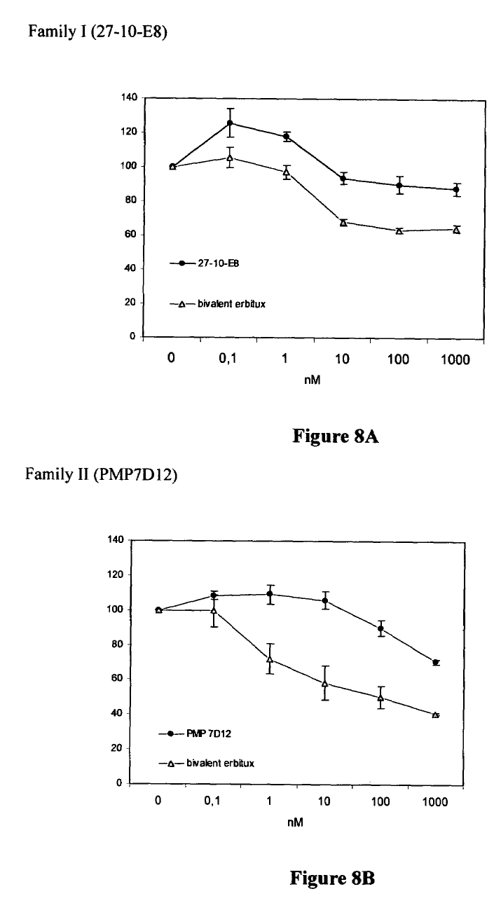

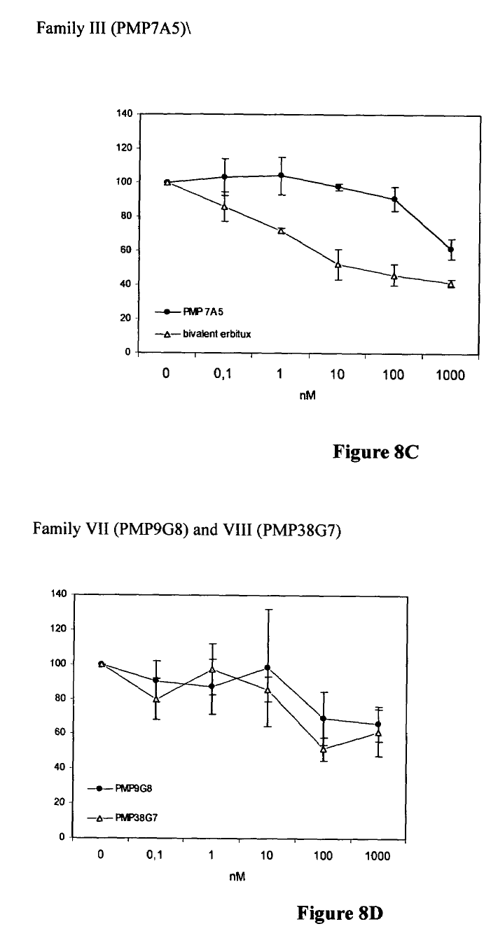

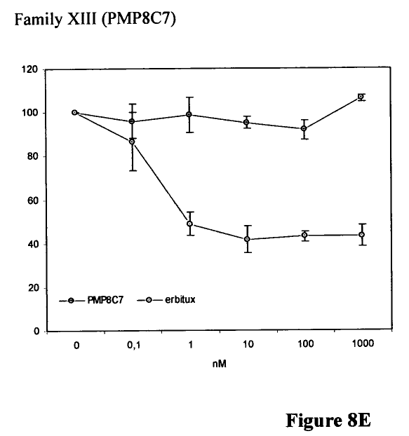

Nanobodies of families I, II, III, VII, VIII and XIII were assessed for their inhibitory capacity (compared to Erbitux, mono or bivalent) and results are depicted in FIG. 8. A strong positive correlation exists between Nanobodies that show significant blocking of ligand binding to receptor (see Examples 5 and 8) and the potency to inhibit A431 in vitro cell growth proliferation.

### Example 10

### Nanobodies Induce Receptor Sequestration from the Cell Surface

To detect sequestration (downregulation) of EGFR from the cell surface, Hela cells were seeded at 100000 cells per well in DMEM supplemented with 10% (v/v) FCS and 2 mM L-glutamine, the day before the assay. After 24 hours, cells were incubated at 37° C. for different time intervals up to 120 minutes with either recombinant human EGF (8 nM) or an excess of antibody or Nanobody (both at 1 μM). After the indicated time intervals, cells were quickly cooled down on ice and total cell lysates were prepared, size separated and blotted as in Example 8. Blots were then stained for total amount of EGFR with a polyclonal rabbit EGFR antiserum (Santa Cruz Biotechnologies). As a loading control, the same blots were stained for actin. Visualization of bound antibody was performed by enhanced chemoluminescence (Example 8).

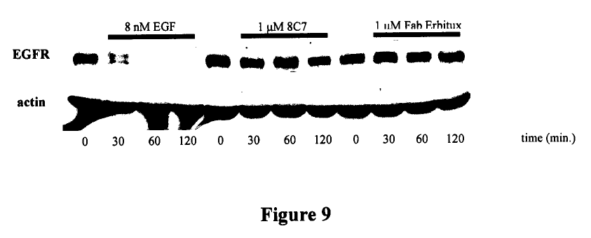

As expected, stimulation of Hela cells with 8 nM exogenous EGF resulted in an almost complete degradation of EGFR after 2 hours (FIG. 9, panel A), while no receptor degradation in Hela cells has been detected for Erbitux and the tested Nanobody families I, II and III (represented by type examples 27-10-E8, PMP7D12 and PMP7A5, respectively). Only for Nanobody family XIII (PMP8C7), minor receptor sequestration was detected at 120 mintes on Hela cells (FIG. 9). Previously, Fan and co-workers (Fan Z, Mendelsohn J, Masui H, Kumar R 1993. Regulation of epidermal growth factor receptor in NIH3T3/HER14 cells by antireceptor monoclonal antibodies. J. Biol. Chem. 268:21073-21079; Fan Z, Lu Y, Wu X, Mendelsohn J 1994. Antibody-induced epidermal growth factor receptor dimerization mediates inhibition of autocrine proliferation of A431 squamous carcinoma cells. J. Biol. Chem. 269:27595-27602) were able to show limited mAb225 dependent EGFR sequestration under their experimental settings. Although our experiment was performed repeatedly, a 2 hour-incubation with the EGF blocking mouse mAb225 (data not shown) or its chimaeric derivative Erbitux (FIG. 9) did not result in a detectable EGF receptor downregulating effect under our tested conditions.

### Example 11

### Tailoring the Nanobody Format to Improve Serum Half Life and Antagonistic Potency

The multivalent/multispecific Nanobodies that have been constructed are listed in Table 4 (SEQ ID NO's: 110-133 and 141-143). To test whether selected Nanobodies have potential as anticancer agents in an animal model, a strategy to increase the serum half life is preferred (as for example described in patent application WO 04/041865), since the serum half life of a mono- or bivalent Nanobody (approximately 15 or 30 KDa, respectively) is not optimal for this therapeutic indication. Here we describe the construction and characterization of trivalent, bispecific Nanobodies consisting of two N-terminally located anti-EGFR molecules fused to a third Nanobody with serum albumin specificity, all separated by a 9 (GS) amino acid linker peptide. Human serum albumin specific Nanobody ALB8, cross reactive with mouse serum albumin, (Table A-9; SEQ ID NO: 33) was chosen. DNA-segments encoding Nanobodies PMP7D12 or PMP7A5 and ALB8 were head-to-tail fused resulting in constructs 7D12-GS-7D12-GS-ALB8 and 7A5-GS-7A5-GS-ALB8 (Table 4; SEQ ID NO's: 126 and 124, respectively) in which the two aEGFR Nanobodies were separated by a 9 (GS) amino acid linker. The two Nanobodies were expressed in E. coli and purified via affinity chromatography (IMAC) on a NiNTA matrix to a purity level of approximately 95%.

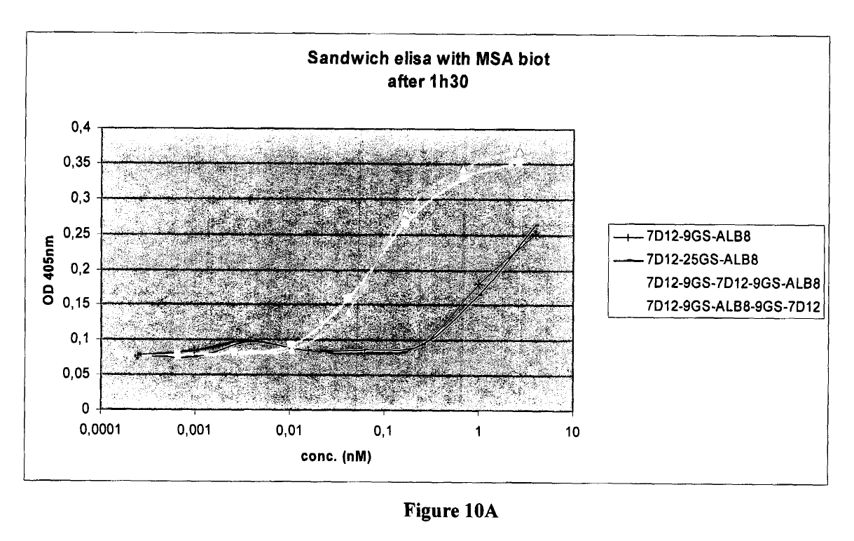

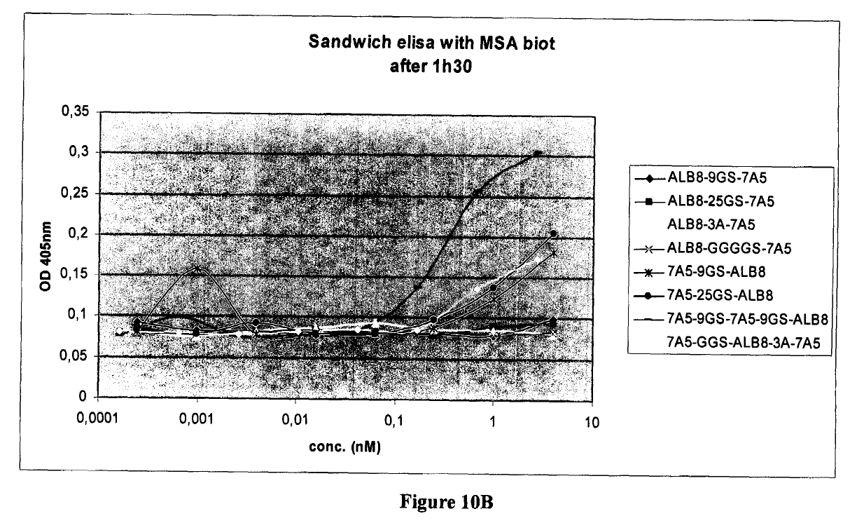

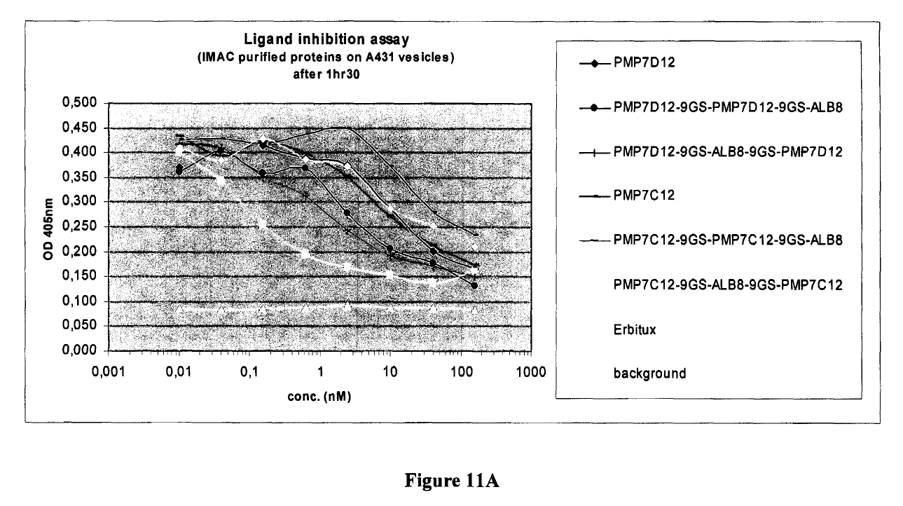

To verify whether the bispecific constructs were able to bind EGFR and MSA simultaneously, a sandwich ELISA was performed. Immobilization of A431 vesicles (5 μg/ml) and blocking of the plate was performed as described in Example 5. After adding a dilution series of the Nanobodies, vesicle bound Nanobody was detected via biotinylated MSA followed by extravidine-alkaline phosphatase conjugate (Sigma) and subsequent colorimetric reaction as in Example 5. We showed that, as expected, only the bispecific trivalent constructs (FIG. 10) but not the bivalent without the albumin specific Nanobody (data not shown) are simultaneously binding A431 vesicles and serum albumin.

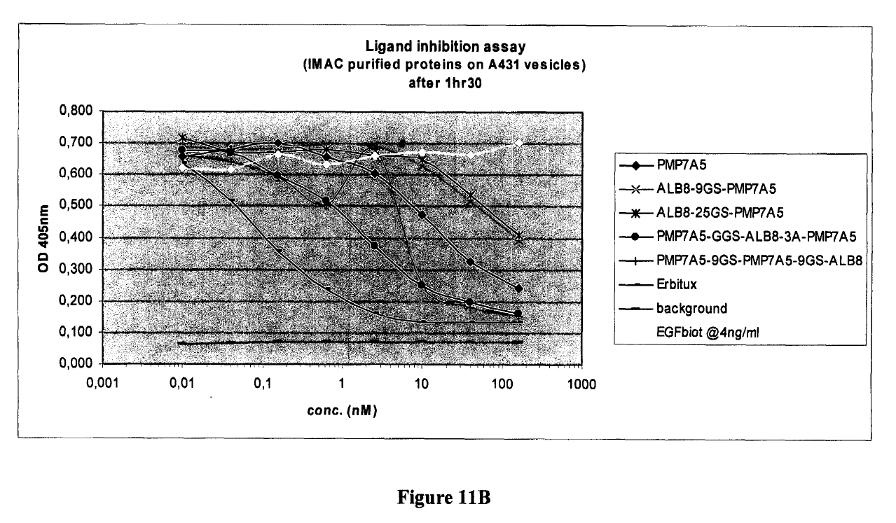

Subsequently, Nanobodies 7D12-GS-7D12-GS-ALB8 and 7A5-GS-7A5-GS-ALB8 were assessed for their ligand blocking capacity via the EGF-A431 vesicle competition ELISA (Example 9). As depicted in FIG. 11, the IC₅₀ values of 7D12-GS-7D12-GS-ALB8 and 7A5-GS-7A5-GS-ALB8 decrease with a factor 5 and 10, respectively, compared to the respective monovalent antiEGFR Nanobody.

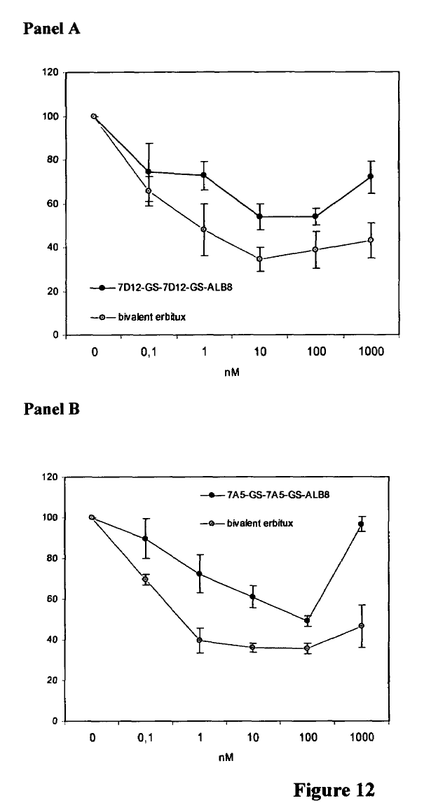

To unequivocally show that our Nanobodies are able to inhibit proliferation of EGFR expressing tumor cells in vitro, Nanobodies 7D12-GS-7D12-GS-ALB8 and 7A5-GS-7A5-GS-ALB8 were applied to the proliferating A431 tumor cells as described in Example 9. As depicted in FIG. 12, the IC₅₀ values of 7D12-GS-7D12-GS-ALB8 and 7A5-GS-7A5-GS-ALB8 are significantly lower than those of the monovalent EGFR Nanobodies PMP7A5 and PMP7D12 (FIG. 8).

Nanobodies can be made which target distinct paratopes on the EGFR (biparatopic Nanobodies) in absence or presence of an entity to improve serum half life. As examples, Nanobodies are made which combine EGFR Nanobodies PMP7D12 and PMP7A5 (belonging to Type B and Type A, respectively) and including serum half life improving Nanobody ALB8 (EGFRPMP7D12-GS-EGFRPMP7A5-GS-ALB8; SEQ ID NO: 142 and EGFRPMP7D12-GS-ALB8-GS-EGFRPMP7A5; SEQ ID NO: 143, Table 4) or without a moiety that improves serum half life (EGFRPMP7D12-GS-EGFRPMP7A5; SEQ ID NO: 141, Table 4). The anti-EGFR biparatopic targeting Nanobodies block ligand binding in a competition ELISA (described in Example 5) and inhibit proliferation of EGFR expressing tumor cells. The moiety to improve half life extension can be an albumin specific Nanobody or an Fc receptor specific Nanobody. In the same constructs, the serum half life function can be replaced by a binding protein with yet another biological function, for example to recruit immune effector functions (e.g. an anti-CD3 Nanobody or a Nanobody that binds to a complement protein).

### Example 12

### Nanobody Formatting is Responsible for Conditional EGFR Binding

A panel of 12 bispecific Nanobody constructs combining a single αEGFR (PMP7D12 or PMP7A5) and an aalbumin Nanobody (ALB8), separated by 3 (3A), 5 (GGGGS), 9 (GS), or 25 (GS5) amino acid linkers were constructed and are summarized in Table 4 (SEQ ID NO's: 110⁻¹²¹).

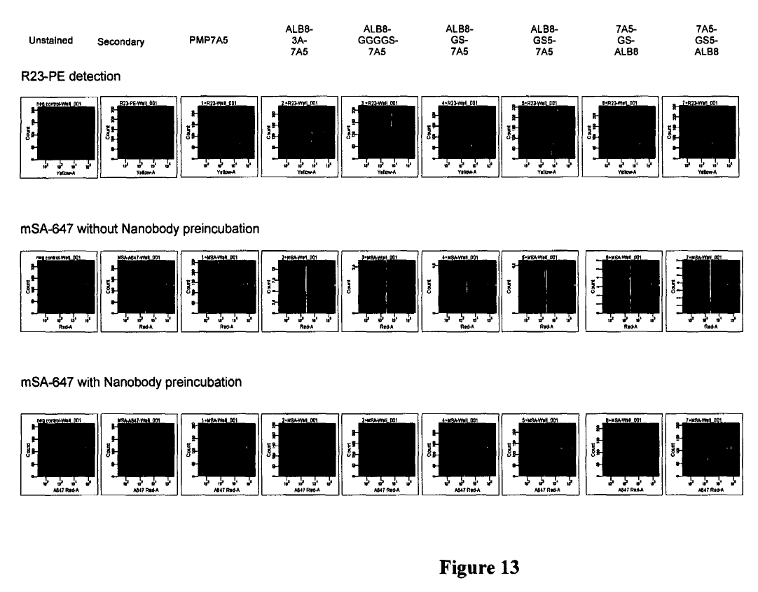

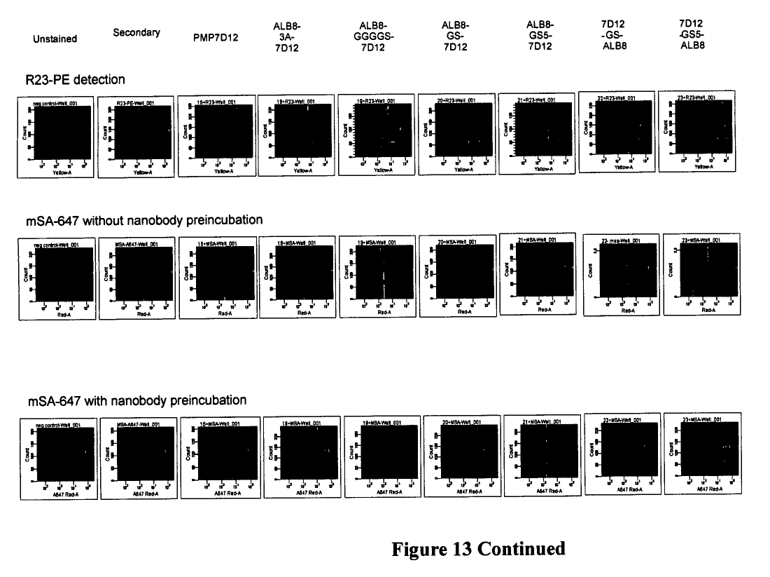

Three parallel flow cytometry experiments, applying equimolecular Nanobody amounts, were conducted with the 14 bispecific Nanobodies evaluating binding to A431 tumor cells using distinct detection methods. In a first experiment, Nanobodies were detected with the polyclonal rabbit α-Nanobody PE conjugate (R23-PE). In a second set of experiments, Nanobodies were incubated with A431 cells prior to detection with Alexa-647 conjugated mouse albumin (mSA-647). This detection method allows to evaluate whether albumin is still able to interact with the A431 bound Nanobody. In a third experiment, Nanobodies and mSA-647 were pre-incubated for 30 minutes (allowing albumin binding prior to EGFR interaction) and subsequently added to the A431 cells to verify whether albumin bound bispecific Nanobody is still able to bind surface expressed EGFR. The obtained FACS results are presented in FIG. 13.

When ALB8 is at the C-terminus in the bispecific constructs, maximal binding to A431 cell surface is detected with R23-PE (mean fluorescence >5×10⁴), irrespective of the αEGFR Nanobody. Performing the same R23-PE detection, N-terminally located ALB8 negatively affects A431 binding (shown by a decreased mean fluorescence compared to ALB8-EGFR constructs) although less pronounced whith increasing linkers lengths. Although for both EGFR Nanobodies tested in the ALB8-EGFR format, clear A431 binding is detected, an N-terminal ALB8 fusion seem to affects EGFR binding more for PMP7D12 than for PMP7A5. This is supported by surface plasmon resonance experiments showing significant reduction of EGFR binding of ALB8-PMP7D12 bispecific Nanobodies compared to PMP7D12-ALB8 formats (data not shown). When detection of A431 binding is performed with mSA-647 without preincubation, mean fluorescence of the bispecific constructs is low (irrespective of EGFR entity and ALB8 position) suggesting that A431 binding is negatively affected. Applying this detection method, linker length seem to positively affect A431 binding of the bispecific construct for ALB8-PMP7D12 constructs (FIG. 13).

Surprisingly however, preincubation with mSA-647 dramatically improves the binding to A431, irrespective of the EGFR Nanobody, ALB8 position or linker lengths tested, suggesting conditional EGFR interaction following albumin binding. Indeed, albumin interaction significantly improves the mean fluorescence of the bispecific Nanobody construct compared to those obtained without mSA-647 preincubation (FIG. 13).

### Example 13

### Nanobodies are Able to Inhibit Binding of IGF-I Ligand to the IGF-IR

The cloned VHH repertoires, described in Example 5 and originating from the A431 cell immunized llamas, were used for the identification of IGF-IR specific Nanobodies. Recombinant human IGF-IR (rhIGFIR) and IGF-I were acquired from Peprotech. Isolation of IGF-IR specific Nanobodies was performed essentially as described in patent WO 05/044858, applying IGF-I ligand to select for Nanobodies that bind the IGF-IR on an epitope which at least overlap with the ligand binding site. Both A431 vesicles, harboring IGF-IR, as rhIGFIR were used as antigen format for selections. Following selections, periplasmic extracts of individual Nanobodies were screened for IGF-IR specificity by ELISA on solid phase coated receptor. Following sequence analysis, this resulted in the identification of a panel of 4 distinct IGF-IR Nanobodies PMP4B11, PMP3G7, PMP2C7 and PMP1C7 (SEQ ID NO's: 106-109, respectively; Table 5) belonging to 4 distinct Nanobody families.

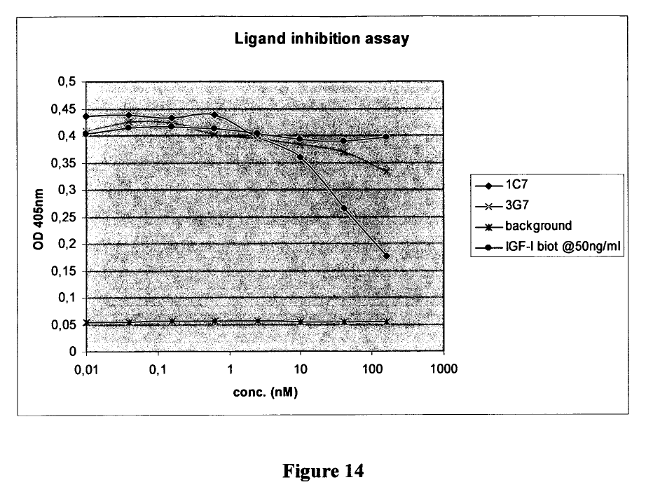

A competition ELISA similar to the one described in Example 5 was implemented to screen for Nanobodies which are able to inhibit the IGF-I interaction with the IGF-IR. IGF-I was biotinylated as described in Example 4 applying a 5-fold molar excess of biotin reagent. Excess of non-reacted biotin was removed by dialysis. rhIGFIR was solid-phase immobilized (1 μg/ml) and binding of biotinylated IGF-I (50 ng/ml) was detected with an extravidin alkaline phosphatase conjugate in absence or presence of competitor. Only PMP 1C7 is able to significantly inhibit the interaction of IGF-I with rhIGFIR as shown in FIG. 14. Subsequent surface plasmon resonance analysis (BIAcore) showed that the dissociation constant of PMP1C7 on immobilized IGF-IR equals 1.9E-3 s⁻¹.

Bispecific anti-IGF-IR Nanobodies separated by a 9 (GS) or 25 (GS5) amino acid linker are constructed (SEQ ID NO's: 134-135, Table 6) and these Nanobodies dramatically improve IGF-I blocking (competition ELISA) and inhibition of IGF-IR expressing tumor cell proliferation compared to the monovalent IGF-IR Nanobodies.

### Example 14

### Bispecific αEGFR-αIGF-IR Nanobodies are Able to Inhibit the IGF-IR and EGFR Signaling Cascades

Nanobodies can be made which target simultaneously epitopes on the EGFR and IGF-IR in absence or presence of an entity to improve serum half life. As examples, Nanobodies are made which combine EGFR Nanobody PMP7D12 and IGF-IR Nanobody PMPIC7 with or without serum albumin Nanobody ALB8 (SEQ ID NO's: 136-140, Table 7). The EGFR-IGF-IR targeting Nanobodies block ligand binding to their respective receptors in a competition ELISA (described in Example 5) and inhibit proliferation of EGFR/IGF-IR expressing tumor cells. The moiety to improve half life extension can be an albumin specific Nanobody or an Fc receptor specific Nanobody. In the same constructs, the serum half life function can be replaced by a binding protein with yet another biological function, for example to recruit immune effector functions (e.g. an anti-CD3 Nanobody or a Nanobody that binds to a complement protein).

### Tables

TABLE 1
Preferred Nanobodies against EGFR

<Name, SEQ ID #; PRT (protein); ->
Sequence

< 2710-E8, SEQ ID NO: 80; PRT; ->
EVQLVESGGGLVQAGGSLRLSCAASGRTFSTYTMAWFRQAPGKEREFVQG
ISRSDGGTYDADSVKGRFTISRDNAKNTVYLQMNSLKPEDTAVYFCAAAS
VKLVYVNPNRYSYWGQGTQVTVSS
< PMP7D12, SEQ ID NO: 81; PRT; ->
QVKLEESGGGSVQTGGSLRLTCAASGRTSRSYGMGWFRQAPGKEREFVSG
ISWRGDSTGYADSVKGRFTISRDNAKNTVDLQMNSLKPEDTAIYYCAAAA
GSAWYGTLYEYDYWGQGTQVTVSS
< PMP7C12, SEQ ID NO: 82; PRT; ->
AVQLVESGGGSVQAGGSLRLTCAASGRTSRSYGMGWFRQAPGKEREFVSG
ISWRGDSTGYADSVKGRFTISRDNAKNTVDLQMNSLKPEDTAIYYCAAAA
GSTWYGTLYEYDYWGQGTQVTVSS
< PMP9C1, SEQ ID NO: 83; PRT; ->
EVQLVESGGGLVQAGGSLRLSCAASGRTFSGFAMGWFRQAPGKEREFVAA
ISWSGGSLLYVDSVKGRFTISRDNAKNTVHLQMNSLKPGDTAVYYCAAMV
GPPPRSLDYGLGNHYEYDYWGQGTQVTVSS
< PMP7A5, SEQ ID NO: 84; PRT; ->
EVQLVESGGGLVQAGGSLRLSCAASDRTFSSNNMGWFRQAPGKEREFVAA
IGWGGLETHYSDSVKGRFTISRDNAKNTVYLQMNSLKPEDTARYYCAVSS
TRTVIYTLPRMYNYWGQGTQVTVSS
< PMP9A7, SEQ ID NO: 85; PRT; ->
EVQLVESGGGLVQAGGSLRLSCAVSGRTFSGDVMGWFRQAPGKEREFVAG
FSRSTSTTHYADSVKGRFTISGDNAKNTVYLQMNSLKPEDTAVYYCAANS
RSSWVIFTIKGQYDRWGQGTQVTVSS
< PMP8B5, SEQ ID NO: 86; PRT; ->
EVQLVESGGGLVQAGGSLRVSCVASIRSFSSYVVGWFRQAPGKDREFVAG
IAWGDGITYYADSVKGRFTISRDHAKNTAYLQMNSLRPEDTAVYYCAVRP
GMIITTIQATYGFWGQGTQVTVSS
< PMP11C9, SEQ ID NO: 87; PRT; ->
EVQLVESGGGLVQAGASLRLSCAASGRYFSDYNMAWFRQAPGKEREFVAH
ISWLGGRTYYRDSVKGRFTISRDNAKDTVYLQMNSLKPEDTAVYSCAAGS
PYGTELPYTRIEQYAYWGQGTQVTVSS
< PMP11H6, SEQ ID NO: 88; PRT; ->
AVQLVESGGGLVQAGGSLRLSCAASGRTFSTYTMAWFRQAPGKEREFVQG
ISRSDGGTYDADSVKGRFTISRDNAKNTVYLQMNSLKPEDTAVYFCAAAN
EYWVYVNPNRYTYWGRGTQVTVSS
< PMP7E12, SEQ ID NO: 89; PRT;>
EVQLVESGGGLVQAGGSLRLSCTASGRTFENNAMAWFRQAAGKEREFVAG
FSGSGGATYYAHSVEGRFTISRDNAKNTVDLQMNSLSLEDTAVYLCAARR
KSGEVVFTIPARYDYWGQGTQVTVSS
< PMP8C7, SEQ ID NO: 90; PRT; ->
QVKLEESGGGLVQAGGSLRLSCAASGRAFSSYVMGWFRQAPGKEREIVGA
ISWRGGSTYYADSVKGRFTISRDNAKNTVYLQMNSLKPEDTAVYYCAAVY
RVGAISEYSGTDYYTDEYDYWGQGTQVTVSS
< PMP9G8, SEQ ID NO: 91; PRT; ->
EVQLVESGGGLVQAGGSLRLSCAASGRTFSSYAMGWFRQAPGKEREFVVA
INWSSGSTYYADSVKGRFTISRDNAKNTMYLQMNSLKPEDTAVYYCAAGY
QINSGNYNFKDYEYDYWGQGTQVTVSS
< PMP38G7, SEQ ID NO: 92; PRT; ->
EVQLVESGGGLVQAGGSLRLSCAASGRTFSSYVMGWFRQATGKEREFVAT
IAWDSGSTYYADSVKGRFTISRDNAKNTVHLQMNSLKPEDTAVYYCAASY
NVYYNNYYYPISRDEYDYWGQGTQVTVSS
< 27-1-H7, SEQ ID NO: 93; PRT; ->
EVQLVESGGGLVQAGGSLRLSCAASGRGAWAWFRQAPGKEREFVGGLSWS
ADSTYYADSVKGRFTISRDNAKNTVFLQMTNLKADDTAVYYCAAHRRPFA
SVFTTTRMYDYWGPGTQVTVSS

TABLE 2
Anti-EGFR Nanobody families

| | Nanobody | | | | |
|---|---|---|---|---|---|
| Nanobody | type | EGF | Fab_{Erbitux} | TGFα | |
| family | example | competitor | competitor | competitor | CDR3 AA sequence |
| I | 27-10-E8 | + | − | + | ASVKLVFVNPHRYIY |
| II | PMP7D12 | + | + | + | AAGSAWYGTLYEYDY |
| III | PMP7A5 | + | − | + | SSTRTVIYTLPRMYNY |
| IV | PMP9A7 | + | Not tested | Not tested | NSRSSWVIFTIKGQYDR |
| V | PMP8B5 | + | − | Not tested | RPGMIITTIQATYGF |
| VI | PMP7E12 | + | − | Not tested | RRKSGEVVFTIPARYDY |
| VII | PMP9G8 | + | − | Not tested | GYQINSGNYNFKDYEYDY |
| VIII | PMP38G7 | + | − | Not tested | SYNVYYNNYYYPISRDEYDY |
| IX | 27-1-H7 | + | − | Not tested | HRRPFASVFTTTRMYDY |
| X | PMP9C1 | - | − | + | MVGPPPRSLDYGLGNHYEYDY |
| XI | PMP11C9 | + | Not tested | Not tested | GSPYGTELPYTRIEQYAY |
| XII | PMP11H6 | + | Not tested | Not tested | ANEYWVYVNPNRYTY |
| XIII | PMP8C7 | − | Not tested | + | VYRVGAISEYSGTDYYTDEYDY |

TABLE 3
Off rates of EGFR specific Nanobodies that block EGF binding

| | Nanobody | |
|---|---|---|
| Nanobody | type | off rate |
| family | example | (s⁻¹) |
| I | 27-10-E8 | 1.7E−3 |
| II | PMP7D12 | 2.5E−3 |
| III | PMP7A5 | 1.5E−3 |
| VI | PMP7E12 | 1.5E−3 |
| VII | PMP9G8 | 4.5E−4 |
| VIII | PMP38G7 | 1.7E−4 |
| IX | 27-1-H7 | 3.1E−3 |

TABLE 4

Multivalent/multispecific
EGFR Nanobody sequences

<Name, SEQ ID #; PRT (protein); ->
Sequence
< ALB8-3A-7A5, SEQ ID NO: 110; PRT; ->
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTLVTVSSAAAEVQLVESGGGLVQAGGSLRLSCAASDRTFSSN
NMGWFRQAPGKEREFVAAIGWGGLETHYSDSVKGRFTISRDNAKNTVYLQ
MNSLKPEDTARYYCAVSSTRTVIYTLPRMYNYWGQGTQVTVSS
< ALB8-GGGGS-7A5, SEQ ID NO: 111; PRT; ->
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTLVTVSSGGGGSEVQLVESGGGLVQAGGSLRLSCAASDRTFS
SNNMGWFRQAPGKEREFVAAIGWGGLETHYSDSVKGRFTISRDNAKNTVY
LQMNSLKPEDTARYYCAVSSTRTVIYTLPRMYNYWGQGTQVTVSS
< ALB8-GS-7A5, SEQ ID NO: 112; PRT; ->
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTQVTVSSGGGGSGGGSEVQLVESGGGLVQAGGSLRLSCAASD
RTFSSNNMGWFRQAPGKEREFVAAIGWGGLETHYSDSVKGRFTISRDNAK
NTVYLQMNSLKPEDTARYYCAVSSTRTVIYTLPRMYNYWGQGTQVTVSS
< ALB8-GS5-7A5, SEQ ID NO: 113; PRT; ->
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTQVTVSSGGGGSGGGGSGGGGSGGGGSGGGGSEVQLVESGGG
LVQAGGSLRLSCAASDRTFSSNNMGWFRQAPGKEREFVAAIGWGGLETHY
SDSVKGRFTISRDNAKNTVYLQMNSLKPEDTARYYCAVSSTRTVIYTLPR
MYNYWGQGTQVTVSS
< ALB8-3A-7D12, SEQ ID NO: 114; PRT; ->
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTLVTVSSAAAEVQLVESGGGSVQTGGSLRLTCAASGRTSRSY
GMGWFRQAPGKEREFVSGISWRGDSTGYADSVKGRFTISRDNAKNTVDLQ
MNSLKPEDTAIYYCAAAAGSAWYGTLYEYDYWGQGTQVTVSS
< ALB8-GGGGS-7D12, SEQ ID NO: 115; PRT; ->
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTLVTVSSGGGGSEVQLVESGGGSVQTGGSLRLTCAASGRTSR
SYGMGWFRQAPGKEREFVSGISWRGDSTGYADSVKGRFTISRDNAKNTVD
LQMNSLKPEDTAIYYCAAAAGSAWYGTLYEYDYWGQGTQVTVSS
< ALB8-GS-7D12, SEQ ID NO: 116; PRT; ->
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTQVTVSSGGGGSGGGSEVQLVESGGGSVQTGGSLRLTCAASG
RTSRSYGMGWFRQAPGKEREFVSGISWRGDSTGYADSVKGRFTISRDNAK
NTVDLQMNSLKPEDTAIYYCAAAAGSAWYGTLYEYDYWGQGTQVTVSS
< ALB8-GS5-7D12, SEQ ID NO: 117; PRT; ->
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTQVTVSSGGGGSGGGGSGGGGSGGGGSGGGGSEVQLVESGGG
SVQTGGSLRLTCAASGRTSRSYGMGWFRQAPGKEREFVSGISWRGDSTGY
ADSVKGRFTISRDNAKNTVDLQMNSLKPEDTAIYYCAAAAGSAWYGTLYE
YDYWGQGTQVTVSS
< 7A5-GS-ALB8, SEQ ID NO: 118; PRT; ->
EVQLVESGGGLVQAGGSLRLSCAASDRTFSSNNMGWFRQAPGKEREFVAA
IGWGGLETHYSDSVKGRFTISRDNAKNTVYLQMNSLKPEDTARYYCAVSS
TRTVIYTLPRMYNYWGQGTQVTVSSGGGGSGGGSEVQLVESGGGLVQPGN
SLRLSCAASGFTFSSFGMSWVRQAPGKGLEWVSSISGSGSDTLYADSVKG
RFTISRDNAKTTLYLQMNSLRPEDTAVYYCTIGGSLSRSSQGTLVTVSS
< 7A5-GS5-ALB8, SEQ ID NO: 119; PRT; ->
EVQLVESGGGLVQAGGSLRLSCAASDRTFSSNNMGWFRQAPGKEREFVAA
IGWGGLETHYSDSVKGRFTISRDNAKNTVYLQMNSLKPEDTARYYCAVSS
TRTVIYTLPRMYNYWGQGTQVTVSSGGGGSGGGGSGGGGSGGGGSGGGGS
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTLVTVSS
< 7D12-GS-ALB8, SEQ ID NO: 120; PRT; ->
QVKLEESGGGSVQTGGSLRLTCAASGRTSRSYGMGWFRQAPGKEREFVSG
ISWRGDSTGYADSVKGRFTISRDNAKNTVDLQMNSLKPEDTAIYYCAAAA
GSAWYGTLYEYDYWGQGTQVTVSSGGGGSGGGSEVQLVESGGGLVQPGNS
LRLSCAASGFTFSSFGMSWVRQAPGKGLEWVSSISGSGSDTLYADSVKGR
FTISRDNAKTTLYLQMNSLRPEDTAVYYCTIGGSLSRSSQGTLVTVSS
< 7D12-GS5-ALB8, SEQ ID NO: 121; PRT; ->
QVKLEESGGGSVQTGGSLRLTCAASGRTSRSYGMGWFRQAPGKEREFVSG
ISWRGDSTGYADSVKGRFTTSRDNAKNTVDLQMNSLKPEDTAIYYCAAAA
GSAWYGTLYEYDYWGQGTQVTVSSGGGGSGGGGSGGGGSGGGGSGGGGSE
VQLVESGGGLVQPGNSLRLSCAASGFTFSSFGMSWVRQAPGKGLEWVSSI
SGSGSDTLYADSVKGRFTISRDNAKTTLYLQMNSLRPEDTAVYYCTIGGS
LSRSSQGTLVTVSS
< 7D12-GS-7D12, SEQ ID NO:122; PRT; ->
QVKLEESGGGSVQTGGSLRLTCAASGRTSRSYGMGWFRQAPGKEREFVSG
ISWRGDSTGYADSVKGRFTISRDNAKNTVDLQMNSLKPEDTAIYYCAAAA
GSAWYGTLYEYDYWGQGTQVTVSSGGGGSGGGSEVQLVESGGGSVQTGGS
LRLTCAASGRTSRSYGMGWFRQAPGKEREFVSGISWRGDSTGYADSVKGR
FTISRDNAKNTVDLQMNSLKPEDTAIYYCAAAAGSAWYGTLYEYDYWGQG
TQVTVSS
< 7D12-GS5-7D12, SEQ ID NO: 123; PRT; ->
QVKLEESGGGSVQTGGSLRLTCAASGRTSRSYGMGWFRQAPGKEREFVSG
ISWRGDSTGYADSVKGRFTISRDNAKNTVDLQMNSLKPEDTAIYYCAAAA
GSAWYGTLYEYDYWGQGTQVTVSSGGGGSGGGGSGGGGSGGGGSGGGGSE
VQLVESGGGSVQTGGSLRLTCAASGRTSRSYGMGWFRQAPGKEREFVSGI
SWRGDSTGYADSVKGRFTISRDNAKNTVDLQMNSLKPEDTAIYYCAAAAG
SAWYGTLYEYDYWGQGTQVTVSS
< 7A5-GS-7A5-GS-ALB8, SEQ ID NO: 124; PRT; ->
EVQLVESGGGLVQAGGSLRLSCAASDRTFSSNNMGWFRQAPGKEREFVAA
IGWGGLETHYSDSVKGRFTISRDNAKNTVYLQMNSLKPEDTARYYCAVSS
TRTVIYTLPRMYNYWGQGTQVTVSSGGGGSGGGSEVQLVESGGGLVQAGG
SLRLSCAASDRTFSSNNMGWFRQAPGKEREFVAAIGWGGLETHYSDSVKG
RFTISRDNAKNTVYLQMNSLKPEDTARYYCAVSSTRTVIYTLPRMYNYWG
QGTQVTVSSGGGGSGGGSEVQLVESGGGLVQPGNSLRLSCAASGFTFSSF
GMSWVRQAPGKGLEWVSSISGSGSDTLYADSVKGRFTISRDNAKTTLYLQ
MNSLRPEDTAVYYCTIGGSLSRSSQGTLVTVSS
< 7A5-GS-7A5-3A-ALB8, SEQ ID NO: 125; PRT; ->
EVQLVESGGGLVQAGGSLRLSCAASDRTFSSNNMGWFRQAPGKEREFVAA
IGWGGLETHYSDSVKGRFTISRDNAKNTVYLQMNSLKPEDTARYYCAVSS
TRTVIYTLPRMYNYWGQGTQVTVSSGGGGSGGGSEVQLVESGGGLVQAGG
SLRLSCAASDRTFSSNNMGWFRQAPGKEREFVAAIGwGGLETHYSDSVKG
RFTISRDNAKNTVYLQMNSLKPEDTARYYCAVSSTRTVIYTLPRMYNYWG
QGTQVTVSSAAAEVQLVESGGGLVQPGNSLRLSCAASGFTFSSFGMSWVR
QAPGKGLEWVSSISGSGSDTLYADSVKGRFTISRDNAKTTLYLQMNSLRP
EDTAVYYCTIGGSLSRSSQGTLVTVSS
< 7D12-GS-7D12-GS-ALB8, SEQ ID NO: 126;PRT; ->
QVKLEESGGGSVQTGGSLRLTCAASGRTSRSYGMGWFRQAPGKEREFVSG
ISWRGDSTGYADSVKGRFTTSRDNAKNTVDLQMNSLKPEDTAIYYCAAAA
GSAWYGTLYEYDYWGQGTQVTVSSGGGGSGGGSEVQLVESGGGSVQTGGS
LRLTCAASGRTSRSYGMGWFRQAPGKEREFVSGISWRGDSTGYADSVKGR
FTISRDNAKNTVDLQMNSLKPEDTAIYYCAAAAGSAWYGTLYEYDYWGQG
TQVTVSSGGGGSGGGSEVQLVESGGGLVQPGNSLRLSCAASGFTFSSFGM
SWVRQAPGKGLEWVSSISGSGSDTLYADSVKGRFTISRDNAKTTLYLQMN
SLRPEDTAVYYCTIGGSLSRSSQGTLVTVSS
< 7C12-GS-7C12-GS-ALB8, SEQ ID NO:127; PRT; ->
AVQLVESGGGSVQAGGSLRLTCAASGRTSRSYGMGWFRQAPGKEREFVSG
ISWRGDSTGYADSVKGRFTISRDNAKNTVDLQMNSLKPEDTAIYYCAAAA
GSTWYGTLYEYDYWGQGTQVTVSSGGGGSGGGSEVQLVESGGGSVQAGGS
LRLTCAASGRTSRSYGMGWFRQAPGKEREFVSGISWRGDSTGYADSVKGR
FTISRDNAKNTVDLQMNSLKPEDTAIYYCAAAAGSTWYGTLYEYDYWGQG
TQVTVSSGGGGSGGGSEVQLVESGGGLVQPGNSLRLSCAASGFTFSSFGM
SWVRQAPGKGLEWVSSISGSGSDTLYADSVKGRFTISRDNAKTTLYLQMN
SLRPEDTAVYYCTIGGSLSRSSQGTLVTVSS
< 7A5-GGS-ALB8-3A-7A5, SEQ ID NO: 128; PRT; ->
EVQLVESGGGLVQAGGSLRLSCAASDRTFSSNNMGWFRQAPGKEREFVAA
IGWGGLETHYSDSVKCRFTISRDNAKNTVYLQMNSLKPEDTARYYCAVSS
TRTVIYTLPRIMYNYWGQGTQVTVSSGGSEVQLVESGGGLVQPGNSLRLS
CAASGFTFSSFGMSWVRQAPGKGLEWVSSISGSGSDTLYADSVKGRFTIS
RDNAKTTLYLQMNSLRPEDTAVYYCTIGGSLSRSSQGTLVTVSSAAAEVQ
LVRSGGGLVQAGGSLRLSCAASDRTFSSNNMGWFRQAPGKEREFVAAIGW
GGLETHYSDSVKGRFTISRDNAKNTVYLQMNSLKPEDTARYYCAVSSTRT
VIYTLPRMYNYWGQGTQVTVSS
< 7A5-GS-ALB8-GS-7A5, SEQ ID NO: 129; PRT; ->
EVQLVESGGGLVQAGGSLRLSCAASDRTFSSNNMGWFRQAPGKEREFVAA
IGWGGLETHYSDSVKGRFTISRDNAKNTVYLQMNSLKPEDTARYYCAVSS
TRTVIYTLPRMYNYWGQGTQVTVSSGGGGSGGGSEVQLVESGGGLVQPGN
SLRLSCAASGFTFSSFGMSWVRQAFGKGLEWVSSISGSGSDTLYADSVKG
RFTISRDNAKTTLYLQMNSLRPEDTAVYYCTIGGSLSRSSQGTQVTVSSG
GGGSGGGSEVQLVESGGGLVQAGGSLRLSCAASDRTFSSNNMGWFRQAPG
KEREFVAAIGWGGLETHYSDSVKGRFTISRDNAKNTVYLQMNSLKPEDTA
RYYCAVSSTRTVIYTLPRMYNYWGQGTQVTVSS
< 7D12-GS-ALB8-GS-7D12, SEQ ID NO: 130; PRT; ->
QVKLEESGGGSVQTGGSLRLTCAASGRTSRSYGMGWFRQAPGKEREFVSG
ISWRGDSTGYADSVKGRFTISRDNAKNTVDLQMNSLKPEDTAIYYCAAAA
GSAWYGTLYEYDYWGQGTQVTVSSGGGGSGGGSEVQLVESGGGLVQPGNS
LRLSCAASGFTFSSFGMSWVRQAPGKGLEWVSSISGSGSDTLYADSVKGR
FTISRDNAKTTLYLQMNSLRPEDTAVYYCTIGGSLSRSSQGTQVTVSSGG
GGSGGGSEVQLVESGGGSVQTGGSLRLTCAASGRTSRSYGMGWFRQAPGK
EREFVSGISWRGDSTGYADSVKGRFTISRDNAKNTVDLQMNSLKPEDTAI
YYCAAAAGSAWYGTLYEYDYWGQGTQVTVSS
< 7C12-GS-ALB8-GS-7C12, SEQ ID NO: 131;PRT; ->
AVQLVESGGGSVQAGGSLRLTCAASGRTSRSYGMGWFRQAPGKEREFVSG
ISWRGDSTGYADSVKGRFTTSRDNAKNTVDLQMNSLKPEDTAIYYCAAAA
GSTWYGTLYEYDYWGQGTQVTVSSGGGGSGGGSEVQLVESGGGLVQPGNS
LRLSCAASGFTFSSFGMSWVRQAPGKGLEWVSSISGSGSDTLYADSVKGR
FTISRDNAKTTLYLQMNSLRPEDTAVYYCTIGGSLSRSSQGTQVTVSSGG
GGSGGGSEVQLVESGGGSVQAGGSLRLTCAASGRTSRSYGMGWERQAPGK
EREFVSGISWRGDSTGYADSVKGRFTISRDNAKNTVDLQMNSLKPEDTAI
YYCAAAAGSTWYGTLYEYDYWGQGTQVTVSS
< ALB8-GS-7A5-GS-7A5, SEQ ID NO: 132; PRT; ->
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTLVTVSSGGGGSGGGSEVQLVESGGGLVQAGGSLRLSCAASD
RTFSSNNMGWFRQAPGKEREFVAAIGWGGLETHYSDSVKGRFPISRDNAK
NTVYLQMNSLKPEDTARYYCAVSSTRTVIYTLPRMYNYWGQGTQVTVSSG
GGGSGGGSEVQLVESGGGLVQAGGSLRLSCAASDRTFSSNNMGWFRQAPG
KEREFVAAIGWGGLETHYSDSVKGRFTISRDNAKNTVYLQMNSLKPEDTA
RYYCAVSSTRTVIYTLPRMYNYWGQGTQVTVSS
< ALB8-GS-7D12-GS-7D12, SEQ ID NO: 133; PRT;>
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTLVTVSSGGGGSGGGSEVQLVESGGGSVQTGGSLRLTCAASG
RTSRSYGMGWFRQAPGKEREFVSGISWRGDSTGYADSVKGRFTISRDNAK
NTVDLQMNSLKPEDTAIYYCAAAAGSAWYGTLYEYDYWGQGTQVTVSSGG
GGSGGGSEVQLVESGGGSVQTGGSLRLTCAASGRTSRSYGMGWFRQAPGK
EREFVSGISWRGDSTGYADSVKGRFTISRDNAKNTVDLQMNSLKPEDTAI
YYCAAAAGSAWYGTLYEYDYWGQGTQVTVSS
< EGFRPMP7D12-GS-EGFRPMP7A5, SEQ ID NO: 141;
PRT; ->
QVKLEESGGGSVQTGGSLRLTCAASGRTSRSYGMGWFRQAPGKEREFVSG
ISWRGDSTGYADSVKGRFTTSRDNAKNTVDLQMNSLKPEDTAIYYCAAAA
GSAWYGTLYEYDYWGQGTQVTVSSGGGGSGGGSQVKLEESGGGLVQAGGS
LRLSCVASGRTFSRTAMAWFRQAPGKEREFVATITWNSGTTRYADSVKGR
FFISKDSAKNTTYLEMNSLEPEDTAVYYCAATAAAVITPTRGYYNYWGQG
TQVTVSS
< EGFRPMP7D12-GS-EGFRPMP7A5-GS-ALB8,
SEQ ID NO: 142; PRT; ->
QVKLEESGGGSVQTGGSLRLTCAASGRTSRSYGMGWFRQAPGKEREFVSG
ISWRGDSTGYADSVKGRFTTSRDNAKNTVDLQMNSLKPEDTAIYYCAAAA
GSAWYGTLYEYDYWGQGTQVTVSSGGGGSGGGSQVKLEESGGGLVQAGGS
LRLSCVASGRTFSRTAMAWFRQAPGKEREFVATITWNSGTTRYADSVKGR
FFISKDSAKNTIYLEMNSLEPEDTAVYYCAATAAAVITPTRGYYNYWGQG
TQVTVSSGGGGSGGGSEVQLVESGGGLVQPGNSLRLSCAASGFTFSSFGM
SWVRQAPGKGLEWVSSISGSGSDTLYADSVKGRFTISRDNAKTTLYLQMN
SLRPEDTAVYYCTIGGSLSRSSQGTLVTVSS
< EGFRPMP7D12-GS-ALB8-GS-EGFRPMP7A5,
SEQ ID NO: 143; PRT; ->
QVKLEESGGGSVQTGGSLRLTCAASGRTSRSYGMGWFRQAPGKEREFVSG
ISWRGDSTGYADSVKGRFTTSRDNAKNTVDLQMNSLKPEDTAIYYCAAAA
GSAWYGTLYEYDYWGQGTQVTVSSGGGGSGGGSEVQLVESGGGLVQPGNS
LRLSCAASGFTFSSFGMSWVRQAPGKGLEWVSSISGSGSDTLYADSVKGR
FTISRDNAKTTLYLQMNSLRPEDTAVYYCTIGGSLSRSSQGTLVTVSSGG
GGSGGGSQVKLEESGGGLVQAGGSLRLSCVASGRTFSRTANAWFRQAPGK
EREFVATITWNSGTTRYADSVKGRFFISKDSAKNTTYLEMNSLEPEDTAV
YYCAATAAAVITPTRGYYNYWGQGTQVTVSS

TABLE 5
Preferred Nanobodies against IGF-IR

<Name, SEQ ID #; PRT (protein); ->
Sequence
< PMP4B11, SEQ ID NO: 106; PRT; ->
EVQLVESGGGLVQPGGSLRLSCAASGSIFTFNAMGWYRQAPGKQRELVAV
IISGGSTHYVDSVKGRFTISRDNAKKMVYLQMNSLKPEDTAVYYCNVKKF
GDYWGQGTQVTVSS
< PMP3G7, SEQ ID NO: 107; PRT; ->
DVQLVESGGGLVQAGGSLRLSCAASESISTINVMAWYRQAPGKQRELVAE
ITRSGRTNYVDSVKGRFTISRDNAKNTMYLQMNSLNLEDTAVYYCRTIDG
SWREYWGQGTQVTVSS
< PMP2C7, SEQ ID NO: 108; PRT; ->
QVKLEESGGGLVQPGGSLRLSCVASGRTFSNYAIVIGWFRQAPGQEREFV
AAINWNSRSTYYADSVKGRFTISRLNARNTVYLQMNRLKPEDTAVYDCAA
SHDSDYGGTNANLYDYWGQGTQVTVSS
< PMP1C7, SEQ ID NO: 109; PRT; ->
QVKLEESGGGLVQAGGSLRLSCVASGRTFSRTANAWFRQAPGKEREFVAT
ITWNSGTTRYADSVKGRFFISKDSAKNTIYLEMNSLEPEDTAVYYCAATA
AAVITPTRGYYNYWGQGTQVTVSS

TABLE 6

Multivalent/multispecific
IGF-IR Nanobody sequences

<Name, SEQ ID #; PRT (protein); ->
Sequence
< PMP1C7-GS-PMP1C7, SEQ ID NO: 134; PRT; ->
QVKLEESGGGLVQAGGSLRLSCVASGRTFSRTAMAWFRQAPGKEREFVAT
ITWNSGTTRYADSVKGRFFISKDSAKNTIYLEMNSLEPEDTAVYYCAATA
AAVITPTRGYYNYWGQGTQVTVSSGGGGSGGGSQVKLEESGGGLVQAGGS
LRLSCVASGRTFSRTANAWFRQAPGKEREFVATITWNSGTTRYADSVKGR
FFISKDSAKNTIYLEMNSLEPEDTAVYYCAATAAAVITPTRGYYNYWGQG
TQVTVSS
< PMP1C7-GS5-PMP1C7, SEQ ID NO: 135; PRT; >
QVKLEESGGGLVQAGGSLRLSCVASGRTFSRTAMAWFRQAPGKEREFVAT
ITWNSGTTRYADSVKGRFFISKDSAKNTIYLEMNSLEPEDTAVYYCAATA
AAVITPTRGYYNYWGQGTQVTVSSGGGGSGGGGSGGGGSGGGGSGGGGSQ
VKLEESGGGLVQAGGSLRLSCVASGRTFSRTAMAWFRQAPGKEREFVATI
TWNSGTTRYADSVKGRFFISKDSAKNTIYLEMNSLEPEDTAVYYCAATAA
AVITPTRGYYNYWGQGTQVTVSS

TABLE 7
Preferred Nanobodies against EGFR and IGF-IR

<Name, SEQ ID #; PRT (protein); ->
Sequence
< EGFRPMP7D12-GS-IGFRPMP1C7, SEQ ID NO: 136;
PRT; ->
VKLEESGGGSVQTGGSLRLTCAASGRTSRSYGMGWFRQAPGKEREFVSG
ISWRGDSTGYADSVKGRFTISRDNAKNTVDLQMNSLKPEDTAIYYCAAAA
GSAWYGTLYEYDYWGQGTQVTVSSGGGGSGGGSQVKLEESGGGLVQAGGS
LRLSCVASGRTFSRTANAWFRQAPGKEREFVATITWNSGTTRYADSVKGR
FFISKDSAKNTIYLEMNSLEPEDTAVYYCAATAAAVITPTRGYYNYWGQG
TQVTVSS
< EGFRPMP7D12-GS5-IGFRPMP1C7, SEQ ID NO: 137;
PRT; ->
QVKLEESGGGSVQTGGSLRLTCAASGRTSRSYGMGWFRQAPGKEREFVSG
ISWRGDSTGYADSVKGRFTISRDNAKNTVDLQMNSLKPEDTAIYYCAAAA
GSAWYGTLYEYDYWGQGTQVTVSSGGGGSGGGGSGGGGSGGGGSGGGGSQ
VKLEESGGGLVQAGGSLRLSCVASGRTFSRTAMAWFRQAPGKEREFVATI
TWNSGTTRYADSVKGRFFISKDSAKNTIYLEMNSLEPEDTAVYYCAATAA
AVITPTRGYYNYWGQGTQVTVSS
< EGFRPMP7D12GS-IGFRPMP1C7-GSALB8, SEQ ID NO: 138;
PRT;>
QVKLEESGGGSVQTGGSLRLTCAASGRTSRSYGMGWFRQAPGKEREFVSG
ISWRGDSTGYADSVKGRFTISRDNAKNTVDLQMNSLKPEDTAIYYCAAAA
GSAWYGTLYEYDYWGQGTQVTVSSGGGGSGGGSQVKLEESGGGLVQAGGS
LRLSCVASGRTFSRTAMAWFRQAPGKEREFVATITWNSGTTRYADSVKGR
FFISKDSAKNTIYLEMNSLEPEDTAVYYCAATAAAVITPTRGYYNYWGQG
TQVTVSSGGGGSGGGSEVQLVESGGGLVQPGNSLRLSCAASGFTFSSFGM
SWVRQAPGKGLEWVSSISGSGSDTLYADSVKGRFTISRDNAKTTLYLQMN
SLRPEDTAVYYCTIGGSLSRSSQGTLVTVSS
< EGFRPMP7D12-GS-ALB8-GS-IGFRPMP1C7,
SEQ ID NO: 139; PRT; ->
QVKLEESGGGSVQTGGSLRLTCAASGRTSRSYGMGWFRQAPGKEREFVSG
ISWRGDSTGYADSVKGRFTISRDNAKNTVDLQMNSLKPEDTAIYYCAAAA
GSAWYGTLYEYDYWGQGTQVTVSSGGGGSGGGSEVQLVESGGGLVQPGNS
LRLSCAASGFTFSSFGMSWVRQAPGKGLEWVSSISGSGSDTLYADSVKGR
FTISRDNAKTTLYLQMNSLRPEDTAVYYCTIGGSLSRSSQGTLVTVSSGG
GGSGGGSQVKLEESGGGLVQAGGSLRLSCVASGRTFSRTAMAWFRQAPGK
EREFVATITWNSGTTRYADSVKGRFFISKDSAKNTIYLEMNSLEPEDTAV
YYCAATAAAVITPTRGYYNYWGQGTQVTVSS
< EGFRPMP7D12-GS5-IGFRPMP1C7-GS-ALB8,
SEQ ID NO: 140; PRT; ->
QVKLEESGGGSVQTGGSLRLTCAASGRTSRSYGMGWFRQAPGKEREFVSG
ISWRGDSTGYADSVKGRFTISRDNAKNTVDLQMNSLKPEDTAIYYCAAAA
GSAWYGTLYEYDYWGQGTQVTVSSGGGGSGGGGSGGGGSGGGGSGGGGSQ
VKLEESGGGLVQAGGSLRLSCVASGRTFSRTAMAWFRQAPGKEREFVATI
TWNSGTTRYADSVKGRFFISKDSAKNTIYLEMNSLEPEDTAVYYCAATAA
AVITPTRGYYNYWGQGTQVTVSSGGGGSGGGSEVQLVESGGGLVQPGNSL
RLSCAASGFTFSSFGMSWVRQAPGKGLEWVSSISGSGSDTLYADSVKGRF
TISRDNAKTTLYLQMNSLRPEDTAVYYCTIGGSLSRSSQGTLVTVSS

TABLE 8
SEQ ID NO's: 1-41

< Name, SEQ ID #; PRT (protein); ->
Sequence
< FR1, SEQ ID NO: 1; PRT; ->
QVQLQESGGGXVQAGGSLRLSCAASG
< FR2, SEQ ID NO: 2; PRT; ->
WXRQAPGKXXEXVA
< FR3, SEQ ID NO: 3; PRT; ->
RFTISRDNAKNTVYLQMNSLXXEDTAVYYCAA
< FR4, SEQ ID NO: 4; PRT; ->
XXQGTXVTVSS
< FR1, SEQ ID NO: 5; PRT; ->
QVQLQESGGGLVQAGGSLRLSCAASG
< FR2, SEQ ID NO: 6; PRT; ->
WFRQAPGKERELVA
< FR2, SEQ ID NO: 7; PRT; ->
WFRQAPGKEREFVA
< FR2, SEQ ID NO: 8; PRT; ->
WFRQAPGKEREGA
< FR2, SEQ ID NO: 9; PRT; ->
WFRQAPGKQRELVA
< FR2, SEQ ID NO: 10; PRT; ->
WFRQAPGKQREFVA
< FR2, SEQ ID NO: 11; PRT; ->
WYRQAPGKGLEWA
< FR3, SEQ ID NO: 12; PRT; ->
RFTISRDNAKNTVYLQMNSLKPEDTAVYYCAA
< FR4, SEQ ID NO: 13; PRT; ->
WGQGTQVTVSS
< FR4, SEQ ID NO: 14; PRT; ->
WGQGTLVTVSS
< CDR1, SEQ ID NO: 15; PRT; ->
SFGMS
< CDR1, SEQ ID NO: 16; PRT; ->
LNLMG
< CDR1, SEQ ID NO: 17; PRT; ->
INLLG
< CDR1, SEQ ID NO: 18; PRT; ->
NYWMY
< CDR2, SEQ ID NO: 19; PRT; ->
SISGSGSDTLYADSVKG
< CDR2, SEQ ID NO: 20; PRT; ->
TITVGDSTNYADSVKG
< CDR2, SEQ ID NO: 21; PRT; ->
TITVGDSTSYADSVKG
< CDR2, SEQ ID NO: 22; PRT; ->
SINGRGDDTRYADSVKG
< CDR2, SEQ ID NO: 23; PRT; ->
AISADSSTKNYADSVKG
< CDR2, SEQ ID NO: 24; PRT; ->
AISADSSDKRYADSVKG
< CDR2, SEQ ID NO: 25; PRT; ->
RISTGGGYSYYADSVKG
< CDR3, SEQ ID NO: 26; PRT; ->
DREAQVDTLDFDY
< CDR3, SEQ ID NO: 27; PRT; ->
GGSLSR
< CDR3, SEQ ID NO: 28; PRT; ->
RRTWHSEL
< CDR3, SEQ ID NO: 29; PRT; ->
GRSVSRS
< CDR3, SEQ ID NO: 30; PRT; ->
GRGSP
< Myc-tag, SEQ ID NO: 31; PRT; ->
AAAEQKLISEEDLNGAA
< PMP 6A6 (ALB-1), SEQ ID NO: 32; PRT; ->
AVQLVESGGGLVQPGNSLRLSCAASGFTFRSFGMSWVRQAPGKEPEWVSS
ISGSGSDTLYADSVKGRFTISRDNAKTTLYLQMNSLKPEDTAVYYCTIGG
SLSRSSQGTQVTVSS
< ALB-8, SEQ ID NO: 33; PRT; ->
EVQLVESGGGLVQPGNSLRLSCAASGFTFSSFGMSWVRQAPGKGLEWVSS
ISGSGSDTLYADSVKGRFTISRDNAKTTLYLQMNSLRPEDTAVYYCTIGG
SLSRSSQGTLVTVSS
< PMP 6A8 (ALB2), SEQ ID NO: 34; PRT; ->
AVQLVESGGGLVQGGGSLRLAVAASERIFDLNLMGWYRQGPGNERELVAT
CITVGDSTNYADSVKGRFTISMOYTKQTVYLHMNSLRPEDTGLYYCKIRR
TWHSELWGQGTQVTVSS
< FC44, SEQ ID NO: 35; PRT; ->
EVQLQASGGGLVQAGGSLRLSCSASVRTFSIYAMGWFRQAPGKEREFVAG
INRSGOVTKYADFVKGRFSISRDNAKNMVYLQMNSLKPEDTALYYCAATW
AYDTVGALTSGYNFWGQGTQVTVSS
< FC5, SEQ ID NO: 36; PRT; ->
EVQLQASGGGLVQAGGSLRLSCAASGFKITHYTMGWFRQAPGKEREFVSR
ITWGGDNTFYSNSVKGRFTISRDNAKNTVYLQMNSLKPEDTADYYCAAGS
TSTATPLRVDYWGKGTQVTVSS
< GS30, SEQ ID NO: 37; PRT; ->
GGGGSGGGGSGGGGSGGGGSGGGGSGGGGS
< GS15, SEQ ID NO: 38; PRT; ->
GGGGSGGGGSGGGGS
< GS9, SEQ ID NO: 39; PRT; ->
GGGGSGGGS
< GS7, SEQ ID NO: 40; PRT; ->
SGGSGGS <
< LLAMA UPPER LONG HINGE REGION, SEQ ID NO: 41;
PRT; ->
EPKTPKPQPAAA

TABLE 9
Preferred CDR's of Nanobodies against EGFR

<Name, SEQ ID #; PRT (protein); ->
Sequence
< CDR1, SEQ ID NO: 42; PRT; ->
TYTMA
< CDR1, SEQ ID NO: 43; PRT; ->
SYGMG
< CDR1, SEQ ID NO: 44; PRT; ->
GFAMG
< CDR1, SEQ ID NO: 45; PRT; ->
SNNMG
< CDR1, SEQ ID NO: 46; PRT; ->
GDVMG
< CDR1, SEQ ID NO: 47; PRT; ->
SYVVG
< CDR1, SEQ ID NO: 48; PRT; ->
DYNMA
< CDR1, SEQ ID NO: 49; PRT; ->
TYTMA
< CDR1, SEQ ID NO: 50; PRT; ->
NNAMA
< CDR1, SEQ ID NO: 51; PRT; ->
SYVMG
< CDR1, SEQ ID NO: 52; PRT; ->
SYAMG
< CDR1, SEQ ID NO: 53; PRT; ->
A
< CDR2, SEQ ID NO: 54; PRT; ->
GISRSDGGTYDADSVKG
< CDR2, SEQ ID NO: 55; PRT; ->
GISWRGDSTGYADSVKG
< CDR2, SEQ ID NO: 56; PRT; ->
AISWSGGSLLYVDSVKG
< CDR2, SEQ ID NO: 57; PRT; ->
AIGWGGLETHYSDSVKG
< CDR2, SEQ ID NO: 58; PRT; ->
GFSRSTSTTHYADSVKG
< CDR2, SEQ ID NO: 59; PRT; ->
GIAWGDGITYYADSVKG
< CDR2, SEQ ID NO: 60; PRT; ->
HISWLGGRTYYRDSVKG
< CDR2, SEQ ID NO: 61; PRT; ->
GFSGSGGATYYAHSVEG
< CDR2, SEQ ID NO: 62; PRT; ->
AISWRGGSTYYADSVKG
< CDR2, SEQ ID NO: 63; PRT; ->
AINWSSGSTYYADSVKG
< CDR2, SEQ ID NO: 64; PRT; ->
TIAWDSGSTYYADSVKG
< CDR2, SEQ ID NO: 65; PRT; ->
GLSWSADSTYYADSVKG
< CDR3, SEQ ID NO: 66; PRT; ->
ASVKLVYVNPNRYSY
< CDR3, SEQ ID NO: 67; PRT; ->
AAGSAWYGTLYEYDY
< CDR3, SEQ ID NO: 68; PRT; ->
AAGSTWYGTLYEYDY
< CDR3, SEQ ID NO: 69; PRT; ->
MVGPPPRSLDYGLGNHYEYDY
< CDR3, SEQ ID NO: 70; PRT; ->
SSTRTVIYTLPRMYNY
< CDR3, SEQ ID NO: 71; PRT; ->
NSRSSWVIFTIKGQYDR
< CDR3, SEQ ID NO: 72; PRT; ->
RPGMIITTIQATYGF
< CDR3, SEQ ID NO: 73; PRT; ->
GSPYGTELPYTRIEQYAY
< CDR3, SEQ ID NO: 74; PRT; ->
ANEYWVYVNPNRYTY
< CDR3, SEQ ID NO: 75; PRT; ->
RRKSGEVVFTIPARYDY
< CDR3, SEQ ID NO: 76; PRT; ->
VYRVGAISEYSGTDYYTDEYDY
< CDR3, SEQ ID NO: 77; PRT; ->
GYQINSGNYNFKDYEYDY
< CDR3, SEQ ID NO: 78; PRT; ->
SYNVYYNNYYYPISRDEYDY
< CDR3, SEQ ID NO: 79; PRT; ->
HRRPFASVFTTTRMYDY

TABLE 10
Preferred CDR's of Nanobodies against IGF-IR

<Name, SEQ ID #; PRT (protein); ->
Sequence
< CDR1, SEQ ID NO: 94; PRT; ->
FNAMG
< CDR1, SEQ ID NO: 95; PRT; ->
INVMA
< CDR1, SEQ ID NO: 96; PRT; ->
NYAMG
< CDR1, SEQ ID NO: 97; PRT; ->
RTAMA
< CDR2, SEQ ID NO :98; PRT; ->
VIISGGSTHYVDSVKG
< CDR2, SEQ ID NO: 99; PRT; ->
EITRSGRTNYVDSVKG
< CDR2, SEQ ID NO: 100; PRT; ->
AINWNSRSTYYADSVKG
< CDR2, SEQ ID NO: 101; PRT; ->
TITWNSGTTRYADSVKG
< CDR3, SEQ ID NO: 102; PRT; ->
KKFGDY
< CDR3, SEQ ID NO: 103; PRT; ->
IDGSWREY
< CDR3, SEQ ID NO: 104; PRT; ->
SHDSDYGGTNANLYDY
< CDR3, SEQ ID NO: 105; PRT; ->
TAAAVITPTRGYYNY

1. Nanobody against EGFR, which comprises or consists of 4 framework regions (FR1 to FR4 respectively) and 3 complementarity determining regions (CDR1 to CDR3 respectively), in which: CDR1 is an amino acid sequence chosen from the group consisting of:

| | |
|---|---|
| TYTMA | [SEQ ID NO: 42] |
| SYGMG | [SEQ ID NO: 43] |
| GFAMG | [SEQ ID NO: 44] |
| SNNMG | [SEQ ID NO: 45] |
| GDVMG | [SEQ ID NO: 46] |
| SYWG | [SEQ ID NO: 47] |
| DYNMA | [SEQ ID NO: 48] |
| TYTMA | [SEQ ID NO: 49] |
| NNAMA | [SEQ ID NO: 50] |
| SYVMG | [SEQ ID NO: 51] |
| SYAMG | [SEQ ID NO: 52] |
| A | [SEQ ID NO: 53] |

or from the group consisting of amino acid sequences that have at least 80%, preferably at least 90%, more preferably at least 95%, even more preferably at least 99% sequence identity (as defined herein) with one of the above amino acid sequences; in which
(i) any amino acid substitution is preferably a conservative amino acid substitution (as defined herein); and/or
(ii) said amino acid sequence preferably only contains amino acid substitutions, and no amino acid deletions or insertions, compared to the above amino acid sequence(s);
and/or from the group consisting of amino acid sequences that have 2 or only 1 “amino acid difference(s)” (as defined herein) with one of the above amino acid sequences, in which:
(i) any amino acid substitution is preferably a conservative amino acid substitution (as defined herein); and/or
(ii) said amino acid sequence preferably only contains amino acid substitutions, and no amino acid deletions or insertions, compared to the above amino acid sequence(s);
and/or in which:
CDR2 is an amino acid sequence chosen from the group consisting of:

| | |
|---|---|
| GISRSDGGTYDADSVKG | [SEQ ID NO: 54] |
| GISWRGDSTGYADSVKG | [SEQ ID NO: 55] |
| AISWSGGSLLYVDSVKG | [SEQ ID NO: 56] |
| AIGWGGLETHYSDSVKG | [SEQ ID NO: 57] |
| GFSRSTSTTHYADSVKG | [SEQ ID NO: 58] |
| GIAWGDGITYYADSVKG | [SEQ ID NO: 59] |
| HISWLGGRTYYRDSVKG | [SEQ ID NO: 60] |
| GFSGSGGATYYAHSVEG | [SEQ ID NO: 61] |
| AISWRGGSTYYADSVKG | [SEQ ID NO: 62] |
| AINWSSGSTYYADSVKG | [SEQ ID NO: 63] |
| TIAWDSGSTYYADSVKG | [SEQ ID NO: 64] |
| GLSWSADSTYYADSVKG | [SEQ ID NO: 65] |

or from the group consisting of amino acid sequences that have at least 80%, preferably at least 90%, more preferably at least 95%, even more preferably at least 99% sequence identity (as defined herein) with one of the above amino acid sequences; in which
(i) any amino acid substitution is preferably a conservative amino acid substitution (as defined herein); and/or
(ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
(i) any amino acid substitution is preferably a conservative amino acid substitution (as defined herein); and/or
(ii) said amino acid sequence preferably only, contains amino acid substitutions, and no amino acid deletions or insertions, compared to the above amino acid sequence(s); and/or in which:
CDR3 is an amino acid sequence chosen from the group consisting of:

| | |
|---|---|
| ASVKLVYVNPNRYSY | [SEQ ID NO: 66] |
| AAGSAWYGTLYEYDY | [SEQ ID NO: 67] |
| AAGSTWYGTLYEYDY | [SEQ ID NO: 68] |
| MVGPPPRSLDYGLGNHYEYDY | [SEQ ID NO: 69] |
| SSTRTVIYTLPRMYNY | [SEQ ID NO: 70] |
| NSRSSWVIFTIKGQYDR | [SEQ ID NO: 71] |
| RPGMIITTIQATYGF | [SEQ ID NO: 72] |
| GSPYGTELPYTRIEQYAY | [SEQ ID NO: 73] |
| ANEYWVYVNPNRYTY | [SEQ ID NO: 74] |
| RRKSGEWFTIPARYDY | [SEQ ID NO: 75] |
| VYRVGAISEYSGTDYYTDEYDY | [SEQ ID NO: 76] |
| GYQINSGNYNFKDYEYDY | [SEQ ID NO: 77] |
| SYNVYYNNYYYPISRDEYDY | [SEQ ID NO: 78] |
| HRRPFASVFTTTRMYDY | [SEQ ID NO: 79] |

or from the group consisting of amino acid sequences that have at least 80%, preferably at least 90%, more preferably at least 95%, even more preferably at least 99% sequence identity (as defined herein) with one of the above amino acid sequences; in which
(i) any amino acid substitution is preferably a conservative amino acid substitution (as defined herein); and/or
(ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
(i) any amino acid substitution is preferably a conservative amino acid substitution (as defined herein); and/or
(ii) said amino acid sequence preferably only contains amino acid substitutions, and no amino acid deletions or insertions, compared to the above amino acid sequence(s).

2. Polypeptide of less than 15 kDa directed against IGF-IR.

3. Polypeptide according to claim 2 which is able to inhibit IGF-I interaction with IGF-IR.

4. Polypeptide according to claim 2 which binds IGF-IR with a binding affinity of at least 10⁷M⁻¹.

5. Polypeptide according to claim 2 which is selected from a single domain antibody, a domain antibody, a “dAb”, a VH, a VHH or a Nanobody.

6. Nanobody against IGF-IR.

7. Nanobody according to claim 6, which comprises or consists of 4 framework regions (FR1 to FR4 respectively) and 3 complementarity determining regions (CDR1 to CDR3 respectively), in which:
CDR1 is an amino acid sequence chosen from the group consisting of:

| | |
|---|---|
| FNAMG | [SEQ ID NO: 94] |
| INVMA | [SEQ ID NO: 95] |
| NYAMG | [SEQ ID NO: 96] |
| RTAMA | [SEQ ID NO: 97] |

or from the group consisting of amino acid sequences that have at least 80%, preferably at least 90%, more preferably at least 95%, even more preferably at least 99% sequence identity (as defined herein) with one of the above amino acid sequences; in which
(i) any amino acid substitution is preferably a conservative amino acid substitution (as defined herein); and/or
(ii) said amino acid sequence preferably only contains amino acid substitutions, and no amino acid deletions or insertions, compared to the above amino acid sequence(s);
and/or from the group consisting of amino acid sequences that have 2 or only 1 “amino acid difference(s)” (as defined herein) with one of the above amino acid sequences, in which:
(i) any amino acid substitution is preferably a conservative amino acid substitution (as defined herein); and/or
(ii) said amino acid sequence preferably only contains amino acid substitutions, and no amino acid deletions or insertions, compared to the above amino acid sequence(s);
and/or in which:
CDR2 is an amino acid sequence chosen from the group consisting of:

| | |
|---|---|
| VIISGGSTHYVDSVKG | [SEQ ID NO: 98] |
| EITRSGRTNYVDSVKG | [SEQ ID NO: 99] |
| AINWNSRSTYYADSVKG | [SEQ ID NO: 100] |
| TITWNSGTTRYADSVKG | [SEQ ID NO: 101] |

or from the group consisting of amino acid sequences that have at least 80%, preferably at least 90%, more preferably at least 95%, even more preferably at least 99% sequence identity (as defined herein) with one of the above amino acid sequences; in which
(i) any amino acid substitution is preferably a conservative amino acid substitution (as defined herein); and/or
(ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
(i) any amino acid substitution is preferably a conservative amino acid substitution (as defined herein); and/or
(ii) said amino acid sequence preferably only contains amino acid substitutions, and no amino acid deletions or insertions, compared to the above amino acid sequence(s);
and/or in which:
CDR3 is an amino acid sequence chosen from the group consisting of:

| | |
|---|---|
| KKFGDY | [SEQ ID NO: 102] |
| IDGSWREY | [SEQ ID NO: 103] |
| SHDSDYGGTNANLYDY | [SEQ ID NO: 104] |
| TAAAVITPTRGYYNY | [SEQ ID NO: 105] |

or from the group consisting of amino acid sequences that have at least 80%, preferably at least 90%, more preferably at least 95%, even more preferably at least 99% sequence identity (as defined herein) with one of the above amino acid sequences; in which
(i) any amino acid substitution is preferably a conservative amino acid substitution (as defined herein); and/or
(ii) said amino acid sequence preferably only contains amino acid substitutions, and no amino acid deletions or insertions, compared to the above amino acid sequence(s);
and/or from the group consisting of amino acid sequences that have 3, 2 or only 1 “amino acid difference(s)” (as defined herein) with one of the above amino acid sequences, in which:
(i) any amino acid substitution is preferably a conservative amino acid substitution (as defined herein); and/or
(ii) said amino acid sequence preferably only contains amino acid substitutions, and no amino acid deletions or insertions, compared to the above amino acid sequence(s).

8. Method for obtaining a Nanobody according to claim 6 comprising the steps of:
a) providing at least one V_{HH} domain directed against IGF-IR, by a method generally comprising the steps of (i) immunizing a mammal belonging to the Camelidae with IGF-IR or a part or fragment thereof, so as to raise an immune response and/or antibodies (and in particular heavy chain antibodies) against IGF-IR; (ii) obtaining a biological sample from the mammal thus immunized, wherein said sample comprises heavy chain antibody sequences and/or VHH sequences that are directed against IGF-IR; and (iii) obtaining (e.g isolating) heavy chain antibody sequences and/or VHH sequences that are directed against IGF-IR from said biological sample; and/or by a method generally comprising the steps of (i) screening a library comprising heavy chain antibody sequences and/or VHH sequences for heavy chain antibody sequences and/or VHH sequences that are directed against IGF-IR or against at least one part or fragment thereof; and (ii) obtaining (e.g. isolating) heavy chain antibody sequences and/or VHH sequences that are directed against IGF-IR from said library;
b) optionally subjecting the heavy chain antibody sequences and/or VHH sequences against IGF-IR thus obtained to affinity maturation, to mutagenesis (e.g. random mutagenesis or site-directed mutagenesis) and/or any other technique(s) for increasing the affinity and/or specificity of the heavy chain antibody sequences and/or V_{HH} sequences for IGF-IR;
c) determining the sequences of the CDR's of the heavy chain antibody sequences and/or VHH sequences against IGF-IR thus obtained; and
d) providing a Nanobody in which at least one, preferably at least two, and more preferably all three of the CDR's (i.e. CDR1, CDR2 and CDR3, and in particular at least CDR3) has a sequence that has been determined in step c).

9. Polypeptide comprising or essentially consisting of at least one Nanobody according to claim 1, or an EGFR binding part or fragment thereof.

10. Polypeptide comprising at least two binding moieties, wherein each of said at least two binding moieties is directed against a tumor associated antigen or epitope.

11. Polypeptide according to claim 10, wherein each of said at least two binding moieties is directed against a different tumor associated antigen.

12. Polypeptide according to claim 10, wherein each of said at least two binding moieties is directed against a different epitope on the same tumor associated antigen.

13. Polypeptide according to claim 10, wherein at least one of the binding moieties is selected from a VH, a VHH, a domain antibody, a single domain antibody, a “dAb” or a Nanobody.

14. Polypeptide according to claim 10, wherein each of the binding moieties is a Nanobody.

15. Polypeptide according to claim 10, comprising at least one Nanobody against EGFR and at least one Nanobody against an EGFR family member selected from the group consisting of HER2, HER3, and HER4.

16. Polypeptide according to claim 10, comprising at least one Nanobody against EGFR and at least one Nanobody against IGF-IR.

17. Polypeptide comprising at least two Nanobodies wherein binding of one of said at least two Nanobodies modulates the binding by the second of said at least two Nanobodies.

18. Polypeptide according to claim 17 wherein binding by the second of said at least two Nanobodies is enhanced.

19. Polypeptide according to claim 17 wherein binding by the second of said at least two Nanobodies is reduced.

20. Polypeptide according to claim 17 wherein binding by the second of said at least two Nanobodies is inhibited.

21. Nucleic acid encoding a a polypeptide according to claim 9.

22. Host cell expressing polypeptide according to claim 9.

23. Method for preparing a a polypeptide, comprising the steps of:
the expression, in a suitable host cell or host organism or in another suitable expression system of a nucleic acid according to claim 21, optionally followed by isolating and/or purifying the polypeptide thus obtained.

24. A composition comprising at least one polypeptide according to claim 9.

25. Pharmaceutical composition comprising at least one polypeptide according to claim 9, and optionally at least one pharmaceutically acceptable carrier.

26. Diagnostic composition comprising at least one polypeptide according to claim 9, and optionally at least one imaging agent.

27. (canceled)

28. Method for the prevention and/or treatment of diseases or disorders associated with EGFR comprising the step of administering to a subject in need thereof a therapeutically effective amount of a polypeptide according to claim 9.

29. Method according to claim 28, wherein the disease or disorder is associated with or characterised by the over-expression of EGFR.

30. Method according to claim 29 wherein the disease or disorder is cancer or a tumor.

31. Method according to claim 28, wherein the disease or disorder relates to inflammatory processes.

32. Method according to claim 31, wherein the disease or disorder is rheumatoid arthritis, psoriasis, or hypersecretion of mucus in the lung.

33. Method for inhibiting the interaction between EGF and EGFR comprising the step of administering to a subject in need thereof a therapeutically effective amount of a polypeptide according to claim 9.

34. Method to enhance anti-EGFR therapy comprising the step of administering to a subject in need thereof a therapeutically effective amount of a polypeptide according to claim 9.

35. Method for the diagnosis of a disease or disorder associated with EGFR comprising the steps of:
(a) contacting a sample with polypeptide according to claim 9,
(b) detecting binding of said polypeptide to said sample, and
(c) comparing the binding detected in step (b) with a standard, wherein a difference in binding relative to said sample is diagnostic of a disease or disorder associated with EGFR.

36. Method for the imaging of EGFR or IGF IR targets comprising the step of administering a polypeptide according to claim 9.

37. A method for the treatment of cancer, tumors, disorders relating to inflammatory process, rheumatoid arthritis, psoriasis, or hypersecretion of mucus in the lung comprising administering to a subject in need thereof a therapeutically amount of a polypeptide according to claim 9.


SEQUENCE LISTING (143 sequences)

SEQ ID NO: 1 - protein, 26 aa, Artificial
  Recombinant Polypeptide
QVQLQESGGGXVQAGGSLRLSCAASG

SEQ ID NO: 2 - protein, 14 aa, Artificial
  Recombinant Polypeptide
WXRQAPGKXXEXVA

SEQ ID NO: 3 - protein, 32 aa, Artificial
  Recombinant Polypeptide
RFTISRDNAKNTVYLQMNSLXXEDTAVYYCAA

SEQ ID NO: 4 - protein, 11 aa, Artificial
  Recombinant Polypeptide
XXQGTXVTVSS

SEQ ID NO: 5 - protein, 26 aa, Artificial
  Recombinant Polypeptide
QVQLQESGGGLVQAGGSLRLSCAASG

SEQ ID NO: 6 - protein, 14 aa, Artificial
  Recombinant Polypeptide
WFRQAPGKERELVA

SEQ ID NO: 7 - protein, 14 aa, Artificial
  Recombinant Polypeptide
WFRQAPGKEREFVA

SEQ ID NO: 8 - protein, 13 aa, Artificial
  Recombinant Polypeptide
WFRQAPGKEREGA

SEQ ID NO: 9 - protein, 14 aa, Artificial
  Recombinant Polypeptide
WFRQAPGKQRELVA

SEQ ID NO: 10 - protein, 14 aa, Artificial
  Recombinant Polypeptide
WFRQAPGKQREFVA

SEQ ID NO: 11 - protein, 13 aa, Artificial
  Recombinant Polypeptide
WYRQAPGKGLEWA

SEQ ID NO: 12 - protein, 32 aa, Artificial
  Recombinant Polypeptide
RFTISRDNAKNTVYLQMNSLKPEDTAVYYCAA

SEQ ID NO: 13 - protein, 11 aa, Artificial
  Recombinant Polypeptide
WGQGTQVTVSS

SEQ ID NO: 14 - protein, 11 aa, Artificial
  Recombinant Polypeptide
WGQGTLVTVSS

SEQ ID NO: 15 - protein, 5 aa, Artificial
  Recombinant Polypeptide
SFGMS

SEQ ID NO: 16 - protein, 5 aa, Artificial
  Recombinant Polypeptide
LNLMG

SEQ ID NO: 17 - protein, 5 aa, Artificial
  Recombinant Polypeptide
INLLG

SEQ ID NO: 18 - protein, 5 aa, Artificial
  Recombinant Polypeptide
NYWMY

SEQ ID NO: 19 - protein, 17 aa, Artificial
  Recombinant Polypeptide
SISGSGSDTLYADSVKG

SEQ ID NO: 20 - protein, 16 aa, Artificial
  Recombinant Polypeptide
TITVGDSTNYADSVKG

SEQ ID NO: 21 - protein, 16 aa, Artificial
  Recombinant Polypeptide
TITVGDSTSYADSVKG

SEQ ID NO: 22 - protein, 17 aa, Artificial
  Recombinant Polypeptide
SINGRGDDTRYADSVKG

SEQ ID NO: 23 - protein, 17 aa, Artificial
  Recombinant Polypeptide
AISADSSTKNYADSVKG

SEQ ID NO: 24 - protein, 17 aa, Artificial
  Recombinant Polypeptide
AISADSSDKRYADSVKG

SEQ ID NO: 25 - protein, 17 aa, Artificial
  Recombinant Polypeptide
RISTGGGYSYYADSVKG

SEQ ID NO: 26 - protein, 13 aa, Artificial
  Recombinant Polypeptide
DREAQVDTLDFDY

SEQ ID NO: 27 - protein, 6 aa, Artificial
  Recombinant Polypeptide
GGSLSR

SEQ ID NO: 28 - protein, 8 aa, Artificial
  Recombinant Polypeptide
RRTWHSEL

SEQ ID NO: 29 - protein, 7 aa, Artificial
  Recombinant Polypeptide
GRSVSRS

SEQ ID NO: 30 - protein, 5 aa, Artificial
  Recombinant Polypeptide
GRGSP

SEQ ID NO: 31 - protein, 17 aa, Artificial
  Recombinant Polypeptide
AAAEQKLISEEDLNGAA

SEQ ID NO: 32 - protein, 115 aa, Artificial [sequence omitted]

SEQ ID NO: 33 - protein, 115 aa, Artificial [sequence omitted]

SEQ ID NO: 34 - protein, 117 aa, Artificial
  Recombinant Polypeptide
AVQLVESGGGLVQGGGSLRLACAASERIFDLNLMGWYRQGPGNERELVATCITVGDSTNYADSVKGRFTISMDYTKQTVYLHMNSLRPEDTGLYYCKIRRTWHSELWGQGTQVTVSS

SEQ ID NO: 35 - protein, 125 aa, Artificial
  Recombinant Polypeptide
EVQLQASGGGLVQAGGSLRLSCSASVRTFSIYAMGWFRQAPGKEREFVAGINRSGDVTKYADFVKGRFSISRDNAKNMVYLQMNSLKPEDTALYYCAATWAYDTVGALTSGYNFWGQGTQVTVSS

SEQ ID NO: 36 - protein, 122 aa, Artificial
  Recombinant Polypeptide
EVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSS

SEQ ID NO: 37 - protein, 30 aa, Artificial
  Recombinant Polypeptide
GGGGSGGGGSGGGGSGGGGSGGGGSGGGGS

SEQ ID NO: 38 - protein, 15 aa, Artificial
  Recombinant Polypeptide
GGGGSGGGGSGGGGS

SEQ ID NO: 39 - protein, 9 aa, Artificial
  Recombinant Polypeptide
GGGGSGGGS

SEQ ID NO: 40 - protein, 7 aa, Artificial
  Recombinant Polypeptide
SGGSGGS

SEQ ID NO: 41 - protein, 12 aa, Artificial
  Recombinant Polypeptide
EPKTPKPQPAAA

SEQ ID NO: 42 - protein, 5 aa, Artificial
  Recombinant Polypeptide
TYTMA

SEQ ID NO: 43 - protein, 5 aa, Artificial
  Recombinant Polypeptide
SYGMG

SEQ ID NO: 44 - protein, 5 aa, Artificial
  Recombinant Polypeptide
GFAMG

SEQ ID NO: 45 - protein, 5 aa, Artificial
  Recombinant Polypeptide
SNNMG

SEQ ID NO: 46 - protein, 5 aa, Artificial
  Recombinant Polypeptide
GDVMG

SEQ ID NO: 47 - protein, 5 aa, Artificial
  Recombinant Polypeptide
SYVVG

SEQ ID NO: 48 - protein, 5 aa, Artificial
  Recombinant Polypeptide
DYNMA

SEQ ID NO: 49 - protein, 5 aa, Artificial
  Recombinant Polypeptide
TYTMA

SEQ ID NO: 50 - protein, 5 aa, Artificial
  Recombinant Polypeptide
NNAMA

SEQ ID NO: 51 - protein, 5 aa, Artificial
  Recombinant Polypeptide
SYVMG

SEQ ID NO: 52 - protein, 5 aa, Artificial
  Recombinant Polypeptide
SYAMG

SEQ ID NO: 53 - protein, 1 aa, Artificial
  Synthetic Amino Acid
A

SEQ ID NO: 54 - protein, 17 aa, Artificial
  Recombinant Polypeptide
GISRSDGGTYDADSVKG

SEQ ID NO: 55 - protein, 17 aa, Artificial
  Recombinant Polypeptide
GISWRGDSTGYADSVKG

SEQ ID NO: 56 - protein, 17 aa, Artificial
  Recombinant Polypeptide
AISWSGGSLLYVDSVKG

SEQ ID NO: 57 - protein, 17 aa, Artificial
  Recombinant Polypeptide
AIGWGGLETHYSDSVKG

SEQ ID NO: 58 - protein, 17 aa, Artificial
  Recombinant Polypeptide
GFSRSTSTTHYADSVKG

SEQ ID NO: 59 - protein, 17 aa, Artificial
  Recombinant Polypeptide
GIAWGDGITYYADSVKG

SEQ ID NO: 60 - protein, 17 aa, Artificial
  Recombinant Polypeptide
HISWLGGRTYYRDSVKG

SEQ ID NO: 61 - protein, 17 aa, Artificial
  Recombinant Polypeptide
GFSGSGGATYYAHSVEG

SEQ ID NO: 62 - protein, 17 aa, Artificial
  Recombinant Polypeptide
AISWRGGSTYYADSVKG

SEQ ID NO: 63 - protein, 17 aa, Artificial
  Recombinant Polypeptide
AINWSSGSTYYADSVKG

SEQ ID NO: 64 - protein, 17 aa, Artificial
  Recombinant Polypeptide
TIAWDSGSTYYADSVKG

SEQ ID NO: 65 - protein, 17 aa, Artificial
  Recombinant Polypeptide
GLSWSADSTYYADSVKG

SEQ ID NO: 66 - protein, 15 aa, Artificial
  Recombinant Polypeptide
ASVKLVYVNPNRYSY

SEQ ID NO: 67 - protein, 15 aa, Artificial
  Recombinant Polypeptide
AAGSAWYGTLYEYDY

SEQ ID NO: 68 - protein, 15 aa, Artificial
  Recombinant Polypeptide
AAGSTWYGTLYEYDY

SEQ ID NO: 69 - protein, 21 aa, Artificial
  Recombinant Polypeptide
MVGPPPRSLDYGLGNHYEYDY

SEQ ID NO: 70 - protein, 16 aa, Artificial
  Recombinant Polypeptide
SSTRTVIYTLPRMYNY

SEQ ID NO: 71 - protein, 17 aa, Artificial
  Recombinant Polypeptide
NSRSSWVIFTIKGQYDR

SEQ ID NO: 72 - protein, 15 aa, Artificial
  Recombinant Polypeptide
RPGMIITTIQATYGF

SEQ ID NO: 73 - protein, 18 aa, Artificial
  Recombinant Polypeptide
GSPYGTELPYTRIEQYAY

SEQ ID NO: 74 - protein, 15 aa, Artificial
  Recombinant Polypeptide
ANEYWVYVNPNRYTY

SEQ ID NO: 75 - protein, 17 aa, Artificial
  Recombinant Polypeptide
RRKSGEVVFTIPARYDY

SEQ ID NO: 76 - protein, 22 aa, Artificial
  Recombinant Polypeptide
VYRVGAISEYSGTDYYTDEYDY

SEQ ID NO: 77 - protein, 18 aa, Artificial
  Recombinant Polypeptide
GYQINSGNYNFKDYEYDY

SEQ ID NO: 78 - protein, 20 aa, Artificial
  Recombinant Polypeptide
SYNVYYNNYYYPISRDEYDY

SEQ ID NO: 79 - protein, 17 aa, Artificial
  Recombinant Polypeptide
HRRPFASVFTTTRMYDY

SEQ ID NO: 80 - protein, 124 aa, Artificial
  Recombinant Polypeptide
EVQLVESGGGLVQAGGSLRLSCAASGRTFSTYTMAWFRQAPGKEREFVQGISRSDGGTYDADSVKGRFTISRDNAKNTVYLQMNSLKPEDTAVYFCAAASVKLVYVNPNRYSYWGQGTQVTVSS

SEQ ID NO: 81 - protein, 124 aa, Artificial
  Recombinant Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 82 - protein, 124 aa, Artificial
  Recombinant Polypeptide
AVQLVESGGGSVQAGGSLRLTCAASGRTSRSYGMGWFRQAPGKEREFVSGISWRGDSTGYADSVKGRFTISRDNAKNTVDLQMNSLKPEDTAIYYCAAAAGSTWYGTLYEYDYWGQGTQVTVSS

SEQ ID NO: 83 - protein, 130 aa, Artificial
  Recombinant Polypeptide
EVQLVESGGGLVQAGGSLRLSCAASGRTFSGFAMGWFRQAPGKEREFVAAISWSGGSLLYVDSVKGRFTISRDNAKNTVHLQMNSLKPGDTAVYYCAAMVGPPPRSLDYGLGNHYEYDYWGQGTQVTVSS

SEQ ID NO: 84 - protein, 125 aa, Artificial
  Recombinant Polypeptide
EVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSS

SEQ ID NO: 85 - protein, 126 aa, Artificial
  Recombinant Polypeptide
EVQLVESGGGLVQAGGSLRLSCAVSGRTFSGDVMGWFRQAPGKEREFVAGFSRSTSTTHYADSVKGRFTISGDNAKNTVYLQMNSLKPEDTAVYYCAANSRSSWVIFTIKGQYDRWGQGTQVTVSS

SEQ ID NO: 86 - protein, 124 aa, Artificial
  Recombinant Polypeptide
EVQLVESGGGLVQAGGSLRVSCVASIRSFSSYVVGWFRQAPGKDREFVAGIAWGDGITYYADSVKGRFTISRDHAKNTAYLQMNSLRPEDTAVYYCAVRPGMIITTIQATYGFWGQGTQVTVSS

SEQ ID NO: 87 - protein, 127 aa, Artificial
  Recombinant Polypeptide
EVQLVESGGGLVQAGASLRLSCAASGRYFSDYNMAWFRQAPGKEREFVAHISWLGGRTYYRDSVKGRFTISRDNAKDTVYLQMNSLKPEDTAVYSCAAGSPYGTELPYTRIEQYAYWGQGTQVTVSS

SEQ ID NO: 88 - protein, 124 aa, Artificial
  Recombinant Polypeptide
AVQLVESGGGLVQAGGSLRLSCAASGRTFSTYTMAWFRQAPGKEREFVQGISRSDGGTYDADSVKGRFTISRDNAKNTVYLQMNSLKPEDTAVYFCAAANEYWVYVNPNRYTYWGRGTQVTVSS

SEQ ID NO: 89 - protein, 126 aa, Artificial
  Recombinant Polypeptide
EVQLVESGGGLVQAGGSLRLSCTASGRTFENNAMAWFRQAAGKEREFVAGFSGSGGATYYAHSVEGRFTISRDNAKNTVDLQMNSLSLEDTAVYLCAARRKSGEVVFTIPARYDYWGQGTQVTVSS

SEQ ID NO: 90 - protein, 131 aa, Artificial
  Recombinant Polypeptide
QVKLEESGGGLVQAGGSLRLSCAASGRAFSSYVMGWFRQAPGKEREIVGAISWRGGSTYYADSVKGRFTISRDNAKNTVYLQMNSLKPEDTAVYYCAAVYRVGAISEYSGTDYYTDEYDYWGQGTQVTVSS

SEQ ID NO: 91 - protein, 127 aa, Artificial
  Recombinant Polypeptide
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 92 - protein, 129 aa, Artificial
  Recombinant Polypeptide
EVQLVESGGGLVQAGGSLRLSCAASGRTFSSYVMGWFRQATGKEREFVATIAWDSGSTYYADSVKGRFTISRDNAKNTVHLQMNSLKPEDTAVYYCAASYNVYYNNYYYPISRDEYDYWGQGTQVTVSS

SEQ ID NO: 93 - protein, 122 aa, Artificial
  Recombinant Polypeptide
EVQLVESGGGLVQAGGSLRLSCAASGRGAWAWFRQAPGKEREFVGGLSWSADSTYYADSVKGRFTISRDNAKNTVFLQMTNLKADDTAVYYCAAHRRPFASVFTTTRMYDYWGPGTQVTVSS

SEQ ID NO: 94 - protein, 5 aa, Artificial
  Recombinant Polypeptide
FNAMG

SEQ ID NO: 95 - protein, 5 aa, Artificial
  Recombinant Polypeptide
INVMA

SEQ ID NO: 96 - protein, 5 aa, Artificial
  Recombinant Polypeptide
NYAMG

SEQ ID NO: 97 - protein, 5 aa, Artificial
  Recombinant Polypeptide
RTAMA

SEQ ID NO: 98 - protein, 16 aa, Artificial
  Recombinant Polypeptide
VIISGGSTHYVDSVKG

SEQ ID NO: 99 - protein, 16 aa, Artificial
  Recombinant Polypeptide
EITRSGRTNYVDSVKG

SEQ ID NO: 100 - protein, 17 aa, Artificial
  Recombinant Polypeptide
AINWNSRSTYYADSVKG

SEQ ID NO: 101 - protein, 17 aa, Artificial
  Recombinant Polypeptide
TITWNSGTTRYADSVKG

SEQ ID NO: 102 - protein, 6 aa, Artificial
  Recombinant Polypeptide
KKFGDY

SEQ ID NO: 103 - protein, 8 aa, Artificial
  Recombinant Polypeptide
IDGSWREY

SEQ ID NO: 104 - protein, 16 aa, Artificial
  Recombinant Polypeptide
SHDSDYGGTNANLYDY

SEQ ID NO: 105 - protein, 15 aa, Artificial
  Recombinant Polypeptide
TAAAVITPTRGYYNY

SEQ ID NO: 106 - protein, 114 aa, Artificial
  Recombinant Polypeptide
EVQLVESGGGLVQPGGSLRLSCAASGSIFTFNAMGWYRQAPGKQRELVAVIISGGSTHYVDSVKGRFTISRDNAKKMVYLQMNSLKPEDTAVYYCNVKKFGDYWGQGTQVTVSS

SEQ ID NO: 107 - protein, 116 aa, Artificial
  Recombinant Polypeptide
DVQLVESGGGLVQAGGSLRLSCAASESISTINVMAWYRQAPGKQRELVAEITRSGRTNYVDSVKGRFTISRDNAKNTMYLQMNSLNLEDTAVYYCRTIDGSWREYWGQGTQVTVSS

SEQ ID NO: 108 - protein, 125 aa, Artificial
  Recombinant Polypeptide
QVKLEESGGGLVQPGGSLRLSCVASGRTFSNYAMGWFRQAPGQEREFVAAINWNSRSTYYADSVKGRFTISRLNARNTVYLQMNRLKPEDTAVYDCAASHDSDYGGTNANLYDYWGQGTQVTVSS

SEQ ID NO: 109 - protein, 124 aa, Artificial
  Recombinant Polypeptide
QVKLEESGGGLVQAGGSLRLSCVASGRTFSRTAMAWFRQAPGKEREFVATITWNSGTTRYADSVKGRFFISKDSAKNTIYLEMNSLEPEDTAVYYCAATAAAVITPTRGYYNYWGQGTQVTVSS

SEQ ID NO: 110 - protein, 243 aa, Artificial
  Recombinant Polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSAAAEVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSS

SEQ ID NO: 111 - protein, 245 aa, Artificial
  Recombinant Polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSEVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSS

SEQ ID NO: 112 - protein, 249 aa, Artificial
  Recombinant Polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTQVTVSSGGGGSGGGSEVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSS

SEQ ID NO: 113 - protein, 265 aa, Artificial
  Recombinant Polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTQVTVSSGGGGSGGGGSGGGGSGGGGSGGGGSEVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSS

SEQ ID NO: 114 - protein, 242 aa, Artificial
  Recombinant Polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSAAAEVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 115 - protein, 244 aa, Artificial
  Recombinant Polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSEVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 116 - protein, 248 aa, Artificial
  Recombinant Polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTQVTVSSGGGGSGGGSEVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 117 - protein, 264 aa, Artificial
  Recombinant Polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTQVTVSSGGGGSGGGGSGGGGSGGGGSGGGGSEVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 118 - protein, 249 aa, Artificial
  Recombinant Polypeptide
EVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 119 - protein, 265 aa, Artificial
  Recombinant Polypeptide
EVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 120 - protein, 248 aa, Artificial
  Recombinant Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 121 - protein, 264 aa, Artificial
  Recombinant Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 122 - protein, 257 aa, Artificial
  Recombinant Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSEVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 123 - protein, 273 aa, Artificial
  Recombinant Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSGGGGSGGGGSEVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 124 - protein, 383 aa, Artificial
  Recombinant Polypeptide
EVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSSGGGGSGGGSEVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 125 - protein, 377 aa, Artificial
  Recombinant Polypeptide
EVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSSGGGGSGGGSEVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSSAAAEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 126 - protein, 381 aa, Artificial
  Recombinant Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSEVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 127 - protein, 381 aa, Artificial
  Recombinant Polypeptide
AVQLVESGGGSVQAGGSLRLTCAASGRTSRSYGMGWFRQAPGKEREFVSGISWRGDSTGYADSVKGRFTISRDNAKNTVDLQMNSLKPEDTAIYYCAAAAGSTWYGTLYEYDYWGQGTQVTVSSGGGGSGGGSEVQLVESGGGSVQAGGSLRLTCAASGRTSRSYGMGWFRQAPGKEREFVSGISWRGDSTGYADSVKGRFTISRDNAKNTVDLQMNSLKPEDTAIYYCAAAAGSTWYGTLYEYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 128 - protein, 371 aa, Artificial
  Recombinant Polypeptide
EVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSSGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSAAAEVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSS

SEQ ID NO: 129 - protein, 383 aa, Artificial
  Recombinant Polypeptide
EVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTQVTVSSGGGGSGGGSEVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSS

SEQ ID NO: 130 - protein, 381 aa, Artificial
  Recombinant Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTQVTVSSGGGGSGGGSEVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 131 - protein, 382 aa, Artificial
  Recombinant Polypeptide
AVQLVESGGGSVQAGGSLRLLTCAASGRTSRSYGMGWFRQAPGKEREFVSGISWRGDSTGYADSVKGRFTISRDNAKNTVDLQMNSLKPEDTAIYYCAAAAGSTWYGTLYEYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTQVTVSSGGGGSGGGSEVQLVESGGGSVQAGGSLRLTCAASGRTSRSYGMGWFRQAPGKEREFVSGISWRGDSTGYADSVKGRFTISRDNAKNTVDLQMNSLKPEDTAIYYCAAAAGSTWYGTLYEYDYWGQGTQVTVSS

SEQ ID NO: 132 - protein, 383 aa, Artificial
  Recombinant Polypeptide
EVQLVRSGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSSGGGGSGGGSEVQLVESGGGLVQAGGSLRLSCAASDRTFSSNNMGWFRQAPGKEREFVAAIGWGGLETHYSDSVKGRFTISRDNAKNTVYLQMNSLKPEDTARYYCAVSSTRTVIYTLPRMYNYWGQGTQVTVSS

SEQ ID NO: 133 - protein, 381 aa, Artificial
  Recombinant Polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSEVQLVESGGGSVQTGGSLRLTCAASGRTSRSYGMGWFRQAPGKEREFVSGISWRGDTSGYADSVKGRFTISRDNAKNTVDLQMNSLKPEDTAIYYCAAAAGSAWYGTLYEYDYWGQGTQVTVSS

SEQ ID NO: 134 - protein, 257 aa, Artificial
  Recombinant Polypeptide
QVKLEESGGGLVQAGGSLRLSCVASGRTFSRTAMAWFRQAPGKEREFVATITWNSGTTRYADSVKGRFFISKDSAKNTIYLEMNSLEPEDTAVYYCAATAAAVITPTRGYYNYWGQGTQVTVSSGGGGSGGGSQVKLEESGGGLVQAGGSLRLSCVASGRTFSRTAMAWFRQAPGKEREFVATITWNSGTTRYADSVKGRFFISKDSAKNTIYLEMNSLEPEDTAVYYCAATAAAVITPTRGYYNYWGQGTQVTVSS

SEQ ID NO: 135 - protein, 273 aa, Artificial
  Recombinant Polypeptide
QVKLEESGGGLVQAGGSLRLSCVASGRTFSRTAMAWFRQAPGKEREFVATITWNSGTTRYADSVKGRFFISKDSAKNTIYLEMNSLEPEDTAVYYCAATAAAVITPTRGYYNYWGQGTQVTVSSGGGGSGGGGSGGGGSGGGGSGGGGSQVKLEESGGGLVQAGGSLRLSCVASGRTFSRTAMAWFRQAPGKEREFVATITWNSGTTRYADSVKGRFFISKDSAKNTIYLEMNSLEPEDTAVYYCAATAAAVITPTRGYYNYWGQGTQVTVSS

SEQ ID NO: 136 - protein, 257 aa, Artificial
  Recombinant Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQVKLEESGGGLVQAGGSLRLSCVASGRTFSRTAMAWFRQAPGKEREFVATITWNSGTTRYADSVKGRFFISKDSAKNTIYLEMNSLEPEDTAVYYCAATAAAVITPTRGYYNYWGQGTQVTVSS

SEQ ID NO: 137 - protein, 273 aa, Artificial
  Recombinant Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSGGGGSGGGGSQVKLEESGGGLVQAGGSLRLSCVASGRTFSRTAMAWFRQAPGKEREFVATITWNSGTTRYADSVKGRFFISKDSAKNTIYLEMNSLEPEDTAVYYCAATAAAVITPTRGYYNYWGQGTQVTVSS

SEQ ID NO: 138 - protein, 381 aa, Artificial
  Recombinant Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQVKLEESGGGLVQAGGSLRLSCVASGRTFSRTAMAWFRQAPGKEREFVATITWNSGTTRYADSVKGRFFISKDSAKNTIYLEMNSLEPEDTAVYYCAATAAAVITPTRGYYNYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 139 - protein, 381 aa, Artificial
  Recombinant Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVKLEESGGGLVQAGGSLRLSCVASGRTFSRTAMAWFRQAPGKEREFVATITWNSGTTRYADSVKGRFFISKDSAKNTIYLEMNSLEPEDTAVYYCAATAAAVITPTRGYYNYWGQGTQVTVSS

SEQ ID NO: 140 - protein, 397 aa, Artificial
  Recombinant Polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSGGGGSGGGGSQVKLEESGGGLVQAGGSLRLSCVASGRTFSRTAMAWFRQAPGKEREFVATITWNSGTTRYADSVKGRFFISKDSAKNTIYLEMNSLEPEDTAVYYCAATAAAVITPTRGYYNYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 141 - protein, 257 aa, Artificial; identical to SEQ ID NO: 136
  Recombinant Polypeptide

SEQ ID NO: 142 - protein, 381 aa, Artificial; identical to SEQ ID NO: 138
  Recombinant Polypeptide

SEQ ID NO: 143 - protein, 381 aa, Artificial; identical to SEQ ID NO: 139
  Recombinant Polypeptide